(19) United States
(12) Patent Application Publication
(10) Pub. No.: US 2025/0269368 A1
(43) Pub. Date: Aug. 28, 2025

(54) METHOD AND SYSTEM COMPRISING A CARTRIDGE FOR SEQUENCING TARGET POLYNUCLEOTIDES

(71) Applicant: DNAE Group Holdings Limited, London (GB)

(72) Inventors: Sam Reed, London (GB); Norman C. Nelson, San Diego, CA (US); David Wooldridge, London (GB); Henry Fatoyinbo, London (GB); Alessandro Marturano, London (GB); Dan Morley, London (GB); Graham Worsley, London (GB); Douglas Standridge, Albuquerque, NM (US); Kamil Andrzej Liqinski, London (GB); Bradley Brown, San Marcos, CA (US); Alex Flamm, Baltimore, MD (US); Ben Lane, Baltimore, MD (US); Melanie Springer, Baltimore, MD (US)

(21) Appl. No.: 18/580,983

(22) PCT Filed: Jul. 21, 2022
(86) PCT No.: PCT/GB2022/051909

§ 371 Date: Jan. 19, 2024

Related U.S. Application Data
(60) Provisional application No. 63/224,116, filed on Jul. 21, 2021.

(30) Foreign Application Priority Data
Jul. 21, 2021 (GB) 2110479.9
Jul. 21, 2021 (GB) 2110485.6

(51) Int. Cl. B01L 3/00; C12Q 1/6874

(52) U.S. Cl. CPC B01L 3/5025; C12Q 1/6874; B01L 2200/028; B01L 2200/16; B01L 2300/0645; B01L 2400/0487

(57) ABSTRACT

Described are methods of identifying a target polynucleotide sequence from a sample using a cartridge-based system and methods of forming an array of amplified nucleic acid molecules on a solid support.

### CROSS-REFERENCE TO RELATED APPLICATIONS

This Application claims priority to and the benefit of U.S. Provisional Application No. 63/224,116, filed Jul. 21, 2021, the content of which is incorporated herein in its entirety.

### TECHNICAL FIELD

The present disclosure is directed generally to sample processing and nucleic acid sequencing.

### BACKGROUND

Nucleic acid sequencing technologies have undergone tremendous advances in recent years. A significant contributor to this advancement has been the advent of next generation sequencing, or NGS (see, for example, The sequence of sequencers: The history of sequencing DNA (2016) Heather and Chain; Genomics 107: 1-8). As an example, as part of the Human Genome Project the first human genome was sequenced in about 13 years at a cost of at least several hundred million US dollars. Today, under certain circumstances it is possible to sequence a human genome in a matter of days at a cost of 1000 US dollars (see https://www.genome.gov/about-genomics/fact-sheets/Sequencing-Human-Genome-cost). Nucleic acid sequencing is now used extensively in many fields and its value is well-appreciated.

In many applications, however, sequencing workflows—the process required to prepare a target polynucleotide contained in a sample for sequencing, conduct sequencing and analyze the resulting data—are tedious, time consuming, complex and often costly. Many of the steps are still performed manually and require highly skilled personnel. Even if specific processes in a workflow are automated, multiple instruments and ancillary components are required and skilled human intervention is required at various points to perform the entire workflow. Furthermore, the time from sample to result is several hours to several days or longer. In addition, the maximum allowable amount of sample input is low, which represents a further current limitation. Thus, the power and value of sequencing—including next generation sequencing—can be greatly diminished in practical use.

### SUMMARY

Systems and methods of the invention recognize an existing need for a sequencing workflow that is fully automated (sample-to-report), requiring no user intervention once the run is initiated, rapid (sample to actionable result in a few hours), sensitive, accurate, cost effective and amenable to use at the point-of-need. As disclosed herein, previously isolated steps for sample preparation, library preparation, and sequencing can be automatically performed in a single cartridge, within a single benchtop instrument.

Disclosed are embodiments useful for the rapid analysis of target polynucleotides, including determining the nucleotide sequence thereof, from a wide range of input sample types and amounts using a fully automated system comprising a cartridge, instrument and operational and analytical software with no human intervention required after the run is started (e.g., sample-to-report). Further embodiments disclose use of a semi-conductor chip for detection as well as methods for the generation of nucleic acid clusters on a surface, including the surface of a semi-conductor chip. The embodiments are useful for a wide variety of applications including clinical diagnostics, epidemiology and surveillance, oncology, genetic and genomic analysis including metagenomics, hospital infection control, basic and applied research, food testing, forensics, environmental testing, biothreat detection, animal health, agricultural testing and the like.

In various embodiments, any of the reagents (e.g., primers, enzymes, and buffers) and/or systems described herein may be combined and provided in one or more kits. Accordingly, kits comprising any of the systems, components thereof (e.g., cartridges or instruments), and/or reagents described herein, and operable to perform any of the methods described herein are contemplated.

Aspects of the invention can include methods for analyzing a target in a sample including steps such as introducing a sample to a cartridge and introducing the cartridge to an instrument. The instrument can then be operable to manipulate the sample within the cartridge to automatically isolate a target nucleic acid from the sample; amplify the isolated target nucleic acid; and sequence the amplified target nucleic acid using next generation sequencing. The sample may remain within the cartridge throughout the isolating, amplifying, and sequencing steps. Furthermore, the entire process may be performed within a single instrument without user intervention. In certain embodiments, the isolating, amplifying, and sequencing steps may be performed within 8 or less hours after introducing the sample to the cartridge.

Systems and methods of the invention can provide excellent sensitivity with, in certain embodiments, the isolated, amplified, and sequenced target nucleic comprising fungal nucleic acid present in the sample at levels as low as 3 copies. In certain embodiments, the target nucleic acid may comprise bacterial nucleic acid present in the sample at levels as low as 3 copies or viral nucleic acid present in the sample at levels as low as a single copy.

Another advantage of the systems and methods described herein is the relatively small footprint of the cartridges, providing more manageable shipping, storage, and handling while allowing for a smaller footprint of the analyzer instrument taking up less expensive laboratory space. As described herein, exterior volumes of the cartridge or instrument may refer to the total 3-dimensional volume the cartridge or instrument occupies (e.g., height×length×width for a rectangular prism-shaped cartridge). In certain embodiments, the cartridge may have an exterior volume of about 3 liters or less. The cartridge can have an exterior volume of about 2.5 liters or less. In some embodiments, the cartridge may have an exterior volume of about 2.1 liters or less. Similarly, in addition to a small relative exterior volume, cartridges of the invention may have a relatively small longest linear dimension (e.g., longest of height, length, or width for a rectangular prism-shaped cartridge) to allow for ease of handling and a compact instrument. In certain embodiments, the cartridge may have a longest linear dimension of about 200 mm or less. In some embodiments, the cartridge can have a longest linear dimension of about 160 mm or less.

Systems and methods of the invention can through, for example, changes in reagents and/or the timing, order, and duration of various steps in a workflow, accommodate a variety of assays as well as different sample types and volumes. In various embodiments, a single cartridge may be capable of performing multiple different assays and a single instrument may accept different cartridges if necessary to perform different assays. The systems and methods of the invention contemplate, should different cartridge internals be required to perform different assays (e.g., a different sample preparation unit to process a biological sample vs. an environmental sample) that the overall size and shape of the different cartridges be substantially the same to permit interoperability with the single instrument. In various embodiments, the sample may be selected from the group consisting of a biological sample, a clinical sample, an environmental sample, and a food sample. A particular advantage of the present invention is the ability to receive a raw or minimally processed sample and automatically proceed through the required workflow steps of the selected assay to provide a sequencing result without any user intervention and without removing the sample from the single cartridge or the single instrument. Accordingly, in certain embodiments, the sample may be a biological sample obtained from a subject and untreated prior to introduction to the cartridge.

Sample processing, which may include isolating a target nucleic acid in a sample, may include digesting proteins in the sample. The proteins may be digested using proteinase K. Isolating may comprise lysing an organism in the sample (e.g., a bacterial, fungal, or host cell such as a human cell) to release the target nucleic acid. Lysing may comprise mechanical lysis. In various embodiments, mechanical lysis may include flowing the sample into a lysis chamber within the cartridge and rotating a paddle within the lysis chamber. Mechanical lysis may further comprise adding zirconium beads to the lysis chamber before rotating the paddle within the lysis chamber. Isolating may comprise denaturing the target nucleic acid. Denaturing may include thermal denaturing. Isolating may include capturing the target nucleic acid by annealing target capture oligonucleotides to the target nucleic acid to form a complex; binding the complex on a solid support; and removing unbound material from the solid support. In certain embodiments, removing unbound material can comprise washing the solid support bound complex with a wash reagent.

In some embodiments, an amplifying step may be performed on the solid support-bound target nucleic acid. That amplification step may be performed within the sample processing unit of the cartridge and may substitute for or be in addition to amplification steps performed as part of library preparation in the library preparation unit of the cartridge (e.g., incorporating any required barcodes, tags, or adapters). In certain embodiments, methods may include eluting the target nucleic acid from the washed solid support to prepare the isolated target nucleic acid. The amplifying step for library preparation may be performed directly on the eluted target nucleic acid without intervening steps.

In certain embodiments, the isolating step may automatically isolate the target nucleic acid from a sample having a volume between about 1 mL and about 25 mL. The isolating step may comprise only one purification step. The isolated nucleic acid can be amplified without quantification.

In some embodiments, the amplifying step can comprise performing a first amplification of the isolated target nucleic acid using a first primer set to produce a first amplification product; diluting the first amplification product and aliquoting into a plurality of aliquots; performing a second amplification of the target nucleic acid in the plurality of aliquots using a second primer set to produce a plurality of second amplification products; and pooling the second amplification products. In various embodiments, one or more primers in the first primer set can be identical to one or more primers in the second primer set. The amplifying step can further comprise purifying the pooled second amplification products to produce the amplified target nucleic acid. One or more of the first and second amplifications may comprise PCR amplification. The plurality of aliquots can include at least 10 separate aliquots. The first PCR amplification and second PCR amplification may be performed without quantification. In some embodiments, the second primer set may be nested relative to the first primer set. The amplifying step can include performing copy control on the amplified target nucleic acid before the sequencing step. The amplifying step may comprise only one purification step.

In certain embodiments, the amplified target nucleic acid may be sequenced without quantification. The sequencing step may comprise immobilizing the amplified target nucleic acid above a semiconductor surface within the cartridge comprising an ion-sensitive field-effector transistor (ISFET) sensor. All products of the amplifying step may be flowed over the semiconductor surface without intervening steps. In some embodiments, the amplified target nucleic acid may be immobilized by a capture oligomer bound above the ISFET sensor wherein said capture oligomer hybridizes to a portion of the target nucleic acid. The surface can comprise an array of ISFET sensors each with a well positioned above it. At least one of the wells may be positioned above a plurality of ISFET sensors in the array of ISFET sensors. One or more of the wells may comprise a surface-bound forward primer that hybridizes to a portion of the target nucleic acid and a surface-bound reverse primer that hybridizes to a portion of the target nucleic acid, and wherein the sequencing step comprises paired-end sequencing. In certain embodiments, one or more of the wells or an interstitial space between one or more of the wells can comprise a plurality of bound inert oligomers that do not hybridize to the target nucleic acid. The amplified target nucleic acid may be immobilized by a universal capture oligomer bound above the ISFET sensor wherein said universal capture oligomer hybridizes to a universal binding site. In some embodiments, the amplifying step can comprise amplifying the isolated target nucleic acid using a primer comprising the universal binding site. In certain embodiments, the amplifying step may comprise ligating an adapter onto the isolated target nucleic acid, said adapter comprising the universal binding site. The sequencing step may comprise clonal amplification of the immobilized target nucleic acid and the clonal amplification can comprise recombinase polymerase amplification, rolling circle amplification, bridge PCR, strand displacement amplification, or loop-mediated isothermal amplification.

In certain aspects, systems of the invention may comprise a sample cartridge comprising: a sample input; a sample preparation unit operable to receive sample from the sample input and isolate a target nucleic acid from the sample; a library preparation unit operable to receive the isolated target nucleic acid from the sample preparation unit and amplify the isolated target nucleic acid; and a sequencing unit operable to receive the amplified target nucleic acid from the library preparation unit and sequence the amplified target nucleic acid. Systems may further include an instrument comprising a cartridge interface comprising physical and electronic connections through which the instrument is operable to drive movement of the sample and reagents within the cartridge and to communicate with the sequencing units. One or more reagents required for isolating the target nucleic acid, amplifying the isolated target nucleic acid, and sequencing the amplified nucleic acid may be dried reagents, the instrument operable to reconstitute the one or more reagents.

Systems of the invention may further comprise one or more reagent cartridges containing one or more reagents required for isolating the target nucleic acid, amplifying the isolated target nucleic acid, and sequencing the amplified nucleic acid. The instrument may be operable to transfer reagents from the one or more reagent cartridges to the sample cartridge. By using a reagent cartridge, the contamination and logistical issues with maintaining on-board reagent wells within an instrument can be avoided. Additionally, reagent cartridges can be sealed after production, thereby avoiding possible contamination or user error in manual refills of reagent wells. Reagent cartridges may be assay-specific and may include the reagents necessary for 1 complete assay or multiple assays. A user may insert the appropriate reagent cartridge along with a sample cartridge at the initiation of an assay run. In certain embodiments, the instrument may monitor the level of reagents within the reagent cartridge, especially in instances where a single reagent cartridge contains reagent amounts for multiple assays and notify the user when reagent levels are low or insufficient to perform the desired assay. The sample cartridge and/or the one or more reagent cartridges may comprise sealing pneumatic interface (SPI) ports and the instrument may be operable to transfer the one or more reagents via the SPI ports from the one or more reagent cartridges to the sample cartridge using one or more pipettes.

In certain embodiments, the instrument may comprise a 3-degree-of-freedom pipette gantry operable to transfer the one or more reagents. Systems may be operable to isolate, amplify, and sequence a target fungal nucleic acid in the sample at levels as low as 3 copies, target bacterial nucleic acid present in the sample at levels as low as 3 copies, and/or target viral nucleic acid present in the sample at levels as low as a single copy.

Instruments of the invention may have a total exterior volume of about 150 liters or less. In some embodiments, exterior volume of the instrument may be about 135 liters or less. The instrument may have a longest linear dimension of about 700 mm or less. In certain embodiments, the instrument can have a longest linear dimension of about 650 mm or less. The sample cartridge may be operable to receive biological, clinical, environmental, and food samples including untreated samples. Isolating the target nucleic acid may include digesting proteins in the sample by, for example, the instrument exposing the sample to proteinase K in the sample preparation unit. The sample preparation unit may be operable to lyse an organism to release the target nucleic acid. To that end, the sample preparation unit may include a lysis chamber comprising a rotating paddle, the instrument operable to flow the sample into the lysis chamber and interface with the sample cartridge to rotate the rotating paddle to mechanically lyse organisms in the sample. The lysis chamber may include comprising zirconium beads to aid in lysis.

The instrument may be operable to provide thermal energy to the sample preparation unit to denature nucleic acid therein. The instrument may be further operable to perform target capture by exposing the sample to target capture oligonucleotides and a solid support in the sample preparation unit to anneal the target capture oligonucleotides to the target nucleic acid to form a complex and bind the complex to the solid support. The instrument may then introduce a wash buffer to the solid support bound complexes and separate the solid support bound complexes from unbound sample. The instrument can then transfer the separated solid support bound complexes to the library preparation unit and amplify the solid support bound target nucleic acid. Instruments of the invention may be operable to introduce elution buffer to the separated solid support bound complexes to elute the target nucleic acid from the solid support and transfer the eluted target nucleic acids to the library preparation unit for amplification. The instrument can further be operable to introduce amplification reagents to the solid support bound complexes and amplify the target nucleic acid within the sample preparation unit.

In various embodiments, the cartridge and instrument may be operable to automatically accommodate sample volumes ranging between about 1 mL and about 25 mL received through the sample input. The instrument may be operable to interface with the library preparation unit of the sample cartridge to introduce required reagents and provide thermal energy to: perform a first amplification of the isolated target nucleic acid using a first primer set to produce a first amplification product; dilute the first amplification product and aliquot it into a plurality of aliquots; perform a second amplification of the target nucleic acid in the plurality of aliquots using a second primer set to produce a plurality of second amplification products; and pool the second amplification products. One or more primers in the first primer set may be identical to one or more primers in the second primer set. The instrument may be further operable to purify the pooled second amplification products to produce the amplified target nucleic acid.

Systems of the invention may be further operable to perform a copy control on one or more of the isolated target nucleic acid and the amplified target nucleic acid and control a number of output copies transferred to the library preparation unit or the sequencing unit respectively. The sequencing unit can comprise a semiconductor surface comprising an array of ion-sensitive field-effector transistor (ISFET) sensors each with a well positioned above it, the instrument operable to immobilize the amplified target nucleic acid above the array of ISFET sensors, the array of ISFET sensors in electronic communication with the instrument through the electronic connections of the cartridge interface when a sample cartridge is positioned therein. The instrument may be operable to flow all output from the library preparation unit into the wells over the semiconductor surface.

The cartridge may comprise a capture oligomer bound above the array of ISFET sensors wherein said capture oligomer is configured to hybridize to a portion of the target nucleic acid. At least one of the wells may be positioned above a plurality of ISFET sensors in the array of ISFET sensors. In some embodiments, one or more of the wells may comprise a surface bound forward primer that hybridizes to a portion of the target nucleic acid and a surface bound reverse primer that hybridizes to a portion of the target nucleic acid, the instrument operable to perform paired-end sequencing. One or more of the wells and interstitial space between the wells can comprise a plurality of bound inert oligomers that do not hybridize to the target nucleic acid. The cartridge may comprise a universal capture oligomer bound above the array of ISFET sensors wherein said universal capture oligomer is configured to hybridize to a universal binding site.

The instrument may be operable to interface with the library preparation unit to amplify the isolated target nucleic acid using a primer comprising the universal binding site. In some embodiments, the instrument can be operable to ligate an adapter onto the isolated target nucleic acid in the sample preparation unit or the library preparation unit, said adapter comprising the universal binding site. The instrument may be operable to interface with the sequencing unit to perform clonal amplification of the immobilized target nucleic acid and the clonal amplification may comprise recombinase polymerase amplification, rolling circle amplification, bridge PCR, strand displacement amplification, or loop-mediated isothermal amplification. The physical and electrical connections between the instrument and the sample and/or reagent cartridges may comprise a pneumatic system for driving fluid movement between and/or within the cartridges. The cartridge interface may further comprise physical and electronic connections through which the instrument is operable communicate with one or more of the sample preparation unit and the library preparation unit.

### BRIEF DESCRIPTION OF THE DRAWINGS

FIGS. 1A-1C illustrate an exemplary capture oligomer according to the disclosure, comprising a capture sequence, blocking moiety, complement of the capture sequence (C′), additional sequence (e.g., third or fourth additional sequence), and target-hybridizing sequence, together with other molecules. In FIG. 1A, the capture oligomer is annealed to a target polynucleotide (target), and the 3′ end of the target polynucleotide is annealed at the 5′ end of the target-hybridizing sequence. The capture sequence is annealed to C′. In FIG. 1B, the 3′ end of the target has been extended up to the blocking moiety and the resulting target extension is annealed to the additional sequence and C′, while the capture sequence has been displaced and has become single stranded. The 3′ end of the capture oligomer has also been extended along the target polynucleotide. In FIG. 1C, the complex of FIG. 1B has annealed to a secondary capture reagent comprising a complement of the capture sequence and a binding partner or solid substrate. Meanwhile, excess capture oligomer remains with the capture sequence annealed to the complement of the capture sequence and does not interact with the secondary capture reagent.

FIG. 2A illustrates an embodiment of the disclosure in which a complex as in FIG. 1B is annealed to a secondary capture reagent comprising a complement of the capture sequence and associated with solid substrate (in this case, a streptavidin-coated magnetic bead). The solid substrate may be part of the secondary capture reagent or may be associated with the secondary capture reagent through interaction with a binding partner (e.g., biotin) of the secondary capture reagent.

FIG. 2B illustrates an embodiment of the disclosure in which the complex of the extended capture oligomer and target from FIG. 2A has been eluted from the secondary capture reagent.

FIG. 3 illustrates an embodiment of a capture oligomer according to the disclosure comprising a stabilizing (clamp) sequence as a first additional sequence, a capture sequence, a linker, an internal extension blocker, a complement of the capture sequence, a stabilizing (clamp) sequence as a third additional sequence, a fourth additional sequence, and a target-hybridizing sequence.

FIG. 4A illustrates exemplary molecules and an exemplary reaction scheme according to the disclosure. A target molecule is provided in which a first strand comprises the sequences Sf at its 5′ end and Sr′ at its 3′ end and a second strand comprises the sequences Sf at its 3′ end and Sr at its 5′ end. Here and throughout, a sequence designation with a′ indicates complementarity to the sequence having the designation without the ′. The target molecule can be, e.g., an amplicon from a previously performed reaction using primers with the sequences Sf and Sr. A first extension cycle (Cycle 1) is performed, in which a capture oligomer according to the disclosure comprising a capture sequence C, an internal extension blocker (filled circle), a complement of the capture sequence C′, a fourth additional sequence A4, and a target-hybridizing sequence THS complementary to Sr′ anneals to the first target strand 1(+). A reverse amplification oligomer comprising an additional sequence A2 and a target-hybridizing sequence Sf*complementary to at least Sf anneals to the second target strand 1(−). Sf*may comprise affinity-enhancing modifications and/or additional complementary nucleotides to the second target strand to enhance its affinity to the target and facilitate competition for binding with a primer having the sequence Sf from a previous reaction if present. Extension of the capture oligomer and the reverse amplification oligomer generates products 2(−) and 2(+), respectively, while the first strand is extended along the capture oligomer to generate product 1(+)e and the second strand is extended along the reverse amplification oligomer to generate product 1(−)e. The capture sequence in the extended capture oligomer 2(−) is displaced essentially as described for FIG. 1B. A second reaction cycle (Cycle 2) is performed in which 2(−) anneals to a reverse amplification oligomer, resulting in extension to generate products 2(−)e and 3.1(+). Meanwhile, 1(+)e anneals to a capture oligomer and extension of the latter generates product 3.1(−). Additional instances of 1(−)e and 2(+), and of 2(−)e and 3.1(+), are also generated from the appropriate hybridization and extension events. This reaction scheme illustrates inclusion of additional sequences at each end of a target together with rendering the target capturable by incorporation of C in a form available for binding, for example with a second capture reagent.

FIG. 4B illustrates exemplary molecules and an exemplary reaction scheme according to the disclosure. A target molecule is provided in which a first strand comprises the sequences Sf at its 5′ end and Sr′ and A4′ at its 3′ end and a second strand comprises the sequences Sf′ at its 3′ end and Sr and A4 at its 5′ end. The target molecule can be, e.g., an amplicon from a previously performed reaction using primers with the sequences Sf and A4-Sr, e.g., wherein A4 is an additional sequence not originally present in the template. A first extension cycle (cycle 1) is performed, in which a capture oligomer according to the disclosure comprising a capture sequence C, an internal extension blocker (filled circle), a complement of the capture sequence C′, a fourth additional sequence A4, and a target-hybridizing sequence THS complementary to A4′ anneals to the first target strand (not shown). A reverse amplification oligomer comprising an additional sequence A2 and a target-hybridizing sequence Sf*complementary to at least Sf anneals to the second target strand (not shown). Sf*may comprise affinity-enhancing modifications and/or additional complementary nucleotides to the second target strand to enhance its affinity to the target and facilitate competition for binding with a primer having the sequence Sf from a previous reaction is present. Extension of these complexes generates an extended capture oligomer 2(−) and an extended first target strand 1(+)e, and an extended second target strand 1(−)e and an extended reverse amplification oligomer 2(+).The capture sequence in the extended capture oligomer 2(−) is displaced essentially as described for FIG. 1B. A second reaction cycle (cycle 2) is performed in which 2(−) anneals to a reverse amplification oligomer, resulting in extension to generate products 2(−)e and 3.1(+). Meanwhile, 1(+)e anneals to a capture oligomer and extension of the latter generates product 3.1(−). Additional instances of 1(-)e and 2(+), and of 2(−)e and 3.1(+), are also generated from the appropriate hybridization and extension events. This reaction scheme illustrates inclusion of an additional sequence at the end of a target distal from the capture oligomer binding site together with rendering the target capturable by incorporation of C (in a form available for binding) using a capture oligomer that can have a universal THS (i.e., that binds the additional sequence A4′ that can be attached to the target in a previous step (e.g., via amplification or ligation).

FIG. 5 illustrates exemplary molecules and an exemplary reaction scheme according to the disclosure. A capture oligomer is provided that comprises, among other things, a 3′ blocking moiety and a target-hybridizing sequence (THS) that binds sequence A1′ in a target strand. A1′ may be an additional sequence attached to the target in a previous step (e.g., via amplification or ligation). The capture oligomer further comprises a sequence x that comprises a complement of the capture sequence of the capture oligomer and may also comprise a third or fourth additional sequence between the complement of the capture sequence and THS. The target strand can be extended along the capture oligomer to displace the capture sequence from the complement of the capture sequence, as discussed elsewhere. The capture oligomer may be provided in a limiting amount (e.g., 10¹² copies) relative to the target (e.g., 10¹⁴ copies). A primer is also provided in excess over the target (e.g., 10¹⁵ copies) which comprises the sequences A2 and Sf. Extension of this primer results in a strand comprising A2 at its 5′ end and A1′ at its 3′ end. The target strand is also extended along the primer to include sequence A2′. If a second extension cycle is performed (downward arrow), a mixture of products is formed including those discussed above and a complex of a target strand with a capture oligomer in which the target strand comprises A2 at its 5′ end as well as A1′ near its 3′ end. This reaction scheme illustrates generation of single stranded capturable products, including (when the second extension cycle is performed) one in which additional sequences have been included in the target strand.

FIG. 6 illustrates (above the dashed line) how hybridization of a capture oligomer with an extendable 3′ end to another capture oligomer can, upon extension, produce a dimer in which the capture sequences are displaced from C′. This dimer is now capturable and may interfere with downstream processes, such as competition with capture of the desired target by occupying a secondary capture reagent (not shown), interference is subsequent analysis (e.g., dimers will become part of a sequencing library and thereby diminishing the output and quality of the subsequent sequencing run) and the like. Sx′ is a complement of part of the target-hybridizing sequence and other elements are as in previous figures. Below the dashed line, a capture oligomer with a blocking moiety at its 3′ end is illustrated (circled x), which prevents formation of the dimer extension product, such that any dimer would not undergo displacement of C.

FIG. 7A illustrates an embodiment in which a capture oligomer comprising a capture sequence, various intermediate elements (indicated by “ . . . ”), a reversible extension blocker (filled circle), and a target-hybridizing sequence (THS) is used. Before unblocking the reversible extension blocker, the capture sequence and the various intermediate elements (if present) are not a template for extension (e.g., of target strands or amplification oligomers). This can facilitate more efficient and more specific extension or amplification by avoiding incorporation of additional sequences complementary to the capture sequence and the various intermediate elements (if present) in the products (e.g., in any mispriming product that may be formed) throughout the extension or amplification process until the reversible extension blocker is unblocked; following unblocking, the capture sequence and the various intermediate elements (if present) can be incorporated.

FIG. 7B illustrates an embodiment in which a first amplification oligomer comprising, from 3′ to 5′, a target hybridizing sequence Sr, a reversible extension blocker (filled square), an additional sequence A1 and optional additional elements (indicated by “ . . . ”), such as an optional capture sequence, is used. A second amplification oligomer comprising, from 3′ to 5′, a target hybridizing sequence Sf, a reversible extension blocker (open square; this can be the same or different than the reversible extension blocker in the first amplification oligomer), an additional sequence A2 and optional additional elements (indicated by “ . . . ”; these can be the same or different than those in the first amplification oligomer), is optionally used (as shown in the figure). Before unblocking the reversible extension blocker or blockers, the additional sequence and optional additional elements (if present) are not a template for extension (e.g., of target strands or amplification oligomers). This can facilitate more efficient and more specific extension or amplification by avoiding incorporation of sequences complementary to the additional sequences and other various elements (if present) in the products (e.g., in any mis-priming product that may be formed) throughout the initial extension or amplification process.

The reversible extension blocker or blockers is/are unblocked (if two are present, unblocking can occur concurrently or separately) and the additional sequence and any other elements present can be incorporated in a later phase of the process, such as a later round of extension.

FIG. 8A illustrates exemplary molecules and an exemplary reaction scheme according to the disclosure. The initial target strands 1(+) and 1(−) are as in FIG. 4A. Below the straight vertical arrow, a capture oligomer is provided comprising a target-hybridizing sequence THS and, as described for the oligomer of FIG. 4A additional elements A4, C′, an internal extension blocker, and C. THS binds at an internal site in a target strand and undergoes extension to generate product 2N(−). A displacer oligomer comprising Sr is provided, and extension thereof displaces 2N(−) from 1(+) and generates 2(−). A reverse amplification oligomer as in FIG. 4A is provided, extension of which along 1(−) generates 2.1(+), and extension of 1(−) along the reverse amplification oligomer generates 1(−)e. Once 2N(−) is displaced (curved arrow at left), the reverse amplification oligomer anneals to 2N(−) and each is extended, resulting in products 2N(-)e and 2.2(+), which now comprises A2′ and in which C is displaced from C′. This reaction scheme illustrates use of a displacer oligomer to facilitate generation in only 1 cycle of a capturable product that contains additional sequence (e.g., adaptors) at both ends of the target sequence. Further, this reaction scheme illustrates an embodiment where the capture oligomer does not bind to a site including the 3′ end of a target strand.

FIG. 8B illustrates additional exemplary molecules and an additional exemplary reaction scheme according to the disclosure. The reaction scheme is substantially similar to that depicted in FIG. 4A with the following exceptions 1) and 2). 1) The initial target strands 1(+) and 1(−) comprise additional sequence comprising target hybridizing sequence THS, optional spacer sequence S and displacer oligomer binding site D. These additional sequences are user-defined, arbitrary sequences and can be incorporated into the target, for example, via an amplification reaction using an amplification oligomer comprising Sr and a sequence tag comprising THS, S and D and an amplification oligomer comprising Sf. 2) The THS of the capture oligomer binds to the user-defined THS site. The reaction otherwise proceeds as illustrated in FIG. 8A, and the resulting products are shown in FIG. 8B. The optional spacer can be useful for improving the extension of the displacer oligomer and subsequent displacement of the capture oligomer. As with the scheme shown in FIG. 4A, this reaction scheme illustrates use of a displacer oligomer to facilitate generation (e.g., in only one cycle) of a capturable product that contains additional sequence (e.g., adaptors) at both ends of the target sequence. Further, this scheme shows the use of additional, user-defined sequence that can function as binding sites for both the capture and displacer oligomers. This design can universalize the approach and allow for a simpler and much more cost-effective means of designing capture and displacer oligomers for use with different targets, including in a multiplex format.

FIG. 9 illustrates the general principle of how a blocker oligomer can prevent hybridization between an additional sequence in an oligomer and the complement thereof in an extension product. An amplification reaction is performed with a forward primer comprising sequence f, which hybridizes to the target strand T(−), and a reverse primer comprising sequence A (an additional sequence not present in the target) and sequence r, which hybridizes to the target strand T(+). Extension generates products 1(−) and 1(+). A blocker oligomer is provided comprising sequence A and a 3′ blocking moiety. In cycle 2, the forward primer is extended along 1(−), generating 2(+), and the reverse primer is extended along 1(+), generating 2(−). In cycles 3 and onward, the blocker oligomer anneals to 2(+), meaning that hybridization of r to r′ is necessary for the reverse primer to prime extension along 2(+). This can be beneficial in case any mispriming events occur that produce a small amount of side product having an imperfect complement of r but which are extended to include A′. Without the blocker oligomer, binding of the reverse amplification oligomer to the misprimed side product would be more favorable by virtue of the interaction between A of the reverse amplification oligomer and A′, leading to more amplification of the side product than would occur when the blocker oligomer is provided. (Meanwhile, the forward primer anneals to 2(−) and undergoes extension.)

FIG. 10A illustrates exemplary molecules and an exemplary reaction scheme according to the disclosure. A combination of (i) a capture oligomer comprising first and second portions of a capture sequence (C1 and C2), an internal extension blocker (filled circle), first and second portions of a spacer sequence (S1 and S2) and a target-hybridizing sequence (THS) that binds a site in a target strand comprising its 3′ end and (ii) a complementary oligomer comprising S1′ and C2′ is provided. Upon hybridization of the capture oligomer to a target and extension of the target along the capture oligomer up to the internal extension blocker, incorporating S′ into the target strand, the complementary oligomer is displaced. The capture oligomer is also extended along the target (note that in other embodiments described herein, the capture oligomer may be blocked and such extension would not occur). A secondary capture reagent is provided comprising a binding partner or solid support (circled B) connected by a linker (zigzag line) to a complement of the capture sequence C′. The secondary capture reagent anneals to capture oligomer bound to the extended target but not to capture oligomer bound to the complementary oligomer, because the latter occupies C2, which is a sufficient amount of the capture sequence to substantially prevent annealing of the secondary capture reagent to the capture oligomer.

FIG. 10B illustrates an embodiment in which a combination of oligomers is useful for capturing a target polynucleotide from a composition, including a certain amount (e.g., a limited amount or an amount less than or equal to a predetermined amount) thereof if desired. The combination comprises a capture oligomer comprising, from 5′ to 3′, a first portion of a capture sequence C1, a second portion of a capture sequence C2, an optional spacer sequence S, a second portion of a target hybridizing sequence THS2 a first portion target hybridizing sequence THS1, and an optional blocking moiety (circled X); a separate complementary oligo comprising, from 5′ to 3′, THS2′, S′ (optional; may or may not be used when S is present in the capture oligomer) and C2′ (wherein the complement of an element is indicated by “′”) and an optional blocking moiety at the 3′ end (circled X); and a secondary capture reagent comprising the complement of the capture sequence which comprises, from 5′ to 3′, C2′, C1′ (C1′ or C2′ may or may not be complementary to the entire length of C1 and C2), and a binding partner (exemplified in this illustration with a biotin molecule, represented as a circled B). In the absence of target polynucleotide, the complementary oligomer binds to the capture oligomer and blocks accessibility to the full capture sequence to a sufficient extent to block binding of the complement of the capture sequence in the secondary capture reagent (see the complex of complementary oligomer to capture oligomer at the top of the figure). In the presence of target, the THS1 region of the capture oligomer binds to the target followed by binding of the THS2 region—which is energetically favored—thus displacing the THS2′ region of the separate complementary oligo from the capture oligomer. When this occurs, the C2′ region of the separate complementary is no longer stable enough to bind to the capture oligomer and therefore becomes unbound, thus leaving the full capture sequence available for binding as shown below the first arrow. The complement of the capture sequence in the secondary capture reagent then binds to the capture sequence of the capture oligomer, as shown below the second arrow. This complex can then be isolated from the mixture—e.g., by means of streptavidin coating magnetic microspheres (as described elsewhere in this disclosure)—thus capturing and purifying the target polynucleotide. Optionally, the capture oligomer may be present in the combination in a greater amount than the secondary capture reagent. Such oligomers and combinations are useful for capturing a certain amount (e.g., a limited amount or an amount less than or equal to a predetermined amount) of a target polynucleotide from a composition.

FIGS. 11A-B illustrate exemplary molecules and an exemplary reaction scheme according to the disclosure. In FIG. 11A, a capture oligomer is provided comprising elements essentially as described for the capture oligomer of FIG. 4A, except that THS binds to a site in a target strand (which may be circular, as shown, or linear) that does not comprise a 3′ end. A complementary oligomer is provided which comprises (i) a target-hybridizing sequence that anneals adjacent to the THS of the capture oligomer and (ii) a complement of at least part of A4. The complement of at least part of A4 is insufficient to anneal to the capture oligomer in the absence of the target strand. In FIG. 111B, the complementary oligomer is undergoing extension, which will displace C and render it capturable using a secondary capture reagent (not shown). This scheme is useful for capturing circular molecules and/or represents another approach for using capture oligomers that do not bind at the 3′ end of a target strand.

FIG. 12 illustrates exemplary molecules and an exemplary reaction scheme according to the disclosure. A capture oligomer comprising elements as in the capture oligomer of FIG. 4A with a second additional sequence A2, which may comprise a mixed-nucleotide segment, between C′ and the internal extension blocker, anneals to a target strand at a site comprising its 3′ end. The target strand also comprises sequence A5 at its 5′ end, which may be an arbitrary sequence, a primer binding site used in a previous amplification reaction, or a sequence added during a previous step (e.g., amplification or ligation). Extension of the target strand along the capture oligomer adds sequences A4′, C, and A2′ to the 3′ end of the target strand. The presence of A4 in the capture oligomer and A4′ in the extended target strand is optional. The extended target strand can then be annealed to a splint oligomer comprising sequences A5′, A2, C′, and A4, wherein when the extended target strand is annealed to the splint oligomer, the target strand 5′ and 3′ ends are immediately adjacent. The extended target strand can then be circularized by ligation. The A2 and A2′ sequences serve to ensure proper juxtaposition of the extended target strand 5′ and 3′ ends. This can be helpful when C and C′ are repetitive sequences (e.g., poly-A and poly-T or vice versa) that may otherwise be prone to slippage that would inhibit formation of a substrate for ligation. This scheme is useful for capturing and then circularizing a target molecule, e.g., for use in a rolling circle amplification procedure.

FIG. 13 illustrates exemplary molecules and an exemplary reaction scheme according to the disclosure. A capture oligomer comprising a capture sequence C (comprising first and second portions C1 and C2; not shown), an internal extension blocker (filled circle), a spacer sequence S (comprising first and second portions S1 and S2; not shown), and a target-hybridizing sequence THS is provided along with a reverse amplification oligomer comprising sequence S2. THS and S2 serve to produce an amplified target (e.g., via PCR). A complementary oligomer is added that comprises a complement of the first portion of the spacer sequence S1′ and a complement of the second portion of the capture sequence C2′. C2′ is insufficient to anneal to C of an amplified target when S′ is annealed to S of the other strand of the amplified target. The complementary oligomer does anneal to capture oligomer that is not annealed to an amplified strand. To capture the amplified target, a secondary capture reagent comprising C′ and a binding partner or solid support (circled B) is added. The secondary capture reagent binds the amplified target but does not bind capture oligomer that is not annealed to an amplified strand, in which C is blocked to a sufficient degree by C2′ of the complementary oligomer.

FIG. 14 shows the fold-difference in output of methods using a capture oligomer with or without a clamp sequence.

FIG. 15. Panel A—Solution-Mediated Surface-Phase Recombinase Polymerase Amplification (SM-RPA) forward primer is present in solution while reverse primer is immobilized to the surface. Upon initial hybridization of the template to the surface primer, concerted action of solution primer, recombinase, single-stranded DNA binding proteins (ssDNA) and a polymerase leads to synthesis of complementary strands. Subsequent recombination and extension of the solution phase primer results in the displacement of one strand into solution. These strands can then be locally recaptured. Panel B—In comparison, in bridge RPA or ExAmp (for example) both primers are co-immobilized to the surface, enabling only surface-phase amplification.

FIG. 16 depicts a model of cluster formation in bridge RPA (Panel A) and SM-RPA on welled chips (Panels B and C). In bridge RPA (as in bridge PCR and ExAmp, for example), clusters are small (Panel A). In SM-RPA, amplification using in-solution primers enables lateral growth of clusters, the size of which is limited by spatial exclusion from neighboring clusters. Amplicons generated in solution are recaptured in the close vicinity of the seeding template molecule, facilitating formation of clonal patches of amplicon which are larger than those generated in bridge amplification (Panel B; clusters from 3 separate targets are indicated by ♦, and ‡).

FIG. 17 is a diagram of an exemplary surface (e.g., on-chip) template circularization method of the current invention. First, target template is adapted during PCR2 in Library Preparation with partial 1st and 2nd RCA primer-binding sites on each end. During the Copy Control phase of the workflow, the 2nd RCA primer-binding site is extended to include the Copy Control adaptor and the second part of the 1st RCA primer-binding site. The template is hybridized to a 1st RCA primer immobilized on the surface, which acts as a splint. This forms a structure with a gap between adjacent 5′ and 3′ ends. Addition of ligase fills that gap, creating a circle capable of facilitating an RCA reaction in situ.

FIG. 18 depicts one embodiment of cluster formation using Rolling Circle Amplification (RCA). (A) Hybridization of both ends of the linear RCA template to immobilized 1st primer molecule enables template circularization by ligase directly on surface. (B) Once the gap is closed by the ligase, an amplification mix is added containing a highly-processive strand-displacing polymerase. (C) Polymerase extends the free 3′-end of the 1st primer using the circle as template. (D) The elongating concatemeric 1st-strand amplicon contains repeating units of 2nd primer binding site. These sites hybridize to the immobilized 2nd primer molecules, serving as template for 2nd strand synthesis. (E) Once synthesis reaches the next unit, the elongating 2nd strands displace one another though strand-displacing activity of the RCA polymerase. (F) Freed 2nd strands hybridize with remaining free immobilized 1st primer molecules, facilitating further synthesis of concatemeric 1st strands. In turn, the elongating 1st strands displace one another, and the cycle continues.

FIG. 19 depicts a method for incorporating a primer specific key sequence as per Embodiment #1 in the specification. Truncation of the 3′ end of Oligo 2 enable the remaining known sequence of Section 1A to be used as a key sequence.

FIG. 20 depicts another variation of a method for incorporating a Target Specific Key Sequence as per Embodiment #1 of the specification, in this case using both 3′ truncation and 5′ extension. Truncation of the 3′-end of Oligo 4 enables the remaining known sequence of Section 3A to be used as a key sequence.

FIG. 21 depicts a method for incorporating a primer specific key sequence as per Embodiment #2 in the specification. In this example, synthetic, non-specific sequence at the 5′ end (Section 6B) of the surface-bound capture oligomer/primer (Oligo 6) can be used as a known, universal key sequence. Step (a): Sequencing template (Oligo 5), provided in solution, hybridizes to the complementary Section 6A of Oligo 6 (surface-bound). Step (b): Following enzyme addition, polymerization can occur from the 3′ end of Oligo 5 through Section 6B of Oligo 6. The resulting sequence may be used to set signal base calling parameters. The 3′ blocking moiety on Oligo 6 prevents polymerization from this terminus. Step (c): The 3′ end of Oligo 6 is unblocked as described. Step (d): Upon addition of additional polymerase (if needed), the sequencing reaction may now progress through the unknown region of Oligo 5.

FIG. 22 is a top view rendering of one preferred embodiment of a sample preparation cartridge. Panels A and B depict two possible reagent/assay configurations of chambers in the main cartridge body.

FIG. 23 is a top view rendering of one preferred embodiment of a sample preparation cartridge, depicting the main body plus 2 additional functional fins. Panels A and B depict alternate microfluidic configurations of the Mag Sep fin. “Mag Sep” means magnetic separation.

FIG. 24A is a 3D CAD rendering of one preferred embodiment of a sample preparation cartridge. FIG. 24B is a photograph of an actual prototype cartridge built according to the design in Panel A.

FIG. 25 is a top view rendering of one preferred embodiment of a library preparation cartridge, depicting the main body plus 1 additional functional fin. Panels A and B depict two possible reagent/assay configurations of chambers in the main cartridge body. “Mag Sep” means magnetic separation.

FIG. 26. Panel A is a 3D CAD rendering of one preferred embodiment of a library preparation cartridge. Panel B is a photograph of an actual prototype cartridge built according to the design in Panel A.

FIG. 27 displays a top view rendering of one preferred embodiment of a cluster generation/sequencing (CA-Seq) cartridge. Panels A and B depict two possible reagent/assay configurations of chambers in the main cartridge body.

FIG. 28. Panel A is a 3D CAD rendering of one preferred embodiment of a cluster generation/sequencing (CA-Seq)cartridge. Panel B is a photograph of an actual prototype cartridge built according to the design in Panel A.

FIG. 29 depicts one example of a flow cell assembly for use in the invention. Valves (one possible configuration shown) can be bonded or welded to the fin. The film can be solvent bonded, heat or laser welded, or via pressure sensitive adhesives. The flow cell is heat-staked through the PCB (printed circuit board). The silicon gasket between the film and the chip is not shown.

FIG. 30 is a 3D rendering of the sequencing reagent cartridge, shown here attached to a manifold for fluidic control and other required functions as well as a cluster generation/sequencing cartridge. One particular configuration of reagent bottles and contents thereof is shown but others are also envisioned.

FIG. 31 is a 3D rendering of an exemplary manifold for the sequencing reagent cartridge, used for fluidic control and other required functions.

FIG. 32 depicts one example of a sequencing reagent delivery system.

FIG. 33 depicts two example designs of an integrated assay cartridge.

FIG. 34 identifies various components contained within the cartridge shown in the preceding figure (not all components are identified or depicted in this drawing). The functions identified (e.g., STC, PCR1 & 2, CC, etc.), are exemplary only. The components can be utilized for a variety of functions as needed for a particular application. The numbered components are (in this example), 1) Sample input ports (2 in this configuration); 2) Mechanical Lysis Unit; 3) Specific Target Capture (STC) chambers; 4) PCR1 and PCR2 aliquoting chambers; 5) PCR1 and PCR2 reaction chambers; 6) Copy Control (CC) chambers; 7) Condensation trap chambers (for STC and CC); 8) Cluster Generation and Sequencing Flow Cell; 9) Pneumatic connections for fluidic control; 10) SPIs for access to fluidic connection to various chambers; 11) STC and CC fin (multiple thermal mixing chambers and bead capture regions); 12) Amplification/dilution fin (double-sided thermal control)

FIG. 35 depicts the following steps of an exemplary mechanical lysis (ML) process for a given application (e.g., detection of pathogens in blood) performed in the integrated cartridge: 1) Blood is withdrawn from vacutainer via liquid handler; 2) Blood is transferred into the specific target capture (STC) chambers, then liquid reagents are added to the blood; 3) Fluid is shuttle mixed between chambers while being incubated via heated instrument interface: 4) Blood and reagent solution is transferred to the mechanical lysis chamber, wherein a motor on the instrument spins the paddle to achieve lysis. Note that not all parts of the cartridge are shown in this figure for ease of viewing the featured components.

FIG. 36 depicts the following steps of an exemplary specific target capture (STC) process for a given application (e.g., detection of pathogens in blood) performed in the integrated cartridge: 1) Lysed blood solution is transferred from the MIL chamber back to the STC chambers; Capture beads are delivered to the chambers via Port 1 using the liquid handler; 2) Beads are thoroughly mixed during a heated incubation step in the STC chambers; 3) A magnet is engaged to contact the serpentine channel as blood is withdrawn by the liquid handler to the collect beads; Wash and elution buffers are introduced via port (1). Note that not all parts of the cartridge are shown in this figure for ease of viewing the featured components.

FIG. 37 depicts the following steps of an exemplary target amplification (e.g., PCR1 and PCR2 for targeted enrichment and tag/adaptor addition) process for a given application (e.g., detection of pathogens in blood) performed in the integrated cartridge: 1) Liquid handler extracts target nucleic acid from STC sub-module; 2) Liquid handler loads target solution into PCR1 chamber, rehydrating lyophilized reagents in fluid path; thermal cycling is conducted on the chambers by the instrument; 3) After PCR1 is completed, the liquid handler extracts PCR1 product and dilutes; 4) Liquid handler delivers diluted PCR1 product to PCR 2 chambers, rehydrating lyophilized reagents in fluid path; thermal cycling is conducted on the chambers by the instrument. Note that not all parts of the cartridge are shown in this figure for ease of viewing the featured components.

FIG. 38 depicts the following steps of an exemplary copy control (CC) process for a given application (e.g., detection of pathogens in blood) performed in the integrated cartridge: 1) PCR2 product pooled via liquid handler; 2) Liquid handler loads PCR2 product into Copy Control chambers; beads are prepared and delivered via liquid handler; 3) Fluid and beads are mixed and incubated via heated instrument interface; 4) A magnet is engaged with cartridge serpentine to collect beads as fluid is pulled into liquid handler; wash and elution buffers introduced into port (2) via liquid handler. Note that not all parts of the cartridge are shown in this figure for ease of viewing the featured components.

FIG. 39 depicts the following steps of an exemplary cluster generation and sequencing process for a given application (e.g., detection of pathogens in blood) performed in the integrated cartridge: 1) Eluted template is collected via liquid handler; 2 Template and cluster generation reagents are introduced to the cluster generation/sequencing flow cell via liquid handler; 3) Flow cell is incubated via heated instrument interface; 4) Sequencing reagents are delivered via instrument fluid manifold and sequencing is performed. Note that not all parts of the cartridge are shown in this figure for ease of viewing the featured components.

FIG. 40 highlights exemplary features of the instrument shown in FIG. 45, here shown for a particular application or set of applications. Features are flexible so as to accommodate a wide range of applications.

FIG. 41 is a cross-section schematic view (left) and a 3D rendering view of the sealing pneumatic interface (SPI) port.

FIG. 42 depicts two example designs of a reagent cartridge. Fluids can be accessed and moved in a number of ways, included via a liquid handler (LH; e.g., pipette system) and a liquid manifold (LM; e.g., on the instrument). Different methods of valving are applicable, including sealing pneumatic interface (SPI) ports. In the center figure, the numbers correspond to, 1) Foil sealed lyophilize reagent storage (LH access): 2) Foil sealed liquid reagent storage (LH access); 3) Waste volume for all sequencing waste (and optionally assay cartridge waste); 4) Waste inlet ports (manifold port and SPI); 5) Nucleotide chambers (each with SPI and manifold ports for fluid and CO₂ scrubbing); 6) Wash chambers (SPI and manifold ports); 7) Soda Lime chamber (for CO₂ scrubbing). SPIs and manifold ports are covered with, for example, either a removable seal or pierceable foil.

FIG. 43 depicts another example design of a reagent cartridge. Section A holds liquid reagents, section B holds dry reagents, section C is an interface to the sequencing reagents and other bulk reagents, as needed, and section W (the negative space of the reagent cartridge) is for waste storage (IM is the connection to the instrument manifold). The liquid and dry reagent chambers/bottles are sealed with foil, which is pierced by a pipette tip of the liquid handler to reconstitute (dry reagents) and transfer reagents. The design of the chambers and bottles allows great flexibility in the reagents that can be stored on-cartridge, allowing for a large number of different assays/processed to be performed on the cartridge. The section B module can be separated during manufacture and filled and dried independently as well as isolated in a low humidity environment for storage. In some embodiments, section A can also be separated during manufacture, filling and storage. In preferred embodiments, one or more of the chambers in the reagent cartridge contains a magnetic stir bar (see figure) which interfaces with a magnetic stirring motor in the instrument when the cartridge is loaded into the instrument (useful, for example, for onboard preparation/mixing of reagents).

FIG. 44 provides more detail regarding the reagent cartridge and functionality thereof.

FIG. 45 is a 3D rendering of one example instrument architecture for use in the System of the present invention.

FIG. 46 highlights some of the cartridge-loading features of the instrument shown in FIG. I-1.

FIG. 47 highlights additional exemplary features of the instrument shown in FIG. 1-1 (viewed from the opposite side), here shown for a particular application or set of applications. Features are flexible so as to accommodate a wide range of applications.

FIG. 48 highlights still additional exemplary features (focused on electronics) of the instrument shown in FIG. 1-1 (viewed from the back), here shown for a particular application or set of applications. Features are flexible so as to accommodate a wide range of applications.

FIG. 49 highlights yet additional exemplary features (focused on cooling) of the instrument shown in FIG. 1-1, here shown for a particular application or set of applications. Features are flexible so as to accommodate a wide range of applications.

FIG. 50 is a graph of qPCR results, demonstrating retrieval of spiked antimicrobial resistance (AMR) targets direct from blood (DfB) Using Specific Target Capture (STC) Oligomers

FIG. 51 is an agarose gel electrophoresis image confirming elution of ssDNA. Lane 1: DNA ladder, Lane 2: 5 ng of dsDNA P3 target and 100 ng of ssDNA (IDT ultramer), Lane 3: 5 ng of dsDNA P31 target and 100 ng of ssDNA (IDT ultramer), Lane 4: 5 ng of dsDNA P48 target and 100 ng of ssDNA (IDT ultramer), Lane 5: 68.1 ng of dsDNA from the PCR2 reactions, Lane 6: 70.3 ng of multiplexed (P3, P31 and P48 targets) full capture ssDNA material, replicate 1, Lane 7: 71.3 ng of multiplexed (P3, P31 and P48 targets) full capture ssDNA material, replicate 2, Lane 8: 78.3 ng multiplexed (P3, P31 and P48 targets) full capture ssDNA material, replicate 3.

FIG. 52 is an agarose gel electrophoresis image confirming ssDNA elution from NaOH eluates. Lane 1: DNA ladder, Lane 2: ssDNA control, Lane 3 and 4: dsDNA controls, Lanes 5 and 6: NaOH ssDNA eluates.

FIG. 53 are fluorescent microscopy images demonstrating in-well amplification of three synthetic DNA templates using SM-RPA. This was shown for three different starting copies of synthetic DNA template, with the amplified patches of target DNA visible by the different intensities seen in images for each target.

FIG. 54 are fluorescent microscopy images confirming in-well clonal amplification products of target nucleic acids using RCA. Two different starting copies of template were tested, with a separate chip used for each template input. For a given template input, the same area was imaged which showed discrete clusters of amplified target DNA products that are distinguishable in non-overlapping regions (shown in separate images for each fluorophore).

FIG. 55 shows the results from the sequencing of synthetic DNA template that was directly immobilized on the chip surface. The scatter plot shows Aligned Read Length on the x axis against Aligned Read Length—Errors on the y axis, with the corresponding histograms on top and to the right, respectively.

FIG. 56 shows the results from the sequencing of synthetic DNA template using the direct hybridization method. The scatter plot shows Aligned Read Length on the x axis against Aligned Read Length—Errors on the y axis, with the corresponding histograms on top and to the right, respectively.

FIG. 57 shows the results from the automated, sample-to-answer sequencing of pathogen spiked in whole blood. Top: The scatter plot shows Aligned Read Length on the x axis against Aligned Read Length—Errors on the y axis (labeled as “effectiveReadLen”), with the corresponding histograms on top and to the right, respectively. Bottom Analysis: Output showing that the spiked pathogen was correctly called.

FIG. 58A shows a perspective view of an exemplary instrument.

FIG. 58B shows a front view of the instrument of FIG. 58A.

FIG. 58C shows a side view of the instrument of FIG. 58A.

FIG. 58D shows cartridge interface assemblies within the instrument of FIG. 58A.

FIG. 58E illustrates an exemplary pneumatic pumping subunit within the instrument of FIG. 58A.

FIG. 58F shows positioning of an exemplary power subunit within the instrument of FIG. 58A.

FIG. 58G illustrates an exemplary air handling and reagent cartridge air intake subsystem within the instrument of FIG. 58A.

FIG. 58H illustrates a liquid cooling subsystem within the instrument of FIG. 58A.

FIG. 581 shows positioning of an exemplary condensation management subsystem within the instrument of FIG. 58A.

FIG. 59 shows a pneumatic subsystem for use in various instruments described herein.

FIG. 60 shows an exemplary sample or assay cartridge according to certain embodiments.

FIG. 61 shows an exemplary reagent cartridge according to certain embodiments.

FIG. 62 shows an exemplary workflow for performing an assay using an instrument and cartridges as described herein.

FIG. 63 shows an exemplary sample or assay cartridge with library preparation unit.

FIG. 64 shows an exemplary sample or assay cartridge with sample input and mechanical lysis subunit.

FIG. 65 shows an exemplary sample or assay cartridge with specific target capture subunit.

FIG. 66 shows an exemplary flow cell and pipette storage within an exemplary assay cartridge.

FIG. 67 shows an exemplary SPI port configured for 1 mL pipette tips.

FIG. 68 shows an exemplary SPI port configured for 5 mL pipette tips.

FIG. 69 shows an exemplary specific target capture subunit according to certain embodiments.

FIG. 70 shows an exemplary library preparation unit or PCR fin.

FIG. 71 shows PCR results illustrating successful mechanical lysis and observation of released target nucleic acids using an exemplary cartridge-compatible mechanical lysis subunit.

FIG. 72 shows PCR results illustrating successful specific target capture using an exemplary cartridge-compatible subsystem.

FIG. 73 shows an electropherogram overlay of various PCR results from Example R.

FIG. 74 shows sequencing results obtained from an amplified template using the Direct Hybridization method in Example S.

### DETAILED DESCRIPTION

The present disclosure provides oligomers, methods, compositions, and kits, useful for the rapid analysis of target polynucleotides, including determining the nucleotide sequence thereof. Analysis can be performed from a wide range of input sample types and amounts using a fully automated system comprising a cartridge, instrument, and operational and analytical software with no human intervention required after the run is started (sample-to-report). Further, complete workflows are disclosed exemplifying chemistry and mechanical aspects as well as other aspects of the invention.

Generally speaking, the workflows comprise one or more of the following segments: 1) Sample Preparation, 2) Library Preparation, 3) Copy Control, 4) Cluster Generation, or 5) Sequencing. Cartridge and instrument designs and concepts are disclosed in which all the chemistry workflow steps of each of the segments can be performed in an automated fashion. Embodiments comprising sequencing performed on the surface of a semiconductor chip are disclosed. Software that controls all the functions of the instrument has been written as well as [software-controlled]algorithms for all phases of data analysis from raw data to final report (i.e., what is the answer to the question for which the test was run).

### 1. Sample Type

A wide range of sample types (an exemplary, non-exhaustive list of sample types is provided in the “Definitions” section) are envisioned as appropriate for use in the disclosed invention. In a preferred embodiment, the sample is in liquid form and the entire sample or a portion thereof is introduced into the cartridge. In another preferred embodiment, the sample is in solid form but is processed to render the sample or a portion thereof into liquid form before introduction into the cartridge. Alternatively, the solid is processed into a suspension, slurry, emulsion or the like and then introduced into the cartridge. In another embodiment, the solid sample is introduced directly into the cartridge and therein processed at or near the beginning of the workflow to render the sample into the form of a liquid, a suspension, an emulsion or the like. Alternatively, the target polynucleotide can be directly extracted from the solid sample, either before or after introduction into the cartridge. In another embodiment, the sample is already in the form of a suspension, a slurry, an emulsion or the like when obtained, and is either introduced directly into the cartridge or is processed before introduction into the cartridge into liquid, solid or gaseous form. In another embodiment, the sample is in gaseous. Typically in gaseous samples the target polynucleotide is present in the sample in the form of an aerosol or suspension, either associated with a cell or not associated with a cell. The gaseous sample can be introduced directly into the cartridge and the polynucleotide can be collected, suspended, or in other ways harvested from the gaseous sample followed by introduction into the cartridge. It is envisaged the sample input port will accommodate the said wide range of sample types. The sample input port can exist in various forms for this to occur, including but not limited to, 1) a universal sample port that directly accommodates all sample types, 2) a sample port designed for a specific sample type or group of sample types, wherein cartridges with different sample input ports are available for different assay types, 3) a quasi-universal sample port that accommodates attachment of an adaptor designed for a given sample type, such that the appropriate adaptor is attached to the cartridge depending on the assay to be performed.

Preferred sample types comprise whole blood, for example, as used in a preferred embodiment of the disclosed invention wherein the system is utilized for the detection of blood stream infections as well as antimicrobial resistance genes (see further details within) and plasma, for example, used in preferred embodiments for detection of cell free DNA (cfDNA), including circulating tumor DNA (ctDNA). Another preferred embodiment wherein the preferred sample type comprises whole blood is detection of targets in plasma, such as but not limited to cfDNA, ctDNA and various viral infections (e.g., HIV). In such embodiments, plasma can be separated from whole blood preferably in the assay cartridge but can alternatively be performed in a sample collection tube or other device which ideally interfaces directly with the sample input port.

### 2. Sample Preparation

A wide range of sample preparation methods and compositions (an exemplary, non-exhaustive list of sample preparation methods is provided in the “Definitions” section) are envisioned as appropriate for use in the disclosed invention.

In one preferred embodiment, whole blood is the sample and target polynucleotide is contained within various infectious agents (e.g., bacteria, fungus, virus) that are present in the whole blood (an exemplary application is detection of bloodstream infections as well as antimicrobial resistance genes). An exemplary sample preparation method of this embodiment comprises the following steps: 1) Mix the whole blood sample with sample preparation reagents (e.g., including reagents useful for sample homogenization and cell lysis; in one aspect of this feature of this preferred embodiment, vigorous mixing helps to solubilize the sample); 2) Further homogenize the sample by incubation at elevated temperature and concurrent digestion of proteins in the sample with Proteinase K; 3) Combine the homogenized, digested sample with bead bashing beads; 4) Mix the beads and sample at relatively high speed (e.g., 8000 RPM) using, for example, a rotating impeller, thus lysing the infectious agents and releasing into solution the target polynucleotide contained within; 5) Heat the sample at high temperature (e.g., 95° C.) to denature target polynucleotide which is in double-stranded form (this step also helps strip away proteins as well as other components and/or structures that surround and/or are bound to the target polynucleotide); 6) Mix the denatured target polynucleotide sample with a set of specific target capture (STC) oligonucleotides (the STC oligos can be designed, for example, to a specific target, a set of specific targets, a broad range of targets, etc., as further discussion within); 7) Heat the STC oligo/target mixture (e.g., 60° C.) to facilitate annealing of the STC oligos with their specific target sequences; 8) Combine the STC oligo/target mixture with streptavidin-derivatized paramagnetic particles; 9) Mix, and then incubate the STC oligo/target mixture (e.g., 45° C.) to facilitate binding of the STC oligo/target mixture (the STC oligos comprise appended biotin molecules) to the beads; 10) Immobilize the STC oligo/target mixture/bead complex using magnets; 11) Wash STC oligo/target mixture/bead complex; 12) Elute the target polynucleotides, which are now ready for further processing in the workflow.

There are many advantages of the above preferred embodiment, including but not limited to, 1) The entire process is automated in a cartridge in a fully automated system; 2) When sample processing is complete, the workflow continues uninterrupted in the closed cartridge so there is not user intervention required to continue processing the test/assay; 3) A large volume of whole blood is accommodated in the cartridge, improving overall test/assay performance, including sensitivity; 4) the sample is homogenized, the cells lysed and the target polynucleotide denatured, ready for further processing, in a rapid and efficient automated protocol; 5) the specific target capture (STC) process affords a high degree of purification in a rapid protocol; 6) the STC process affords a high degree of specificity, modulated, among other parameters, by capture oligomer design and reaction conditions (mixture composition, temperature, etc.); 7) the STC process affords a high degree of inclusivity, wherein DNA sequences all along the phylogenetic tree can be capture or excluded as desired, again using capture oligomer design, reaction conditions, etc.; 8) the eluted sample is ready for further processing without the need for analysis.

In other embodiments, various features of the preferred sample preparation embodiment described in the paragraph immediately can be substituted, in various combinations, with alternative features/methods, including for example, 1) Proteinase K can be replaced or augmented with one or more alternate enzymes, detergents, chaotropes (e.g., GuSCN), chemical agents, including reducing agents, and the like; 2) Further sample homogenization after mixing with lysis reagents may not be required; 3) Cell lysis via bead bashing with zirconium beads may be replaced or augmented with alternate beads, balls, or other grinding media, sonication, detergents, chaotropes, reducing agents (DTT, beta-mercaptoethanol) and/or other chemical agents; 4) Homogenization and cell lysis can be conducted concurrently; 5) The step of denaturing the double stranded target polynucleotide can be omitted if the target is otherwise available for further processing; 6) The sample or a fraction thereof can be utilized directly for further processing at this point if target capture/separation/isolation is not required for a given application; 7) STC [alone]can be replaced with, for example, a) Non-specific target capture (e.g., the Boom method; see other exemplary methods in the “Definitions” section), b) A combination of non-specific and specific target capture methods (e.g., the Boom method followed by the STC method), c) Capture using aptamers, d) Filtration, e) Isoelectric focusing, f) Other methods listed in the “Definition” section, g) Combinations thereof.

In some embodiments, target capture oligomers (TCOs) may serve functions in addition to target capture alone. For example, TCOs can comprise one or more tags that are useful in downstream processes (e.g., Unique Molecular Identifiers (UMIs), universal amplification primer sites, promoters (e.g., T7 RNA polymerase promoter), adaptors for sequencing, etc.).

Also, TCOs may serve as primers for extension and amplification (which may work in unison with the one or more tags to accomplish a desired function). In some workflows, annealing of a TCO and extension thereof by a polymerase may take place in the same reaction mixture, either concurrently or at different times (or overlapping times). In others, annealing of a TCO may occur first followed by extension of the TCO, for example, upon addition of or combination with another reagent. In others, annealing followed by separation of the TCO/target complex may occur first, followed by extension in a subsequent step, for example, while the TCO/target complex is still immobilized to a solid support. In some of these workflows last described, extension may occur followed by final washing of the immobilized TCO/target complex and then elution. In this case, for example, the target polynucleotide is now ready to move to the next part of the process already purified, tagged and extended, saving time, steps, etc. in the overall workflow.

In some embodiments, no sample prep is required (i.e., the target polynucleotide can be used “direct from sample” in the first step of the process, for example, amplification).

With the wide range of reaction chambers, storage chambers, fluidic interconnectivity, valving configurations, reagent delivery options, functionalities (mixing, stirring, heating, transporting, separating, including magnetically, etc.) disclosed or envisioned with the cartridge of the present invention, each one of these sample prep workflow options can be accommodated within the framework of a rapid, fully automated workflow.

### 3. Library Preparation

A wide range of library preparation methods and compositions (an exemplary, non-exhaustive list of library preparation methods is provided in the “Definitions” section) are envisioned as appropriate for use in the disclosed invention. Target polynucleotide can be provided as input for library preparation by a variety of pathways, comprising directly from sample or in the form of the output from a wide variety of sample preparation methods. In one preferred embodiment, a portion or portions of a target polynucleotide are selectively enriched using amplification (which, for example, supports a targeted sequencing approach). Other optional features of this preferred embodiment comprise incorporation of tags, optionally comprising adapters, two or more separate rounds of amplification, optionally comprising the use of nested primers for the second and/or, if used, later rounds, and copy control (see elsewhere herein; described as a separate feature, but can also overlap with library preparation). An exemplary application is detection of bloodstream infections as well as antimicrobial resistance genes. An exemplary library preparation method of this embodiment comprises the following steps: 1) Combine the target polynucleotide with a first amplification reagent (as stated elsewhere herein, the target polynucleotide can come from a variety of sources; one preferred source is the output of the sample preparation method described above, which utilizes whole blood as the sample type); 2) Conduct a first amplification using PCR; 3) Dilute the product of the first amplification; 4) Mix an aliquot of the diluted first amplification product with a second amplification reagent; 5) Conduct a second amplification using PCR; 6) Combine the product of the second amplification with a capture reagent (comprising magnetic capture beads) and mix; 7) Incubate the second amplification product/capture reagent mixture at ambient temperature (about 20-26° C.) for 10 minutes with constant mixing; 8) Immobilize the second amplification product/bead complex using magnets; 11) Wash the second amplification product/bead complex; 12) Add elution reagent to the washed second amplification product/bead complex, mix and incubate at ambient temperature (about 20-26° C.) for 1 minute. The eluted library molecules are now ready for further processing in the workflow.

Important features and advantages of the library preparation workflow preferred embodiment summarized in the paragraph above comprise: 1) A very wide range of sample input types and amounts are acceptable; 2) The first amplification reaction (PCR1) is designed to have high sensitivity and fidelity; 3) The primers utilized in PCR1 are designed to broadly amplify across the spectrum of bacterial and fungal targets potentially contained within the sample, targeting bacteria 16S and 23S, fungal 28S, and specific Antimicrobial Resistant (AMR) genes; 4) No purification of the PCR1 amplicon is required before moving into the second amplification (PCR2), just a simple [automated]dilution (this is different from prior art protocols which require a purification step); 5) The primers used for PCR2 are nested within the primer sites for PCR1 and are designed to yield high specificity for the target polynucleotide or nucleotides of interest; 6) Multiple different PCR2 reactions can be performed (for example, one embodiment of the cartridge design (see “Assay Cartridge” section below) shows 10 separate chambers dedicated for separate PCR2 reactions) using aliquots from the same dilution of the PCR1 product, increasing the specificity of each reaction by reducing complexity (i.e., the desired number of targets are covered in different 10 reactions, for example, rather than 1, thereby reducing the number of primers in any given PCR2 reaction) and/or increasing the multiplexing capability of the system; 7) In one case of this preferred embodiment, at least one of the primers of a primer pair used in PCR2 is equipped with one or more biotin molecules, enabling immobilization of the amplicon product thereof using streptavidin-conjugated microspheres (or the like); 8) One or more tags, including adapters, can be added in PCR1, PCR2 or a combination therefore, including incorporation on both ends of the amplicon if desired; 9) A copy control process can be added after the library preparation workflow steps summarized above or designed to overlap with/be incorporated into the PCR2 step or even earlier in the workflow (see more details elsewhere within). In summary, the method can use a wide variety of target polynucleotide input types and amounts, is highly sensitive and specific, can be used to easily incorporate tags, including adaptors, can be easily interfaced (or overlap) with a copy control process, is simple (e.g., only 1 purification step), rapid and easy to automate.

In other preferred embodiments, various features of the preferred library preparation embodiment described in the paragraph immediately can be substituted, in various combinations, with alternative features/methods, including for example, 1) Different nested amplification configurations can be used, for example, a) A primer is nested only on 1 end of the target region, b) no nesting is used, c) the nesting pattern is different for different target polynucleotides, 2) PCR can be replaced with another amplification method; 3) One amplification reaction can be performed instead of 2 (when the required performance, e.g., sensitivity, specificity, multiplexing capability, can be achieved and tags, including adapters, can still be incorporated if needed), rendering the workflow even simpler and more rapid; 4) Aspects of library preparation can overlap with sample preparation (discussed in more detail in the paragraph below); 4) Tags, including adapters, including Unique Molecular Identifies (UMIs), can be attached to target regions using methods other than amplification, e.g., ligation; 5) Copy control methods can be added immediately at the end of the workflow cited in the embodiments described above, or immediately before the purification step, or [starting with]the PCR2 (or alternate amplification method) step or even with the last one or few cycles of the second amplification step, or PCR1 (or alternate amplification method) step, or the 1 amplification step (if only 1), or even as early as sample prep (copy control aspects also discussed elsewhere within). Other library preparation methods that can be used in the current disclosed method are listed in the “Definitions” section.

As mentioned above, aspects of library preparation can overlap with sample preparation. The following are some examples to illustrate this concept. 1) An oligomer that can function as a primer is annealed to target polynucleotide during sample prep. The primer can also serve other functions as well, such as the function of a target capture oligomer. The oligo can anneal to the target polynucleotide with the desired level of specificity, adding at this early stage (sample preparation) to the overall specificity of the assay and thereby improving its performance. The oligo can further comprise a tag, including an adapter, including UMIs, a universal primer site, etc., if desired. At some point, e.g., concurrent with annealing, after annealing in a second step (e.g., combining the annealing reaction mixture with an extension reaction mixture), after immobilization of the oligo/target complex (e.g., while still immobilized), after elution, etc., or in essence as part of a first phase of library prep, the oligomer is extended. This extension product can now enter into the remainder of the workflow, already equipped with a tag and with a level of specificity already achieved, potentially saving steps and time in the overall assay. Also in this mode, the first (or only) amplification step can utilize a specific opposing primer (with any desired level of specificity, even potentially non-specific by the use of randomers) and, if desired, a universal primer which binds to [at least a portion of]the tag sequence of the oligomer. Furthermore, incorporation of a tag, including an adapter, including UMIs, during sample preparation potentially allows the product [of sample preparation]to by-pass library preparation and go directly to other steps of the workflow, e.g., cluster generation and/or sequencing (in applications where sensitivity is sufficient and no additional tags, etc. are required). 2) Using a variation of method 1 immediately above, an oligo can be annealed to each of the strands of double-stranded target polynucleotides, each with the desired level of specificity and one or both comprising tag (including adapters, including UMIs). This potentially completes even more of the workflow at this early stage, saving steps and time, and increases overall specificity. Furthermore, the first (potentially only) step of library preparation could be universal amplification using primers that anneal to tag sequences on both ends of the target. These can be the same or different. Also, other elements needed for other steps of the workflow could be incorporated into the target, during the sample preparation step and/or at the universal amplification step (with universal primers equipped with tags). 3) Tags (including adapters, including UMIs) can be ligated to target polynucleotides during sample preparation.

With the wide range of reaction chambers, storage chambers, fluidic interconnectivity, valving configurations, reagent delivery options, functionalities (mixing, stirring, heating, transporting, separating, including magnetically, etc.) disclosed or envisioned with the cartridge of the present invention, each one of these library preparation workflow options can be accommodated within the framework of a rapid, fully automated workflow.

### 4. Copy Control

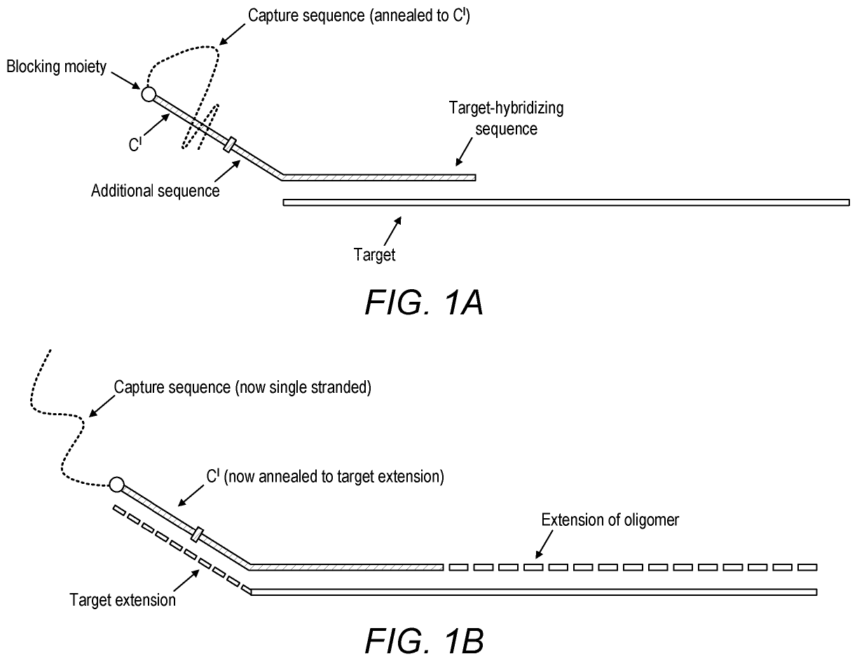

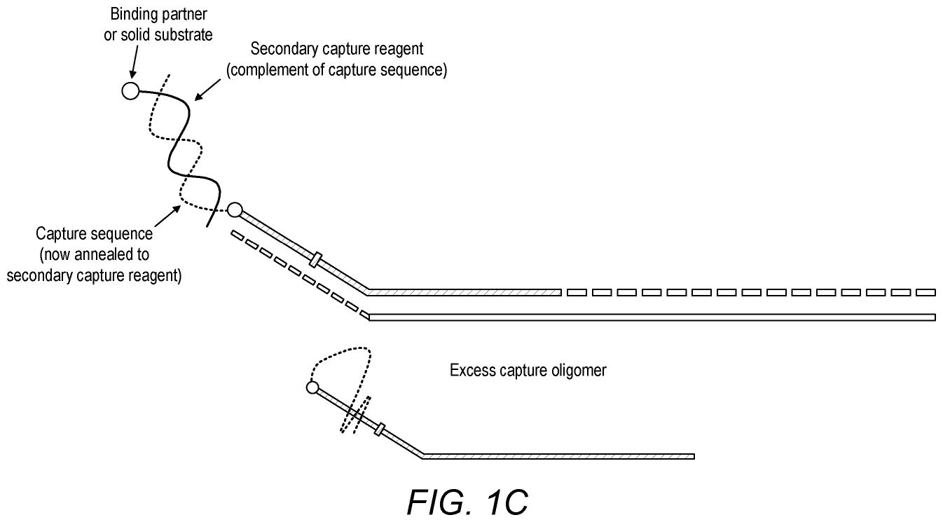

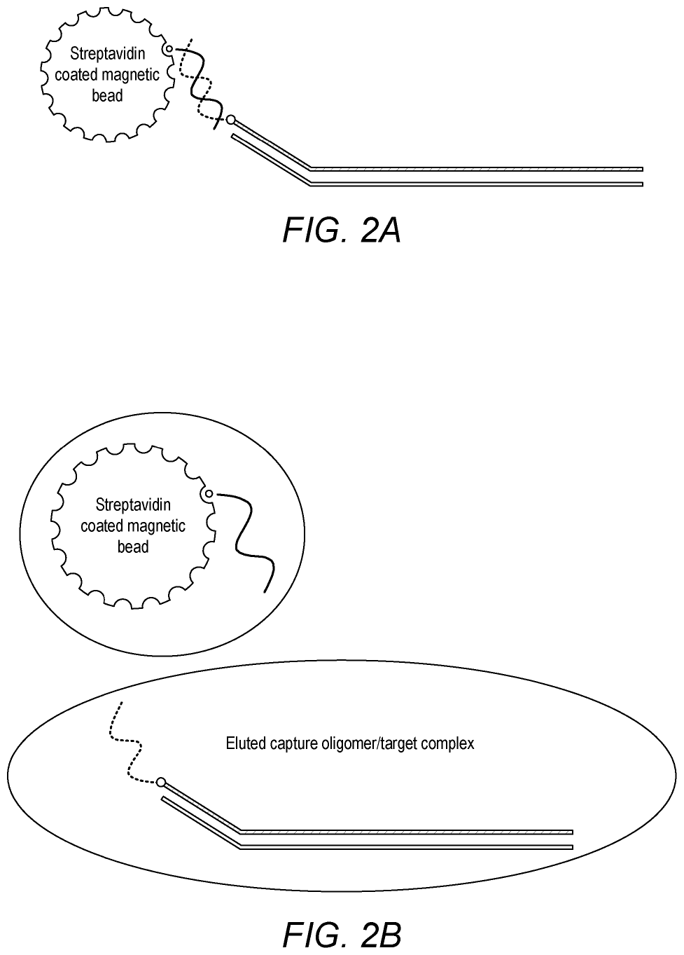

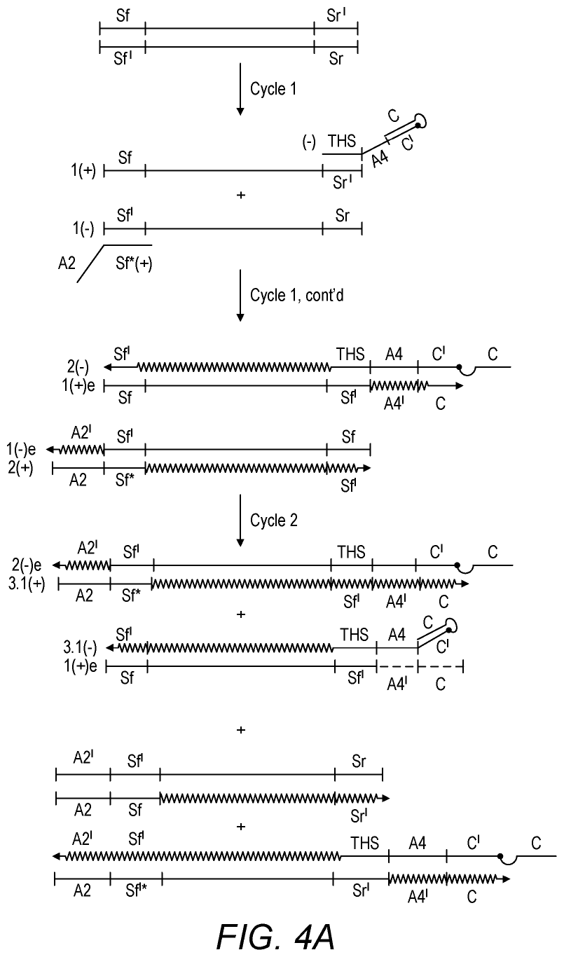

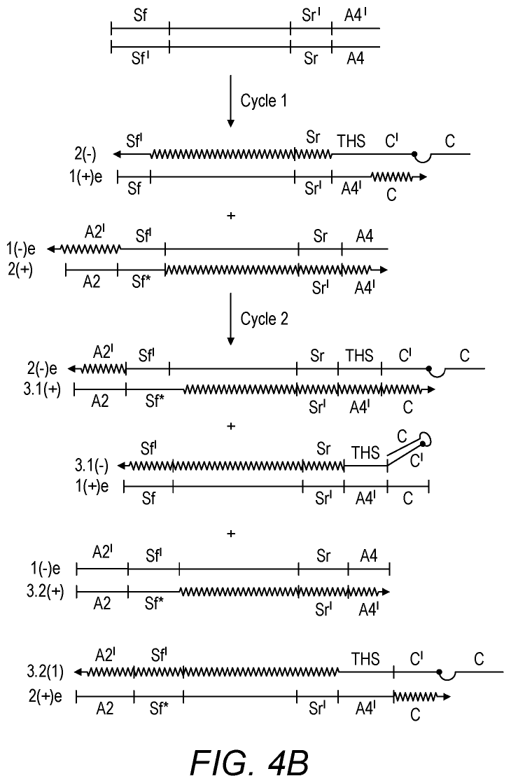

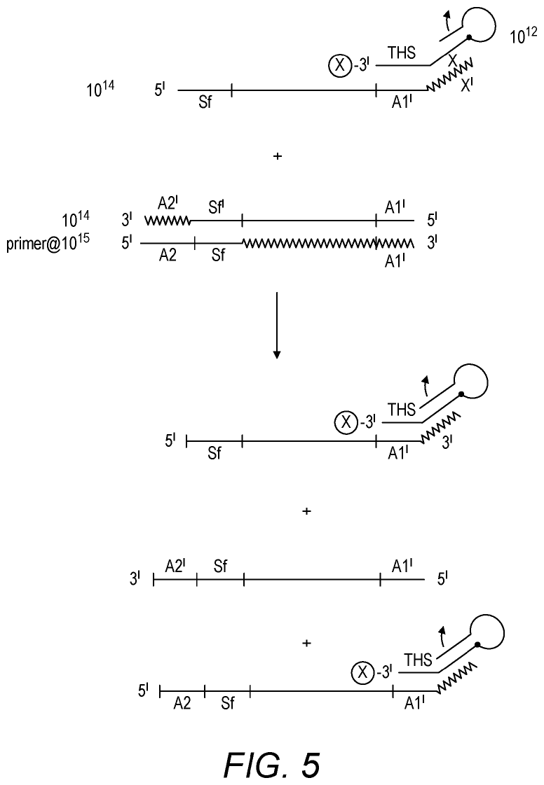

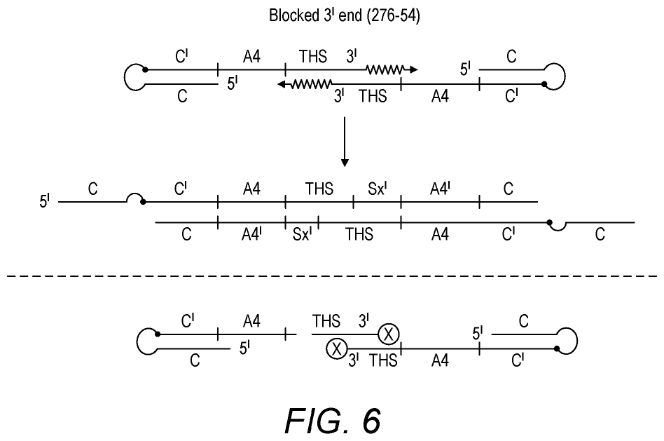

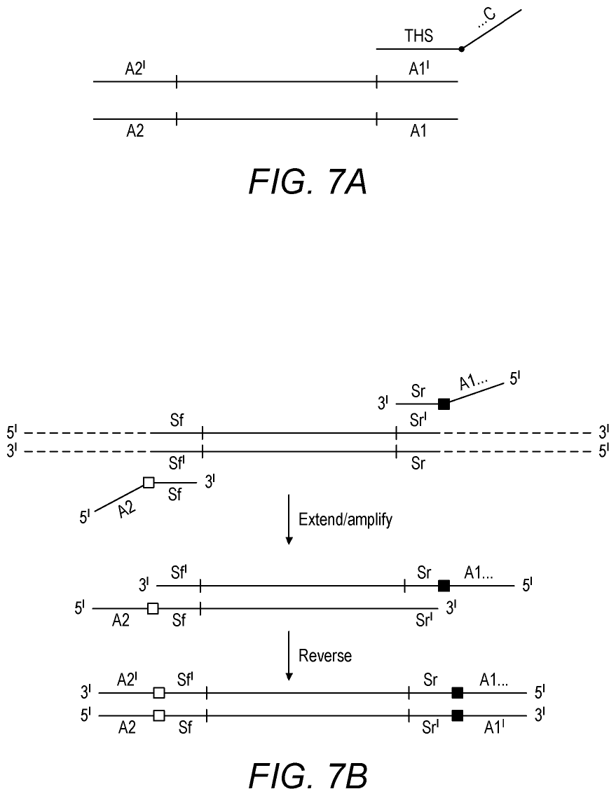

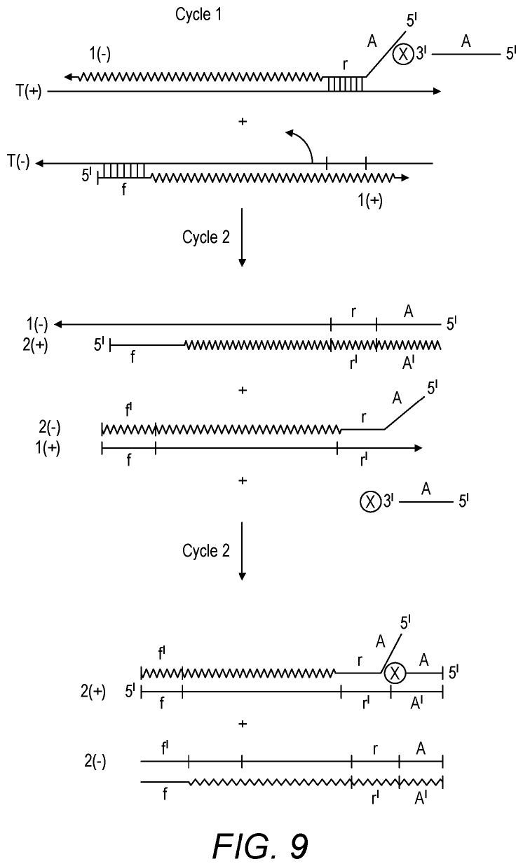

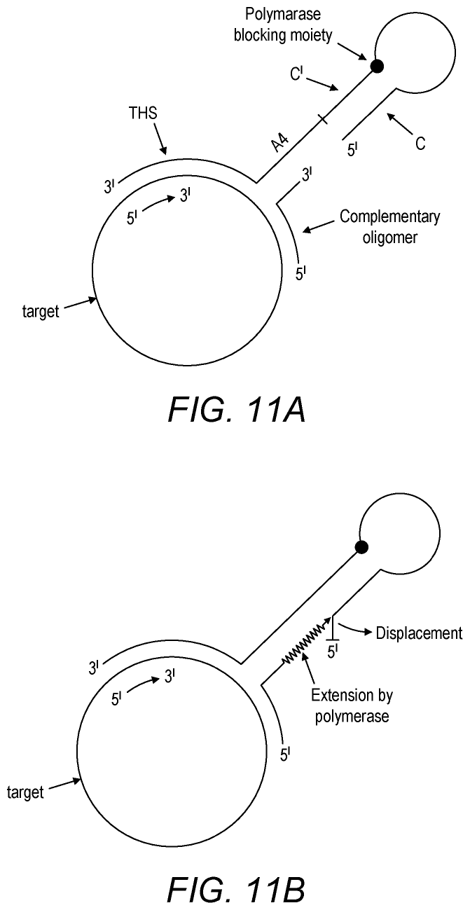

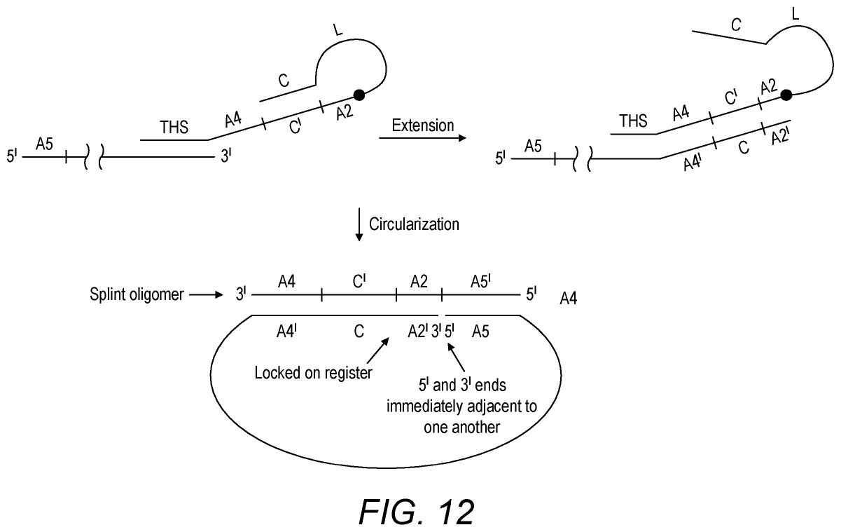

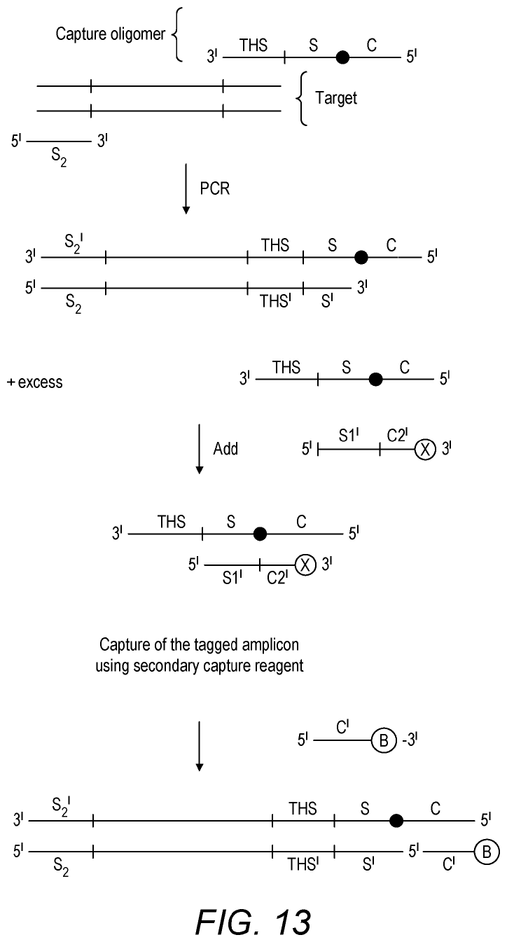

By “copy control” (CC) is meant compositions and methods by which the number of copies of molecules that are the output of a given process are controlled in a predetermined manner (e.g., limited amounts, i.e., up to but not exceeding a predetermined amount; or, a specific, predetermined amount). Further, in certain workflows it is desirable to incorporate additional sequences into a target polynucleotide (e.g., tags), such as incorporation of adapters into a sequencing library. This can also be accomplished in CC compositions and methods (see “Definition” as well as Figures and associated brief descriptions thereof for additional detail). CC is included in many of the workflows disclosed herein. A wide variety of CC compositions and methods are disclosed in, “COMPOSITIONS, KITS AND METHODS FOR ISOLATING TARGET POLYNUCLEOTIDES”, PCT/GB2021/050098, incorporated by reference herein in its entirety. All of the CC compositions and methods disclosed therein are applicable to the workflows of this invention. Furthermore, examples of CC methods are summarized in FIGS. 1-14 contained herein (see accompanying description in “Brief Description of the Drawings”).

A preferred CC embodiment comprises a capture oligomer comprising, in the 5′ to 3′ direction: a capture sequence, an internal extension blocker, a complement of the capture sequence, and a target-hybridizing sequence, wherein the complement of the capture sequence is configured to anneal to the capture sequence in the absence of an extended target sequence annealed to the target-hybridizing sequence and the complement of the capture sequence. In some such preferred embodiments the capture oligomer has the formula 5′-A1-C-L-B-A2-C′-A3-RB-A4-THS-X-3′, wherein A1 is an optionally present first additional sequence, C is the capture sequence, L is an optionally present linker, B is the internal extension blocker, A2 is an optionally present second additional sequence, C′ is the complement of the capture sequence, A3 is an optionally present third additional sequence, RB is an optionally present reversible extension blocker, A4 is an optionally present fourth additional sequence, THS is the target-hybridizing sequence; and X is an optionally present blocking moiety. In some of these preferred embodiments, the capture sequence comprises a poly A or poly T sequence and the complement of the capture sequence comprises a poly T or poly A sequence.

Another preferred embodiment comprises a combination comprising a capture oligomer and a complementary oligomer, wherein, (a) the capture oligomer comprises, in the 5′ to 3′ direction: a capture sequence comprising first and second portions, an internal extension blocker, a spacer sequence comprising first and second portions, and a target-hybridizing sequence; and (b) the complementary oligomer comprises, in the 3′ to 5′ direction: a complement of the second portion of the capture sequence, and a complement of at least the first portion of the spacer sequence, wherein the complement of the second portion of the capture sequence and the complement of the at least first portion of the spacer sequence are configured to anneal simultaneously to the capture oligomer in the absence of a complement of the spacer sequence.

In some such preferred embodiments the capture oligomer has the formula: 5′-A1-C1-C2-B-A2-S1-S2-A3-RB-A4-THS-X-3′, wherein A1 is an optionally present first additional sequence, C1 is the first portion of the capture sequence, C2 is the second portion of the capture sequence, B is the internal extension blocker, A2 is an optionally present second additional sequence, S1 is the first portion of the spacer sequence, S2 is the second portion of the spacer sequence, A3 is an optionally present third additional sequence, RB is an optionally present reversible extension blocker, A4 is an optionally present fourth additional sequence, THS is the target-hybridizing sequence, and X is an optionally present blocking moiety. In some such preferred embodiments the complementary oligomer has the formula: 5′-S1′-A2′-L-C2′-X-3′, wherein S1′ is the complement of at least the first portion of the spacer sequence, A2′ is an optionally present complement of a second additional sequence which is optionally present in the capture oligomer; L is an optionally present linker, C2′ is the complement of the second portion of the capture sequence, and X is an optionally present blocking moiety.

Yet another preferred embodiment comprises a combination comprising a capture oligomer and a complementary oligomer, wherein, (a) the capture oligomer comprises, in the 5′ to 3′ direction: a capture sequence comprising first and second portions, and a target-hybridizing sequence comprising second and first portions; and (b) the complementary oligomer comprises, in the 3′ to 5′ direction: a complement of the second portion of the capture sequence, and a complement of the second portion of the target-hybridizing sequence, wherein the complement of the second portion of the capture sequence and the complement of the second portion of the target-hybridizing sequence are configured to anneal simultaneously to the capture oligomer in the absence of a complement of the target-hybridizing sequence. In some such preferred embodiments the capture oligomer has the formula: 5′-A1-C1-C2-A2-S-A3-THS2-THS1-X-3′, wherein A1 is an optionally present first additional sequence, C1 is the first portion of the capture sequence, C2 is the second portion of the capture sequence, A2 is an optionally present second additional sequence, S is an optionally present spacer sequence, A3 is an optionally present third additional sequence, THS2 is the second portion of the target-hybridizing sequence, THS1 is the first portion of the target-hybridizing sequence, and X is an optionally present blocking moiety. In some such preferred embodiments the complementary oligomer has the formula: 5′-THS2′-A3‘-S’-A2′-C2′-X-3′, wherein THS2′ is the complement of the second portion of the target-hybridizing sequence, A3′ is an optionally present complement of a third additional sequence which is optionally present in the capture oligomer; S′ is an optionally present complement of a spacer which is optionally present in the capture oligomer, A2′ is an optionally present complement of a second additional sequence which is optionally present in the capture oligomer; C2′ is the complement of the second portion of the capture sequence, and X is an optionally present blocking moiety.

Another preferred embodiment is a method of capturing a target polynucleotide from a composition, the method comprising: contacting the target polynucleotide with a capture oligomer as described above and others disclosed in PCT/GB2021/050098, wherein the target-hybridizing sequence of the capture oligomer anneals to the target polynucleotide at a site comprising the 3′ end of the target polynucleotide; extending the 3′ end of the target polynucleotide with a DNA polymerase with strand-displacement activity, thereby forming a complement of the complement of the capture sequence, which is annealed to the capture oligomer, such that the capture sequence of the capture oligomer is available for binding; contacting the capture sequence of the capture oligomer with a secondary capture reagent comprising a complement of the capture sequence and (i) a binding partner or (ii) a solid support, thereby forming a complex comprising the target polynucleotide, the capture oligomer, and the secondary capture reagent; and isolating the complex from the composition, thereby capturing the target polynucleotide.

Another preferred embodiment is a method of capturing a target polynucleotide from a composition, the method comprising: contacting the composition with the combination capture oligomer and complementary oligomer as described above and others disclosed in PCT/GB2021/050098 (any one of claims 28-30 or 34-51), wherein the target-hybridizing sequence of the capture oligomer anneals to the target polynucleotide at a site comprising the 3′ end of the target polynucleotide; extending the 3′ end of the target polynucleotide with a DNA polymerase with strand-displacement activity, thereby forming a complement of the spacer sequence, which is annealed to the capture oligomer, such that the complementary oligomer is displaced to an extent sufficient for the capture sequence of the capture oligomer to be available for binding; contacting the capture sequence of the capture oligomer with a secondary capture reagent comprising a complement of the capture sequence and (i) a binding partner or (ii) a solid support, thereby forming a complex comprising the target polynucleotide, the capture oligomer, and the secondary capture reagent; and isolating the complex from the composition, thereby capturing the target polynucleotide.

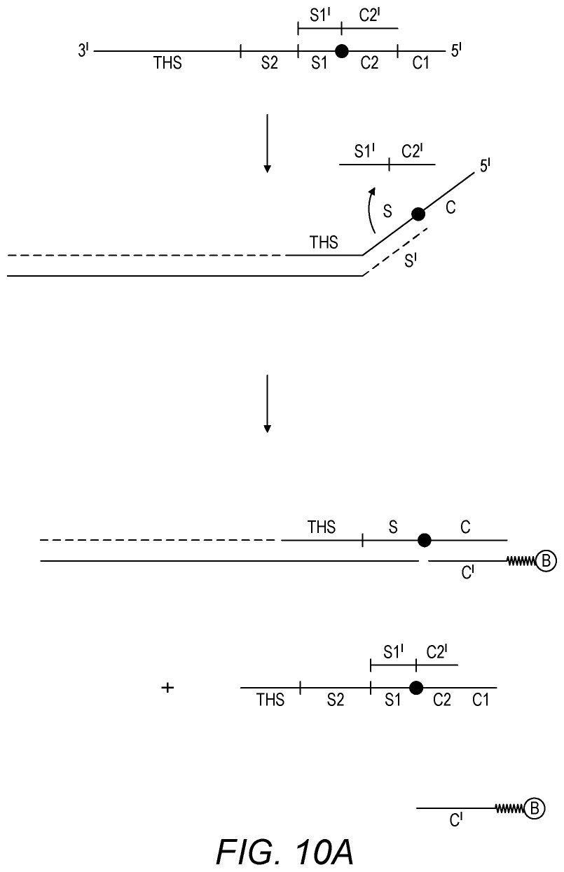

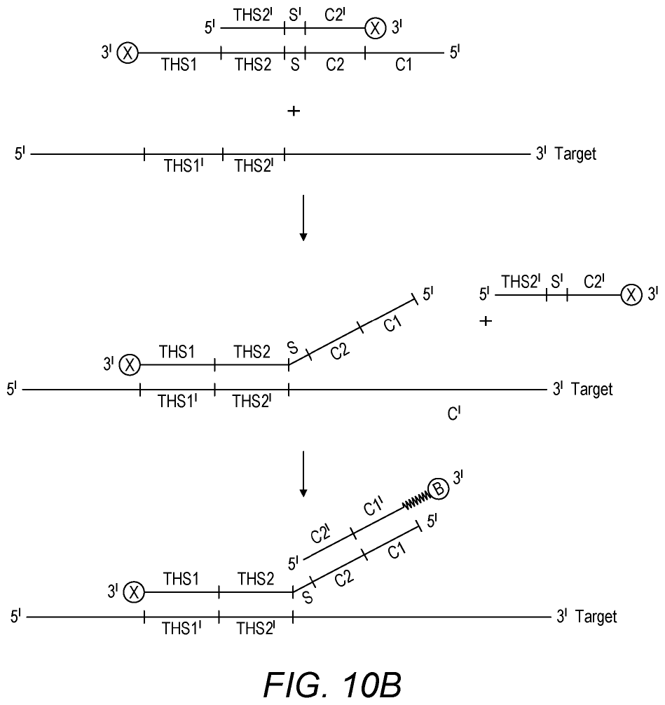

In some other preferred embodiments, a combination is provided, comprising a capture oligomer and a complementary oligomer, wherein:
(a) the capture oligomer comprises, in the 5′ to 3′ direction:
a capture sequence comprising first and second portions, and
a target-hybridizing sequence comprising second and first portions; and
(b) the complementary oligomer comprises, in the 3′ to 5′ direction:
a complement of the second portion of the capture sequence, and
a complement of the second portion of the target-hybridizing sequence, wherein the complement of the second portion of the capture sequence and the complement of the second portion of the target-hybridizing sequence are configured to anneal simultaneously to the capture oligomer in the absence of a complement of the target-hybridizing sequence. FIG. 10B provides an illustration of exemplary oligomers in accordance with these embodiments. Optional additional elements may be present as described in the further embodiments listed above and/or as illustrated in FIG. 10B (e.g., any individual element in FIG. 10B or any combination thereof).

The combination can be used to perform limited capture in that the complementary oligomer can be configured to bind free capture oligomer but not capture oligomer bound to a target polynucleotide. For example, binding of the target-hybridizing sequence of the capture oligomer to the target polynucleotide can be more energetically favorable than binding of the complement of the second portion of the capture sequence to the second portion of the target-hybridizing sequence. In the absence of target polynucleotide, the complementary oligomer binds to the capture oligomer and blocks accessibility to the capture sequence (C1+C2 in FIG. 10B) to a sufficient extent to block binding of the capture sequence by a complement of the capture sequence in a secondary capture reagent, which can be any of the secondary capture reagents described elsewhere herein.

Accordingly, also provided is a method of capturing a target polynucleotide from a composition, the method comprising:
contacting the composition with the combination described above or any further embodiment thereof described herein, wherein the target-hybridizing sequence of the capture oligomer anneals to the target polynucleotide;
contacting the capture oligomer with the complementary oligomer before or after the capture oligomer anneals to the target polynucleotide, wherein the complementary oligomer anneals to free capture oligomer and partially occupies its capture sequence, wherein the complementary oligomer does not anneal to a complex comprising the capture oligomer annealed to the target polynucleotide and wherein if contacting the capture oligomer with the complementary oligomer occurs before the capture oligomer anneals to the target polynucleotide, then the annealing of the target-hybridizing sequence to the target polynucleotide results in dissociation of the complementary oligomer from the capture oligomer;
contacting the capture sequence of capture oligomer complexed with the target polynucleotide with a secondary capture reagent comprising a complement of the capture sequence and (i) a binding partner or (ii) a solid support, thereby forming a complex comprising the target polynucleotide, the capture oligomer, and the secondary capture reagent; and isolating the complex from the composition, thereby capturing the target polynucleotide.

Optional additional elements may be present as described in the further embodiments listed above and/or as illustrated in FIG. 10B (e.g., any individual element in FIG. 10B or any combination thereof).

Another exemplary CC method in the current disclosed invention comprises the following steps: 1) Mix the PCR2 amplicon (see details elsewhere within) with CC reagents comprising a CC capture oligonucleotide as described herein and PCT/GB2021/050098, 2) Incubate the mixture at high temperature (e.g., about 95° C.) for about 3-5 minutes to denature the double-stranded target nucleic acid (amplicon) present, 3) Incubate the mixture at moderate temperature (e.g., about 60° C.) for about 5-10 minutes to promote annealing of the CC capture oligomer to the target, 4) Extend the 3′-end of the amplicon along the CC capture oligomer, thereby displacing the complement to the target capture sequence (the extension can occur during the annealing step when the appropriate reagents are present in the reaction mixture, or as a subsequent step, in which case reagents for extension are added separately), 5) Mix the extension reaction mixture with a secondary capture reagent comprising a complement of the capture sequence, present in a user-defined amount and (i) a binding partner or (ii) a solid support, 6) Incubate at moderate temperature (e.g., about 45° C.) for about 3-10 minutes, thereby forming a complex comprising the target nucleic acid, the capture oligomer, and the secondary capture reagent, 7) isolating the complex from the composition, thereby capturing the target polynucleotide, 8) Wash the beads, 9) Add elution reagent to the washed beads, mix and incubate at ambient temperature (22-26° C.) for 1 minute. The eluted copy-controlled molecules are now ready for further processing in the workflow.

Alternatives to the above exemplary CC workflow include but are not limited to, 1) Conduct the steps above after PCR1 (i.e., when PCR2 is not performed in a given workflow), 2) Conduct the steps above with target amplicon produced by a method other than PCR, 3) During PCR1 or PCR2 (or alternate amplification method), add a universal sequence site as a tag; use this universal tag acts as a universal binding site for the CC capture oligo (advantages include but are not limited to, a) design of only 1 CC capture oligomer for use with multiple targets, b) higher multiplexing capability, c) consistent annealing characteristics across target amplicons, etc.), 4) Add tags (instead of or in addition to the universal sequence tag just described) to amplicons during PCR1 and/or PCR2 (or alternate amp) using a CC-based strategy (as disclosed in, 5) Add all desired tags, including adapters, as part of the CC process. Note that CC methods (including tag addition) generally described above and elsewhere within are disclosed in more detail in GB2021/050098.

### 5. Cluster Generation

By “cluster” is meant a grouping of molecules, e.g., nucleic acid molecules, bound to a solid support. By “cluster generation” is meant the process by which a cluster is produced. Examples of cluster generation processes include amplification-based, such as clonal amplification, and non-amplification-based, such as hybridization of target molecules to an oligonucleotide immobilized to a solid support in a known, specific region (e.g., a spot). A cluster can be monoclonal (typically the preferred configuration in this disclosure) or polyclonal.

One widely accepted method of cluster generation is clonal amplification by performing emulsion PCR of beads. In next generation sequencing (NGS) applications, after clonal amplification the beads are arrayed on the surface of a flow cell (see U.S. Pat. No. 8,012,690B2). This technology has been incorporated in multiple NGS platforms, including the Ion Torrent (Thermo) system, ABI SOLiD and Roche 454. Emulsification encapsulates beads, amplification reagents, and individual template DNA molecules in isolated aqueous droplets (micelles), preventing cross-contamination. While emulsion PCR is a proven technology, the workflow is complex, time consuming (many hours) and difficult to automate in a cartridge format. Other widely accepted methods, such as those developed by Illumina (originally Solexa and Manteia), perform clonal amplification directly on the surface of the flow cell using bridge amplification. The first generation of methods for cluster generation using bridge amplification were based on a form of isothermal polymerase chain reaction facilitated by cycles of reagent flows (isothermal bridge PCR; see U.S. Ser. No. 10/370,652B2 and U.S. Pat. No. 7,972,820B2). In these methods, two PCR primers are co-immobilized to the surface of the flow cell and a population of target DNA molecules containing matching adaptors on both ends is hybridized. Next, denaturation and extension reagents are flown in consecutive cycles, producing discrete clonal clusters of usually less than 1 micron in diameter. These small clusters contain a relatively low number of target nucleic acid copies which makes their use unacceptable with platforms that require higher amounts of target nucleic acid to achieve the required sequencing performance. Similarly to emulsion PCR, isothermal bridge amplification is time consuming (usually more than 4 hours) and requires high reagent volumes. The second generation of methods using bridge amplification is based on Recombinase Polymerase Amplification (RPA) in the form of a method called Exclusion Amplification (ExAmp) (see U.S. Pat. No. 9,169,513B2). RPA is an isothermal DNA amplification method that uses two primers of a similar design to PCR (see U.S. Pat. No. 7,270,981B2). In ExAmp, both primers are immobilized to the surface of an arrayed (patterned) flow cell. Areas between amplification sites are void of primers and are used to prevent mixing between clonal populations. Instead of hybridizing template DNA molecules to the surface before start of the amplification process, a plurality of targets is added together with the amplification mix. As a result, template hybridization and amplification happen at the same time, and cluster clonality is achieved by amplification occurring at a faster rate than hybridization. The process is still relatively time consuming (about 3 hours), however it results is a higher density of clusters, which is beneficial to sequencing output. However, these again are small clusters that contain a relatively low number of target nucleic acid copies.

Apart from emulsion PCR and bridge amplification, another method to create clonal clusters is through Rolling Circle Amplification (RCA). In this method, template DNA molecule is circularized prior to hybridizing with and extending a single amplification primer. This produces long amplicon molecules containing concatemers of target sequence, providing large amounts of DNA for downstream analyses. A sequencing platform developed by BGI and Complete Genomics uses a form of solution-phase RCA to create DNA nanoballs, which are then hybridized to a patterned array (DOI: 10.1126/science.1181498). Creating and manipulating DNA nanoballs requires precision and appropriate quality controls, which complicates application to a rapid, cartridge-based format.

Aside from solution-phase approaches, QIAGEN invested in development of exponential surface-phase RCA for their GeneReader platform (US2018/0105871, US2018/0112251, EP1916311, U.S. Pat. No. 9,683,255B2, US2018/0087099). The method is based on exponential RCA, where two (or more) amplification primers are used rather than one (U.S. Pat. Nos. 5,854,033, 6,143,495, 1101/gr.180501, U.S. Pat. No. 6,323,009). In this approach, a plurality of targets is modified such that they contain adapter sequences on both ends, which are then used to ligate templates into circles. DNA circles are then hybridized to the surface of the flow cell, where two amplification primers are co-immobilized. Amplification primers are complementary to adapters present in the circular template, which allows for a “branched” exponential RCA reaction (DOI: 10.1093/biomethods/bpx007). This protocol contains many steps and can be time consuming. A simpler, faster workflow is needed for effective application to a rapid, cartridge-based system.

### A. Immobilization of Oligonucleotides

Cluster generation on a solid support typically requires immobilization of one or more oligonucleotides or polynucleotides to the said solid support. For the purposes of this disclosure, any method of immobilization that supports the associated step or steps of the disclosed embodiments is acceptable. This includes but is not limited to covalent and non-covalent methods; direct and indirect methods; attaching to specific areas of the surface (e.g., spotting, arrays, etc.) and broadly immobilizing across a surface; attachment to planar surfaces, surfaces comprising features (e.g., wells, pillars, pads), particles, etc.; and the like.

In several preferred embodiments disclosed within, oligonucleotides were attached to the surface of wells fabricated on top of a semiconductor chip. A common method used for such attachment comprises Click Chemistry (see, for example, Click Chemistry, a Powerful Tool for Pharmaceutical Sciences (2008) Hein, et al.; Pharm Res 25(10): 2216-2230 and A Hitchhiker's Guide to Click-Chemistry with Nucleic Acids (2021) Fantoni et al., Chem Rev, 121: 7122-7154). In certain embodiments, the oligonucleotide(s) to be attached comprised a 5′-DBCO=5′-terminal dibenzocyclooctyl (DBCO) moiety (e.g., see example N, “Sequencing of a Template Generated UsingIn-Well ClonalAmphfication”). In such embodiments, one preferred method of conjugation comprises activation of the chip surface with an acrylamide-based polymer coating followed by covalent attachment of the 5′ modified oligonucleotide(s). An exemplary protocol comprises the following general steps: 1) Clean the surface of the semiconductor chip surface by immersion in a 2% Decon 90 solution (Decon Laboratories LTD) for 5 minutes; 2) Rinse the surface with 18 MΩ water followed by a 5 minute incubation in 0.1 M HCl, then rinse again in 18 MΩ water and blow dry with nitrogen; 3) Incubate the chip surface for 30 minutes in a 0.6% wt/vol solution of MCP-click polymer with a 5% azide content in 7% ammonium sulfate (NH₄)₂SO₄; 4) Rinse in 18 MΩ water, blow dry with nitrogen and then bake for 15 minutes at 80° C.; 5) Cool the chip to room temperature; 6) Couple the oligonucleotide(s) to the surface by, A) Microarray spotting—Deposit on the surface spots of the oligonucleotide(s) in 150 mM sodium phosphate buffer pH 8.5 (Na₂HPO₄ and NaH₂PO₄) and sucrose monolaurate 0.01% wt/vol.; in some embodiments spot volumes are about 200-300 μL and oligonucleotide concentrations are about 40-200 μM; spotted samples are incubated for a minimum of about 16 hours at 75% relative humidity; B) Flood filling—An oligonucleotide solution (same buffer composition and oligo concentration as in Method A above) is flooded over the surface of the chip in a flow cell; the input and output ports of the flow cell are sealed and the chip is incubated for a minimum of about 16 hours; 7) Wash the chip (either Method A or B) at 50° C. in 0.1 M Tris buffer ((HOCH₂)₃CNH₂), pH 9 for 15 minutes, then rinse in 18 MΩ water, blow dry with nitrogen and store in the dark at 4% relative humidity until required.

Other conjugation methods that have been successfully demonstrated for use with selected embodiments within include but are not limited to, 1) Polyacrylamide-based coating containing succinimide groups (e.g., CodeLink from Surmodics IVD, Inc) for the conjugation of primary amine derivatized oligonucleotides, 2) A physiosorbed polymer coating containing bromoacetamide (reactive monomer N-(5-bromoacetamidylpentyl)acrylamide) for the conjugation of thiophosphate containing oligonucleotides, and 3) A conjugation approach utilizing UV irradiation and Poly(T)poly(C) 5′-tagged oligonucleotides.

b. Cluster generation using Solution-Mediated Recombinase Polymerase Amplification (SM-RPA)

Herein we disclose Solution-Mediated Recombinase Polymerase Amplification (SM-RPA), which can be used to create clonal nucleic acid clusters from a plurality of targets directly on solid support in typically 20-60 minutes. This is faster than current RPA-based (and other) methods in the art. Additionally, the clusters thus generated create “patches” of clonally amplified nucleic acid of a larger size and a higher number of molecules than the small clusters generated by other methods, which is beneficial for applications requiring higher amounts of the immobilized material.

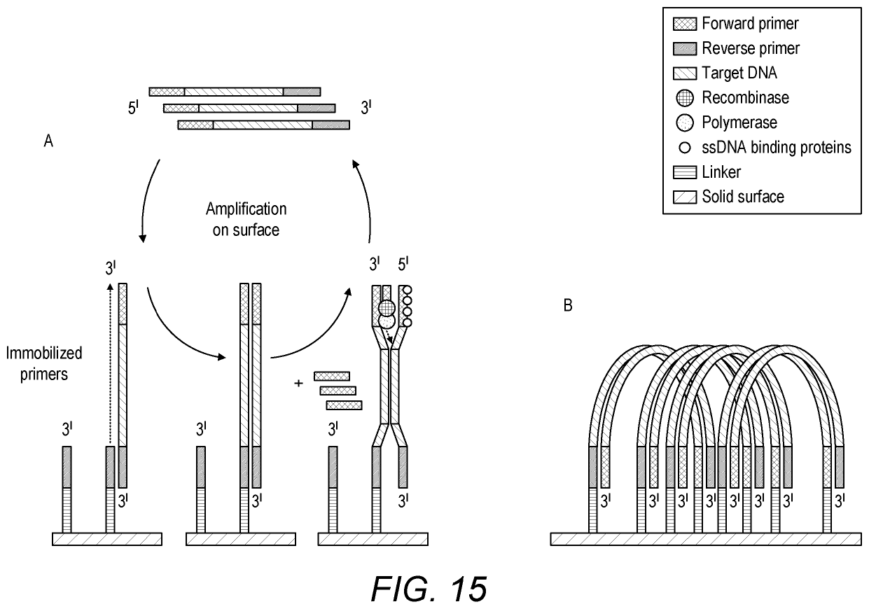

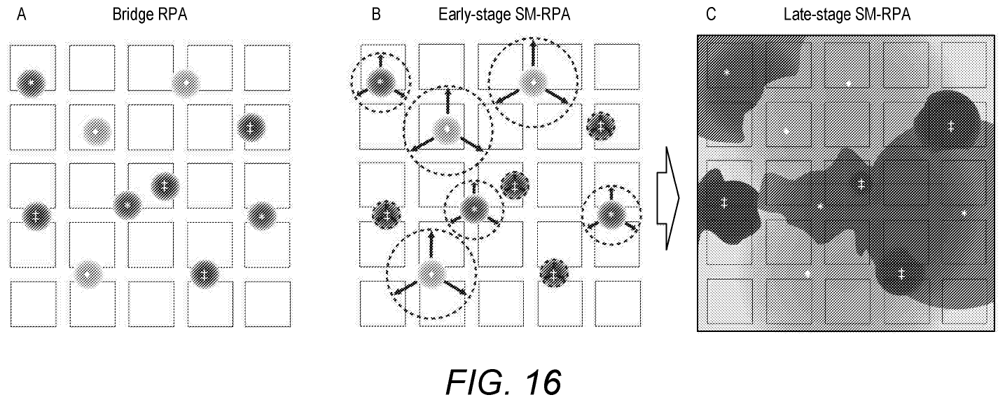

In a preferred embodiment of SM-RPA, one of two primers (e.g, the reverse primer) is immobilized on a solid support and the other primer (e.g., forward primer) is present in the solution phase. As a result, amplification occurs on the surface with strands being released into liquid phase, which may be re hybridized locally. FIG. 15 illustrates the primer configuration and mechanism of SM-RPA and compares this with bridge amplification. The resulting clonal populations present as a “patchwork” of immobilized DNA, as depicted in FIG. 16 where different patches from clonal amplification of 3 different target molecules are shown. The products “grow” laterally, expanding in all directions across the solid surface, until they meet neighboring “patches”. This preferred embodiment has been demonstrated from a plurality of targets on planar surfaces, such as glass slides, and on welled semiconductor chips. Resulting clusters were analyzed using a variety of methods, included post-amp hybridization with fluorescently-labeled probes as well as sequencing using ISFET signal detection. An experimental demonstration of SM-RPA is summarized in Example J, “In-well amplification of target nucleic acids using Solution-Mediated Recombinase Polymerase Amplification (SM-RPA)”.

c. Cluster generation using branched surface-phase Rolling Circle Amplification (RCA)

Herein we disclose a method of Rolling Circle Amplification and related compositions which can be used to create clonal nucleic acid clusters from a plurality of targets directly on solid support. In a preferred embodiment, addition of adapters needed for ligation and amplification is completed transparently during other parts of the workflow. Furthermore, this method allows creation of circularized template directly on the surface of the flow cell, where the ligation splint oligomer acts as the first amplification primer. All these aspects simplify and render the workflow more efficient as well as shortening the duration of the overall workflow, creating an advantage over current on-surface RCA methods.

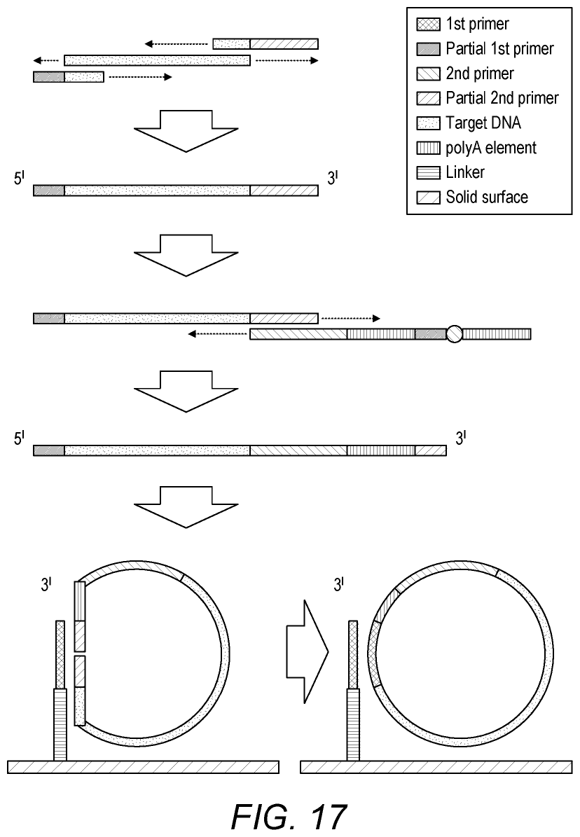

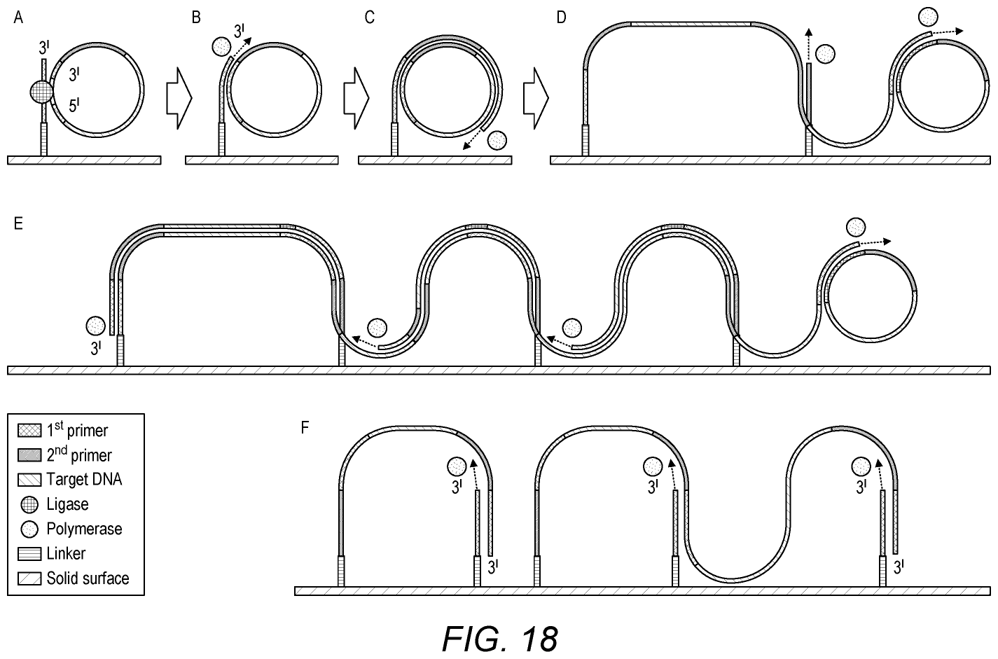

FIG. 17 illustrates a preferred embodiment in which creation of the circularized template for use in clonal amplification occurs transparently across 3 different process steps (see figure legend for description). FIG. 18 illustrates the actual branched surface-phase RCA clonal amplification process (see figure legend for description). This method produces discrete clusters of clonally amplified DNA composed of concatemeric repeats of the target sequences, offering high signal-to-noise ratio. Of further note, both strands of target molecule sequence are produced by extension of the two amplification primers. A specific experiment which demonstrates this embodiment is summarized in Example K, “In-well amplification of target nucleic acids using Rolling Circle Amplification (RCA)”.

d. Cluster generation using hybridization

As stated elsewhere herein, clusters can also be generated using non-amplification-based methods, such as hybridization of target molecules to an oligonucleotide immobilized to a solid support in a known, specific region (e.g., a spot). In a preferred embodiment of the current disclosure, an array of target-specific oligonucleotides are spotted (immobilized) in wells on top of a semiconductor chip. These target-specific oligonucleotides serve as both capture oligomers and sequencing primers. Target polynucleotide sequences are flowed into the wells and monoclonal clusters are formed where target molecules of a given sequence hybridize specifically with the complementary immobilized oligonucleotide. The clusters are then sequenced using the immobilized oligonucleotides as sequencing primers. In a preferred embodiment of the sequencing phase, the semiconductor chip comprises an array of ISFET sensors which serve as the detection modality in the sequencing reaction. A specific experiment which demonstrates this embodiment is summarized in Example M, “Sequencing ofa Synthetic Template Using the Direct Hybridization Method”.

When applicable (i.e., under circumstances where this approach achieves the objectives of the analysis/assay/test), cluster generation using hybridization offers some distinct advantages. For one, the clonal amplification step is not required, thereby typically saving steps, reagents and time. For another, in a number of applications the copy control step would not be required either, again typically saving steps, reagents and time. In still other circumstances, the library preparation step could be simplified, e.g., less amplification required, fewer or no tag/adapter steps required, could potentially forego a purification step, etc. In circumstances where target polynucleotide is plentiful and the sample type creates little or no inhibition, it may even be possible to go directly from sample to cluster generation, and then analysis (e.g., sequencing). Also, if sequencing is the selected analysis method, the steps of denaturing the target and annealing the sequencing primer typically performed after clonal amplification would not be required as the capture oligomer serves as the sequencing primer as well.

### 6. Sequencing

Preferred embodiments of the current invention conduct sequencing using a semiconductor chip comprising an array of ISFET sensors (for example, see U.S. Pat. No. 7,686,929 B2). The chip also typically comprises an array of wells attached to the surface of the chip and positioned over the ISFET sensors. A preferred method of sequencing generally comprises the following steps (sequencing by synthesis): 1) Immobilize the nucleic acid template to be sequenced in one or more wells (methods of immobilization in wells include clonal amplification and direct hybridization; see elsewhere within for details); 2) Anneal a sequencing primer to the template (in some embodiments, annealing of the sequencing primer is part of the immobilization step; 3) Bind sequencing enzyme (polymerase) to the template/sequencing primer complex (disclosed below is also a new method by which sequencing primer and enzyme are added in the same step); 4) Flood the wells with a given dNTP (in some cases, multiple dNTPs can be included together in a single flow); 5) Incubate (in some cases the flow of dNTP stops and in other cases it does not); if the complementary nucleotide is present in the template, the introduced dNTP will be incorporated and protons (1 per nucleotide incorporation) will be released and the ISFET(s) under the well(s) will detect this release; 6) Wash; Repeat steps 4-6 with each of the other dNTPs; this completes one “cycle” (other cycle configuration can also be utilized); 7) Repeat cycling to obtain the sequence of the template.

### A. Concurrent Annealing of Sequencing Primer and Binding of Sequencing Enzyme

As summarized above, annealing of sequencing primer to the nucleic acid template and binding of sequencing enzyme to the resulting complex are performed in separate steps. This is because high temperatures (up to about 95° C.) are required for the annealing step (which typically also comprises denaturation of the template, which is often double stranded at this point in the workflow) and sequencing enzymes are not stable at these temperatures. We disclose a method by which the 2 steps are combined, comprising use of a thermostable sequencing polymerase (e.g., Tin(exo-) LF DNA Polymerase from Optigene). Briefly, all components required for sequencing primer and enzyme are combined in one reaction mixture with the template. The temperature is increased (e.g., (up to about 95° C.) to denature the double stranded template then lowered (e.g., to about 60° C.) to support annealing of the sequencing primer and subsequent binding of the sequencing enzyme to the primer/template complex (the binding of the sequencing enzyme may also occur, at least to a degree, later in the process if and when the temperature is lowered even more, e.g., between about 20 and 45° C.).

B. Sequencing after Cluster Generation by Clonal Amplification

In one preferred embodiment of a sequencing workflow comprising a semiconductor chip as described above, sequencing is performed after cluster generation by clonal amplification (see Section III.B.5 above and elsewhere herein). Also as described elsewhere herein, cluster generation is followed by sequencing primer hybridization and sequencing enzyme binding and then sequencing. In some embodiments, the sequencing primer may be universal to all potential targets, utilizing a universal primer binding site incorporated into the template during earlier steps of the process (e.g., library preparation). Alternatively, a multiplex of sequencing primers may be used to initiate sequencing from specific targets. Experiments exemplifying this mode are summarized in Example N, “Sequencing of a Template Generated Using In-Well Clonal Amplification”.

C. Sequencing after Cluster Generation by Direct Hybridization

In another preferred embodiment of a sequencing workflow comprising a semiconductor chip as described above, sequencing is performed after cluster generation by direct hybridization (see Section III.B.5 above and elsewhere herein). Also as described elsewhere herein, in the direct hybridization method the immobilized capture oligomer also serves as the sequencing primer. Therefore, steps of the method after template immobilization (hybridization) comprise sequencing enzyme binding and then sequencing. Experiments exemplifying this mode are summarized in Example M, “Sequencing of a Synthetic Template Using the Direct Hybridization Method”.

### D. Key Sequences

i. Introduction

For semiconductor sequencing, using a known sequence of DNA to calibrate base calling parameters, such as setting thresholds for null incorporation events (i.e., “0-mers”), single-base incorporation events (i.e., “1-mers”) and homopolymer incorporations (i.e., “2-mers”, “3-mers”, etc.), is commonly achieved through the use of a universal key sequence (see for example Genome sequencing in microfabricated high-density picolitre reactors (2005) Marguilies, et al., Nature, 437:376-380). This known sequence can be incorporated into the template at an earlier stage of the workflow (e.g., tag/adapter addition during library preparation). Although such methods are in common practice, they typically add reagent cost, complexity and time to the workflow. We disclose herein two alternative methods for introducing key sequences into a sequencing workflow.

### ii. Embodiment #1. Target Specific Key Sequences

In the first approach, a section of the target itself may be used as a calibrating key sequence. For example, where an upstream amplification step employs the use of primers to enrich particular regions (AKA targeted enrichment), the sequence of the primer itself is typically known. When that is the case it is possible to use part of the known sequence of the primer as a key sequence to determine thresholds and other signal processing parameters. This can be achieved through the truncation of the oligonucleotide that will be used as a sequencing primer, i.e., the surface immobilized capture oligomer/primer in the direct hybridization method of cluster generation followed by semiconductor sequencing.

Where an array of target specific, surface immobilized capture oligomers/primers are used for the direct hybridization approach, each capture oligomer/primer may be truncated by x-bases at its 3′end (i.e., the end distal to the surface). The value of x can be determined specifically for each oligomer based on the sequence. The bases that are truncated will form the key sequence for each specific primer, hereafter referred to as the Target Specific Key Sequence. Ideally, the Target Specific Key Sequence will generate at least 0-mer, 1-mer and 2-mer outputs (see “Introduction” paragraph above). However, it is extremely unlikely that every, or indeed any, primer in a given panel will have a sequence that can generate such outputs. Thus, the exact Target Specific Key Sequence must be determined on a case-by-case basis, according to the characteristics deemed to be most desirable for particular signal processing approaches.

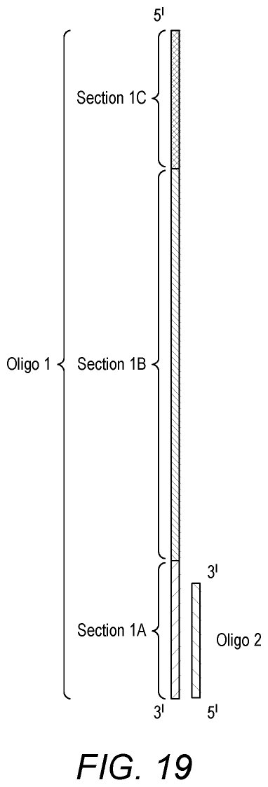

FIG. 19 shows an example of a basic Target Specific Key Sequence. For Oligo 1, Section 1A and 1C describe, respectively, the forward and reverse primer binding sites used for targeted upstream amplification. The specific sequence of these sections is therefore known. Oligo 1, Section 1B describes the region of interest, i.e. the unknown section of the template which is to be sequenced. Note that for the purposes of this example, the actual sequence of Oligo 1, Sections 1B and 1C are not relevant and therefore not described. Oligo 2 is designed to be complementary to Oligo 1, Section 1A, with the notable exception of the nucleotide truncation at the 3′ end. In the embodiment described here, Oligo 2 is immobilized to a solid support. Oligo 1 is provided in solution and Section 1A thereof, with the exception of the key sequence nucleotides on the 5′-end, hybridizes specifically to Oligo 2.

Table E below provides example sequences for use in this embodiment. Note that the final base of the known primer sequence, i.e., the final base at the 5′ end of Oligo 1, Section 1A, may not be usable as part of the key sequence. This is because there is a possibility that this base will be identical to the first base of the unknown section (Oligo 1, Section 1B) that is to be sequenced. Thus, it is known that there will be an incorporation of said base but it is unknown whether this will be a 1-mer incorporation or a homopolymer incorporation event, and therefore the incorporation event may not be useful for threshold setting and incorrectly using it as such may result in errors.

TABLE E
Example of oligonucleotide sequences applicable for use in the scheme
shown in FIG. 19.

| Name/Description | Sequence |
|---|---|
| Oligo 1 Section 1A (SEQ ID No. 1) | 3′ CCGTACGGATTGTGTACGTTCA 5′ |
| Oligo 2 (SEQ ID No. 2) | 5′ GGCATGCCTAACACA 3′ |
| Truncated (removed) section of Oligo 2 | 5′ TGCAAGT 3′ |
| Target specific key sequence for Oligo 2 | 5′ TGCAAG 3′ |

Following 3′ truncation, the surface-phase primer may no longer possess the same characteristics important for hybridization with template as it did prior to truncation, such as nucleotide length, melting temperature (Tm) and percentage GC content (% GC). In particular, the melting temperature will now be lowered due to the removal of bases. If a panel of primers are truncated to varying amounts to provide optimal Target Specific Key Sequences, then the template hybridization kinetics and thermodynamics may be significantly altered such that the overall efficiency of hybridization may be reduced. Where a multiplex of templates are designed to hybridize to a multiplex of surface capture oligomers/primers, the altered hybridization properties may lead to differential hybridization efficiency across the panel. In this case, it may be necessary to further modify the surface capture oligomer/primer to counteract the effects of its 3′ truncation. This may be achieved in a number of ways, including but not limited to, 1) The addition of bases at the 5′ end, i.e., proximal to the surface, to increase primer length and increase melting temperature, 2) The use of nucleic acid analogues such as locked nucleic acids (LNAs) or peptide nucleic acids (PNAs) within the surface bound capture oligomer/primer to elevate the melting temperature.

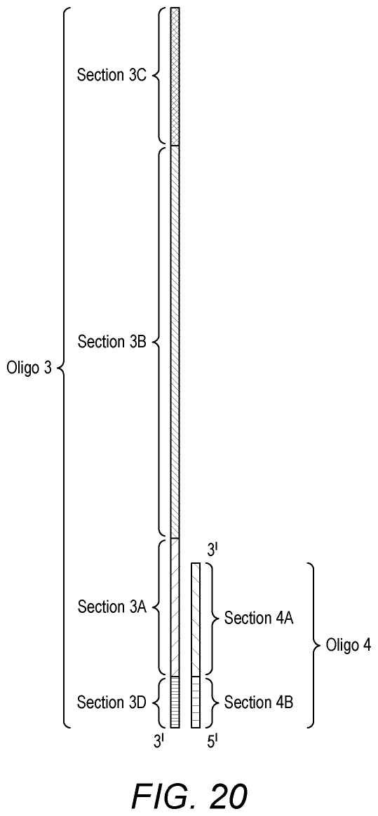

FIG. 20 provides another yet similar example of the Target Specific Key Sequence approach. Oligo 3 Sections 3A and 3C describe, respectively, the forward and reverse primer binding sites used for targeted upstream amplification. In this example, a tailed primer approach has been employed during upstream amplification to extend Oligo 3 with the additional, synthetic Section 3D. This section is not target specific, and therefore Section 3D may be either (a) universal, or (b) target specific, as required. In this method, the additional section 3D is specifically designed to counteract the impact of the 3′ truncation required to generate the Target Specific Key Sequence. Oligo 3, Section 3B describes the region of interest, i.e. the unknown section of the template which is to be sequenced.

Oligo 4 is designed to be complementary to Oligo 3, comprising complementarity to both the target specific section (Oligo 3, Section 3A) and the additional section (Oligo 3, Section 3D) of Oligo 3. The truncation at the 3′ end enables generation of the Target Specific Key Sequence.

In the embodiment described here, Oligo 4 is immobilized to a solid support. Oligo 3 is provided in solution and Sections 3A and 3D thereof, with the exception of the key sequence nucleotides on the 5′-end, hybridizes specifically to Oligo 4.

Table F below provides example sequences for use in the aspect of this embodiment depicted in FIG. 20.

TABLE F
Example of oligonucleotide sequences applicable for use in the scheme shown
in FIG. 20.

| Name/Description | Sequence |
|---|---|
| Oligo 3; Section 3A (SEQ ID No. 3) | 3′ CCGTACGGATTGTGTACGTTCA 5′ |
| Oligo 3; Section 3D | 3′ GGC 5′ |
| Oligo 4; Section 4A and 4B (SEQ ID No. 4) | 5′ CCGGGCATGCCTAACACA 3′ |
| Oligo 4; Section 4A (SEQ ID No. 5) | 5′ GGCATGCCTAACACA 3′ |
| Oligo 4; Section 4B | 5′ CCG 3′ |
| Truncated (removed)section of Oligo 4 | 5′ TGCAAGT 3′ |
| Target specific key sequence for Oligo 4 | 5′ TGCAAG 3′ |

### iii. Embodiment #2. Immobilized Universal Key Sequences

In the second approach, an additional 5′ section of the surface immobilized capture oligo/primer may be used to generate a key sequence by enabling polymerization from the 3′ end of the hybridized template whilst temporarily preventing polymerization from the 3′ end of the surface immobilized oligo.

In this embodiment, rather than being truncated the 3′ end of the surface-bound capture oligomer/primer is equipped with a reversable blocking chemical group to prevent polymerization. Examples of such blocking groups include but are not limited to: 3′-O-(2-nitrobenzyl), 3′-hydroxyamine and 3′-O-azidomethyl. At the 5′ end, the surface-bound capture oligomer/primer comprises an additional, known sequence that is relevant for use as a key sequence (see discussion of possible key sequence elements above). Since this known sequence is additional, it will have a limited influence at most on the specificity of template hybridization to the sequence-specific section of the surface-bound capture oligomer/primer. Therefore, it can be common or universal to all surface-bound capture oligomer/primers in the panel. Upon template hybridization, a sequencing polymerase enzyme is added. Since the 3′-end of the surface immobilized capture oligomer/primer is blocked, sequencing will not be initiated from this terminus. However, the 3′ end of the template is unmodified and thus the sequencing reaction will be initiated, using the additional 5′ section of the immobilized capture oligomer/primer as a template. The sequencing output from this additional 5′ section of the surface-bound capture oligomer/primer will serve as a universal key sequence for setting relevant signal processing and base calling parameters.

Once sequencing of the 5′ section is complete, the 3′ blocking moiety is reversed/removed using an appropriate method. For example, the 3′-O-(2-nitrobenzyl) group can be photocleaved with exposure to 340 nm light, 3′ hydroxyamine can be deblocked with aqueous sodium nitrite, and 3′-O-azidomethyl can be removed by reduction with tris(2-carboxyethyl)phosphine. Following removal of the blocking moiety, additional polymerase is added to ensure all primer-template complexes are bound with polymerase, and sequencing is re-started from the unblocked 3′ end of the surface-bound capture oligomer/primer. The sequencing data thus generated is analyzed using the signal processing and base calling parameters set using the data from the preceding sequencing of the universal 5′ section of the capture oligomer/primer itself.

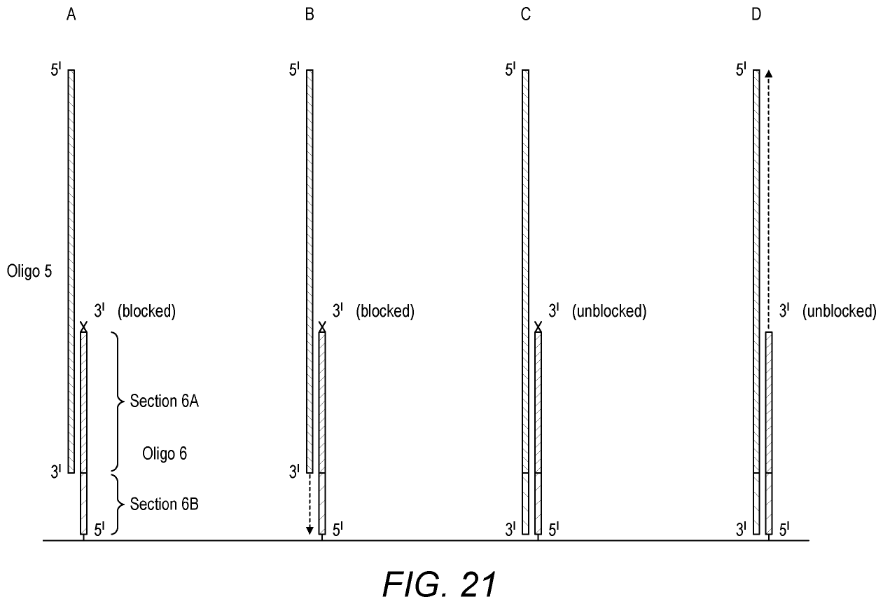

A diagrammatic representation of the this second key sequence embodiment is shown in FIG. 21. In step (a), sequencing target Oligo 5 hybridizes specifically to the fully complementary Section 6A of Oligo 6. Section 6A may be identical to a primer used upstream in the workflow to amplify the target. Oligo 6 is reversibly blocked at the 3′ end, preventing extension. Upon addition of a polymerase, the 3′ end of Oligo 5 is able to polymerize using Section 6B of Oligo 6 as a template. Section 6B may be designed to be used as a key sequence, such that as sequencing progresses through this section, 0-mer and 1-mer and other nucleotide incorporation events are reported as required. Once sequencing of Section 6B is complete, the blocking moiety at the 3′ end of Oligo 6 is removed, as per Step (c). Upon further polymerase addition, if required, sequencing now progresses from the 3′-end of Oligo 6 through the unknown target region of Oligo 5.

### 7. System

Disclosed throughout are embodiments useful for the rapid analysis of target polynucleotides, including determining the nucleotide sequence thereof, from a wide range of input sample types and amounts using an automated system. The system comprises an instrument and at least one assay cartridge removably insertable within the instrument. In preferred embodiments, the system further comprises at least one reagent cartridge removably insertable within the instrument. In still further preferred embodiments, the system comprises a semi-conductor chip, wherein in some embodiments the chip is embedded within the cartridge. In particularly preferred embodiments, the system still further comprises a flow cell mounted on top of the chip (with or without wells) useful for the delivery of fluids to the chip and removal of fluids therefrom. In preferred embodiments, the system comprises software. In particularly preferred embodiments, the software comprises operational software (for controlling the system) and analytical software (for receiving, processing and analyzing the output of the system).

### A. Semiconductor Chip

Use of a semiconductor chip comprising a field effect transistors (FET) array for sensing chemical and/or biological reactions, including sequencing reactions, is well known in the art (e.g., U.S. Pat. No. 7,686,929 B2; U.S. Pat. No. 8,685,228 B2; U.S. Pat. No. 8,986,525 B2; US 2010/0137143 A1). In the present disclosure, a preferred embodiment comprises a semiconductor chip comprising an ion sensitive field effect transistor (ISFET) array useful as a sensing device for various reactions, including nucleic acid sequencing reactions. In particularly preferred embodiments, the chip further comprises an array of wells positioned above the ISFET array and in fluid contact therewith. In these embodiments, the sequencing reaction typically occurs within the wells and the release of ions is detected by the ISFET sensors. In particularly preferred embodiments, the chip still further comprises a flow cell mounted on top of the chip (with or without wells) useful for the delivery of fluids to the chip/ISFET array and removal of fluids therefrom. In preferred workflow/system embodiments, the chip is integrated into the cartridge, within which the entire workflow is performed.

### B. Assay Cartridge

As used within, the terms “assay cartridge” or “sample cartridge” refer to a device in which the steps of a particular test, assay or segments thereof are performed. Typically such a device comprises chambers which are in fluid connection with one another to various degrees.

Disclosed within are exemplary designs of assay cartridges useful for performing an entire sequencing workflow from sample entry to result. A wide variety of sequencing-based assays as well as other complex assays can be performed within such disclosed cartridges. In one set of embodiments, different phases of the workflow are performed in “sub-system” cartridges, while in another set of embodiments all phases of the workflow are performed in an integrated cartridge. In some cases, an assay may be performed in conjunction with a reagent cartridge (exemplary designs also enclosed within). Typically tests are performed in such cartridges in conjunction with an instrument, but in some cases tests and cartridge configurations that require no automation or minimal automation can be performed.

### I. Sub-System Cartridges

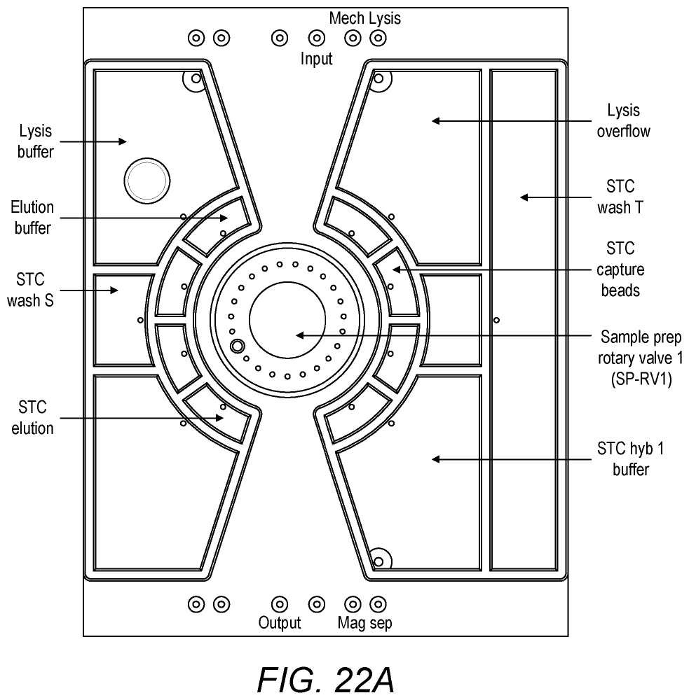

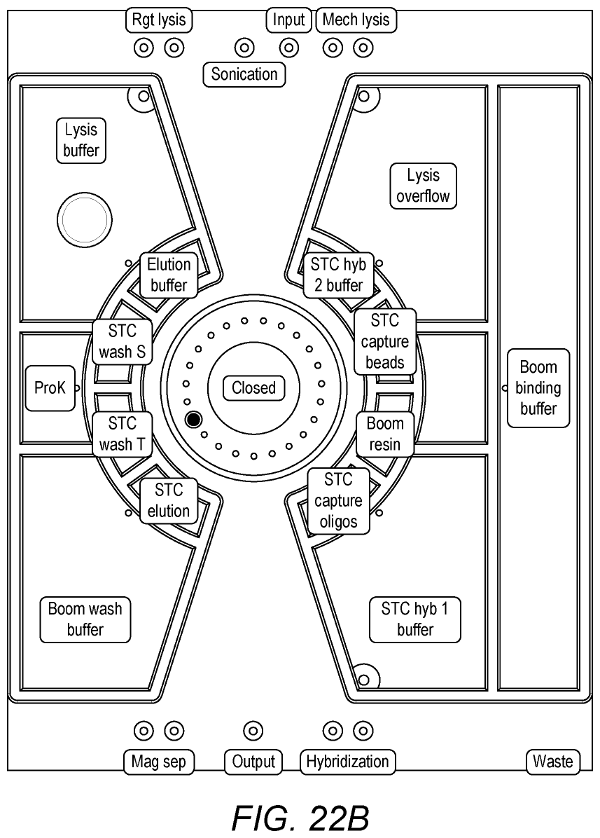

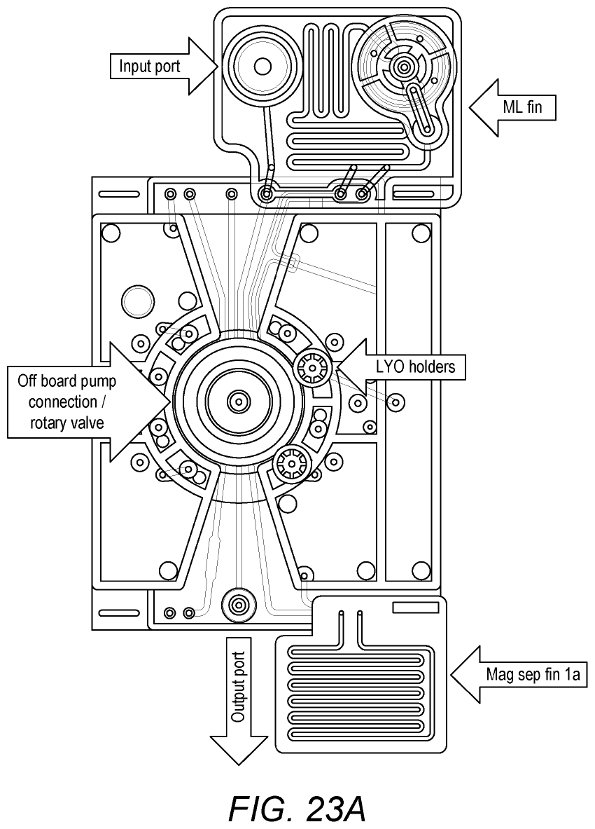

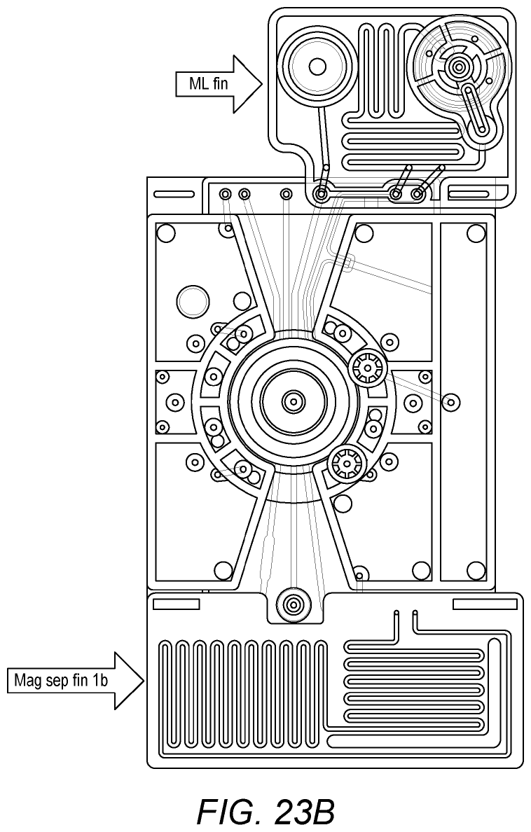

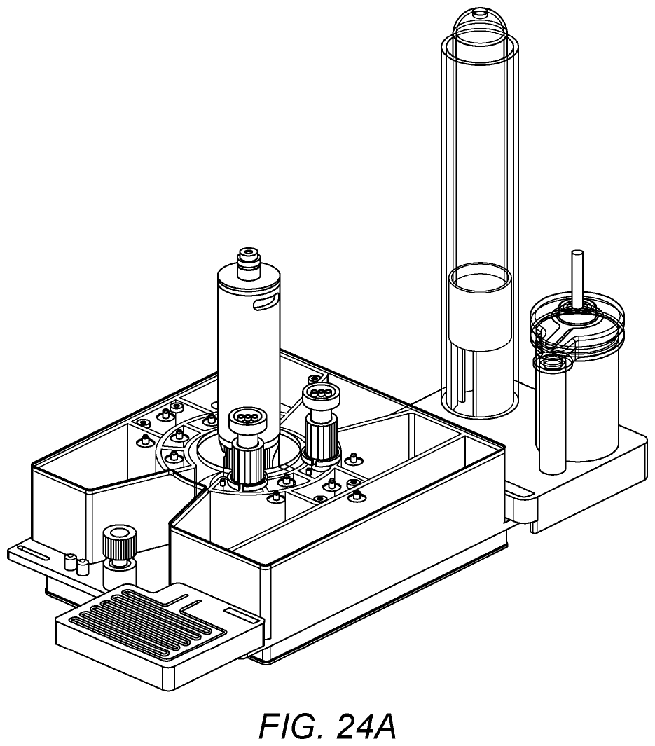

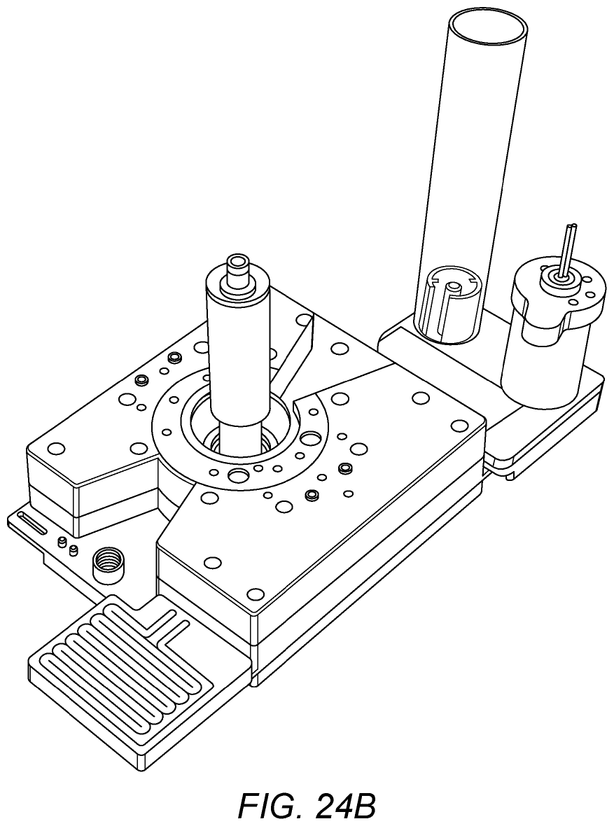

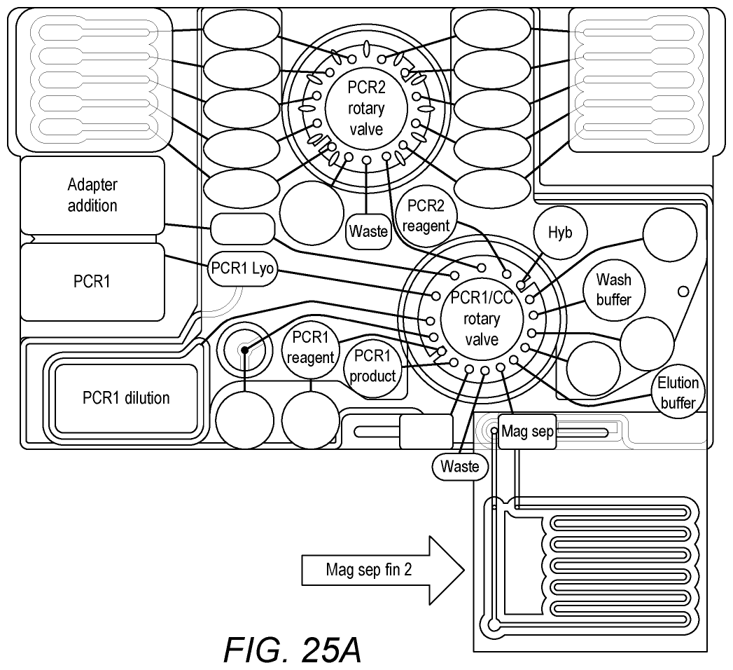

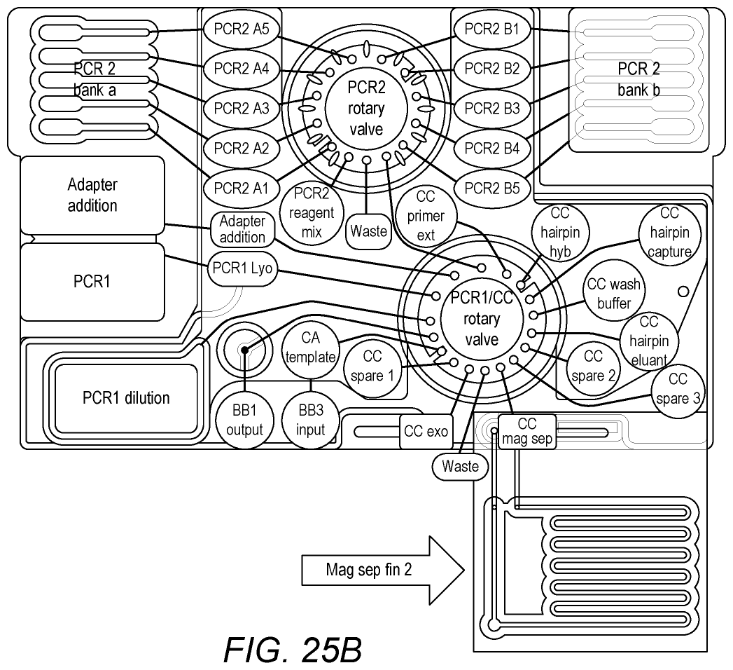

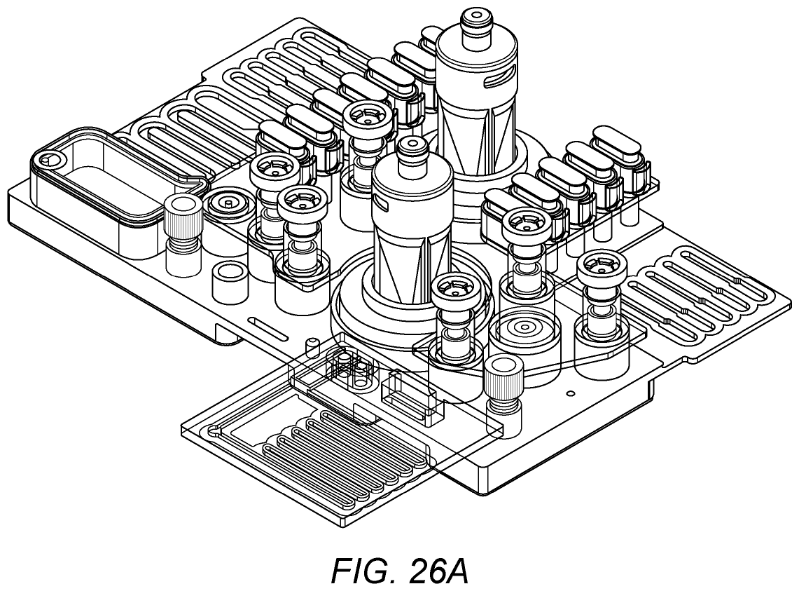

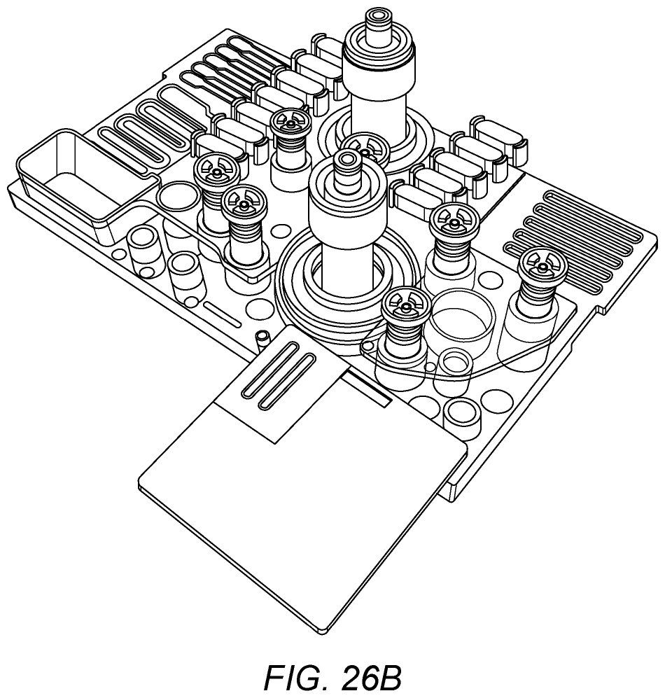

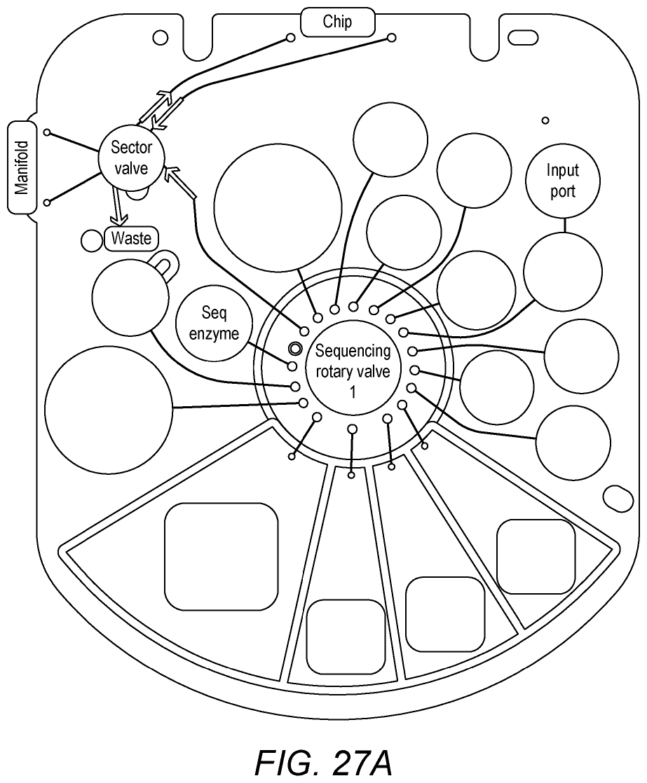

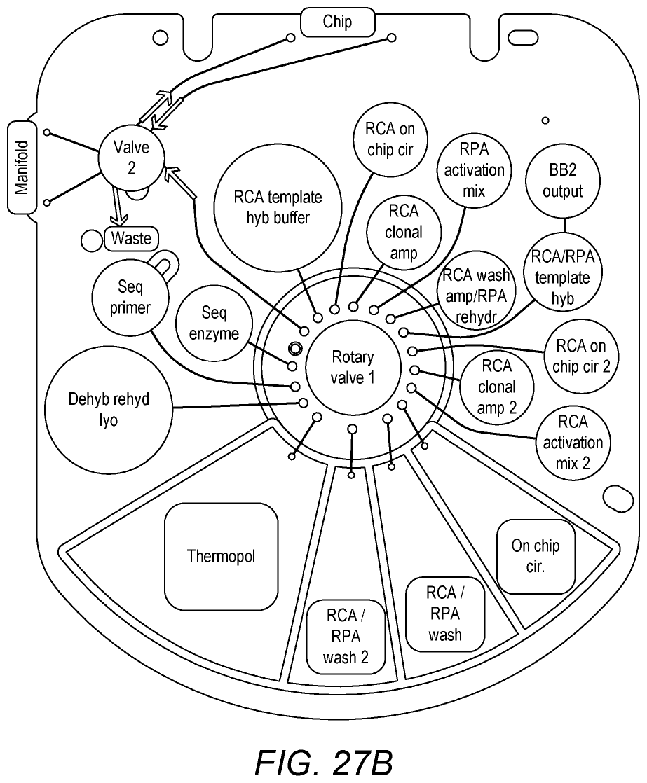

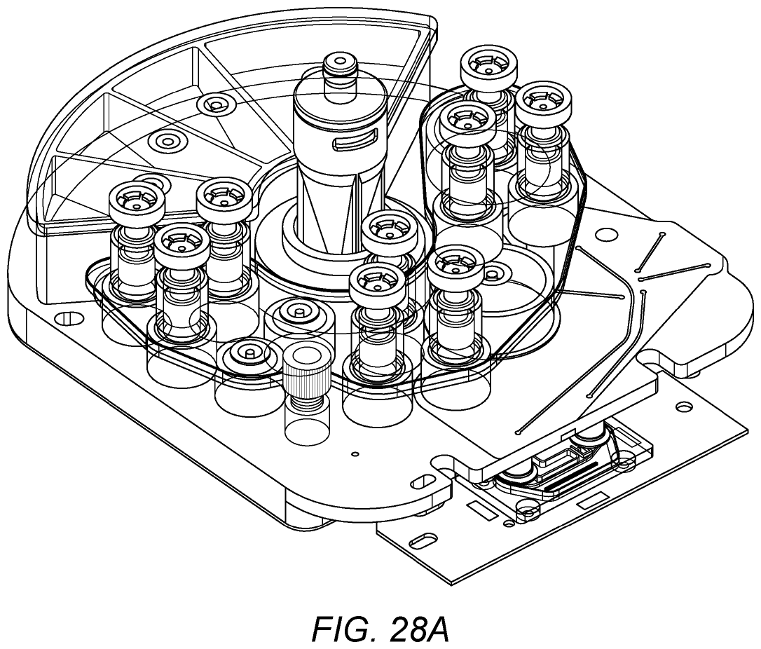

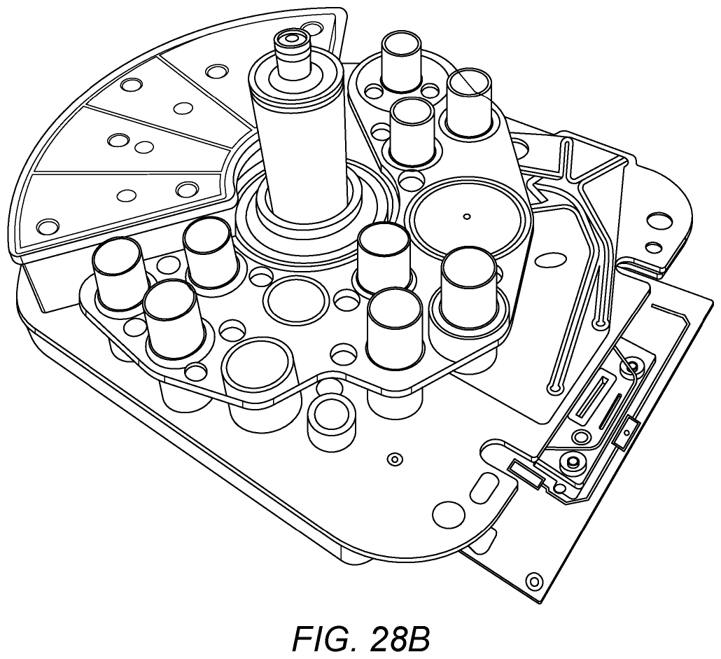

Disclosed within are exemplary designs for sample preparation (see, for example, FIGS. 22 through 24), library preparation (including copy control; see, for example, FIGS. 25 through 26) and cluster generation/sequencing (see, for example, FIGS. 27 through 28). In some cases, these processes (sample preparation, library preparation, cluster generation/sequencing) may be performed individually or in various combinations, depending on the overall goals and requirements of the test being performed. In some cases where 2 or 3 of the cartridges are used for a given test, the output of one cartridge can be transferred manually into the next cartridge, or the transfer can be performed automatically with the aid of an instrument.

Key features of the sample preparation cartridge designs include but are not limited to, 1) A flexible input sample system that can accommodate relatively large sample volumes; 2) A unique design chamber layout that allows accommodation of relatively large sample and assay volumes in a relatively compact cartridge; 3) A fully integrated rotary valve that can withdraw and deliver fluids to a large number of chambers and channels; 4) Separate but connected appendages or “fins” that can perform complex operations and yet afford flexible cartridge design between assay types; 5) Serpentine channels to improve heating and magnetic separation characteristics; 6) Chambers and associated features to allow on-cartridge storage of both liquid and dry reagents; 7) A large number of chambers, again within the context of a relatively compact cartridge, thus allowing a large number of variations of sample preparation protocols to be performed.

Key features of the library preparation cartridge include but are not limited to, 1) Two embedded rotary valves, supporting complex assay flows with delivery and withdrawal of fluids in multiple combinations of chambers and channels; 2) Multiple amplification chambers/stations, allowing multiple reactions to accommodate complex workflows, diluted samples, high levels of multiplexing, etc.; 3) Chambers and associated features to accommodate tag/adapter addition; 4) Chambers and features to accommodate copy control; 5) A separate but connected appendage or “fin” that can perform complex operations and yet afford flexible cartridge design between assay types (one or more additional fins can easily be added); 6) Serpentine channels to improve heating and magnetic separation characteristics; 7) Chambers and associated features to allow on-cartridge storage of both liquid and dry reagents; 8) A large number of chambers, again within the context of a relatively compact cartridge, thus allowing a large number of variations of library preparation protocols to be performed.

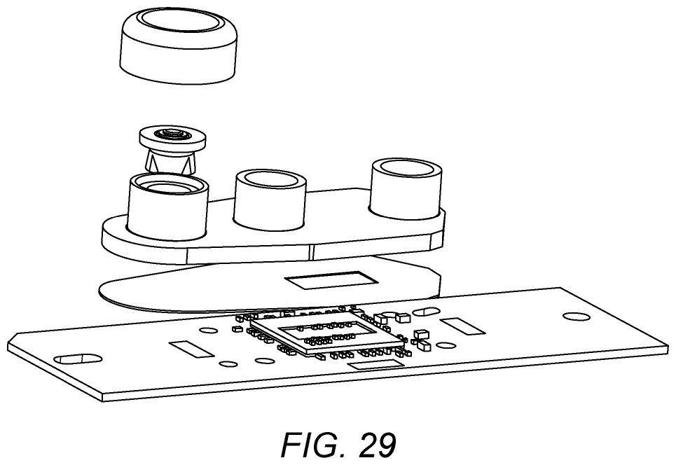

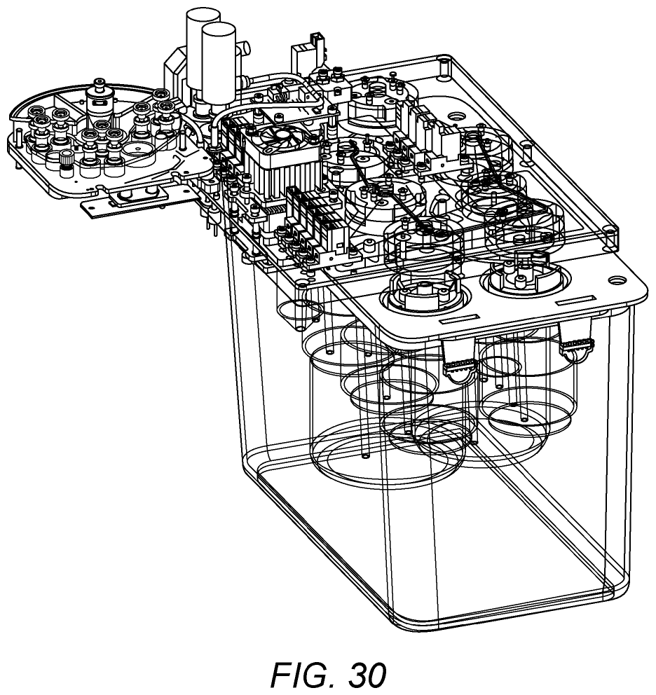

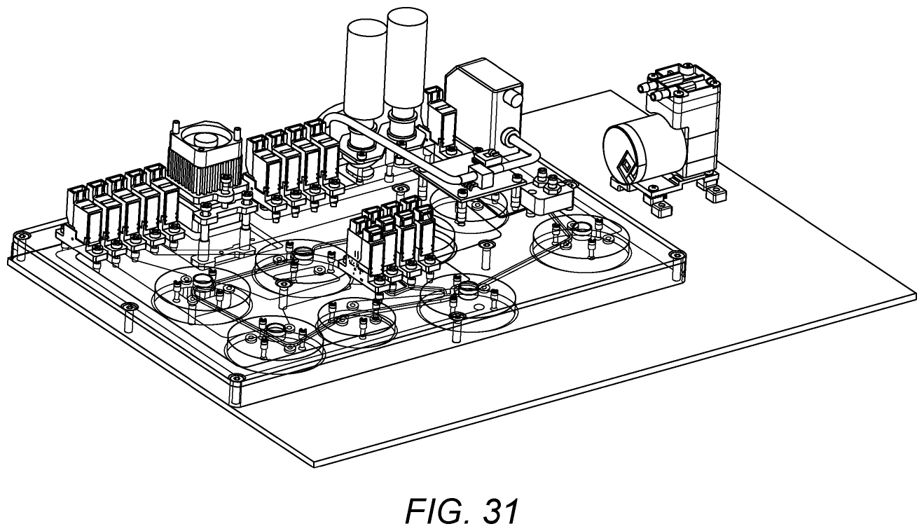

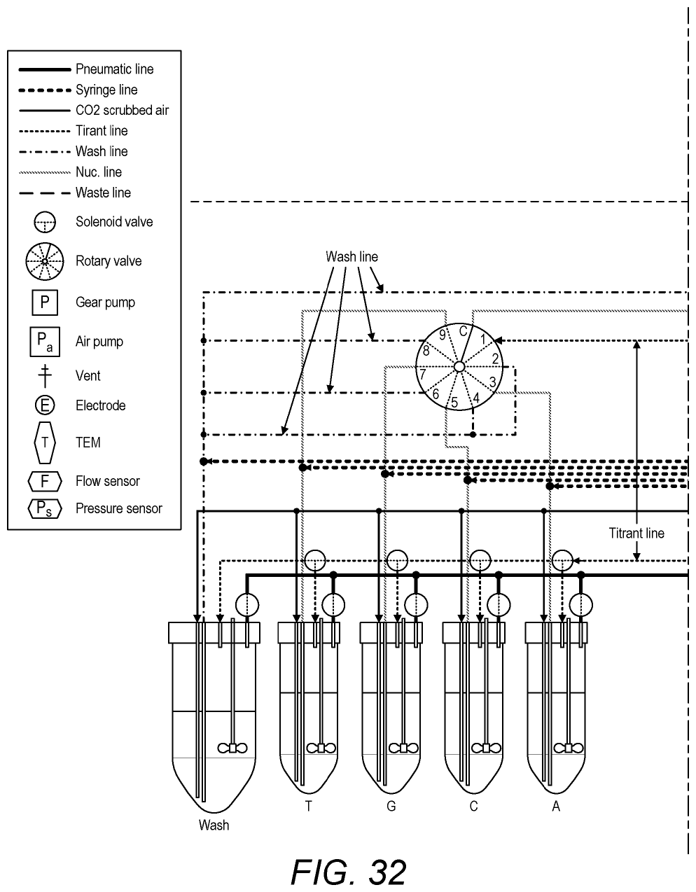

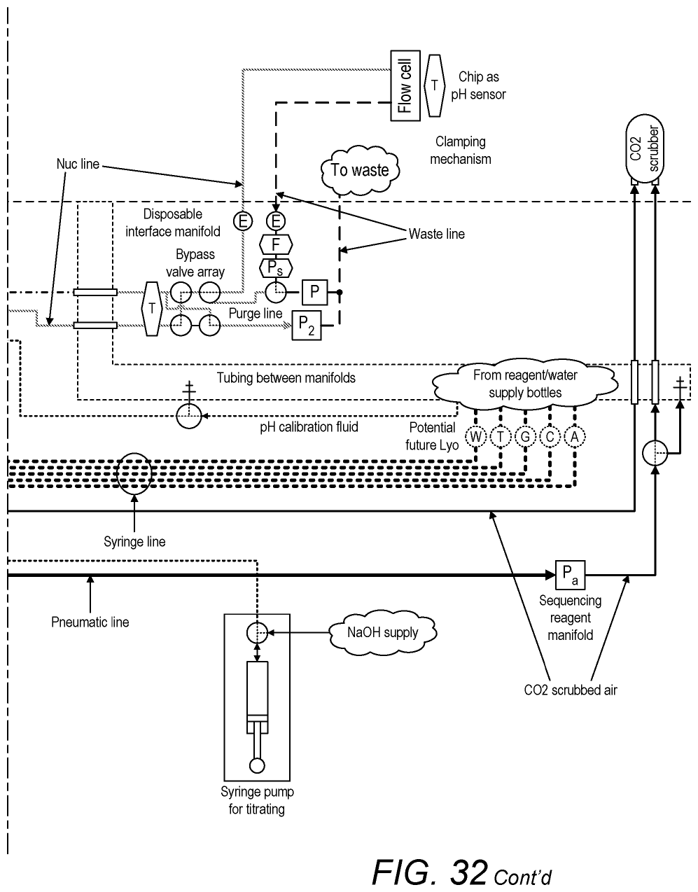

Key features of the cluster generation/sequencing cartridge include but are not limited to, 1) A fully integrated rotary valve that can withdraw and deliver fluids to a large number of chambers and channels; 2) An onboard “selector” valve, in addition to the rotary valve, to afford even more flexibility and options for fluid delivery; 3) A large number of chambers in a relatively compact format that can support multiple forms of cluster generation as well as sequencing; 4) Chambers and associated features to allow on-cartridge storage of both liquid and dry reagents; 5) Full fluid connectivity to a flow-cell for delivery and withdrawal of fluids from a solid support (which in preferred embodiments comprises a semi-conductor chip; see, for example, FIG. 29); 6) Full fluid connectivity, when selected as an option, to a reagent cartridge (see elsewhere within) for delivery of larger volumes of required fluids, such as sequencing reagents (e.g., dNTP solutions)(see, for example, FIGS. 30, 31 and 32).

Details of one particular assay—in this case detection of a pathogen(s) from blood-performed across the above described 3 sub-system cartridges is given in Example O, “Automated sample-to-answer sequencing of pathogen spiked into whole blood”.

ii. Integrated cartridge

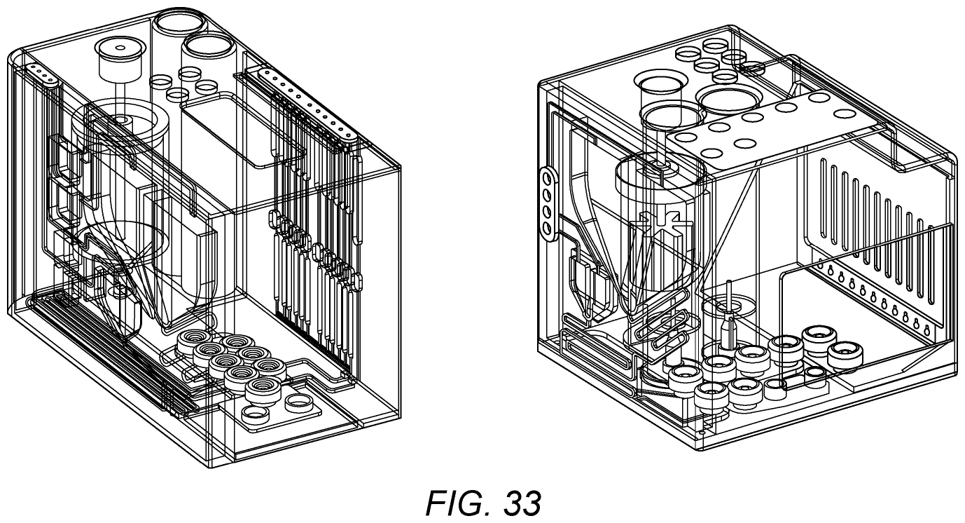

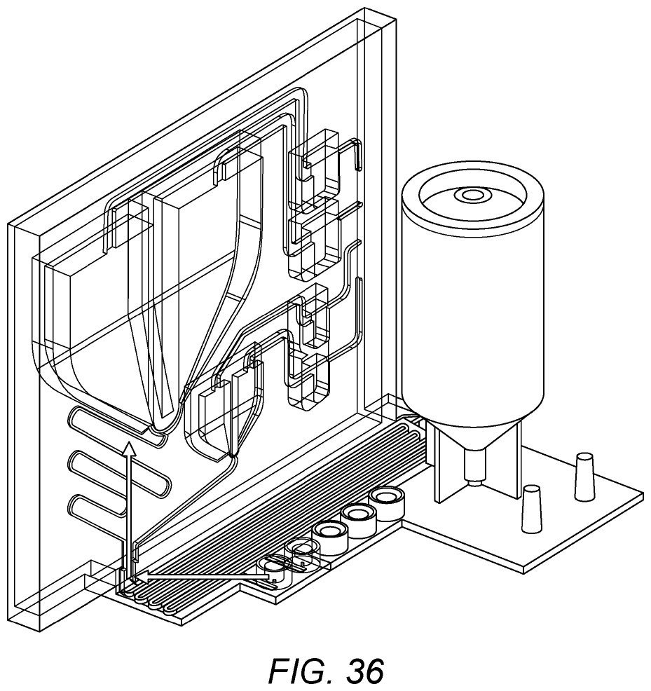

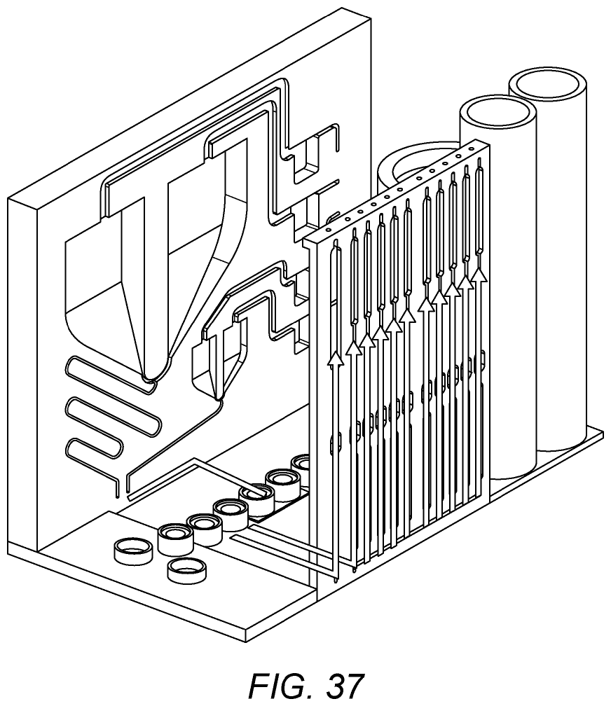

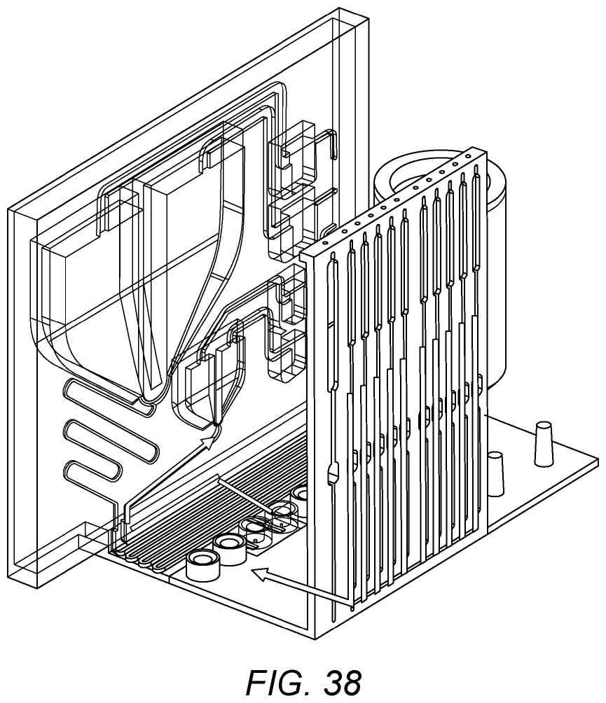

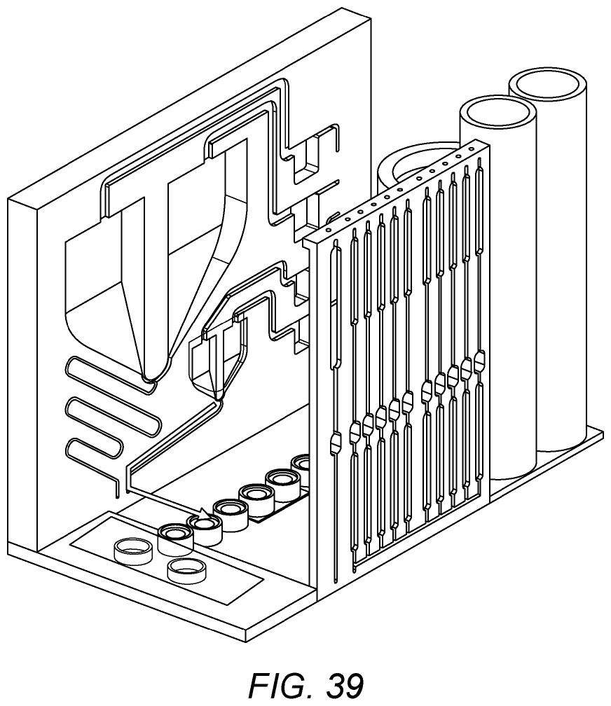

Disclosed within are also exemplary designs for a fully integrated cartridge in which all steps of a workflow can be performed, including sample preparation, library preparation, copy control, cluster generation and sequencing (see, for example, FIGS. 33 through 39). When coupled with a reagent cartridge and instrument (see elsewhere within), an entire next generation sequencing workflow can be performed from sample to result in a fully automated format with no user intervention after the run is started. This is performed rapidly in a cartridge and instrument system of relatively small size, making it useful in a wide variety of settings. This heretofore has not been accomplished in the art and as such represents a new and novel system and associated workflows.

The integrated cartridge designs include several key features. The following is a brief discussion regarding some of these features. A 3-dimensional design of the cartridge was selected to accommodate the large number of required features in a relatively small footprint/volume. In FIG. 33, two form factor options for such a cartridge are shown. Both provide good functionality in a relatively small volume. The design depicted on the left provides a narrower form factor which reduces the instrument width without increasing instrument depth. Rotating the STC fin and associated thermal interface inwards also reduces instrument width. The ergonomics for cartridge installation into the instrument are ideal for both form factors.

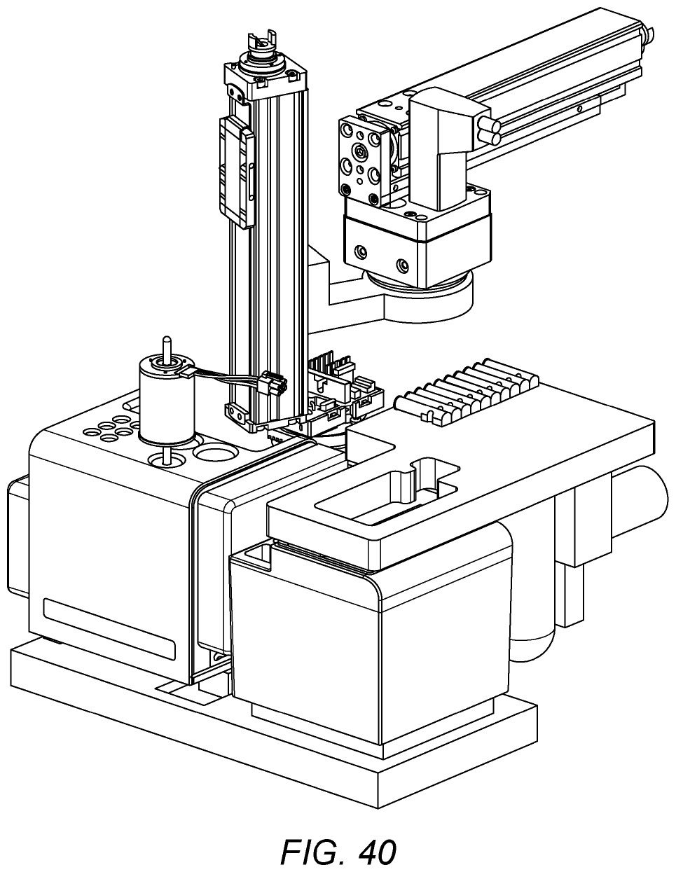

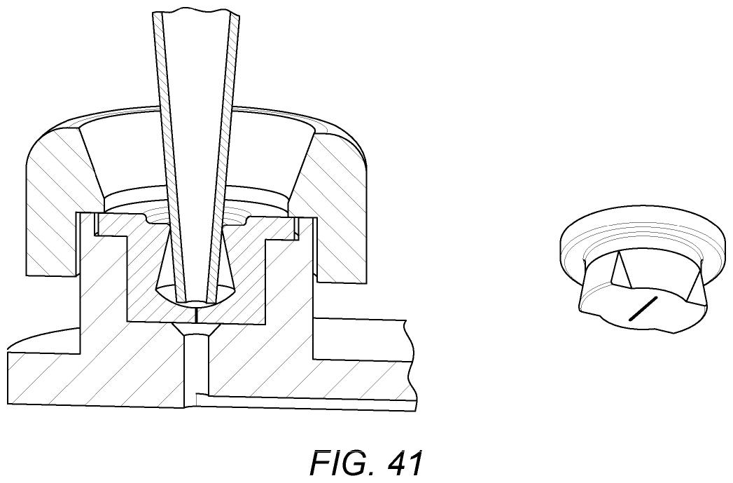

In some preferred embodiments of the cartridge, fluid handling comprises [direct]pneumatic pressure and precision pipetting using a compact, 3 degree-of-freedom (DOF) gantry situated in the associated instrument (see, for example, FIG. 40). Access of the pipette tip to chambers/channels in the cartridge is enabled through sealing pneumatic interface (SPI) ports (see FIG. 41). SPI ports may be configured with a cap 6701 as shown in FIGS. 67 and 68 to align the tip with the SPI port. The interior diameter of the cap 6701 opening may be keyed to the tip size specified for the fluid transfer step (e.g., a 1 mL tip in FIG. 67 and a 5 mL tip in FIG. 68). The SPI port may include a stepped pipette tip interface 6703 as shown in FIGS. 67 and 68 to accept and seal against different sized pipette tips. As depicted in the exemplary design in FIG. 33, the SPI valves are clustered closely together to minimize the range of travel required for the 3-DOF gantry, thus reducing engineering complexity, cost and use of real estate in the instrument. Access of the pipette tip to the SPI valves is enabled via an opening in the cartridge body directly above the valves (see figure). This opening is covered until use with a pierceable foil seal to keep contamination from entering the cartridge. There are additional round holes depicted in the figure, which are for staging of pipette tips to be used in the assay. Sample input ports (2 shown in FIG. 33) are situated to allow access on the top portion of the cartridge. Shown are 2 cylinders that accommodate (among other sample tubes) vacutainer tubes, showing the system can accommodate large volumes of input sample. Furthermore, each sample port comprises a separate SPI, allowing access to either sample or a combination of the samples at any point in the assay. In addition, the sample ports are located in very close proximity to the lysis chamber to minimize the path of travel for sample to the lysis chamber, if so accommodated, in the chemistry workflow.

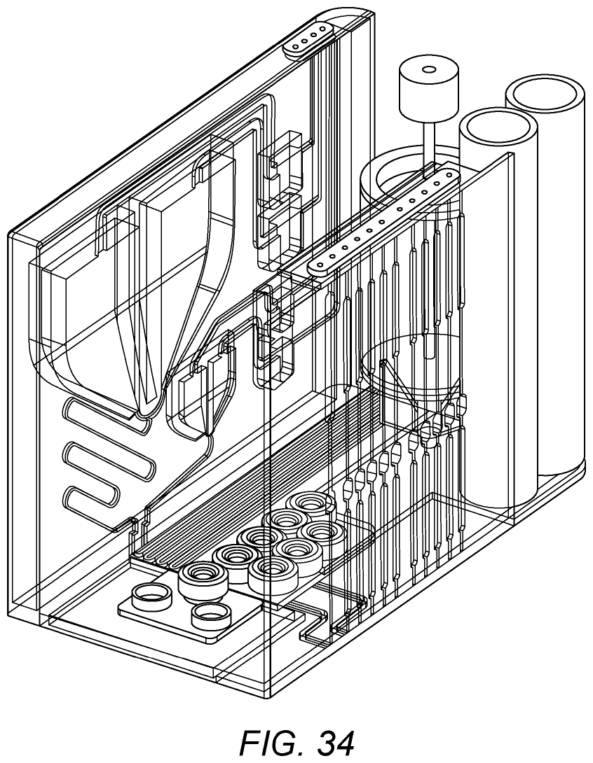

FIG. 34 depicts features of the cartridge in even more detail. As stated elsewhere herein, the functions associated with each of the features in this exemplary diagram are based on a particular assay or assay type, but functionality is flexible and is easily repurposed, reprogrammed or otherwise modulated to accommodate a wide variety of applications. The cartridge is designed in sub-module sections, each with a specified function or combination of functions. This is less complex and more cost effective for the manufacture and loading of reagents. It also allows for simpler modification of the cartridge for different applications (e.g., designing a new cartridge for an application that had a distinct sample preparation step would comprise redesign and manufacture of only the sample preparation sub-module as compared to the entire cartridge). The different sub-module sections are then easily assembled into a single, integrated cartridge. It should also be noted that additional advantages in the use of a pipettor for delivery of reagents include the ability to achieve simple sample dilution in the pipette tip, effective mixing by pipetting reagents in and out of chambers/channels (multiple times if necessary), delivering pressure to a chamber/channel (pipette tip inserted in SPI and air “dispensed”) to drive fluid movement, etc.

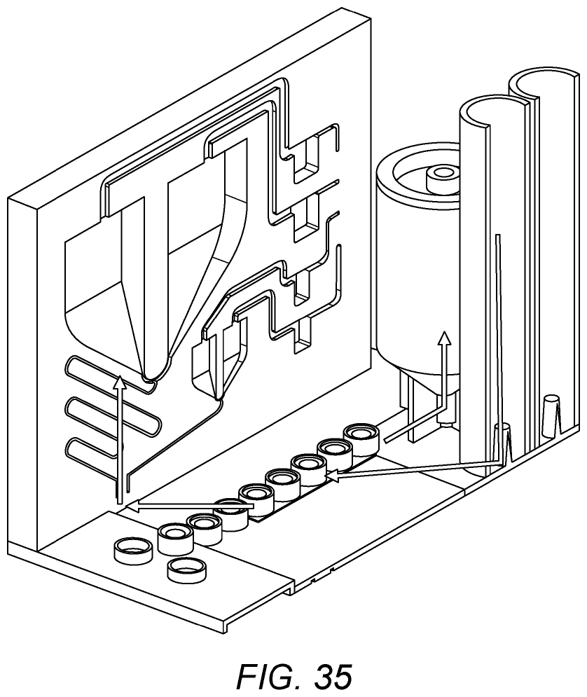

FIGS. 35 through 39 depict some of the basic steps of a workflow/assay (e.g., pathogen detection in blood) as they would be performed on the cartridge. The figure legends summarize these basic steps. As stated in the legends, not all parts of the cartridge are shown in this figure for ease of viewing the featured components. Also, not all steps of the workflow are summarized. It should be noted that assay chemistry/reactions can be conducted in channels as well as in chambers. It should also be noted here (as it is elsewhere within), the cartridge is designed to have high flexibility and is able to perform all the process steps of many different workflow configurations. Furthermore, as stated above, in some cases one or more of the sub-module fins in the integrated cartridge can be swapped out to accommodate an even wider range of workflows and applications.

Another exemplary assay or sample cartridge is shown in FIGS. 60 and 63-65 highlighting major fluid processing subsystems, units, or “fins”. FIG. 63 shows an exemplary library preparation unit 6300 or PCR fin with copy control function. FIG. 64 shows an exemplary sample input and mechanical lysis fin 6400 and FIG. 65 shows an exemplary specific target capture (STC) fin 6500. The STC fin 6500 may include thermal zones for heating as needed during target capture and elution steps. In certain embodiments, thermal energy may be applied to the STC fin 6500 from one side (e.g., from inside the cartridge). The library preparation unit 6300 may include two-sided heating for PCR and copy control thermal steps. The STC fin 6500 of FIG. 65 is shown in more detail in FIG. 69. STC chambers 6901 are used for sample preparation heating and mixing and, in the illustrated embodiment, may be about 9.3 mL in volume and designed to hold up to about 6 mL of volume. Additional fluidic functions of the STC fin 6500 are carried out using an elution chamber 6903 for elution heating, and an auxiliary chamber 6905 that can be used for PCR 1 dilution, PCR 2 pooling and copy control dilution where applicable. Lyophilized reagent pockets 6907 can be positioned to permit loading after fin construction and sealed with a film. The STC fin 6500 may include condensation traps 6909 to contain any condensation that forms during heating and mixing steps in the STC fin. An STC chamber inlet channel 6911 feeds the STC chambers 6901 and keeps fluid within the heated region during mixing and heating steps. An STC inlet pneumatic line 6913 allows air to be dispensed through the STC inlet to, for example, push fluid back into the STC chambers 6901 without the use of a pipette tip. STC and elution serpentine channels 6915 are included for magnetic bead capture steps.

An exemplary library preparation unit 6300 or PCR fin of FIG. 63 is shown in greater detail in FIG. 70. Various thermal chambers 7001 are included for PCR thermocycling and direct hybridization elution steps as needed. The thermal chambers 7001 are positioned to allow heating from both sides of the cartridge within the instrument for faster thermal ramping as required for PCR amplification. Lyophilized reagent pockets 7005 can permit loading of required reagents after fin construction followed by sealing with a film. A direct hybridization chamber 7003 may be included for mixing and incubation (at room temperature where specified) for assays using direct hybridization methods. A direct hybridization magnetic serpentine 7013 can also be included for magnetic bead capture as required. The library preparation unit 6300 may include a PCR2 bypass channel 7011 to allow PCR2 channels to be directly filled from a single SPI valve. PCR optical sensors 7007 and metering controls 7009 may be included for closed loop control as used for fluid positioning and metering.

Sample input may include openings or docking interfaces 6403 for receiving sample containers such as vacutainers or vials to allow the cartridge to take in samples for assaying. Where the assay uses mechanical lysis (e.g., through a rotating paddle), a mechanical interface 6405 may be included allowing the instrument to drive the lysis unit via, for example, a motor and shaft that operably couples to the interface when the cartridge is inserted in the instrument. The sample input, mechanical lysis, and STC steps may be combined generally in a sample preparation step and those functions may be part of a sample preparation unit within the sample cartridge. After processing in the library preparation unit (as exemplified in FIG. 63), amplified nucleic acid may be directed to a sequencing unit comprising, for example, a flow cell as described herein for sequencing and analysis of isolated and amplified target nucleic acids from the original sample. An exemplary sequencing unit/flow cell 6600 is shown positioned within the sample cartridge in FIG. 66. The sequencing unit/flow cell 6600 may be heated from below the cartridge as required for any sequencing steps. Additionally, FIG. 66 illustrates exemplary pipette storage 6603 within the assay or sample cartridge. As discussed herein, various fluid transfer operations within the sample cartridge between the reagent cartridge or other external sources and the sample cartridge may be performed automatically by the instrument using, for example, a pipetting gantry and SPI ports as described below. By including the necessary pipette tips, compatible with the required volumes and the SPI ports used in the system within a sealed cartridge, ease of operation is increased while risk of user error or contamination is reduced.

### C. Reagent Cartridge

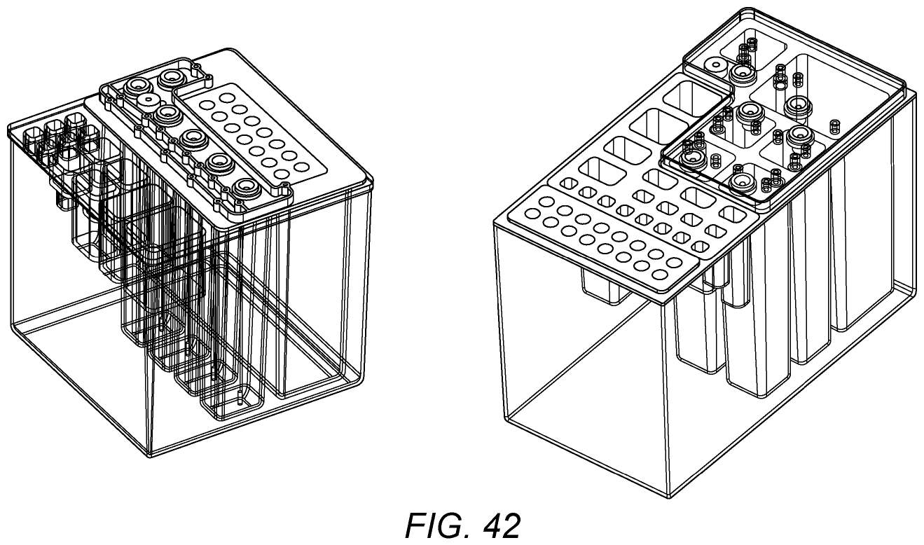

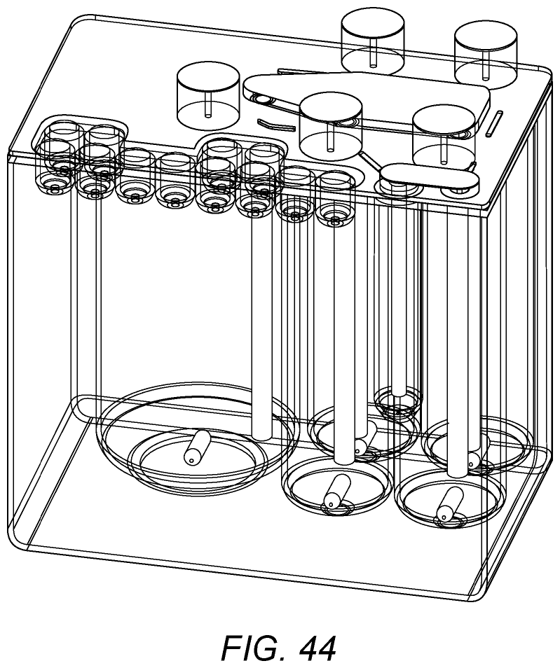

In preferred embodiments, a separate reagent cartridge is utilized in conjunction with an assay cartridge to perform a given test/assay. Exemplary designs of reagent cartridges are depicted in FIGS. 42 through 44. Fluids can be accessed and moved in a number of ways, included via a liquid handler (LH; e.g., pipette system) and a liquid manifold (LM). Different methods of valving are applicable, including sealing pneumatic interface (SPI) ports. Reagents stored in the reagent cartridge can include liquid and dry reagents, assay specific and general use reagents, bulk reagents (reagents typically required in larger amounts), and other reagents and components as needed (e.g., soda lime for use in CO₂ scrubbing of selected reagents). Furthermore, the body of the reagent cartridge provides a relatively large volume which can be efficiently used for liquid waste that is generated during performance of a test/assay. Still furthermore, the number of storage vessels/units/wells/chambers can be modified as needed to fulfill the requirements of a given or set of tests/assays. Also, a given configuration of reagent cartridge can be filled with a variety of reagents that will support a number of different tests/assays, even though all reagents will not be used for every test.

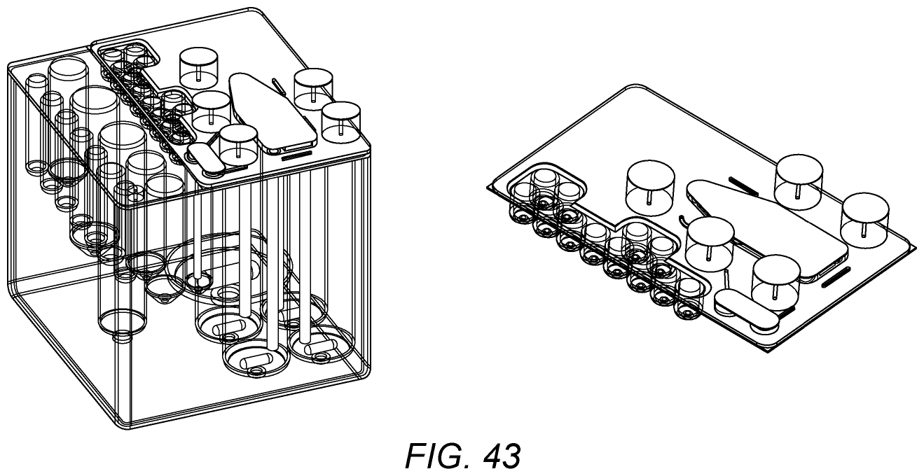

There are several advantages in using a separate reagent cartridge, including but not limited to, 1) Storing reagents on a separate reagent cartridge greatly decreases the size and complexity of the assay cartridge (especially evident when considering the volumes required for bulk reagents, such as sequencing reagents), thus increasing efficiency and decreasing cost of manufacture of the assay cartridge; 2) Assay and Reagent cartridges can be manufactured, filled and stored separately, reducing complexity and cost and increasing efficiency; 3) The preparation and storage of dried reagents is much more efficient when assigned to the reagent cartridge compared with the assay cartridge, especially when Section B of the reagent cartridge (used for dry reagents; see FIG. 43 and associated figure legend) is manufactured, filled, dried and stored in isolation; 4) The preparation and storage of liquid reagents is more efficient when assigned to the reagent cartridge compared with the assay cartridge, especially when Section A of the reagent cartridge (used for liquid reagents; see FIG. 43 and associated figure legend) is manufactured, filled and stored separately; 5) In some embodiments, the same reagent cartridge can be used with different assay cartridges; 6) The reagent cartridge can be rapidly assembled with sub-component parts (for example but not limited to, Sections A and B filled, dried (if applicable) and stored separately; bulk reagents (e.g., for sequencing) filled separately) for a given test/assay-specific application, so that the sub-component parts can be manufactured and stored most efficiently as well as manufactured in volumes consistent with demand; 7) Dry reagents can be reconstituted directly in the reagent cartridge before transfer to the assay cartridge; 8) In preferred embodiments, the majority of the reagents in the reagent cartridge are in dry form, such that the majority of the liquid, and in some embodiments all of the liquid, in the reagent cartridge is water; 9) In preferred embodiments, one or more of the chambers in the reagent cartridge contains a magnetic stir bar which interfaces with a magnetic stirring motor in the instrument when the cartridge is loaded into the instrument (useful, for example, for onboard preparation/mixing of reagents). Another exemplary reagent cartridge is shown in FIG. 61.

d. Instrument

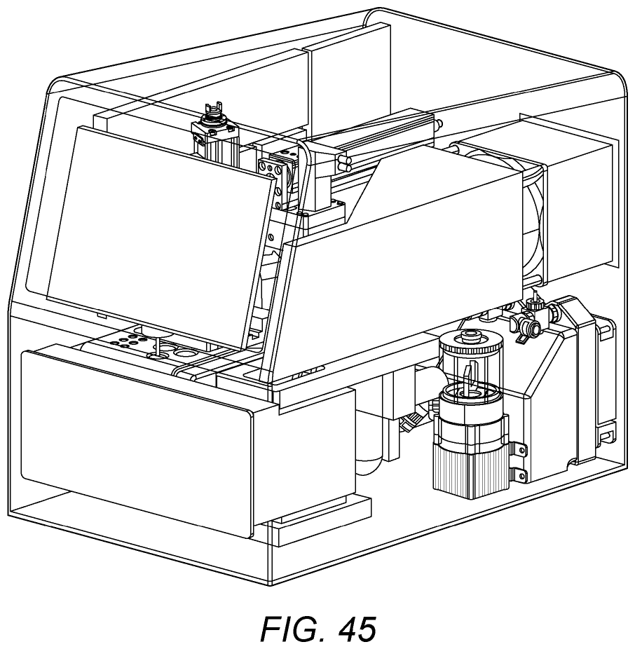

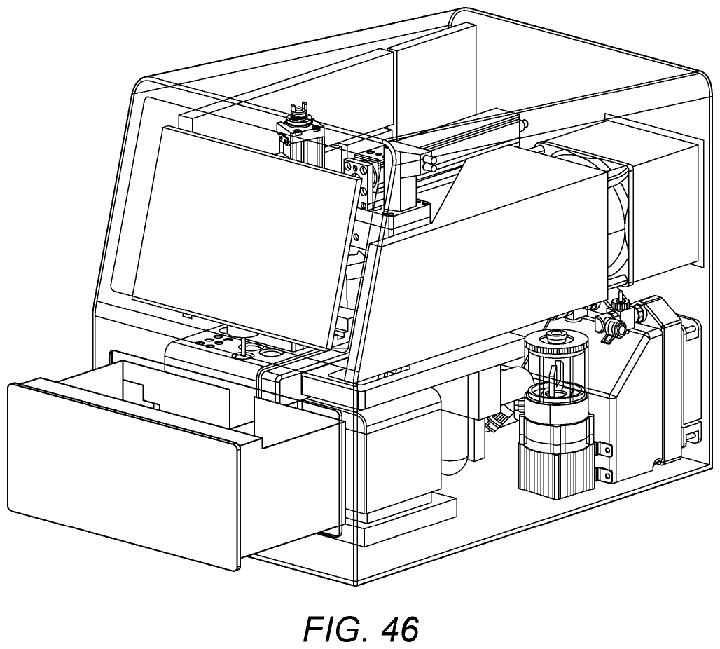

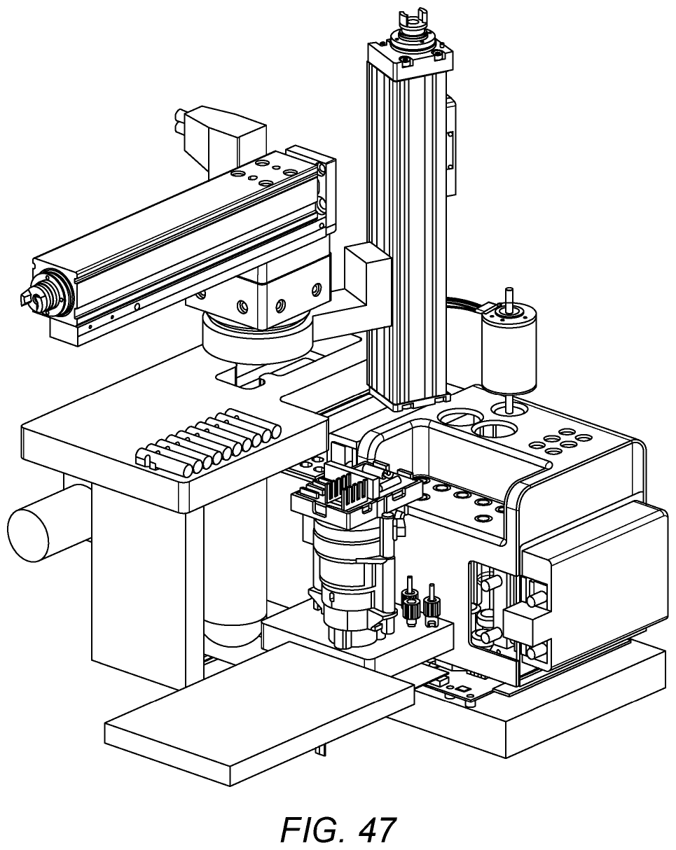

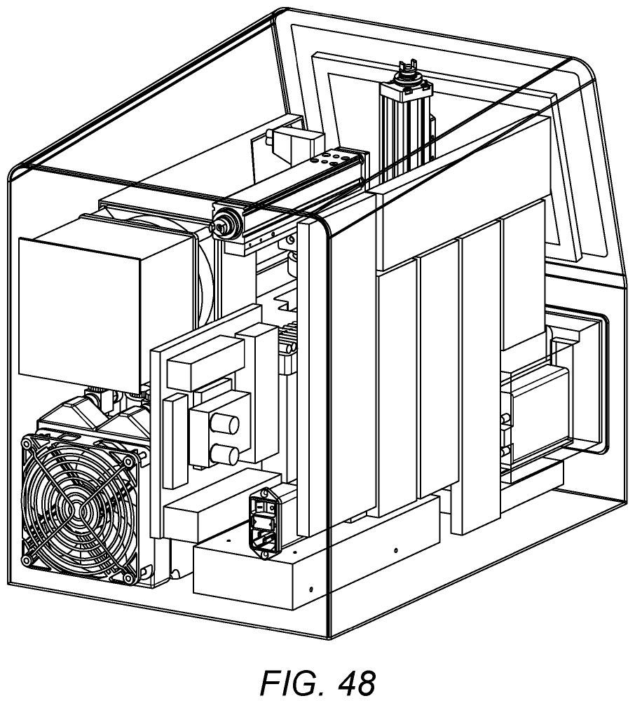

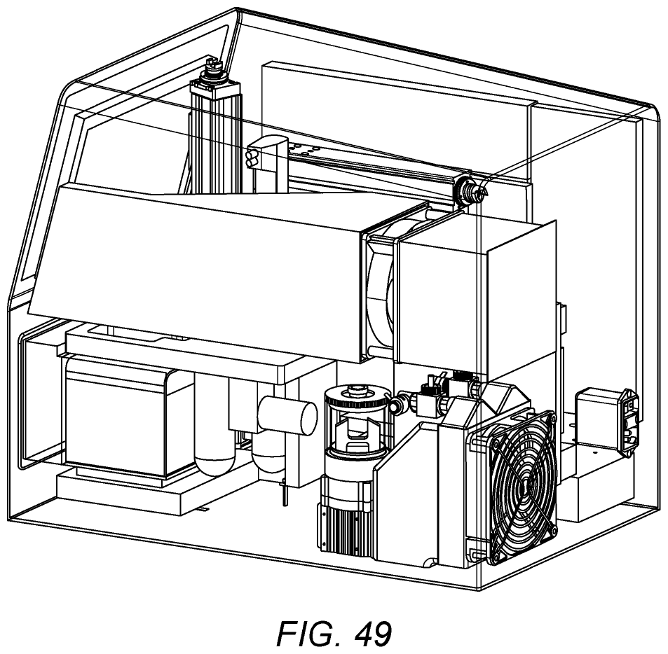

Disclosed in section (e) immediately above and elsewhere herein are a wide variety of cartridge embodiments. Disclosed in this section (and elsewhere herein) are instrument embodiments useful for automating the performance of tests/assays within said cartridges. Examples of instrument design and function are given in FIGS. 40, 45 through 49 and the associated figure legends. The instrument is designed to have a relatively small footprint, rendering it useful in a large number of settings. Further, it is capable of automating all the functions required to perform a full, complex workflow, including nucleic acid sequencing in preferred embodiments, in a cartridge format from sample entry to final report, all without user intervention once the run has been started. In preferred embodiments, the instrument is equipped with a compact, 3 degree-of-freedom (DOF) pipetting gantry utilized to deliver to the cartridge as well as perform a variety of functions including mixing, diluting, reconstituting (dried reagents) and moving fluids/reagents. As stated elsewhere within, in preferred embodiments pipette tip access to chambers and channels is via SPI ports. In preferred embodiments of the cartridge, the SPI valves are clustered closely together to minimize the range of travel required for the 3-DOF gantry, thus reducing engineering complexity, cost and use of real estate in the instrument. The instrument design comprises one or more of the following capabilities: heating, cooling (including for CPU), magnetic separation, magnetic stirring, lysis impeller rotation (for mechanical lysis in the cartridge), generation and controlled use of pressurized gas (pneumatic system), sensing (e.g., temperature, pressure, flow, fluid level, etc.). It also comprises full CPU/computer control of functions and features, including gathering and analysis of output data, such as sequencing signals from a semiconductor chip comprising an ISFET array. Other features are highlighted in the figures and associated legends.

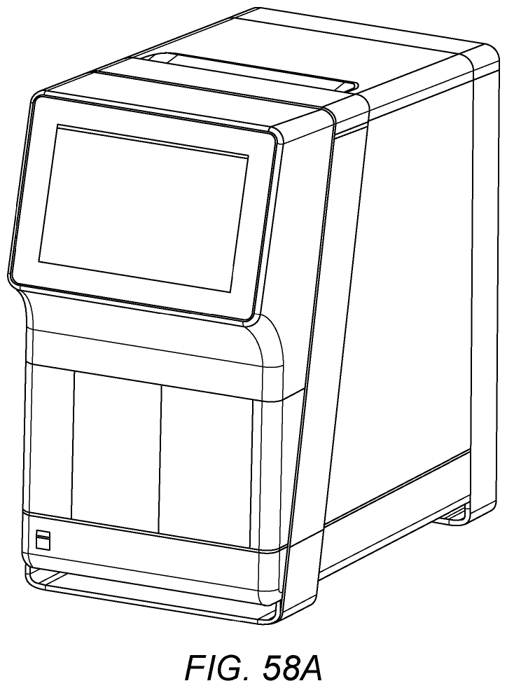

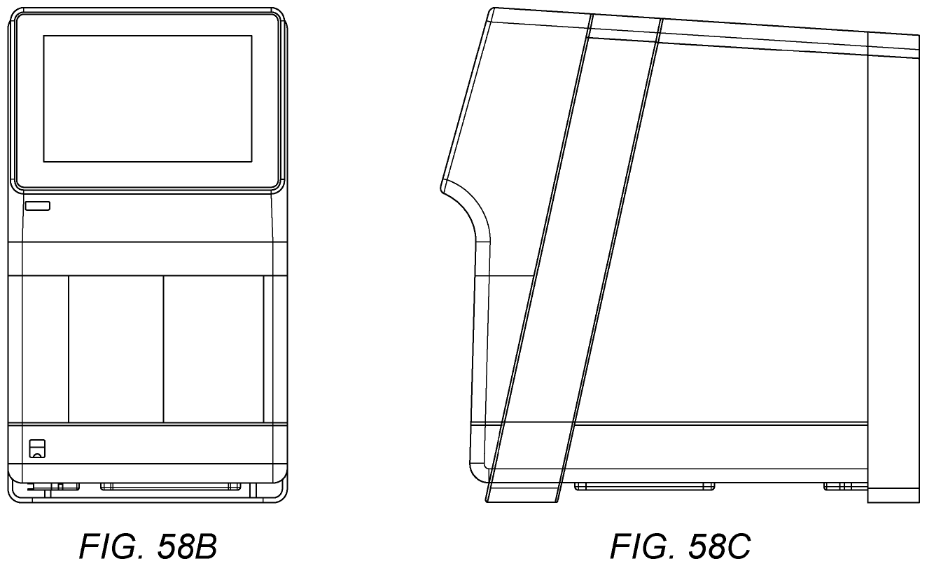

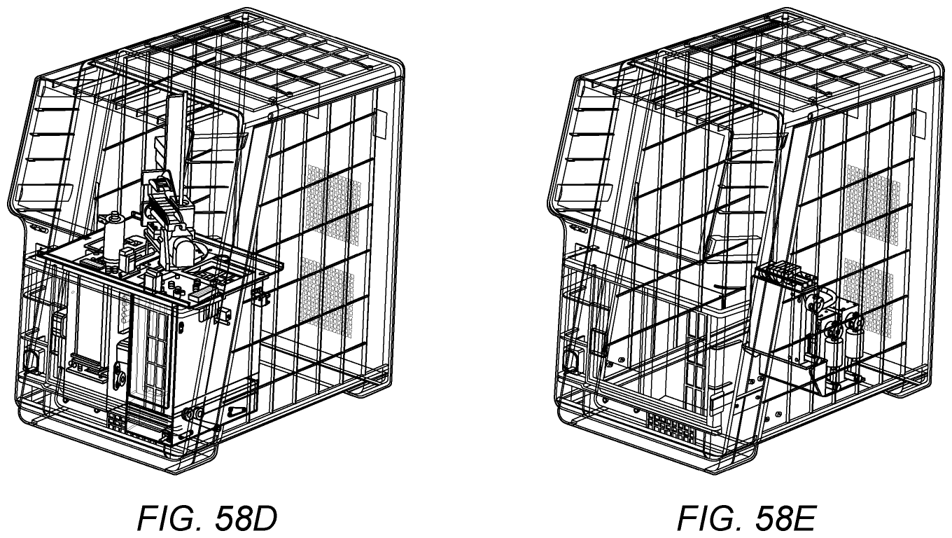

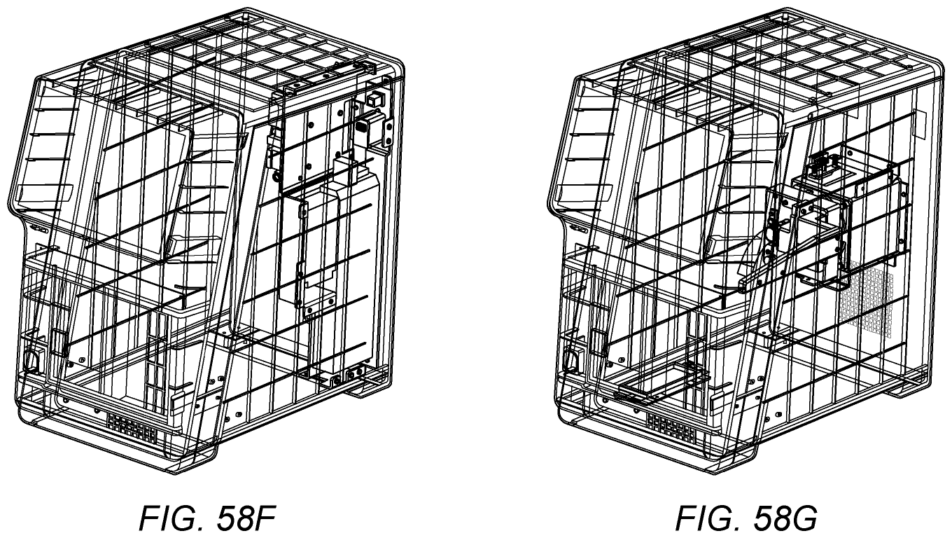

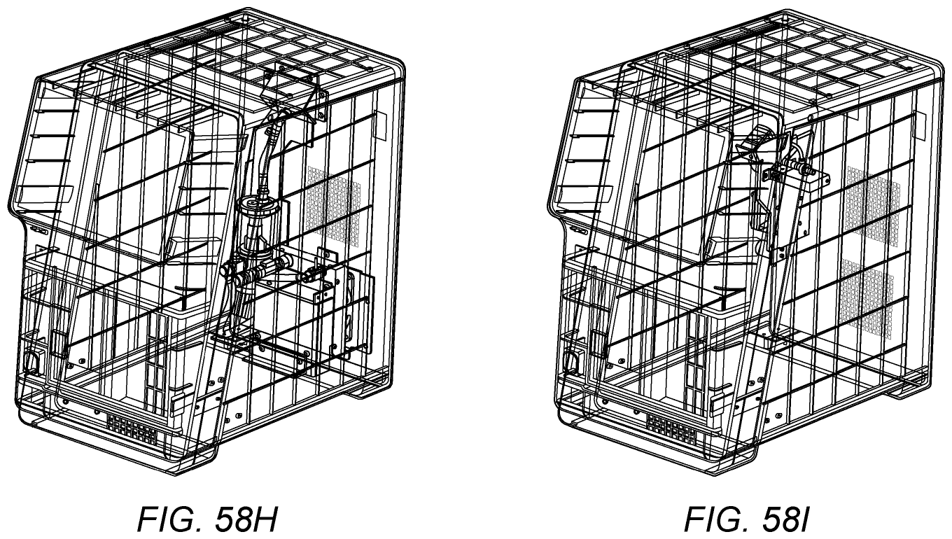

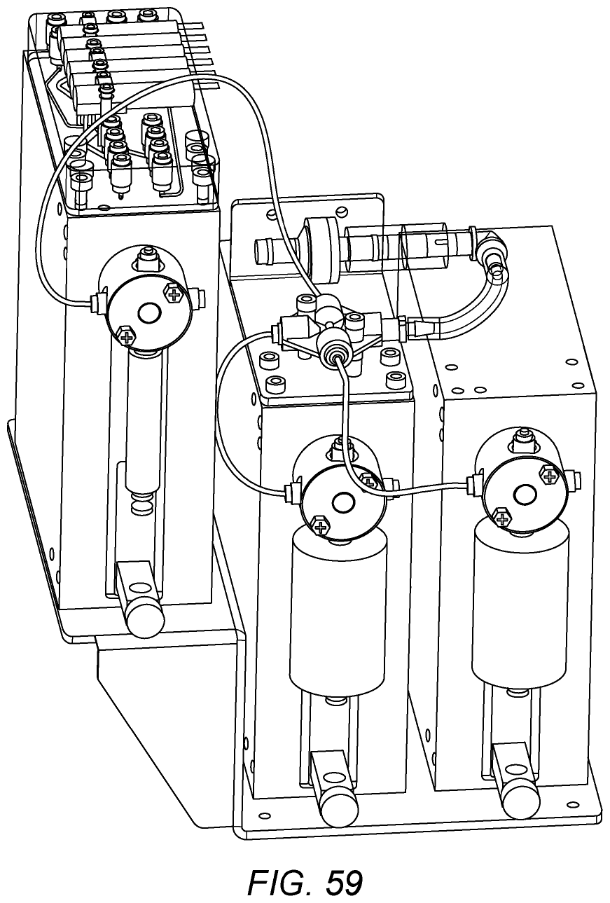

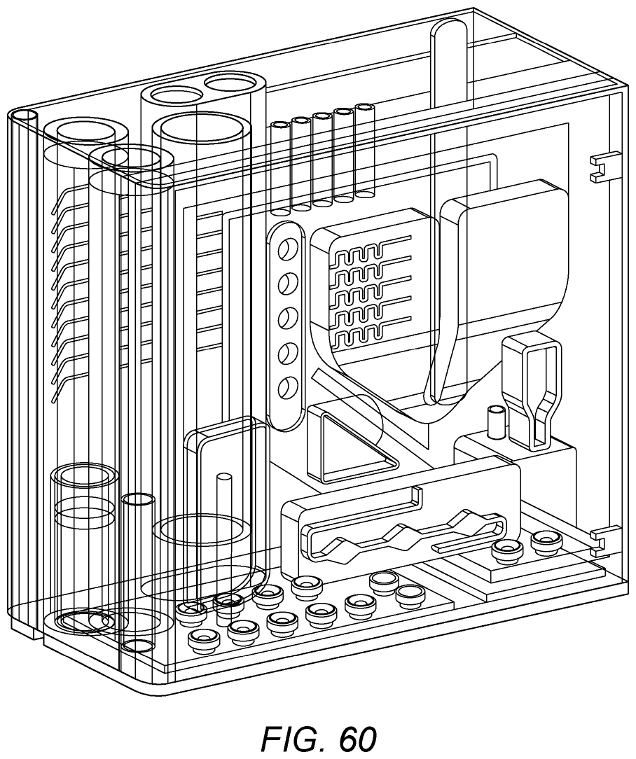

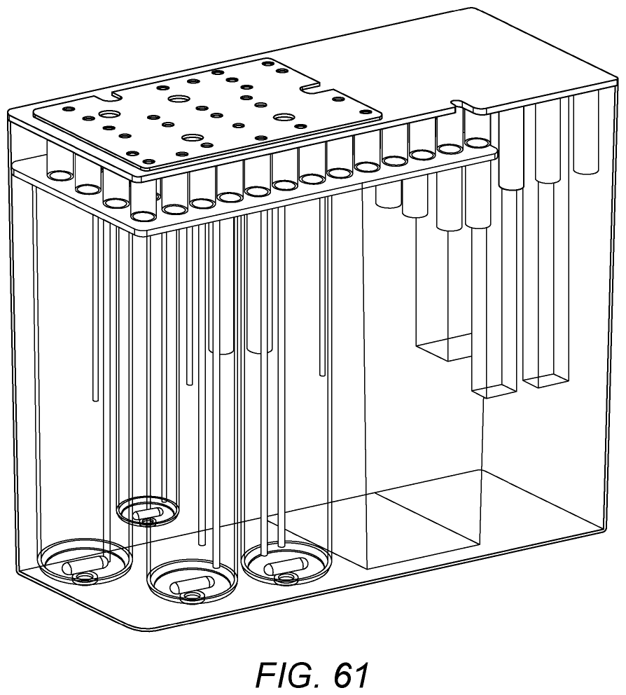

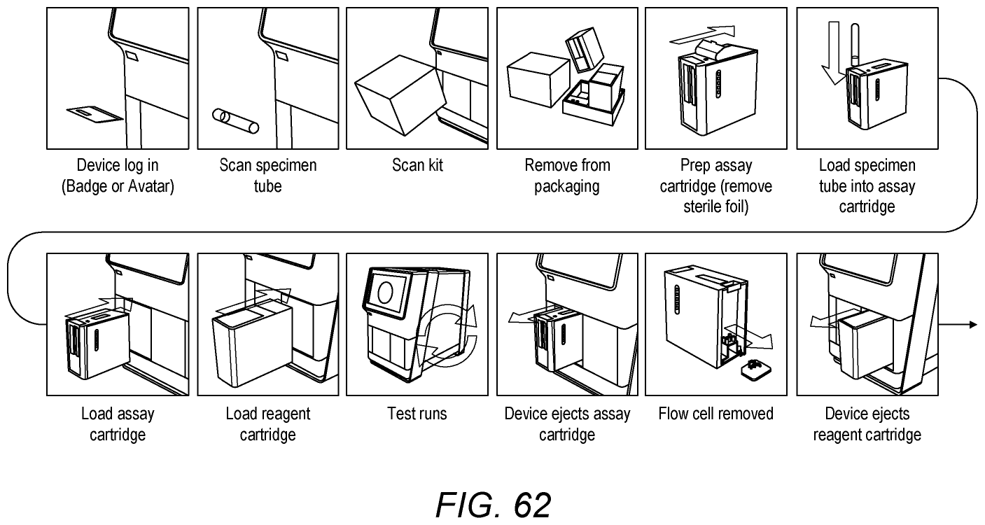

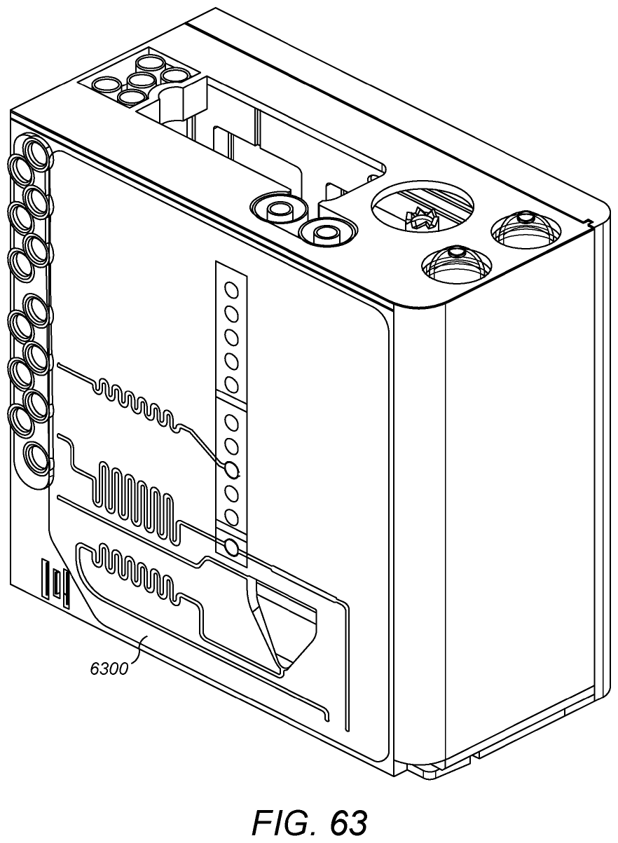

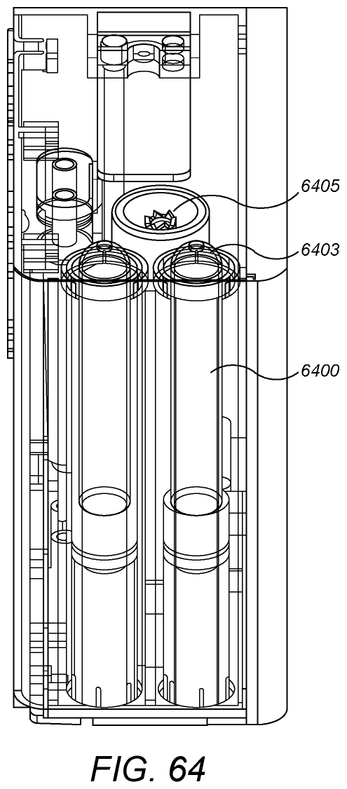

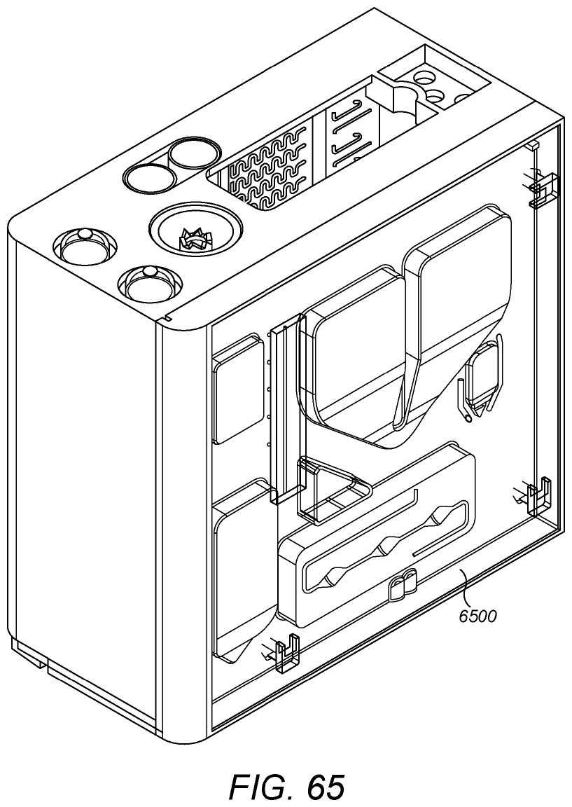

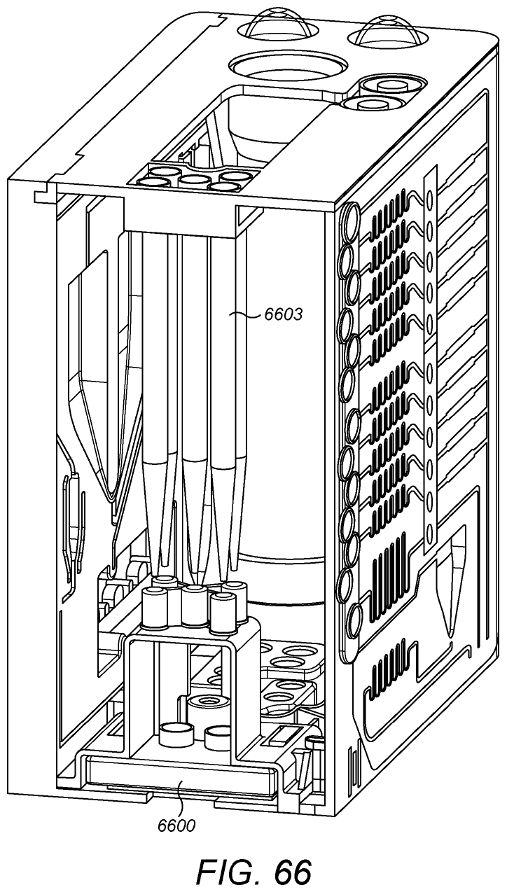

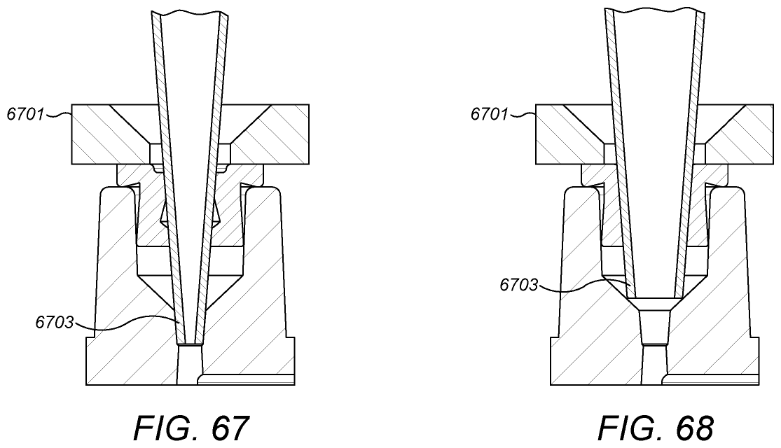

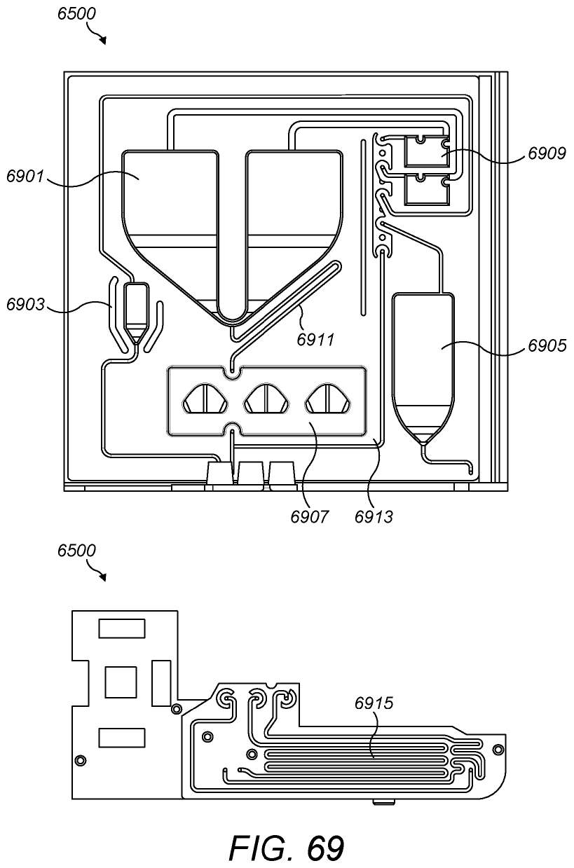

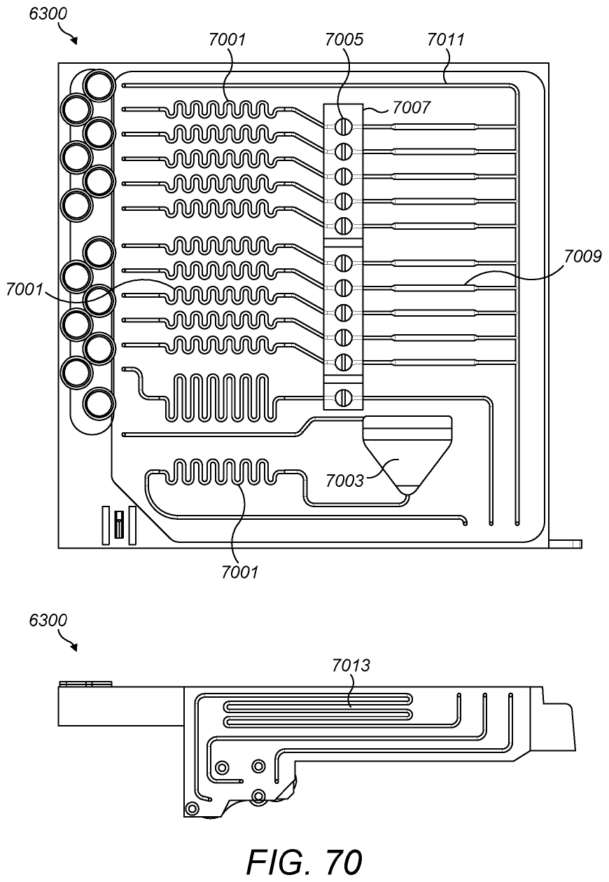

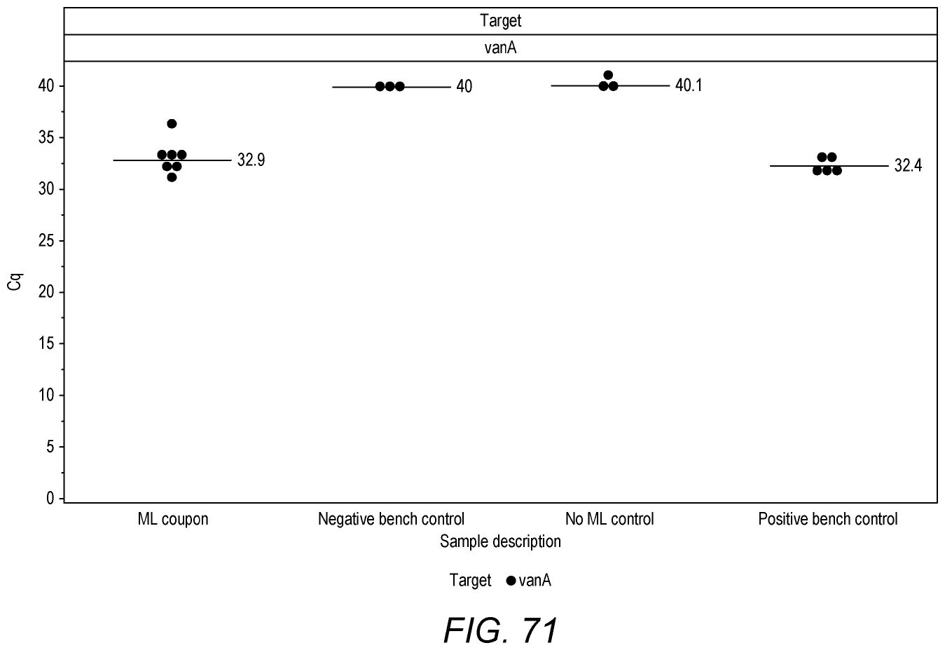

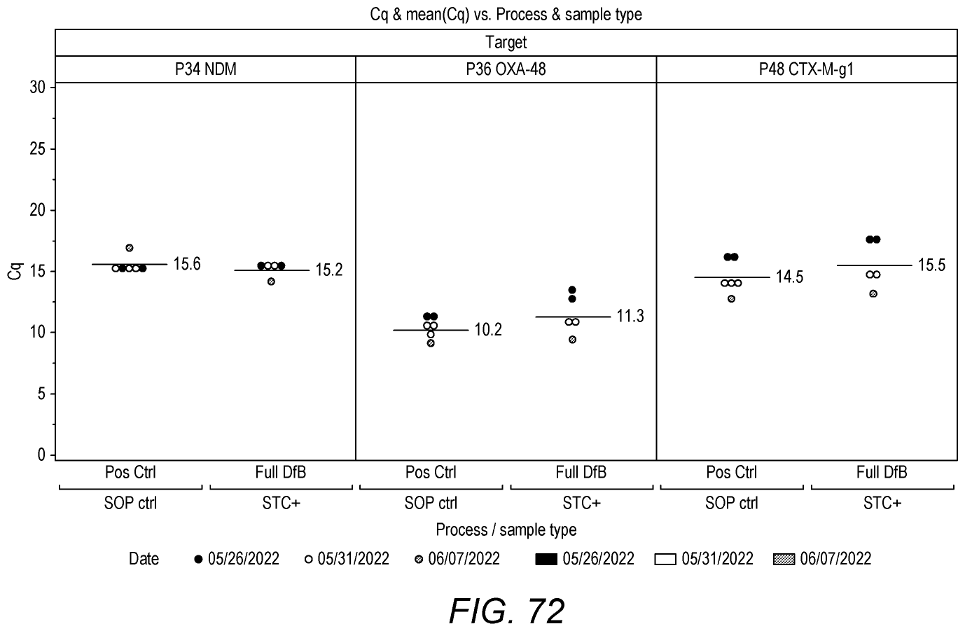

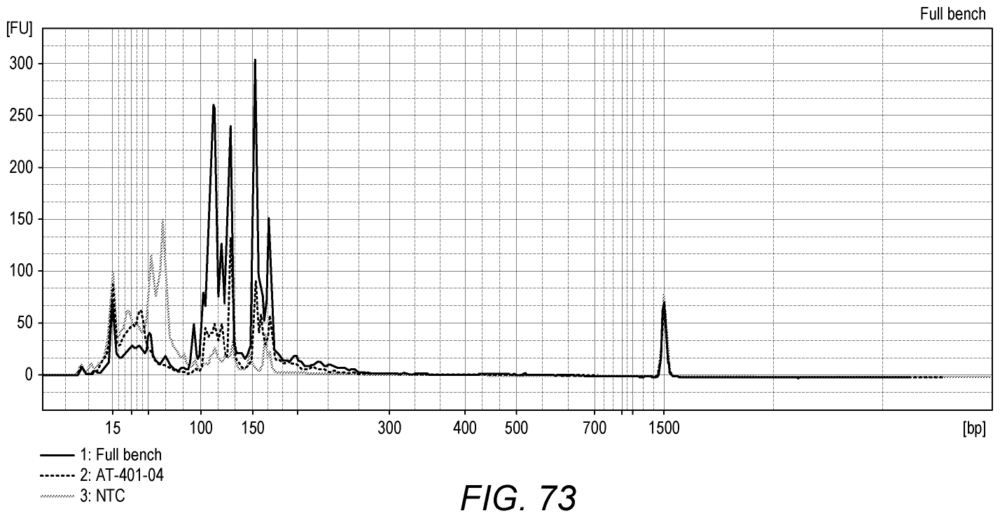

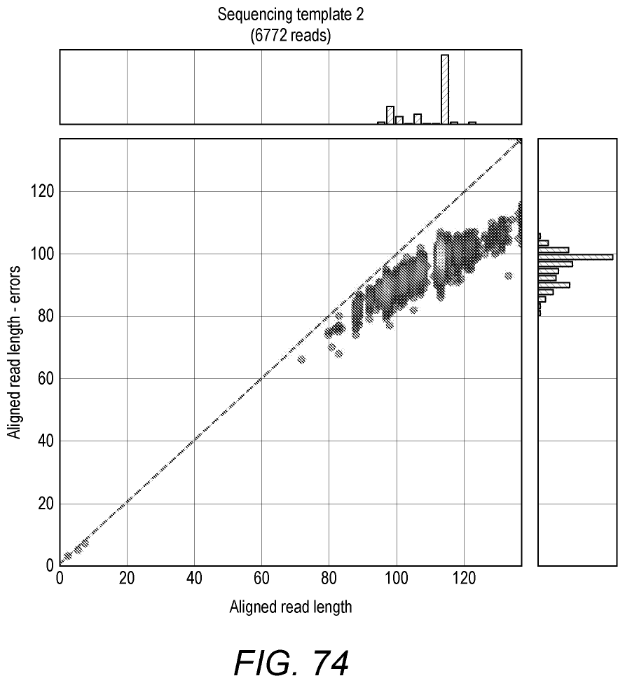

Another exemplary instrument and components therein are shown in FIGS. 58A-59. FIG. 58A shows a perspective view of an exemplary instrument with a display/user interface, a barcode scanner, and assay and reagent cartridge doors are shown. Dimensions of the exemplary instrument are shown in the front and side views of FIGS. 58B and 58C. The layout of various internal subsystems of the exemplary instrument are shown in FIGS. 58D through 58I. Cartridge interface assemblies for receiving and interfacing with a sample or assay cartridge and reagent cartridge are shown in FIG. 58D. The cartridge interface may include doors that may be manually or automatically opened or closed to permit insertion of the cartridges by a user but permit a closed, controlled environment for assay processing after insertion. In various embodiments, the cartridge interface may include fluidic and electronic connections to permit the instrument to control fluid movements within the cartridge and to communicate with the cartridge and various units therein (e.g., controlling sequencing and receiving sequencing data for processing). In certain embodiments, fluidic control may be pneumatic. FIG. 58E illustrates an exemplary pneumatic pumping subunit positioned within the instrument for supplying pneumatic pressure to be controlled by the analyzer or instrument to drive fluid movement within the cartridges. Such a pneumatic subsystem is further illustrated in FIG. 59 and may include syringes of various sizes (e.g., macro and micro) in order to permit bulk fluid movement as well as fine control thereof. FIG. 58F shows positioning of an exemplary power subunit for providing electrical power to the instrument. FIG. 58G illustrates an exemplary air handling and reagent cartridge air intake subsystem for controlling and treating any air entering the instrument and cartridge. FIG. 58H illustrates a liquid cooling subsystem for providing thermal management, for example, to cool processors or other heat-generating units within the instrument. FIG. 581 shows positioning of an exemplary condensation management subsystem for further controlling the environment within the system.

### 8. Methods of Using the System

As stated elsewhere within, prior art sequencing workflows—the process required to prepare a target polynucleotide contained in a sample for sequencing, conduct sequencing and analyze the resulting data—are tedious, time consuming, complex and often costly. Many of the steps are still performed manually and require highly skilled personnel. Even if specific processes in a workflow are automated, multiple instruments and ancillary components are required and skilled human intervention is required at various points to perform the entire workflow. Furthermore, the time from sample to result is several hours to several days or longer.

In addition, the maximum allowable amount of sample input is low, which represents a further current limitation. Thus, the power and value of sequencing—including next generation sequencing—can be greatly diminished in practical use. Accordingly, there exists a need for a sequencing workflow that is fully automated (sample-to-report), requiring no user intervention once the run is initiated, rapid (sample to actionable result in a few hours), sensitive, accurate, cost effective and amenable to use at the point-of-need.

Disclosed throughout are embodiments useful for just such a rapid analysis of target polynucleotides, including determining the nucleotide sequence thereof, from a wide range of input sample types and amounts using an automated system. Also as disclosed elsewhere herein, the system comprises an instrument and at least one assay cartridge removably insertable within the instrument. In preferred embodiments, the system further comprises at least one reagent cartridge removably insertable within the instrument. In still further preferred embodiments, the system comprises a semi-conductor chip, wherein in some embodiments the chip is embedded within the cartridge. In particularly preferred embodiments, the system still further comprises a flow cell mounted on top of the chip (with or without wells) useful for the delivery of fluids to the chip and removal of fluids therefrom. In preferred embodiments, the system comprises software. In particularly preferred embodiments, the software comprises operational software (for controlling the system) and analytical software (for receiving, processing and analyzing the output of the system).

In this section are disclosed a number of methods of using the above system. These are exemplary and are not meant to limit the scope of potential methods and associated applications. Specific examples of the execution of some of these methods of using the system are disclosed in the “Examples” section below.

### A. Overview of an Exemplary General Workflow

As stated above and elsewhere within, workflow means the process required to prepare a target polynucleotide contained in a sample for sequencing, conduct sequencing and analyze the resulting data. More specifically, preferred embodiments can include one or more (in various combinations) steps of sample processing; library preparation; copy control; cluster generation; sequencing; data acquisition; primary, secondary, tertiary data analysis; assay call (answering the question(s) which the test/assay was performed to answer); and report generation. The disclosed cartridge(s) and instrument(s) present a new and novel solution to a heretofore unsolved challenge, that is, the full end-to-end automation of all the steps of the workflow with a rapid time to result and within limits of a relatively small instrument (cartridge included) footprint.

As a first step, sample containing the target polynucleotide(s) is introduced into the cartridge. In preferred embodiments, this is achieved through a sample input port on the cartridge. Further, the cartridge is equipped with a component built into or attached onto the cartridge to help facilitate the transfer of sample in a safe, efficient and contamination-free manner. For example, in some embodiments the cartridge is equipped with a cylindrical structure in which a tube containing the sample (e.g., a standard vacutainer tube) is inserted. In preferred embodiments, placed at the bottom of the cylinder is a needle which is in fluid contact with at least one chamber in the cartridge via the input port. In these embodiments, the sample tube can be, for example, placed top-down into the cylinder and pushed onto the needle, the needle thereby penetrating the cap (making a tight seal around the needle and maintaining a tight seal between the cap and the tube) and the contents of the tube, or a portion thereof, are transferred into the cartridge. Additional exemplary components to help facilitate transfer of a sample include but are not limited to a luer lock; an external chamber/vessel (which is in fluid connection with at least one chamber in the cartridge via the port) in which liquid samples can be introduced (e.g., via pipetting), which is then sealed (e.g., via a lid) and the contents of the chamber/vessel transferred into the cartridge when the cartridge is inserted into the instrument; a pierceable septum through which a sample can be inserted into the cartridge via a syringe equipped with a needle that pieces the septum; etc. In some cases, unprocessed sample is loaded into the cartridge. In some cases the sample has undergone user-selected pre-processing (e.g., processing that occurs in a sample collection tube, or processing performed routinely on certain sample types as standard practice before analysis). In some cases, samples are the output of other, user-selected methods, such as the output of cell culture, cloning and expression, amplification, nucleic acid extraction, expression of swabs in transport media, liquification/homogenization of solid samples, concentration of samples (including sample in gas, such as air) media, cell lysis, separation (e.g., phase separation, settling, centrifugation, fractionation (such as whole blood into plasma or serum, buffy coat and erythrocytes), filtration and the like. Still further off-board routine processing methods include lysis of organisms, such as lysis of chlamydial reticulate bodies in detergent-based liquid (e.g., as a transport media in a collection tube), lysis of organisms by vortexing sample containing the organisms with or without the presence of beads, lysis by freeze/thawing in the collection tube, solubilization of the sample in detergent, chaotrope, organic solvent, denaturant, etc., with or without applied heat and/or agitation/vortexing, etc. These are just examples of the breadth of options, but the cartridge and instrument are designed to perform tests/assays using the primary sample directly. The external component (if used)/sample entry port/cartridge can accept a wide range of sample input volumes, for example in some cases low microliters to one milliliter, in some cases 2-10 mL, in some cases 4-20 ml, and in some cases even higher amounts. Likewise, the cartridge and instrument are designed to accommodate this volume range and perform the desired test/assay successfully. This is accomplished in a number of ways, including unique chamber designs (including a combination of large and small chambers as well as uniquely shaped chambers), dynamic fluidic control, processing performed “on the fly” as fluid flows past/through/over processing elements (e.g., heaters, magnets, etc.), excellent mixing capabilities and the like. This unique ability to accommodate such a large range of samples volumes (including relatively high volumes) fulfills a need in the field and distinguishes this disclosure from the prior art.

In some embodiments, once loaded into the cartridge the sample undergoes a variety of potential processing steps to prepare it for further downstream processing and/or analysis (AKA, sample preparation). In other embodiments, such as in cases where the sample matrix itself is relatively non-complex and the target polynucleotide is already in a form amenable to further processing and/or detection, the sample can bypass the sample processing step and move to a later step in the overall workflow, such as library preparation for example. In some tests/assays the sample type is whole blood and the target polynucleotide is within a cell, e.g., a pathogenic organism infecting the blood (as in sepsis, for example). In a preferred embodiment, sample preparation comprises the following general steps/processes: 1) The blood is mixed with a reagent(s) that support sample homogenization and lysis of the cells. 2) The sample is heated (with or without continuous movement and/or mixing). This helps to solubilize the sample (wherein in a further preferred embodiment, the method comprises turbulent mixing; features in the chambers/channels of the cartridge can optionally be included to improve turbulent mixing, such as 3-dimensional features, such as pillars, a narrow junction between mixing chambers, etc.). In some further preferred embodiments, the reagent comprises an enzyme, such as proteinase-K, that break down components of the sample enzymatically (for proteinase-K, the heating helps activate the enzyme). 3) The cells are lysed. This can be achieved using a number of methods (examples listed elsewhere within). In one particularly preferred embodiment, lysis is achieved using mechanical lysis, including mixing the sample at relatively high speed in the presence of beads. The cartridge is uniquely designed so as to incorporate a high volume mechanical lysis chamber, equipped with an impeller that engages with a motor in the instrument when the cartridge is loaded into the instrument. 4) The nucleic acid is liberated and denatured. Depending on the cell type and exact lysis methods, the target polynucleotide may still be associated with/bound to/trapped by features in the cell and/or sample. Furthermore, the target nucleic acid may be in double stranded form and must be rendered single stranded (denatured) for the next step of the process to function properly. In a preferred embodiment, both the liberation and denaturation are accomplished by heating to relatively high temperature (e.g., about 95° C.). These processes can be further aided by reagent composition, for example the inclusion of a detergent(s), a chaotrope(s) or a denaturant (or combination thereof). This/these can be included in the lysis reagent or added after lysis. Furthermore, the heating to relatively high temperature can occur by heating the entire lysate as a whole or by heating the lysate a portion at a time, e.g., by flowing it through a heated channel, e.g., a serpentine channel. 5) The target polynucleotide is isolated. A preferred embodiment is target capture one or more specific target capture oligomers (specific target capture, or STC). The denatured lysate is mixed with hybridization reagent containing the capture oligomers and the mix is incubated at elevated temperature (e.g., at about 60° C.), during which time the capture oligomers anneal to the target polynucleotide(s) at the user-defined level of specificity (via capture oligomer design). In some embodiments, the lysate is mixed with the hybridization reagent containing the capture oligomers prior to liberation/heat denaturation. The sample is then heated to about 95° C., for example, for liberation/denaturation, and then the temperature is lowered to about 60° C., for example, for annealing of the capture oligomers. In a preferred embodiment, after annealing the target polynucleotide/capture oligomer complex is capture onto magnetic microspheres, the microspheres are collected out of the mixture using a magnet and the remaining lysate is removed (sent to waste in the cartridge). Exemplary steps of how this is accomplished are included elsewhere herein. The beads are then optionally washed and the target polynucleotide is eluted. The target polynucleotide is now prepared for further downstream processing and/or analysis.

There are multiple advantages of the sample preparation embodiments described and discussed above elsewhere herein that distinguish them from the prior art, including but not limited to, 1) An entire sample preparation workflow, including starting with a relatively large volume of a complex biological sample such as blood, is performed in a fully automated format in a cartridge format, wherein the cartridge is an integrated cartridge that is also used for the remainder of the test/assay (i.e., the prepared sample does not need to be transferred to a different cartridge, different device, etc., to continue the process); 2) Full mechanical lysis is accomplished on-cartridge; 3) Complete liberation and denaturation of the target polynucleotide even in large volumes is achieved on-cartridge in a short time frame by using superior heating techniques for cartridge-based systems (including heating/incubating the sample as it passes through a channel, e.g., a serpentine channel, in contact with an effective heating element; 4) Specific target capture as configured in the disclosed cartridge compositions affords many advantages in and of itself, including high purification efficiency, high volume reduction capability, high specificity, and, even more broadly, high specificity control (can specifically capture all along the phylogenetic tree, including at the sub-species, species, genus, family, etc., level (via user-defined oligomer design; furthermore, the oligomers can be designed to specifically exclude unwanted polynucleotides in the sample, such as human DNA); 5) The STC method is highly scalable and supports a high level of multiplexing; further, the STC method is very flexible in that new applications/assays can easily and rapidly developed by simply designing new oligo sets; 6) Only 1 purification step is included in the entire process (sample preparation for next generation sequencing typically comprise multiple purification steps, which add time, complexity and cost); 7) The entire eluted volume of isolated target polynucleotide can go on directly into the next step of the process (e.g., library prep; for typical next generation sequencing workflows, at this step the target polynucleotide needs to be quantitated and only a fraction of the prepared target enters the library preparation process)

In some embodiments, after sample preparation (or, in some other embodiments, using a sample or a portion thereof directly, as described elsewhere herein), the sample undergoes a variety of potential processing steps to prepare it for further downstream processing and/or analysis (commonly referred to as library preparation). In other embodiments, the sample can bypass the library preparation step and move to a later step in the overall workflow, such as cluster generation or sequencing, for example. As stated above, in some tests/assays the sample type is whole blood and the target polynucleotide is within a cell, e.g., a pathogenic organism infecting the blood (as in sepsis, for example). In these cases the input to library preparation is target polynucleotide prepared from cells within the whole blood sample (examples of preferred sample preparation methods summarized above). In a preferred embodiment, library preparation comprises the following general steps/processes: 1) The input sample is mixed with a first amplification reagent. In preferred embodiments, the first amplification reagent is stored as a dried reagent on the cartridge (either in the assay or the reagent cartridge) and is reconstituted on-cartridge (using liquid stored on the cartridge (assay or reagent cartridge), e.g., water). 2) A region of interest (ROI) in the target polynucleotide is amplified in a first amplification reaction. This increases the number of copies of the ROI in the sample as well as increases the relative abundance of the ROI in comparison to other polynucleotides and/or polynucleotide regions in the mixture. In a preferred embodiment, primers are designed to amplify a broad range of pathogenic organisms, if present in the sample. For example, the primers are designed to amplify any bacteria or fungus in the sample, or a subset thereof of each depending on the requirements of the test/assay. This will enrich these bacterial and/or fungal ROI over human sequences or other potentially interfering sequences. In some embodiments, one or more tags/adaptors are incorporated into the amplicon product(s). One exemplary amplification method is PCR. 3) The product of the first amplification reaction (AKA, PCR1 amplicon) is diluted. 4) An aliquot of the diluted PCR1 amplicon is amplified in a second amplification reaction. An exemplary amplification method is PCR (e.g., PCR2 in this case). In a preferred embodiment, at least one primer is nested compared with the corresponding primer in PCR1. This adds another level of specificity. In another preferred embodiment, more than one second amplification reaction is performed (e.g., PCR2.1, PCR2.2, PCR2.3; 2-10 or more second amplification reactions can be performed on-cartridge). Each of the second amplification reactions can use different primer sets than in the first amplification reaction (to cover a broad range of target polynucleotides) or the same primer set as in the first amplification reaction (e.g., to make more of the final product) or any combination thereof across the different second amplification reactions. It should be noted that the first amplification reaction and/or one or more of the second amplification reactions can be configured to amplify 2 or more targets (i.e., multiplexed). In some embodiments, one or more tags/adaptors are incorporated into the amplicon product(s) of one or more of the second amplification reactions. 5) The amplicon products of all the second amplification reactions are pooled together. 6) In some embodiments, the pooled products from the second amplification reactions are purified directly (whereas in other embodiments, a copy control process is performed first; see below). The output solution is mixed with a target capture reagent, the mixture is incubated, the desired amplicons are bound to paramagnetic beads, the beads are immobilized using a magnet, the beads are washed and the target amplicons are eluted. Details of one such procedure are given in Example O. In some embodiments, amplicons are tagged with biotin (via biotinylated primers, for example). In such embodiments, the target amplicons are bound to strepavidin-coated magnetic beads in the incubation step listed above. In some embodiments, the amplicons are equipped with a common tag sequence and a capture oligomer is designed which is complementary to this common sequence. In some embodiments, the capture oligomer is biotinylated. In such embodiments, the biotinylated capture oligomer is bound to amplicons comprising the common tag sequence in a first incubation step. This reaction mixture is then combined with a second capture reagent comprising streptavidin-coated magnetic beads and the resulting mixture is incubated in a second incubation reaction during which time the amplicon/capture oligomer complexes are bound to the beads. In some embodiments, all components of the first and second capture reagents are in a single capture reagent and the first and second incubations are combined into only 1 incubation.

As stated above, in some embodiments a copy control (CC) protocol is performed on the pooled PCR2 amplicons. Acceptable CC protocols for use in the disclosed invention comprise one of any CC protocol disclosed in PCT/GB2021/050098 can be utilized (various combinations of disclosed features are also contemplated). Details of various CC compositions and methods are disclosed elsewhere herein. In a preferred embodiment, the pooled output from the PCR2 reactions is mixed with a CC reagent comprising one or more CC oligomers. The mixture is then heated at about 92-95° C. to denature the target amplicons. The mixture is then heated to promote annealing of the CC oligomer(s) and complement(s) (if present) and extension of the CC oligomers and target amplicons (3′-end of the strand hybridized with the CC oligomer(s); examples of various heating scheme as well compositions and reaction schemes given in the Examples section, elsewhere herein and as described in PCT/GB2021/050098). The resulting mixture is then mixed with a capture oligomer and incubated to promote hybridization with the capture sequence(s) incorporated into the target amplicons/complex. The resulting mixture is then mixed with magnetic beads, incubated, the bead/target complexes are immobilized using magnets, the beads washed and the targets eluted. In some embodiments, some, most or all of the copy control process is performed concurrent with the first amplification and/or one or more of the second amplification reactions.

There are multiple advantages of the library preparation embodiments (including copy control) described and discussed above and elsewhere herein that distinguish them from the prior art, including but not limited to, 1) In embodiments wherein the library preparation input sample is the output from sample processing methods disclosed within, the sample processing output material can be used directly with no quantitation or other characterization. In some preferred embodiments the entire volume of the sample processing output is used as input and in other embodiments a portion of the output is used as input. In some embodiments, the primary sample of a portion thereof is used as the input material for library preparation. 2) The system is capable of rapid and effective mixing of the initial input sample and required reagents as well as other combinations of reactants throughout the process. 3) The method of targeted enrichment provides a high degree of leverage to achieve the desired selectivity and specificity for a wide range of test/assay applications (two sequential amplification reactions, with nested priming an option in the second amplification and with seamless integration of the 2 reactions (including fully automated dilution, aliquoting and distribution of aliquots to individual reaction chambers)) as well as flexibility (e.g., in some embodiments, only 1 amplification reaction is performed; in some embodiments each of the separate second amplification reactions is configured (e.g., via primer design) to yield a different selectivity, specificity, target set, etc.). 4) High multiplexing capability. 5) Extreme flexibility and capability to add tags/adapters. For instance, tags/adapters can be added via tagging primers in any combination (e.g., in some embodiments both PCR1 and multiple PCR2 reactions are performed; each can be multiplexed, and each primer or primer set can comprise the same or different tags as another primer or primer set). In other embodiments, tags/adaptors can be added via ligation (in the system with full automation). In other embodiments, tags/adaptors are added via a combination of ligation and use of tagged primers. Furthermore, tagging can be combined, to a predetermined degree, with the copy control process, further simplifying and streamlining the overall process, thereby reducing complexity of the workflow and reducing processing time. 5) The system (cartridge+instrument) provides novel methods to dilute, aliquot and distribute a reaction mixture (e.g., an amplification reaction, such as PCR1) into multiple new reaction mixtures (e.g., a second amplification reaction, such as PCR2). 6) In preferred embodiments, the entire process utilizes only a single purification step (e.g., “direct purification” for downstream use in the disclosed direct hybridization method of cluster generation, purification as part of the copy control process, etc.). Furthermore, no pre-analysis (e.g., quantitation) of either the input sample (from various disclosed sample preparation processes, for example) or the first amplification reaction and dilution thereof is required. These features distinguishes the disclosed methods from the prior art, which require multiple purification steps and [typically]pre-analysis steps. 7) The disclosed copy control processes are novel and are transparently and fully automated using the disclosed system. 8) Required reagents can be stored and ultimately reconstituted (if required) and mixed with other reactants in various combinations and with various methods in the assay and reagent cartridges, providing unique and efficient methods/routes to reagent storage and use. 9) The entire process is fully integrated and automated by the system. 10) The process is rapid.

In some embodiments, after library preparation (with or without copy control), the sample undergoes cluster generation on the surface of a solid support. In other embodiments, the sample can bypass the library preparation step and move directly to cluster generation. In preferred embodiments, cluster generation is performed on the surface of a semiconductor chip. In further embodiments, the semiconductor chip comprises an array of ISFET sensors. In still further embodiment, the surface of the semiconductor chip comprises wells. In some embodiments, cluster generation comprises direct hybridization of target polynucleotides to an array of specific target capture oligomers on the surface of the solid support. In some embodiments thereof, the capture oligomer can also function as a sequencing primer when the method of analysis is sequencing. An exemplary method for cluster generation using the direct hybridization method (and in this case followed by sequencing) is summarized in Example M. In other methods, cluster generation comprises clonal amplification. An exemplary method for clonal amplification using Recombinase Polymerase Amplification (RPA) is summarized in Example J. An exemplary method for clonal amplification using Rolling Circle Amplification (RCA) is summarized in Example K.

There are multiple advantages of the cluster generation embodiments (including copy control) described and discussed above and elsewhere herein that distinguish them from the prior art, including but not limited to, 1) The output of the library preparation step (with or without copy control) can be used directly in the cluster generation method. In some embodiments, the output material (solution) is moved directly into the flow cell that covers the solid support. In this case, the volume of the flow cell will prescribe how much of the output solution is utilized in the cluster generation step. The output solution does not require any additional manipulation, such as quantitation, dilution, aliquoting and the like. 2) In preferred embodiments, cluster formation occurs directly on the surface of a semiconductor chip, upon which is also conducted sequencing. This is unprecedented in the prior art. 3) As described and discussed elsewhere herein, the disclosed method of performing clonal amplification using RPA is novel and distinguished from the art; 4) As described and discussed elsewhere herein, the disclosed method of circularized template formation (starting in the library preparation phase) and performing clonal amplification using RCA is novel and distinguished from the art; 5) The entire process is fully integrated and automated by the system. 6) The process is rapid.

In some embodiments, after cluster generation, target nucleic acid is analyzed by sequencing. In preferred embodiments, sequencing is performed on the surface of a semiconductor chip. In further preferred embodiments, the semiconductor chip comprises an array of ISFET sensors. In still further preferred embodiments, the surface of the semiconductor chip comprises wells. Exemplary methods for sequencing using a semiconductor chip comprising an ISFET sensor array and further comprising wells on the surface are summarized in Examples L (Sequencing of a Synthetic Template Directly Immobilized on the Chip Surface), M (Sequencing of a Synthetic Template Using the Direct Hybridization Method), N (Sequencing of a Template Generated Using In-Well Clonal Amplification) and O (Automated sample-to-answer sequencing of pathogen spiked into whole blood; entire workflow, starting with a whole blood sample and ending with sequencing and analysis of results).

Many advantages of semiconductor sequencing are well appreciated and documented in the prior art. These advantages included speed (faster than most sequencing-by-synthesis sequencing methods), no modified/labeled nucleotides are required, no optics are required for detection, fewer nucleotide and wash flows are required per a given length of sequence, etc. There are multiple additional advantages of the sequencing embodiments disclosed herein, including but not limited to, 1) The entire process, including cluster formation on the surface of the semiconductor chip and all sample and library prep steps prior to cluster generation (if required) are fully automated in a system comprising a cartridge and an instrument. 2) All required reagents for sequencing are containing in single use reagent cartridge and all waste fluid is contained within the reagent cartridge after the sequencing run. This affords simple and efficient use as well as safe disposal of used components after the run. 3) The disclosed method of concurrent sequencing primer annealing, and sequencing enzyme binding affords simplicity and reduced run time. 4) The disclosed methods of key sequence introduction and use afford a novel method for calibrating the sequencing run, etc. 5) The system is capable of onboard pH titration of reagents used for sequencing, which is important for ISFET-based sequencing.

Further embodiments of the invention comprise an onboard computer and auxiliary devices/electronics as well as software. Uses of the computer and software comprise control of the system and collection and analysis of the signal/data generated by the system.

Exemplary user-interfacing steps of a general workflow are shown in FIG. 62. First a user may use the barcode scanner on the front of the instrument to scan various information into the instrument. For example, an ID badge can be scanned to register a user or to permit access to certain assays/functions. The user information can be stored along with any subsequent assay data to control access and/or for quality control or analysis purposes. Specimen or sample containers may be scanned to read and record sample data for the assay. For example, patient information tied to a blood sample may be encoded on a barcode on a vacutainer and, by scanning with the instrument before entering into the sample or assay cartridge, the system can tie any subsequent assay data to a specific sample or patient. The user can then scan the kit which can provide information to the instrument on what assay is to be performed. This can eliminate user error by automatically generating an assay workflow for the desired assay or may be used as a quality control check to ensure that the proper kit is being used for the user-selected assay entered in the user interface. A user can then remove the cartridge or cartridges from the packaging and prepare them for insertion. In certain embodiments, the only user handling involved in the assay is opening of the cartridge, removal of sterile foil covering any ports, and specimen loading before inserting the cartridge into the instrument for automated processing of the assay. The user can insert, for example, a vacutainer into a sample cartridge through a sample input subunit specifically designed to accept a specific size/type of sample container. The cartridge(s) can then be loaded into the instrument through, for example, automatically opened doors. The instrument can then automatically run the desired assay and provide results through a user interface (e.g., a display on the instrument) or via a network to other computers. The instrument can then eject the cartridge(s) with all waste contained therein. The risk of user error and any contamination is thus reduced and the instrument is ready for the next test without the need for cleaning or onboard reagent refills as all fluidics are contained within the cartridges.

B. Options within General Workflow

The system is design to have unparalleled processing capability and flexibility regarding performing tests/assays, including in the areas of molecular testing, including sequencing. A single integrated system comprises one or more cartridges, wherein are many chambers of different sizes and functionalities, a high degree of fluidic connectivity, large volume capacity, novel and varied means of moving, transporting, combining, mixing, reconstituting and otherwise handling liquids (and gases, and solids as in reconstitution of dried, glassified or otherwise solid reagents or components), means of heating, magnetically separating, disrupting, blending, sensing, titrating (e.g., pH titration), detection and analyzing. All these are controlled via an onboard computer, and a wide number of processing steps in varied order, durations, conditions (e.g., temperature), etc. are possible. Within this context, a wide variety of different tests/assays can be run, and a wide range of options for different steps of each test/assay are possible.

Many options for sample preparation are feasible within the scope of the claimed invention. In some samples, the primary sample loaded into the instrument may need an initial processing step for example, if the sample is too viscous, inhomogeneous, fibrous, gelatinous and the like. Within the scope of the claimed invention, examples of methods for initial processing of a sample include but are not limited to, mixing with a reagent, including turbulent mixing, tortuous mixing, stirring mixing, and the like, including with reagents such as detergent, denaturant, chaotrope, organic solvent, buffers, salts, and the like and various combinations thereof; and/or enzymatic digestion, such as with Proteinase K (as described in the general workflow above) and/or other protease, nucleases, lipases and the like; and/or chemical compounds, including dithiothreitol, beta-mercaptoethanol, other reducing agents, oxidizing agents, acids, bases and the like. During the initial processing step(s) the sample may be mixed, heated, sonicated and the like. In some samples, no initial processing is required and the sample can enter directly into the next step. In some whole blood samples, the whole blood loaded into the cartridge is separated into plasma and its other components using a separation system integrated into the cartridge and which functions seamlessly within the system. All the above are envisioned to be enabled within the context of the disclosed system.

In some samples, the target polynucleotide is containing within cells, including within the nucleus, and/or within other structures. Within the scope of the claimed invention, examples of methods for Cell lysis/liberation of target polynucleotide include but are not limited to, mechanical lysis (as described above), with or without bead present (e.g., bead bashing), sonication, heating, mixing (e.g., turbulent mixing), shearing (e.g., by passing through a small orifice) and the like. Each of these processes may also comprise the action of a reagent, mixed with the sample, which participates in the lysis mechanism (detergents, denaturants, solvents, etc.; see above for more options). In cases where the target polynucleotide is included in the nucleus of a cell, a gentle lysis of the outer cell membrane may be performed first and the contents of the cell separated from the nucleus, and then the nucleus is lysed using a more stringent method. In some cases, the sample does not require lysis (e.g., the cells were lysed before the sample was loaded into the cartridge, the cells were lysed in the initial processing steps or the target nucleic acid was not in cells in the sample, etc.). In these cases, the sample can enter directly into the next step of the process. All the above are envisioned to be enabled within the context of the disclosed system.

In other protocols used when the target polynucleotide is containing within cells, including within the nucleus, and/or within other structures include those that capture intact cells (e.g., via affinity capture using magnetic beads). The remainder of the sample is washed away, and then the cell is lysed (examples of lysis methods given above). All the above are envisioned to be enabled within the context of the disclosed system.

In some protocols, target nucleic acid must be denatured (e.g., double stranded to single stranded) and/or liberated from association with other structures (e.g., DNA coiled around histones). Methods to do so include heating, treating with reagents (chemical and/or biological), mixing, sonicating, and the like. In some cases this is not required and the sample can enter directly into the next step of the process. All these are envisioned with the context of the disclosed system.

In some cases the target polynucleotide is isolated. Within the scope of the claimed invention, examples of methods for isolation of target polynucleotide include but are not limited to, 1) Specific target capture (STC) as described elsewhere within. This includes limited target capture as describe elsewhere herein as well as in PCT/GB2021/050098; 2) Non-specific capture methods, such as solid phase extraction methods, examples of which included but are not limited to solid-phase reversible immobilization (SPRI), solid-phase microextraction (SPME), silica-based methods, including the Boom method, the AMPure method, and the like; 3) A combination of non-specific capture (e.g., solid phase extraction) and specific target capture techniques (e.g., Boom method first followed by STC as described herein); 4) Hybrid Capture target enrichment strategies, such as the Agilent SureSelect method which utilizes RNA capture probes or “baits” to pull down regions of interest. Many different such hybridization-based methods exist, as well as other strategies for targeted enrichment, including but not limited to transposon-mediated fragmentation (tagmentation), molecular inversion probes (MIPs), and focused amplification procedures (e.g., as described elsewhere within). It should be noted that many of these procedures utilize fragment nucleic acid targets. It should also be noted that many of these procedures can be combined with library preparation or be used after library prep, but are described here in context of methods that can be used in the sample preparation phase in the disclosed system (they will also be mentioned in the library preparation section). It should also be noted that amplification or the first step or early steps of amplification can be performed on target polynucleotide that is captured onto beads (in sample prep, library prep, copy control and cluster generation). All the above are envisioned to be enabled within the context of the disclosed system.

In some cases, other processes can be performed during sample processing segment of the workflow within the scope of the claimed invention, examples including but are not limited to, 1) Fragmentation of the target polynucleotide; 2) Addition of tags/adaptors to the target polynucleotide, for example, via tagged oligonucleotides that anneal to the target polynucleotide and are extended (during sample preparation and/or library preparation) or by ligation. 3) Unique Molecular Identifiers can be incorporated to some or all of the target polynucleotides in a sample. In some cases the target polynucleotide is already in fragmented form and in otherwise very short pieces in the sample, such as fragmented DNA in urine, circulating tumor DNA (ctDNA) in the blood, cell free DNA (various sample types), small RNAs (various sample types), and the like. Methods to process these polynucleotide samples, including but not limited to tag/adaptor addition, amplification, reassembly, capture and the like, can be performed on the system. All the above are envisioned to be enabled within the context of the disclosed system.

Many options for library preparation are feasible within the scope of the claimed invention. In some embodiments, the input for the library preparation process is the output of the sample preparation method and in some embodiments it is the primary sample itself. Preferred embodiments of amplification-based methods of targeted enrichment are describe elsewhere herein. Variations of these methods that are envisioned for use in the disclosed system include but are not limited to, 1) One or three (or more) separate amplification reactions; 2) Tags/adapters incorporated in a wide range of scenarios (only in one of the amplification reactions, in a combination of amplification reactions, incorporated differentially between ends of the same target polynucleotide and from target polynucleotide to target polynucleotide, as part of the copy control process (as described elsewhere herein and in PCT/GB2021/050098), as a combination of processes in the sample preparation phase (see above) and the library preparation phase), etc. Amplification can be achieved using a wide variety of methods known in art, including but not limited to Polymerase Chain Reaction (PCR), Reverse Transcription PCR (RT-PCR), Nicking Endonuclease Amplification Reaction (NEAR), Transcription-Mediated Amplification (TMA); Loop-Mediated Isothermal Amplification (LAMP); Helicase-Dependent Amplification (HDA); Clustered Regularly Interspaced Short Palindromic Repeats (CRISPR); Strand Displacement Amplification (SDA); Recombinase Polymerase Amplification (RPA), Ligase Chain Reaction (LCR), and the like. There are a wide range of other library preparation methods known in the art that are envisioned for use in the disclosed system. A few examples of general workflows include but are not limited to, 1) Fragmentation, ligation of adapters, amplification (typically comprising additional adapter addition); 2) Fragmentation, amplification (random, semi-random, specific; can include adapter addition), adapter addition; optional amplification (often comprising additional adapter addition); 3 Amplification (can include the examples listed above as well as whole genome amplification (e.g., Picoseq, DOPlify, REPLI-g (based on Multiple Displacement Amplification, or MDA) and Ampli-1 WGA), long-range PCR, amplification using semi-random and/or degenerate primers, etc.), fragmentation, adaptor addition, optional amplification (often comprising additional adapter addition); 3) Transposon-Mediated Fragmentation (tagmentation); 4) Molecular Inversion Probe-based methods (MIPs); and the like. Several of the above techniques of library preparation can be followed with targeted enrichment (some techniques listed above, such as SureSelect). As stated above, these the steps of the library preparation and sample preparation process can overlap and be performed across different units of the cartridge. Also as stated above, UMIs can be added at various stages of the sample preparation and/or library preparation processes (where in the process depends on the exact protocol). All the above are envisioned to be enabled within the context of the disclosed system.

Copy control can be performed after library preparation, overlapping with the library preparation process, or not at all (depending on the application). In some cases, tag/adapters utilized in a copy control process can even be incorporated during sample preparation. A wide variety of novel copy control methods have been described. Furthermore, other copy control methods known in the art can also be performed on the system.

Cluster generation methods have been described herein. Other methods could also be utilized, including the use of different surfaces and/or surface geometries, difference surface immobilization chemistries, and different amplification methods. Other methods we have demonstrated to support cluster generation in the claimed invention include PCR, HDA, SLAM (a proprietary surface-phase amplification procedure; patent pending) and EM-Seq (a proprietary displacement-mediated amplification procedure; patent pending).

Sequencing on the semiconductor chip can be performed on essentially any type of sequencing library using any targeting strategy (see above for examples), i.e., not just sequencing of amplicons from, for example, targeted enrichment. Also, sequencing can be performed after cluster generation using the direct hybridization (described above) with any number of targets and target sources (not just those used as examples). High density arrays of capture oligomers with different degrees of specificity can be applied to the surface of the chip and utilized in this method.

A wide variety of data analysis methods have been developed, including analysis via triangulation (a triangulation strategy is also utilized in oligonucleotide design, including STC, targeted enrichment and key sequence development).

### Definitions

It is to be understood that the disclosure is not limited to specific compositions or process steps, as such may vary. It should be noted that, as used in this specification and the appended claims, the singular form “a,” “an,” and “the” include plural references, and expressions such as “one or more items” include singular references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context. When “at least one” member of a class (e.g., oligomer) is present, reference to “the” member (e.g., oligomer) refers to the present member (if only one) or at least one of the members (e.g., oligomers) present (if more than one).

It will be appreciated that there is an implied “about” prior to the temperatures, concentrations, quantities, times, etc. discussed in the present disclosure, such that slight and insubstantial deviations are within the scope of the present teachings herein. In general, the term “about” indicates insubstantial variation in a quantity of a component of a composition not having any significant effect on the activity or stability of the composition, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques. All ranges are to be interpreted as encompassing the endpoints in the absence of express exclusions such as “not including the endpoints”; thus, for example, “within 10-15” includes the values 10 and 15 and all intervening integer and (where appropriate) non-integer values. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. Section headings are provided solely for the convenience of the reader and do not limit the disclosure. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. “Consisting essentially of” means that additional component(s), composition(s) or method step(s) that do not materially change the basic and novel characteristics of the compositions and methods described herein can be included in those compositions or methods. Such characteristics include the ability to, e.g., hybridize to a target polynucleotide and undergo further binding and/or extension reactions as described herein, as the case may be.

A “sample” refers to material that may contain a target polynucleotide, including but not limited to biological, clinical, environmental, and food samples. Environmental samples include environmental material such as surface matter, soil, water, sludge, air and industrial samples, as well as samples obtained from food and dairy processing instruments, apparatus, equipment, utensils, disposable and non-disposable items. “Biological” or “clinical” samples refer to a tissue, liquid or other material derived from a living or dead human, animal, or other organism which may contain a target polynucleotide, including, for example, tissue samples, swabs, washes, aspirates, exudates, biopsy tissue, or fluids such as blood, spinal fluid, excretions, semen or urine. A sample can be treated to physically or mechanically disrupt tissue or cell structure to release intracellular nucleic acids into a solution which may contain enzymes, buffers, salts, detergents and the like, to prepare the sample for analysis. A sample can also be an aqueous or organic solvent or combination thereof, with or without other components (e.g., buffer, salt, detergent, emulsifying agent, EDTA, etc.) that contains a target polynucleotide. These examples are not to be construed as limiting the sample types applicable to the present disclosure.

Sample preparation refers to a method or combination of methods by which a sample containing target polynucleotide is manipulated in order to prepare the target polynucleotide for further downstream processing and/or analysis. Such methods include but are not limited to methods to release, render accessible, digest, remove bound components from, concentrate, enrich, capture, separate, and/or isolate target polynucleotide in a sample. Such methods also include methods to remove, neutralize or otherwise render less effective potentially competing, interfering, obscuring or otherwise detrimental to downstream analysis substances, components, contaminants, organisms (including dead or alive and/or debris from such organisms) or other biological, organic or inorganic material from the sample containing the target polynucleotide. Such methods include but are not limited to, 1) Methods to solubilize, dissolve, homogenize, digest or otherwise alter the physical or chemical properties of the sample to aid in the preparation of target polynucleotide, such methods including but not limited to heating, cooling, freezing, freeze-thawing, digesting—including using chemical or biological means, including enzymatic means—sonicating, dissolving using solvents, reagents and/or other chemical or biological means, stirring, shearing, mechanically agitating, and the like; 2) Filtering the sample; 3) Concentrating the sample; 4) Tagging, labeling, capturing, concentrating, isolating or otherwise processing cells suspected of containing target polynucleotide, such methods including but not limited to tagging with cell-specific components, including antibodies, lecithins, nucleic acids, proteins, peptides, aptamers, dendrimers, other cells, viruses, macrophages, other biological components and the like, labeling the cells with fluorescent dyes, radioactive labels, luminescent labels, mass labels, and the like, capturing, concentrating or isolating the cells using precipitation, centrifugation, including the use of a density gradient, filtration, affinity capture, including via at least one of the cell-specific components listed in the tagging section above, including with binding to a solid support directly or indirectly, cell sorting and the like; 5) Lysing, digesting, rupturing, partially dissolving, shearing or otherwise manipulating cells or other structures containing or otherwise associated with target polynucleotide to render the target polynucleotide accessible or otherwise more available for further processing or analysis, such methods including but not limited to heating, cooling, freezing, freeze-thawing, boiling, exposing cells to osmotic shock, treating with solvents, chemicals or other reagents, including in combination with heating, sonicating, sonicating with concurrent heating, beating/bashing, enzymatic treatment, stirring, shearing, including mechanical shearing, and the like; 6) Methods to solubilize, dissolve, homogenize, digest or otherwise alter the physical or chemical properties of the target polynucleotide to aid in the further processing and/or analysis of target polynucleotide, such methods including but not limited to at least one of the methods listed in section (1) above in this paragraph; 7) Tagging, labeling, concentrating, enriching, capturing, separating, and/or isolating target polynucleotide, such methods including but not limited to methods to tag, bind to, affix, couple, incorporate or otherwise attach to, whether directly or indirectly, covalently or non-covalently, a nucleic acid, a nucleic acid segment, multiple nucleic acid segments, protein, enzyme, aptamer, lecithin, dendrimer, element, molecule, or any other substance or moiety, including chemical, biological, organic, and/or inorganic, that aids in the further preparation, processing (including amplification) and/or analysis of the target polynucleotide, including all tagging methods commonly known in the art, labeling with fluorescent dyes, radioactive labels, luminescent labels, mass labels, and the like, precipitation, extraction, including GuSCN, CTAB, Chelex (and other resin types) and alkaline extraction, chromatography, including column chromatography, filtration, centrifugation, including the use of a density gradient, isoelectric focusing and other focusing techniques known in the art, capture on a solid support, including magnetic microspheres and other solid support materials commonly known in the art, directly and indirectly, including using non-specific target capture methods such as solid-phase extraction (SPE), ion-exchange SPE, solid-phase reversible immobilization (SPRI), solid-phase microextraction (SPME), silica-based methods, including the Boom method, the AMPure method, as well as using random or semi-random target capture oligomers in order to capture various targets in a sample, specific target capture methods, including methods that utilize one or more oligonucleotides specific for a polynucleotide or group of polynucleotides of interest wherein the oligonucleotide(s) is/are annealed to the target nucleic acid(s) and this/these complex(es) is/are immobilized onto a solid support, wherein some methods of enriching, capturing, separating, and/or isolating target polynucleotide or in any other way purifying a target nucleic acid include one or more wash steps and some such methods include an elution step, and also wherein some methods also use target nucleic acid directly from the sample for further downstream processing (e.g., amplification) and/or analysis; 8) Removing, neutralizing or otherwise rendering less effective potentially competing, interfering, obscuring or otherwise detrimental to downstream analysis substances, components, contaminants, organisms (including dead or alive and/or debris from such organisms) or other biological, organic or inorganic material from the sample, such methods including depletion of non-target polynucleotides, including genomic DNA, including human genomic DNA, depletion of RNA, including rRNA, removal, digestion, inactivation or the like of proteins, enzymes, lipids, carbohydrates, biological material, organic material, inorganic material, cells, including whole cells and partially or completed lysed or otherwise degraded cells, and other components that can potentially interfere with downstream processing and/or analysis. Additional methods of sample preparation are described in J. Dapprich, et al., The next generation of target capture technologies-large DNA fragment enrichment and sequencing determines regional genomic variation of high complexity (BMC Genomics (2016), 17:486) and N Ali, et al., Current Nucleic Acid Extraction Methods and Their Implications to Point-of-Care Diagnostics (BioMed Research International (2017), Article ID 9306564, 13 pages), hereby incorporated by reference in their entirety.

Specific target capture (STC) methods refer to methods or combinations thereof that are useful for tagging, separating, isolating or otherwise differentiating specific target polynucleotides within a broader mixture of polynucleotides. This is in contrast to non-specific capture methods, which generally operate on all polynucleotides in a mixture (although there can be certain types of discrimination, for example, on the basis of length). STC methods typically differentiate between polynucleotides on the basis of nucleotide sequence (although other methods of discrimination are acceptable if the desired level of specificity is achieved). A preferred method in this closure is the use of STC oligonucleotides (oligos) to discriminate between targets on the basis of sequence. An oligo or set of oligos is/are designed to anneal specifically to a given target, group of targets, set of targets, etc., without annealing at any significant level to non-target polynucleotides potentially present in the sample mixture. Essentially any level of specificity can be achieved across the taxonomic spectrum through STC oligo design coupled with the selected reaction conditions. For example, target polynucleotides can be discriminated at the sub-species/strain, species, genus, family, order, class and/or phylum levels, and even at kingdom and domain levels. In a preferred embodiment of the disclosed invention wherein the system is utilized for the detection of blood stream infections as well as antimicrobial resistance genes, STC oligos are designed to bind to and selectively capture a wide range of bacterial and fungal targets and specific Antimicrobial Resistant (AMR) genes. Once STC oligos anneal to the intended targets, the resulting complexes can be captured, immobilized, separated, isolated and the like using a number of different methods, several examples of which are discussed elsewhere in this disclosure.

“Nucleic acid” and “polynucleotide” refer to a multimeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., inosine, or others; see The Biochemistry of the Nucleic Acids 5-36, Adams et al., ed., 11^{th} ed., 1992), derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that can affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates). Methylated cytosines such as 5-methylcytosines can be used in conjunction with any of the foregoing backbones/sugars/linkages including RNA or DNA backbones (or mixtures thereof) unless otherwise indicated. RNA and DNA equivalents have different sugar moieties (i.e., ribose versus deoxyribose) and can differ by the presence of uracil in RNA and thymine in DNA. The differences between RNA and DNA equivalents do not contribute to differences in homology because the equivalents have the same degree of complementarity to a particular sequence. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24, and 25). T residues are understood to be interchangeable with U residues, and vice versa, unless otherwise indicated. The orientation of a nucleic acid polymer strand can be described as plus (+) sense (or positive sense, or simply the sense or coding strand) or negative (−) sense (or minus, or simply the antisense or not-coding strand).

A “target polynucleotide” refers to a polynucleotide sought to be prepared, separated, captured, isolated, enriched, amplified, detected, identified and/or sequenced, and the like, using a composition or method described herein. In some embodiments, the target polynucleotide comprises a sequence of a DNA or RNA from an organism (e.g., any virus, prokaryote, eukaryote, protist, plant, fungus, insect, animal, mammal, or other biological entity, which may be living or formerly living). Exemplary DNAs include genomic DNA, circulating tumor DNA, episomal or plasmid DNA, and mitochondrial DNA. Exemplary RNAs include messenger RNA, transcribed RNA more generally, ribosomal RNA, transfer RNA, small nuclear RNA, regulatory RNA, transfer-messenger RNA, small nucleolar RNA, guide RNA, interfering RNA, micro RNA, other regulatory RNAs, non-coding RNA, etc. (and genomic RNA where applicable, e.g., in the case of certain viruses). Target polynucleotides can be positive sense, negative sense or both positive and negative sense (e.g., when both strands of a polynucleotide are targeted). Target polynucleotides also include one or more copies of the nucleic acids discussed above, to which additional sequences (such as any additional sequence described herein) may have been added. In some embodiments, a target polynucleotide comprises a non-naturally occurring sequence, e.g., resulting from in vitro synthesis, ligation, site-directed mutagenesis, recombination, or the like.

An “oligomer” or “oligonucleotide” refers to a nucleic acid of generally less than 1,000 nucleotides (nt), including those in a size range having a lower limit of about 2 to 5 nt and an upper limit of about 500 to 900 nt. Some particular embodiments are oligomers in a size range with a lower limit of about 5 to 35 nt and an upper limit of about 50 to 600 nt, and other particular embodiments are in a size range with a lower limit of about 5 to 20 nt and an upper limit of about 30 to 1500 nt. Oligomers can be purified from naturally occurring sources, but can be synthesized by using any well known enzymatic or chemical method. Oligomers can be referred to by a functional name (e.g., capture oligomer, primer, promoter primer or detection probe) but those skilled in the art will understand that such terms refer to oligomers. Oligomers can form secondary and tertiary structures by self-hybridizing or by hybridizing to other oligonucleotides or polynucleotides. Such structures can include, but are not limited to, duplexes, hairpins, cruciforms, bends, triplexes and tetraplexes. Oligomers can contain modifications, including those described elsewhere in this disclosure. In some case, oligomers can refer to non-nucleic-based polymers, such as some aptamers and non-nucleotide-based binding partners (e.g., see Winnacker, M,, & Kool, E. T. (2013). Artificial Genetic Sets Composed of Size-Expanded Base Pairs. ANGEWANDTE CHEMIE-INTERNATIONAL EDITION, 52(48), 12498---508.). Oligomers may be generated in any manner, including chemical synthesis, DNA replication, reverse transcription, PCR, or a combination thereof. In some embodiments, oligomers that form invasive cleavage structures are generated in a reaction (e.g., by extension of a primer in an enzymatic extension reaction).

“Arbitrary sequence” refers to any sequence that is chosen, selected, determined, designed, etc., by the user (with or without the aid of a computer program), typically to serve a desired function or purpose in a downstream process. In a preferred mode, the arbitrary sequence is designed to be non-complementary to or otherwise non-reactive with a target sequence or sequences under given conditions of a process. In some embodiments, the arbitrary sequence can be a randomly generated sequence or set of sequences, for example for use as a unique molecular identifier or a universal primer.

“Capture oligomer,” “capture oligonucleotide,” “capture probe,” “target capture oligomer,” and “capture probe oligomer” are used interchangeably to refer to a nucleic acid oligomer or derivative thereof, that comprises a target-binding sequence (TBS) capable of binding one or more target sequences in a target nucleic acid. The binding can occur at different, user-selected levels of specificity depending on the design of the system and the desired application and outcome. One mode of binding comprises hybridizing to the target nucleic acid, again with different, user-selected levels of specificity, from high specificity to low specificity (including designing the capture oligomer(s) to capture the desired targets at any point in the taxonomic order; see discussed elsewhere in this disclosure). Target capture oligomers can also comprise segments (or the entire oligo) of random or semi-random sequences. The capture oligomer can also comprise one or more of, (i) an extendable 3′-terminus, (ii) non-extendable (e.g., blocked) 3′-terminus, (iii) a ligatable 5′-terminus, (iv) a first ligand of a ligand pair (e.g., biotin of the ligand pair biotin/streptavidin), of which there may be one or more copies, (v) a tag sequence appended to the 3′-end, 5′-end or both and/or inserted into the THS, (vi) a combination of a tag sequence or sequences and one or more ligands. An exemplary tag sequence comprises a capture sequence capable of hybridizing to a secondary oligomer, e.g., immobilized on a solid support or a non-immobilized secondary oligomer linked to a binding partner to facilitate isolation of a complex comprising the capture oligomer, the target, and the secondary oligomer from other molecules in a composition. Other exemplary tag sequence options are described in the “tag” definition section below. The nucleic acid components of the capture oligomer comprise any of the forms or combinations thereof described in the “nucleic acid” definition section above. A capture oligomer can in certain cases function as more than a capture agent, including but not limited to a primer, an amplification oligomer, a blocker, part of a site for cleavage/digestion, a displacer, part of a recognition site, and the like. A capture oligomer can also be any of the capture oligomers described in “COMPOSITIONS, KITS AND METHODS FOR ISOLATING TARGET POLYNUCLEOTIDES” (PCT/GB2021/050098), incorporated herein by reference in its entirety. This reference describes in detail copy control-related compositions, kits and methods (discussed elsewhere within).

By “nucleic acid amplification” or “amplification” (within the clear context of nucleic acids; “amplification” can have a different meaning in a different context, e.g., the production of 1 or more copies of non-nucleic acid molecules, the increase of a detection signal, such as fluorescence, the increase in electrical signal, etc.) is meant the production of 1 or more copies of a target nucleic acid (or target polynucleotide, parent molecule, template, template molecule, etc.) sequence, or its complementary sequence, or a portion thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), the polymerase chain reaction (PCR) (e.g., U.S. Pat. Nos. 4,683,195, 4,683,202, and 4,800,159), rolling circle amplification (RCA) (e.g., U.S. Pat. Nos. 5,854,033 and 6,143,495), recombinase polymerase amplification (RPA) (e.g., U.S. Pat. No. 7,666,598), ligase chain reaction (LCR) (e.g., EP Pat. App. 0320308), loop-mediated amplification (e.g., Loop-mediated isothermal amplification of DNA (2000) Nucleic Acid Res, 28 (12): e63) and strand-displacement amplification (SDA) (e.g., U.S. Pat. No. 5,422,252). Replicase-mediated amplification uses self-replicating RNA molecules, and a replicase such as QB-replicase. PCR amplification uses DNA polymerase, primers, and cycling steps (typically thermal cycling, but other types of cycling, such as chemical cycling, can also be used) to synthesize multiple copies of the two complementary strands of DNA or cDNA. The copies of the two complementary strands can be produced in a ratio other than 1-to-1, for example, in asymmetric PCR (e.g., Asyninietric PCR. In: Capinera J. L. (eds) Encyclopedia of Entomology, 2008, Springer, Dordrecht.). LCR amplification uses at least four separate oligonucleotides to amplify a target and its complementary strand by using multiple cycles of hybridization, ligation, and denaturation. SDA uses a primer that contains a recognition site for a restriction endonuclease that will nick one strand of a hemimodified DNA duplex that includes the target sequence, followed by amplification in a series of primer extension and strand displacement steps. Particular embodiments use PCR, but it will be apparent to persons of ordinary skill in the art that oligomers disclosed herein can be readily used as primers in other amplification methods and that in general other amplification methods and primers can be used.

By “amplicon” or “amplification product” is meant a nucleic acid molecule generated in a nucleic acid amplification reaction and which is derived from a template nucleic acid. An amplicon or amplification product contains an amplified nucleic acid sequence (e.g., target polynucleotide/nucleic acid) that can be of the same or opposite sense as the template nucleic acid, comprises DNA or RNA, and comprises single-stranded or double-stranded product. In some embodiments, an amplicon has a length of about 100-30,000 nucleotides, about 100-10,000 nucleotides, about 100-5000 nucleotides, 100-2000 nucleotides, about 100-1500 nucleotides, about 100-1000 nucleotides, about 100-800 nucleotides, about 100-700 nucleotides, about 100-600 nucleotides, or about 50-500 nucleotides.

An “amplification oligonucleotide” or “amplification oligomer” refers to an oligonucleotide that hybridizes to a target nucleic acid, or its complement, or a tag sequence and the like, and participates in a nucleic acid extension or amplification reaction, e.g., serving as a primer and/or promoter-primer, a displacer (with or without extension), a blocker (e.g., blocking binding or extension), to help facilitate cleavage or degradation, to disrupt structure, and the like. Some capture oligomers can also serve as amplification oligomers (see described elsewhere in this specification) and some amplification oligomers can also serve as capture oligomers. Amplification oligomers also encompass promoter-providers, which contain a promoter from which transcription can be initiated but are not necessarily extendable by a DNA polymerase and may comprise a 3′ blocking moiety. Particular amplification oligomers contain a target-, complement- or tag-hybridizing sequence of at least about 5 contiguous bases, and optionally at least 10, 11, 12, 13, 14, 15, 16, 17, 18, 19, 20, 21, 22, 23, 24 or 25 contiguous bases, that are complementary to a region of the target nucleic acid sequence or a tag sequence, or the complementary strands thereof. Other exemplary lengths or ranges of lengths for target- or tag-hybridizing sequences are described elsewhere herein and can apply to amplification oligomers. The contiguous bases can be at least about 70%, at least about 80%, at least about 90%, or completely complementary to the target sequence to which the amplification oligomer binds. In some embodiments, an amplification oligomer comprises an intervening linker or non-complementary sequence between two segments of complementary sequence, e.g., wherein the two complementary segments of the oligomer collectively comprise at least about 5 complementary bases, and optionally at least 10, 11, 12, 13, 14, 15, 16, 17, 18, 19, or 20 complementary bases. In some embodiments, amplification oligomers are about 10 to about 80 bases long and optionally can include modified nucleotides. An amplification oligomer can be optionally modified, e.g., by including a 5′ region that is non-complementary to the target sequence. Such modification can include functional additions, such as tags, promoters, or other sequences or moieties used or useful for manipulating, amplifying, capturing, immobilizing or otherwise processing the primer, tag or target molecules.

A “primer” refers to an oligomer that hybridizes to a template nucleic acid and has a 3′ end that is extended by polymerization. A primer can be optionally modified, e.g., by including a 5′ region that is non-complementary to the target sequence. Such modification can include functional additions, such as tags, promoters, or other sequences, or other moieties, all of which can be used or are useful for manipulating, amplifying, capturing, separating, immobilizing or otherwise processing the primer or target oligonucleotide or complements thereof “Opposed primers” means at least one primer that is positive (+) sense and at least one primer that is minus (−) sense, each complementary to one of the strands of the target polynucleotide or a copy of one of the strands of the target polynucleotide, such that when used together in an amplification reaction they participate in the production of amplicon (e.g., a primer pair in PCR).

An amplification oligomer or primer can in certain cases function as more than an amplification oligomer and primer, including but not limited to a capture oligomer, a blocker, part of a site for cleavage, digestion, a displacer, part of a recognition site, and the like.

Unless otherwise stated to the contrary, a first sequence is a “complement” of a second sequence (or, equivalently, is “complementary” to the second sequence) where the first sequence has a length and content sufficient to anneal to the second sequence under reasonable binding conditions, which may be but are not necessarily stringent hybridization conditions described herein and also encompass, e.g., annealing conditions as used in standard PCR and other techniques involving primer or probe binding and extension.

A “tag” refers to any additional nucleic acid sequence other than a target-hybridizing sequence that may be included in an oligomer or added to or inserted into a target polynucleotide. Any arbitrary sequence that is present in addition to a target-hybridizing sequence can serve as a tag. In some usages a tag can also refer to a moiety other than nucleic acid attached to or otherwise included in an oligomer or target polynucleotide. A tag can be included, added, inserted, appended or the like into/to a target polynucleotide or a fragment(s) thereof using any method known in the art, including but not limited to incorporation via extension or amplification utilizing an oligomer containing one or more tags and that is hybridized specifically, semi-specifically or non-specifically to a target polynucleotide or a fragment(s) thereof, ligation, via transposome chemistry, etc. A tag includes but is not limited to an adaptor (see below). Additional examples of tags are promoters, mixed-nucleotide elements described elsewhere herein, elements used for sample preparation and target capture, and stabilizing sequences including clamps. Still additional examples of tags are given in the “Sample Preparation” definition section above and also described elsewhere within.

An “adaptor” or “adapter” (these 2 terms are used interchangeably in this disclosure and the definitions are equivalent) is a sequence that adapts the molecule to which it is added to provide one or more additional functionalities. For example, an adaptor provides a binding site for another molecule, e.g., an amplification oligomer, a sequencing primer, or a capture oligomer. The binding site may be a universal binding site (e.g., for multiple capture oligomers, all with the same binding site specificity, e.g., the same sequence, in a multiplex format, or for a universal primer). Additional examples of binding sites are a binding site for a displacer oligomer, probe, or nucleic acid modifying enzyme (e.g., RNA polymerase, primase, ligase, RNAse (such as RNAse H) or a restriction enzyme), or for attachment to a solid phase (including via a solid phase primer or capture oligomer) including for use in clonal amplification, or other functional element or elements useful in a downstream application, e.g., enrichment, library preparation, clonal amplification or sequencing. Thus, sample barcodes or index sequences, key or calibrator sequences, molecular barcodes (including unique molecular identifier), sites for downstream cloning and sites for circularization of a target molecule are additional examples of elements that can be included in adaptors.

By “library preparation” is meant in the most general sense to be a process to make ready a group of target polynucleotides for further downstream processing and/or analysis. Cluster generation, including via clonal amplification, is one example of a downstream processing step.

Sequencing is one example of downstream analysis. Library preparation methods will often include a step or steps for the addition of adapter sequences or other tag sequences to some or all of the molecules in the library. This is not always the case, however, as some of the disclosed embodiments do not require adapters to be added to the library molecules. An example of this includes some modes of the Direct Hybridization sequencing method, which is described elsewhere in this disclosure. Library preparation often also includes an amplification step or steps, for example, to enrich one or more regions of a target polynucleotide, to add tags, including adapters, to library molecules and to increase the copy numbers of target regions and/or molecules containing tags, including adapters. Adapters and tags can be added without amplification, for example, via ligation. Library preparation steps can overlap with sample preparation steps (for example, tags, including adapters, can be added during sample preparation and/or a first extension product from a targeted region can be made (see “Sample Preparation” sections herein)) and can also be associated/overlap with copy control (see elsewhere herein, including immediately below).

By “copy control” is meant compositions and methods by which the number of copies of molecules that are the output of a given process are controlled in a predetermined manner. For example, in certain workflows it is desirable to capture (or amplify and capture) or otherwise isolate an amount of a target polynucleotide (which for example may be a natural DNA or RNA or an amplicon) no greater than a predetermined amount (e.g., a maximum desirable value for a downstream application, such as sequencing library preparation; can in some cases be referred to as “limited capture”, which still falls under the umbrella definition of copy control). Similarly, in certain workflows it is desirable to capture (or amplify and capture) or otherwise isolate a predetermined specific amount (e.g., a specific number of molecules or copies of molecules) of a target polynucleotide (which for example may be a natural DNA or RNA, an amplicon or a sequencing library) for use in a downstream application (e.g., clonal amplification, including in next generation sequencing workflows). Further, in certain workflows it is desirable to incorporate additional sequences into a target polynucleotide (tags), such as incorporation of adaptors into a sequencing library. This can also be accomplished in copy control compositions and methods. The present disclosure provides oligomers, compositions, and kits, useful for isolating target polynucleotides and/or attaching tags such as adaptors. Isolation includes isolation in limited amounts (limited capture) and specific amounts (copy control).

By “clonal” or “monoclonal” is meant a population of identical units or copies of a (at least one) progenitor molecule, nucleic acid, polynucleotide, gene, genetic material, cell and the like. In this disclosure it most often refers to a population of identical copies made from one or more copies of the same nucleic acid/polynucleotide template. On occasion in this disclosure it can refer to a collection of identical molecules in a population that, for example, are clustered together (see below). In the most preferred embodiment, monoclonality is about 100% (i.e., all or nearly all of the copies are identical). In other preferred embodiments, monoclonality is about 90% or higher, about 80% or higher or 70% or higher. In some embodiments, monoclonality is between about 50 and 70%. By “polyclonal” is meant a population of units or copies that are not all identical and that are derived from at least 2 different progenitor molecules or the like. By clonal amplification is meant nucleic acid amplification of typically a single progenitor nucleic acid molecule (although in some cases it can be 2 or more identical progenitor molecules) to produce an identical set of copies. By “cluster” is meant a grouping of molecules, e.g., nucleic acid molecules, bound to a solid support. By “cluster generation” is meant the process by which a cluster is produced. Examples of cluster generation processes include amplification-based, such as clonal amplification, and non-amplification-based, such as hybridization of target molecules to an oligonucleotide immobilized to a solid support in a known, specific region (e.g., a spot). A cluster can be monoclonal (typically the preferred configuration in this disclosure) or polyclonal.

A “linker” is a sequence or non-sequence element or a combination thereof that connects one portion of an oligomer to another. In some embodiments, a sequence linker comprises sequence that does not hybridize to a target polynucleotide and/or to other oligomers in a combination or composition. In some embodiments, a non-sequence linker comprises alkyl, alkenyl, amido, or polyethylene glycol groups [(—CH₂CH₂O—)ₙ].

A “stabilizing sequence” is a clamp, mixed-nucleotide region, or other sequence that functions to increase the stability of a duplex region and/or control the register of hybridization (e.g., when located adjacent to a sequence prone to slippage, such as a containing repeating nucleotides, e.g., a poly-dA or poly-dT sequence). An “aligning sequence” is a stabilizing sequence that controls the register of hybridization. In addition to clamps and mixed-nucleotide regions described elsewhere herein, stabilizing sequences include GC-rich sequences and sequences containing affinity-enhancing modifications.

An “internal extension blocker” is an element located within the sequence of a nucleic acid or bound to the nucleic acid that prevents extension of a complementary strand along the nucleic acid. Examples include a non-nucleotide linker or one or more abasic sites, non-natural nucleotides, or chemically modified natural nucleotides, as well as reversible extension blockers discussed below.

A “reversible extension blocker” is an internal extension blocker that can have its blocking function reversed, i.e., be rendered permissive for extension of the complementary strand. An exemplary reversible extension blocker is a non-natural nucleotide that has a complementary nucleotide that is accepted by a polymerase and that exhibits specificity relative to natural nucleotides (i.e., a polymerase will not add a natural base across from the reversible extension blocker). Providing the complementary nucleotide reverses the blocking function. Examples of non-natural base pairs, where either member of the pair can serve as the reversible extension blocker, are Iso-dC or Iso-dG; xanthine or 5-(2,4 diaminopyrimidine); 2-amino-6-(N,N-dimethylamino)purine or pyridine-2-one; 4-Methylbenzimidizole or 2,4 Difluorotoluene; 7-Azaindole or Isocarbostyril; dMMO2 or d5SICS; or dF or dQ. Other examples of a reversible extension blocker are a chemically modified nucleotide or nucleotides wherein the modification is attached via a reversible linkage, wherein the linkage can be reversed by providing any one or more of a chemical, an enzyme, a temperature change, a reagent composition change, etc.; a reversible nucleic acid structural feature; or a molecule reversibly bound to the capture oligomer, optionally wherein the reversibly bound molecule is a protein, an enzyme, a lipid, a carbohydrate, or a chemical moiety.

By “hybridization” or “hybridize” is meant the ability of two completely or partially complementary nucleic acid strands to come together under specified hybridization assay conditions in a parallel or antiparallel orientation to form a stable structure having a double-stranded region. “Hybridization” and “hybridize” are synonymous with “annealing” and “anneal,” respectively. The two constituent strands of this double-stranded structure, sometimes called a hybrid, are held together by hydrogen bonds. Although these hydrogen bonds most commonly form between nucleotides containing the bases adenine and thymine or uracil (A and T or U) or cytosine and guanine (C and G) on single nucleic acid strands, base pairing can also form between bases which are not members of these “canonical” pairs. Non-canonical base pairing is well-known in the art. (See, e.g., R. L. P. Adams et al., The Biochemistry of the Nucleic Acids (11th ed. 1992).) In addition, a triple-stranded region can also be formed by hybridizing a third strand to a B-form DNA duplex via Hoosteen base pairing.

As used herein, the term “specifically hybridizes” means that under given hybridization conditions a probe, primer, or other oligomer (e.g., capture oligomer) detectably hybridizes substantially only to its target sequence(s) in a sample comprising the target sequence(s) (i.e., there is little or no detectable hybridization to non-targeted sequences). Notably, an oligomer can be configured to specifically hybridize to any one of a set of targets (e.g., sequences from organisms of a particular taxonomic group, e.g., species, genus, or higher). In some embodiments, a probe, primer, or other oligomer (e.g., capture oligomer) can hybridize to its target nucleic acid to form a stable oligomer:target hybrid, but not form a sufficient number of stable oligomer:non-target hybrids for amplification or capture as the case may be. Amplification and capture oligomers that specifically hybridize to a target nucleic acid are useful to amplify and capture target nucleic acids, but not non-targeted nucleic acids, especially non-targeted nucleic acids of phylogenetically closely related organisms. Thus, the oligomer hybridizes to target nucleic acid to a sufficiently greater extent than to non-target nucleic acid to enable one having ordinary skill in the art to accurately capture, amplify, and/or detect the presence (or absence) of nucleic acid derived from the specified target (e.g., a particular pathogen) as appropriate. In general, reducing the degree of complementarity between an oligonucleotide sequence and its target sequence will decrease the degree or rate of hybridization of the oligonucleotide to its target region. However, the inclusion of one or more non-complementary nucleosides or nucleobases may facilitate the ability of an oligonucleotide to discriminate against non-targeted nucleic acid sequences.

By “stringent hybridization conditions,” or “stringent conditions” is meant conditions (1) permitting an oligomer to preferentially hybridize to a target nucleic acid as opposed to a different nucleic acid (e.g., a nucleic acid having as little as 1 nucleotide difference in identity to the target nucleic acid) or (2) permitting only an oligomer with a higher affinity target-hybridizing sequence (relative to an oligomer with a lower affinity target-hybridizing sequence) to hybridize to a target, e.g., wherein the higher affinity target-hybridizing sequence is longer than the lower affinity target-hybridizing sequence and/or comprises affinity-enhancing modifications that the lower affinity target-hybridizing sequence does not comprise. While the definition of stringent hybridization conditions does not vary, the actual reaction environment that can be used for stringent hybridization may vary depending upon factors including the GC content and length of the oligomer, the degree of similarity between the oligomer sequence and sequences of targeted and non-targeted nucleic acids that may be present in the test sample.

Hybridization conditions include the temperature and the composition of the hybridization reagents or solutions. Exemplary stringent hybridization conditions with the oligomers of the present disclosure correspond to a temperature of about 40° C. to 75° C., e.g., 40° C. to 50° C., 50° C. to 60° C., or 60° C. to 75° C., when the monovalent cation concentration is in the range of about 0.4-1 M and the divalent cation concentration is in the range of about 0-10 mM and the pH is in the range of about 5-9. Additional details of hybridization conditions are set forth in the Examples section. Other acceptable stringent hybridization conditions could be easily ascertained by those having ordinary skill in the art.

“Label” or “detectable label” refers to a moiety or compound joined directly or indirectly to an oligomer that is detected or leads to a detectable signal. Any detectable moiety can be used, e.g., radionuclide, ligand such as biotin or avidin, enzyme, enzyme substrate, reactive group, chromophore such as a dye or particle (e.g., latex or metal bead) that imparts a detectable color, luminescent compound (e.g. bioluminescent, phosphorescent, or chemiluminescent compound), and fluorescent compound (i.e., fluorophore). Embodiments of fluorophores include those that absorb light (e.g., have a peak absorption wavelength) in the range of about 495 to 690 nm and emit light (e.g., have a peak emission wavelength) in the range of about 520 to 710 nm, which include those known as FAM™, TET™, HEX, CAL FLUOR™ (Orange or Red), CY, and QUASAR™ compounds. Fluorophores can be used in combination with a quencher molecule that absorbs light when in close proximity to the fluorophore to diminish background fluorescence. Such quenchers are well known in the art and include, e.g., BLACK HOLE QUENCHER™ (or BHQ™), Blackberry Quencher® (or BBQ-650®), Eclipse®, or TAMRA™ compounds.

A “non-extendable” oligomer or an oligomer comprising a “blocking moiety at its 3′ end” includes a blocking moiety sufficiently close to its 3′-terminus (also referred to as the 3′ end) to prevent extension. Any blocking moiety that is sufficiently close to the 3′-terminus to block extension is considered to be “at” the 3′-terminus for purposes of the present disclosure even if it is not bound to or present in place of a 3′ hydroxyl or oxygen. A blocking moiety near the 3′ end is in some embodiments within five residues of the 3′ end and is sufficiently large to limit binding of a polymerase to the oligomer, and other embodiments contain a blocking moiety covalently attached to the 3′ terminus. Many different chemical groups can be used to block the 3′ end, e.g., alkyl groups, non-nucleotide linkers, alkane-diol dideoxynucleotide residues (e.g., 3′-hexanediol residues), and cordycepin. Further examples of blocking moieties include a 3′-deoxy nucleotide (e.g., a 2′,3′-dideoxy nucleotide); a 3′-phosphorylated nucleotide; a fluorophore, quencher, or other label that interferes with extension; an inverted nucleotide (e.g., linked to the preceding nucleotide through a 3′-to-3′ phosphodiester, optionally with an exposed 5′-OH or phosphate); or a protein or peptide joined to the oligonucleotide so as to prevent further extension of a nascent nucleic acid chain by a polymerase. A non-extendable oligonucleotide of the present disclosure can be at least 10 bases in length, and can be up to 15, 20, 25, 30, 35, 40, 50 or more nucleotides in length. Non-extendable oligonucleotides that comprise a detectable label can be used as probes.

A “binding partner” is a member of a pair of moieties that can be used to form a noncovalent association. An exemplary set of binding partners is biotin and a biotin-binding agent. Further examples of binding partners include, but are not limited to digoxygenin/anti-digoxygenin and more generally an antibody and its target.

A “biotin-binding agent” is an agent (e.g., polypeptide) that can specifically bind biotin. Streptavidin, avidin, and NeutrAvidin represent examples of biotin-binding agents. An anti-biotin antibody is also considered a biotin-binding agent.

The term “antibody” encompasses any polypeptide comprising a functional antigen-binding region having complementarity-determining regions and framework regions (e.g., a VH and VL domain), including without limitation scFv, Fab, and full-length antibodies (e.g., IgA, IgG, IgD, IgE, or IgM antibodies).

The terms “Unique Molecular Identifiers (UMI),” “Unique Identifiers (UID),” “molecular barcode,” “randomer,” “random molecular tag,” “random barcode,” “primer ID,” “molecular labels,” “single molecule barcode” and “single molecule identifier (SMI)” are used interchangeably to refer to polynucleotide sequences, the sequence of which may be random, non-random, partially degenerate or degenerate. UMIs may be used to barcode DNA molecules prior to PCR or sequencing methods so that individual DNA strands can be identified. Amplicons containing the same UMIs are assumed to originate from the same DNA molecule. UMIs can identify between true errors and those arising due to PCR or the sequencing method. UMIs may be between about 5 and 100 nucleotides in length or longer as required to facilitate differentiating between a larger number of DNA strands and may be of variable or uniform length. In some implementations, the UMIs may be introduced in the first two PCR cycles or using methods such as ligation, transposition through polymerase, endonuclease, transposase or any other method known in the art.

The term “triangulation” means combining two or more results or data from independent analyses to determine an answer with increased confidence.

As used herein, a “combination” of oligomers refers to any plurality of oligomers in proximity to each other, e.g., in different containers or the same container in a kit, or in a composition or set of compositions juxtaposed to one another, e.g., in a plate, rack, or other container.

Unless defined otherwise, all scientific and technical terms used herein have the same meaning as commonly understood by those skilled in the relevant art. General definitions can be found in technical books relevant to the art of molecular biology, e.g., DICTIONARY OF MICROBIOLOGY AND MOLECULAR BIOLOGY, 2nd ed. (Singleton et al., 1994, John Wiley & Sons, New York, NY) or THE HARPER COLLINS DICTIONARY OF BIOLOGY (Hale & Marham, 1991, Harper Perennial, New York, NY).

### EXAMPLES

The following examples are provided to illustrate certain disclosed embodiments and are not to be construed as limiting the scope of this disclosure in any way.

A. Retrieval of Spiked Antimicrobial Resistance (AMR) Targets Direct from Blood (DfB) Using Specific Target Capture (STC) Oligomers

Oligomers (all supplied by IDT; 3′invdT and 3′invdC are inverted nucleotides on the 3′-terminus)

Bacterial Target Organisms—Klebsiella pneumoniae, ATCC strain BAA-1898; contains AMR carbapenemase gene (KPC); Staphylococcus aureus, ATCC strain BAA-2094; contains AMR mecA gene (mecA)

Specific Target Capture (STC) oligomers—KPC STC oligomers were designed to bind to AMR KPC gene with the following sequence:

KPC_STC_F (SEQ ID No. 6):
5′-Biotin/AAAAACACCGCGCTGACCAACCTC/3′invdT
KPC_STC_R (SEQ ID No. 7):
5′-Biotin/AAAAACACAGCGGCAGCAAGAAAGC/3′invdT

mecA STC oligomers were designed to bind to mecA with the following sequence:

mecA_STC_F-INT (SEQ ID No. 8):
5′-Biotin/AAAAAAGGTACTGCTATCCACCCTCAAACAGGT/
3′invdT
mecA_STC_R2 (SEQ ID No. 9):
5′-Biotin/AAAAATTGAGTTGAACCTGGTGAAGTTGTAATCTGG/
3′invdT

The STC oligomers were designed to capture targets with the following sequence: mecA: 5′-Biotin/sequence/3′invdC

Quantitative PCR (qPCR) was performed with primers with the following sequences:

PCR 1
KPC_P28_PCR1_F (SEQ ID No. 10):
5′-AACCATTCGCTAAACTCGAACAGG-3′
KPC_P28_PCR1_R (SEQ ID No. 11):
5′-CCTTGAATGAGCTGCACAGTGG-3′
mecA_P40_PCR1_F (SEQ ID No. 12):
5′-CATGAAAAATGATTATGGCTCAGGTAC-3′
mecA_P40_PCR1_R (SEQ ID No. 13):
5′-TGGAACTTGTTGAGCAGAGGTTC-3′
PCR 2
KPC_P28_PCR2_F (SEQ ID No. 14):
5′-CTTTGGCGGCTCCATCGG-3′
KPC_P28_PCR2_R (SEQ ID No. 15):
5′-CTCCTCAGCGCGGTAACTTAC-3′
mecA_P40_PCR2_F (SEQ ID No. 16):
5′-GCTATCCACCCTCAAACAGGTGAAT-3′
mecA_P40_PCR2_R (SEQ ID No. 17):
5′-ATTCTTCGTTACTCATGCCATACATA-3′

### Protocol Reaction Conditions

The reaction was performed in a 15 mL Falcon conical polypropylene tube (Coming 352097) by adding 50 μL, 25 μL or 15 μL pathogen spike (10, 5 or 3 CFU/mL respectively), 1 mg Proteinase K (20 mg/mL, Promega MC5008), 30 μL STC oligo pool (20 μmol/5 μL), 100 μL antifoam Y-30 emulsion (Sigma-Aldrich A6457-100ML) and 1.667 mL lysis NS4× buffer formulation E (100 mM Tris pH 8.0, 16.675% SDS) to 5 mL of whole blood.

The reaction mixture was mixed by vortexing and inverting for about 15 seconds per sample, and pulse spun up to 700 relative centrifugal force (RCF) for a total duration of 10 seconds using a swing bucket centrifuge (Centrifuge 5810, Eppendorf).

Digestion of proteins followed by cell lysis—The reaction mixture was incubated for 15 minutes in a custom laboratory heat block (custom, DNAe) pre-heated to 75° C. where the sample reached an internal temperature of about 60° C. after 15 minutes of incubation.

The mixture was then transferred to an 8 mL polypropylene screwcap tube (Fischer Scientific, NC9691446) containing pre-measured 4 gr of 0.1 mm VHD ZrO mechanical lysis (ML) beads (GlenMills Grinding Media) and mechanical lysis (ML) was performed using an OMNI Bead Ruptor Elite (OMNI International) using the “blood” program (3 cycles of 90 seconds on “mix” and 20 seconds off “dwell”, at 6.6 m/s speed).

The mechanically lysed sample was allowed to cool at room temperature (about 20-26° C.) for 5 minutes. The ML tube was then centrifuged for 1 minute at 700 RCF and the liquid transferred back to the original 15 mL tube, leaving the ML beads behind. Sample tube was pulse spun in a centrifuge up to 700 RCF for a total time of 10 seconds.

Deactivation of Proteinase K and DNA denaturation—The tube was placed on a heat block pre-heated to 100° C. and incubated for 30 minutes.

Target DNA capture using biotinylated STC oligos—The tube was then transferred to a heat block pre-heated to 60° C. and incubated for 40 minutes.

Capture of target DNA-STC oligo with streptavidin beads—The tube was removed from the 60° C. heat block, the contents were allowed to cool slightly to ambient temperature (about 20-26° C.) and 1.2 mg streptavidin beads (custom Streptavidin beads, DNAe) were added. The tube was then incubated in a heating/cooling shaking incubator (Benchmark Scientific, model HC5000-HC) for 10 minutes at 45° C. with constant mixing (1500 RPM).

Bead separation—The sample was pulse spun in a centrifuge up to 700 RCF up to 10 seconds and the sample tube was placed in magnetic rack (Invitrogen, DynaMag-15) for 5 minutes and the supernatant was aspirated and discarded.

Washing of beads with wash-S buffer—The beads were washed by adding 1 mL of Wash-S buffer (50 mM Tris pH 8.0, 0.1% SDS, 150 mM NaCl) and agitated using the magnet. The washed beads and buffer were then transferred from the 15 mL tube to a new 1.5 mL tube, then magnetized for 2 minutes and the Wash-S buffer removed and discarded. The beads were then washed again by adding 1 mL Wash-S buffer, magnetizing for 2 minutes, then removing and discarding the Wash-S buffer.

Washing of beads with Wash-T buffer (10 mM Tris pH 8.0, 0.01% Tween-20)—The sample was next washed with 1 mL Wash-T buffer, magnetized for 2 minutes then the buffer removed and discarded. The bead washing with Wash-T buffer was then repeated once more.

Target elution from the beads—The magnet was removed from the 1.5 mL and 50 μL IDTE elution buffer (10 mM Tris pH 7.5, 0.1 mM EDTA; Integrated DNA Technologies) added. The sample was then mixed, pulse spun and incubated at 75° C. for 3 minutes to elute the target DNA from the streptavidin beads. The tube was then magnetized for 2 minutes, then the eluant containing the target DNA transferred to a new 1.5 mL tube (DNA LoBind Tube, 022431021, Eppendorf).

Quantitation—Capture of mecA and KPC targets from the whole blood was confirmed using two qPCR reactions using the primers shown above.

### Results and Conclusions

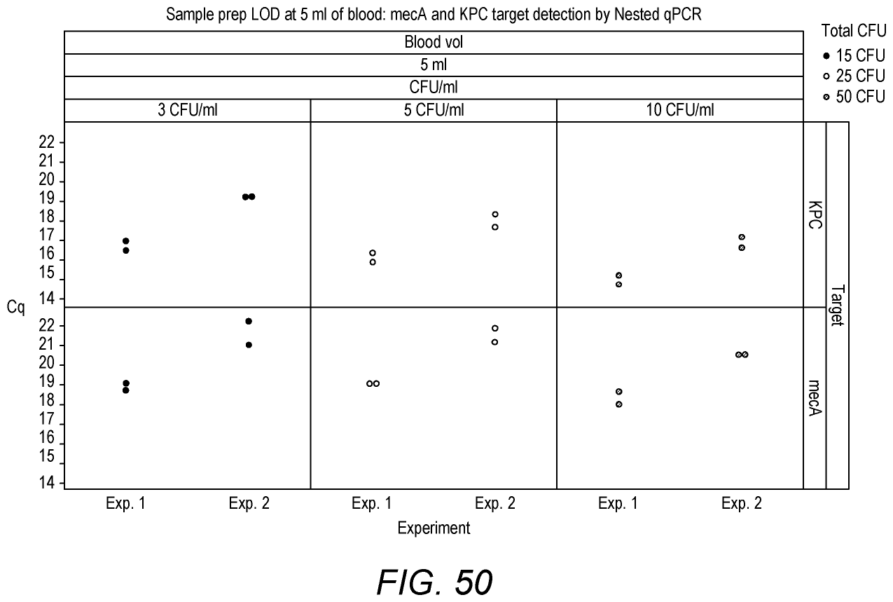

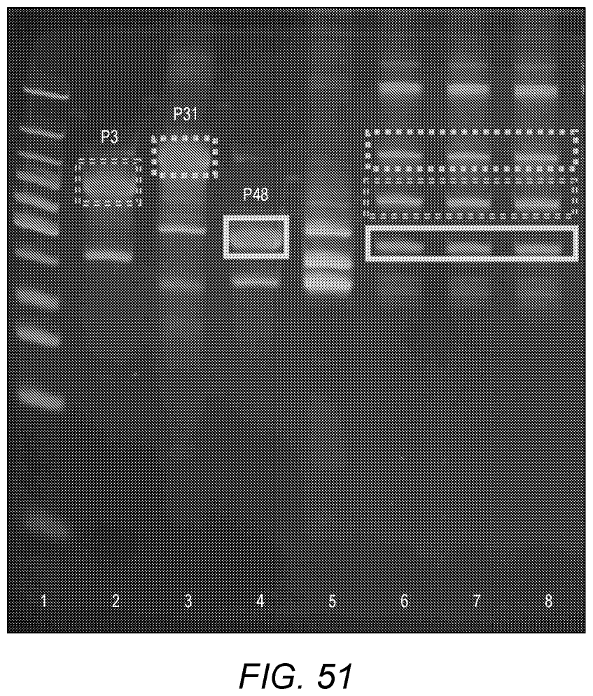

Two organisms containing AMR targets—Klebsiella pneumonia, containing KPC, and Staphylococcus aureus, containing mecA—were each spiked at either 10, 5 or 3 CFU/mL into 5 mL of whole blood and the aforementioned protocol (steps 6-19 above) performed. The qPCR data in FIG. 50 shows the desired target was captured from the whole blood.

B. Multiplex full capture protocol

### Oligomers

The following nested PCR2 primers (all supplied by IDT; “52-Bio” represents two biotin groups on the 5′-end of the oligomer) were used to amplify each of the targets specified:

16s rRNA target
P3F (SEQ ID No. 18):
5′-AAAACGAGACATGCCGAGCATCCGCTTTAAGTCCCGCAACGAGCG
CAA-3′
P3R (SEQ ID No. 19):
/52-Bio/ACCGTGCTGCCTTGGCTTCATTGTGGTCTTGACGTCATCC
CCACCTTCCTC-3′
23s rRNA target
P31F1 (SEQ ID No. 20):
5′-AAAACGAGACATGCCGAGCATCCGCCGCATGTGTAGGATAGGTGG
GAG-3′
P31F2 (SEQ ID No. 21):
5′-AAAACGAGACATGCCGAGCATCCGCCGCATGTACAGGATAGGTAG
GAG-3′
P31R (SEQ ID No. 22):
/52-Bio/GAGACCGCCCCAGTCAAACT-3′
CTX-M Group 1 target
P48F (SEQ ID No. 23):
/52-Bio/AAAACGAGACATGCCGAGCATCCGCTGTTAGGAAGTGTGC
CGCTG-3′
P48R (SEQ ID No. 24):
5′-ACCGTGCTGCCTTGGCTTCATTGTGGTCTCCCGACTGCYGCTCTA
AT-3′
(‘Y’ is a mix of C and T)

### Protocol Reaction Conditions

1000 genomic copies of E. Coli genomic DNA (gDNA) was spiked into the PCR2 singleplex reactions for P3F and P3R, P31F1, P3 1F2 and P311R, and P48F and P48R. The reaction was prepared in a 0.2 mL tube according to the formulation shown in Table 1.

TABLE 1

| | | Stock | | Final | | Per 50.0 μL | |
|---|---|---|---|---|---|---|---|
| Component | Manufacturer | concentration | | concentration | | reaction | |
| 5X SuperFi Buffer | ThermoFisher | 10X | | 1X | | 5 | μL |
| Forward Primer | IDT | 100 | μM | 1.5 | μM¹ | 0.75 | μL |
| Reverse Primer | IDT | 100 | μM | 1.5 | μM | 0.75 | μL |
| dNTP Mix² | NEB | 10 | mM³ | 0.4 | mM³ | 2 | μL |
| MgCl₂ | ThermoFisher | 1000 | mM | 1.75 | mM | 0.0875 | μL |
| Platinum ™ SuperFi ™ | ThermoFisher | 2 | U/μL | 0.02 | U/μL | 0.5 | μL |
| DNA Polymerase | | | | | | | |

| SYBR Green (X) | ThermoFisher | 100X | 1X | | 0.5 | μL |
|---|---|---|---|---|---|---|
| Template | — | — | 1,000 | copies | 5 | μL |
| H₂O | — | — | — | | 35.4125 | μL |

¹P31F1 and P31F2 were each used at 0.75 μM of in the master mix;
²An equimolar mixture of dATP, dCTP, dGTP and dTTP;
³Concentration of each nucleotide in the mix

PCR2 amplification was performed using the following thermal protocol, with steps b, c and d performed repeatedly in order for 40 cycles: a. 98° C. for 30 sec, b. 98° C. for 5 sec, c. 58° C. for 10 sec, d. 72° C. for 30 sec, e. Hold at 4° C.

Reaction pooling—40 μL of each singleplex PCR2 reaction was pooled.

Full capture of PCR product—Streptavidin beads (MyOne C1, ThermoFisher Scientific) were resuspended at 8.33 mg/mL in bead resuspension buffer [1.50 M NaCl (Invitrogen), 10 mM Tris-HCl (pH 7.5) (Invitrogen), 0.10% Tween 20 (ThermoFisher Scientific)].

Binding of biotinylated target DNA to streptavidin beads—Equal volumes (120 μL) of the resuspended beads and the PCR2 product were combined with pipette mixing in a 0.2 mL tube. The tube was incubated at room temperature (about 20-26° C.) for 5 minutes with gentle mixing by vortexing, followed by collection of the beads using a magnetic rack.

Washing of Streptavidin beads with bound template—The beads were then washed two times by pipette mixing with 200 μL wash buffer (1 M NaCl, 5 mM Tris-HCl (pH 7.5), 0.05% Tween 20, 0.5 mg/mL BSA), discarding the supernatant between each wash.

Elution with NaOH—50 μL of 40 mM NaOH was added to the 0.2 mL tube, vortexed for 10 seconds, and allowed to stand for 30 seconds. The beads were collected using a magnetic rack for two minutes, then the eluate was transferred to a 0.2 mL tube.

### Results and Conclusion

Target DNA was enriched using PCR reactions and the resulting biotinylated PCR2 products pooled and captured using streptavidin beads, followed by elution of ssDNA with NaOH. Elution of the desired ssDNA from three NaOH eluates was confirmed on a TBE (Tris-Borate-EDTA) gel at 200 V until reference dye reached the bottom of the gel. The gel was then stained in 1× SYBR gold for at least 20 minutes and visualized on a UV station as shown in FIG. 51.

### C. Target Polynucleotide Enrichment Using Multiplex Polymerase Chain Reaction (PCR)

Oligomers (all supplied by IDT; “Y” is a mix of C and T, “W” is a mix of A and T, “S” is a mix of C or G “R” is a mix of A and G)

The following polymerase chain reaction (PCR) 1 primers were used to amplify each of the targets specified:

16s rRNA target
P1 (SEQ ID No. 25):
5′-TGTAGCGGTGAAATGCGYAGA-3′
P1 (SEQ ID No. 26):
5′-CGGTCGACTTAACGCGTTAGCT-3′
P1 (SEQ ID No. 27):
5′-CGGAGTGCTTAATGCGTTWGCT-3′
P2 (SEQ ID No. 28):
5′-CGCAAGGTTGAAACTCAAAGGAATTG-3′
P2 (SEQ ID No. 29):
5′-CCGCAAGGTTAAAACTCAAATGAATTG-3′
P2 (SEQ ID No. 30):
5′-GGGACTTAACCCAACATYTCAC-3′
P29 (SEQ ID No. 31):
5′-CCTGGCTCAGAATGAACGCT-3′
P29 (SEQ ID No. 32):
5′-CCTGGCTCAGGACGAACGCT-3′
P29 (SEQ ID No. 33):
5′-GAGTCTGGACCGTGTCTCAGT-3′
P29 (SEQ ID No. 34):
5′-GAGTCTGGGCCGTGTCTCAGT-3′
P3 (SEQ ID No. 35):
5′-CGTGTGTAGCCCAGGTCATAAGG-3′
P3 (SEQ ID No. 36):
5′-CACGTGTGTAGCCCAAATCATAAGG-3′
P3 (SEQ ID No. 37):
5′-TGTGTAGCCCTGGTCGTAAGG-3′
P3 (SEQ ID No. 38):
5′-TCAGCTCGTGTCGTGAGATGTT-3′
P3 (SEQ ID No. 39):
5′-CGTCAGCTCGTGTTGTGAAATGTT-3′
P30 (SEQ ID No. 40):
5′-CTCCTACGGGAGGCAGCAGT-3′
P30 (SEQ ID No. 41):
5′-CCTCCGTATTACCGCGGCTG-3′
23S rRNA target
P31 (SEQ ID No. 42):
5′-GAAAGACCCCGTGAACCTTTACT-3′
P31 (SEQ ID No. 43):
5′-GAAAGACCCCGTGGAGCTTTACT-3′
P31 (SEQ ID No. 44):
5′-CCTTCGTGCTCCTCCGTTAC-3′
P31 (SEQ ID No. 45):
5′-CCTTTGAGCGCCTCCGTTAC-3′
P4 (SEQ ID No. 46):
5′-ACACAGGTCTCTGCTAAACCGTAAG-3′
P4 (SEQ ID No. 47):
5′-ACACAGGTCTCTGCAAAATCGTAAG-3′
P4 (SEQ ID No. 48):
5′-ACACAGCACTGTGCAAACACGAAAG-3′
P4 (SEQ ID No. 49):
5′-TACCCGACAAGGAATTTCGCTACC-3′
Internal Control target
P25 (SEQ ID No. 50):
5′-TGGCAGCTTCACTTTCTCTTGC-3′
P25 (SEQ ID No. 51):
5′-CCAGCTCCAATCACACCAACA-3′
SHV target
P26 (SEQ ID No. 52):
5′-CAGCTGCTGCAGTGGATGGT-3′
P26 (SEQ ID No. 53):
5′-CCGGSGTATCCCGCAGATA-3′
KPC target
P28 (SEQ ID No. 54):
5′-AACCATTCGCTAAACTCGAACAGG-3′
P28 (SEQ ID No. 55):
5′-CCTTGAATGAGCTGCACAGTGG-3′
mecC target
P32 (SEQ ID No. 56):
5′-GCCGTAATAGTACCTGGTTTGAA-3′
P32 (SEQ ID No. 57):
5′-GCCYTTYGGGTGTTTTGTTAGG-3′
MCR-1 target
P33 (SEQ ID No. 58):
5′-TCTGCAACACCAATCCTTATAACG-3′
P33 (SEQ ID No. 59):
5′-CATCATATCGCTTAAAATACGCAGGC-3′
NDM target
P34 (SEQ ID No. 60):
5′-AGATTGCCGAGCGACTTGGC-3′
P34 (SEQ ID No. 61):
5′-CAACTTTGGCCCGCTCAAGG-3′
OXA-23-like target
P35 (SEQ ID No. 62):
5′-ACAGAATATGTGCCAGCCTCTACA-3′
P35 (SEQ ID No. 63):
5′-CATGGCTTCTCCTAGTGTCATGTCT-3′
OXA-48-like target
P36 (SEQ ID No. 64):
5′-GCGGTAGCAAAGGAATGGCA-3′
P36 (SEQ ID No. 65):
5′-TGCTTGGTTCGCCCGTTTA-3′
OXA-51-like
P37 (SEQ ID No. 66):
5′-AACGAAGCACACACTACGGGTGT-3′
P37 (SEQ ID No. 67):
5′-TGCTCAAGGCCGATCAAAGCATT-3′
gyrA target
P39 (SEQ ID No. 68):
5′-GCAATGACTGGAACAAAGCCTA-3′
P39 (SEQ ID No. 69):
5′-ACCAGCATGTAACGCAGCGA-3′
mecA target
P40 (SEQ ID No. 70):
5′-CATGAAAAATGATTATGGCTCAGGTAC-3′
P40 (SEQ ID No. 71):
5′-TGGAACTTGTTGAGCAGAGGTTC-3′
vanA target
P41 (SEQ ID No. 72):
5′-GGCTGCGATATTCAAAGCTCAG-3′
P41 (SEQ ID No. 73):
5′-CTGAACGCGCCGGCTTAAC-3′
vanB target
P42 (SEQ ID No. 74):
5′-GTATGGAAGCTATGCAAGAAGCC-3′
P42 (SEQ ID No. 75):
5′-CATGCAAAACCGGGAAAGCCA-3′
TEM E104K target
P45 (SEQ ID No. 76):
5′-GCGGTATTATCCCGTGTTGACG-3′
P45 (SEQ ID No. 77):
5′-TCACTCATGGTTATGGCAGCA-3′
TEM G238S target
P46 (SEQ ID No. 78):
5′-GATAAAGTTGCAGGACCACTTCTG-3′
P46 (SEQ ID No. 79):
5′-CCCCGTCRTGTAGATAACTACGA-3′
CTX-M Group 1 target
P48 (SEQ ID No. 80):
5′-CGGCARCCGTCACGCTGT-3′
P48 (SEQ ID No. 81):
5′-CATCAGCACGATAAAGTATTTGCGA-3′
CTX-M Group 2 target
P49 (SEQ ID No. 82):
5′-TGCATGCGCAGRCGAACA-3′
P49 (SEQ ID No. 83):
5′-CCTTACTGGTACTGCACATCGC-3′
P49 (SEQ ID No. 84):
5′-TTGCTGGTGCTGCACATCGC-3′
CTX-M Group 8-25 target
P50 (SEQ ID No. 85):
5′-TACCACCACGCCRTTAGCGA-3′
P50 (SEQ ID No. 86):
5′-ACAACCCACGATGTGGGTAG-3′
CTX-M Group 9 target
P51 (SEQ ID No. 87):
5′-GTGCTTTATCGCGGTGATGAAC-3′
P51 (SEQ ID No. 88):
5′-GTTAACCAGATCGGCAGGCT-3′
28S rRNA H13-20 target
P52/53 (SEQ ID No. 89):
5′-ACTGTACTTGTGCGCTATCGGT-3′
P52/53 (SEQ ID No. 90):
5′-TCCTCAGTAACGGCGAGTGAAGC-3′
28S rRNA H26-31
P54 (SEQ ID No. 91):
5′-CCGTCTTGAAACACGGACCA-3′
P54 (SEQ ID No. 92):
5′-GTTTCCTCTGGCTTCACCCTATTC-3′
28S rRNA H45-46 target
P56 (SEQ ID No. 93):
5′-AACAACTCACCGGCCGAATG-3′
P56 (SEQ ID No. 94):
5′-ATGGAACCTTTCCCCACTTCAGT-3′
28S rRNA H78-79 target
P57 (SEQ ID No. 95):
5′-CCCTGTTGAGCTTGACTCTAGTTTGA-3′
P57 (SEQ ID No. 96):
5′-CTGCGTTATGGTTTAACAGATGTGC-3′
IMP Group Discrimination Reg 1 target
P59 (SEQ ID No. 97):
5′-GACGCCTATCTGATTGAYACTCCA-3′
P59 (SEQ ID No. 98):
5′-CATTTGTTAATTCAGATGCATAYGTGG-3′
P59 (SEQ ID No. 99):
5′-GAGGCTTACCTAATTGACACTCCA-3′
P59 (SEQ ID No. 100):
5′-CTGAAGCTTATCTAATTGACACTCCA-3′
P59 (SEQ ID No. 101):
5′-CTGATGCCTATATAATTGACACTCCA-3′
P59 (SEQ ID No. 102):
5′-CATTAGTTAATTCAGACGCATACGTGG-3′
IMP Group Discrimination Reg 2 target
P60 (SEQ ID No. 103):
5′-GCAAATTTAGAAGCTTGGCCAAAGTCY-3′
P60 (SEQ ID No. 104):
5′-GCCTTTACTTTCATTTAGCCCTTTAA-3′
P60 (SEQ ID No. 105):
5′-AAATGTTGAAGCATGGCCACATTCG-3′
P60 (SEQ ID No. 106):
5′-GCCTTTTGCTTTCATTAAGCCCTTTTA-3′
VIM-groups target
P61 (SEQ ID No. 107):
5′-GGTGTTTGGTCGCATATCGCAAC-3′
P61 (SEQ ID No. 108):
5′-GCGATCGTCATGAAAGTGCGT-3′
gyrB target
P62 (SEQ ID No. 109):
5′-TCCTATAAAGTGTCCGGCGGTC-3′
P62 (SEQ ID No. 110):
5′-TCTCGCCGGTAACCGCCA-3′
P63 (SEQ ID No. 111):
5′-AACCAGGCGATTCTGCCG-3′
P63 (SEQ ID No. 112):
5′-GCAGCTTGTCCGGGTTGTA-3′
P64 (SEQ ID No. 113):
5′-GCACCATTTAGTGTGGGAAATTGTCG-3′
P64 (SEQ ID No. 114):
5′-TAACTTCGACAGCTGGACGT-3′
P65 (SEQ ID No. 115):
5′-GGCGGTGGCGGATACAAAGTAT-3′
P65 (SEQ ID No. 116):
5′-ACCTGTCTTATCAGTTGTGCCAAC-3′

The following nested PCR2 primers were used to amplify each of the targets specified:

16S rRNA target
P1 (SEQ ID No. 117):
5′-TAGAACACCGATGGCGAAGGC-3′
P1 (SEQ ID No. 118):
5′-TCGTGGACTACCAGGGTATCTA-3′
P2 (SEQ ID No. 119):
5′-TTTCGATGCAACGCGAAGAACCT-3′
P2 (SEQ ID No. 120):
5′-TACGAGCTGACGACAGCCATG-3′
KPC target
P28 (SEQ ID No. 121):
5′-CTTTGGCGGCTCCATCGG-3′
P28 (SEQ ID No. 122):
5′-CTCCTCAGCGCGGTAACTTAC-3′
MCR-1 target
P33 (SEQ ID No. 123):
5′-CGGTATGCTCGTTGGCTTAGATG-3′
P33 (SEQ ID No. 124):
5′-GTGATTGCCCATTTGGTGCAG-3′
vanA target
P41 (SEQ ID No. 125):
5′-TTGTATGGACAAATCGTTGACATACA-3′
P41 (SEQ ID No. 126):
5′-GTAGCTGCCACCGGCCTAT-3′
28S rRNA H45-46 target
P56 (SEQ ID No. 127):
5′-AATGGATGGCGCTCAAGCGT-3′
P56 (SEQ ID No. 128):
5′-ACTGCCACCAAGATCTGCACTAG-3′

### Protocol Reaction Conditions

100 genomic copies of each organism were spiked into “PCR1 master mix” (see Table 2 for formulation).

TABLE 2

| | Supplier | Stock | | Final | |
|---|---|---|---|---|---|
| Reagent | (catalogue no.) | Conc. | | Conc. | |
| Water | Sigma | — | | — | |
| Platinum SuperFi | Thermo Fisher | 2 | units/μL | 0.05 | units/μL |
| DNA polymerase | Scientific | | | | |
| PCR1 primer mix | IDT | 100 | μM | 0.4 | μM |
| (39-plex) | | | | | |
| SuperFi ™ buffer | Thermo Fisher | 5X | | 1X | |
| | Scientific | | | | |
| Deoxynucleotide | New England | 10 | mM | 0.40 | mM |
| (dNTP) solution mix | Biolabs | | | | |
| Magnesium Chloride | Thermo Fisher | 1000 | mM | 1.75 | mM |
| (MgCl₂) | Scientific | | | | |

PCR1-PCR amplification was performed using the following three-step thermal, with steps b and c performed repeatedly in order for 25 cycles:
a. 98° C. for 30 sec (initial denature step), b. 98° C. for 5 sec (denature step), c. 65° C. for 25 sec (anneal/extend step).

Sample dilution—The sample was then diluted 40-fold using molecular grade water (ThermoFisher Scientific) to reduce off-target PCR amplicon levels.

Addition of PCR2 reagents—10 μL of diluted PCR1 material was added to 40 μL of “PCR2 master mix” to create a 1:200 final dilution (see Table 3 for PCR2 master mix formulation). In this example, PCR2 master mix comprises 1.50 μM of each primer pair (P1, P28, P33 and P56).

TABLE 3

| | Supplier | Stock | | Final | |
|---|---|---|---|---|---|
| Reagent | (catalogue no.) | Conc. | | Conc. | |
| Water | Sigma | — | | — | |
| Platinum SuperFi | Thermo Fisher | 2 | units/μL | 0.05 | units/μL |
| DNA polymerase | Scientific | | | | |
| PCR2 primer mix | NEB | 100 | μM | 1.50 | μM |
| SuperFi ™ buffer | Thermo Fisher | 5X | | 1X | |
| | Scientific | | | | |
| Deoxynucleotide | New England | 10 | mM | 0.4 | mM |
| (dNTP) solution mix | Biolabs | | | | |
| Magnesium Chloride | Thermo Fisher | 1000 | mM | 1.75 | mM |
| (MgCl₂) | Scientific | | | | |

PCR2-The PCR2 amplification was performed using the following three-step thermal protocol, with steps b and c performed repeatedly in order for 40 cycles:
a. 98° C. for 30 sec (initial denature step), b. 98° C. for 5 sec (denature step), c. 65° C. for 25 sec (anneal/extend step).

The PCR2 products were confirmed using Bioanalyzer sizing and quantification.

### Results and Conclusion

The target DNA was enriched using two multiplexed PCR reactions. The resulting PCR2 products were resolved and quantified using Bioanalyzer quantification. Table 4 shows the resulting concentrations from PCR 2 targets after successful PCR amplification.

TABLE 4

| Organism Spike | Target | μM |
|---|---|---|
| K. Pneumoniae | P1 | 1.00 |
| K. Pneumoniae | P28 | 1.26 |
| C. Albicans | P56 | 1.23 |
| E. Coli | P33 | 1.20 |
| E. Coli | P1 | 1.15 |

In conclusion, 100 genomic copies of each organism was enriched using multiplex PCR, resulting in desired dsDNA.

### D. Target Nucleic Acid Enrichment Using Multiplex Polymerase Chain Reaction (PCR) and Full Capture

Oligomers (all supplied by IDT; “52-Bio” represents two biotin groups on the 5′-end of the oligomer; “Y” is a mix of C and T; “W” is a mix of A and T)

The following PCR1 primers were used to amplify the specified targets:

16s rRNA target
P1 (SEQ ID No. 25):
5′-TGTAGCGGTGAAATGCGYAGA-3′
P1 (SEQ ID No. 26):
5′-CGGTCGACTTAACGCGTTAGCT-3′
P1 (SEQ ID No. 27):
5′-CGGAGTGCTTAATGCGTTWGCT-3′
P2 (SEQ ID No. 28):
5′-CGCAAGGTTGAAACTCAAAGGAATTG-3′
P2 (SEQ ID No. 29):
5′-CCGCAAGGTTAAAACTCAAATGAATTG-3′
P2 (SEQ ID No. 30):
5′-GGGACTTAACCCAACATYTCAC-3′
vanA target
P41 (SEQ ID No. 72):
5′-GGCTGCGATATTCAAAGCTCAG-3′
P41 (SEQ ID No. 73):
5′-CTGAACGCGCCGGCTTAAC-3′

The following nested PCR2 primers were used to amplify each of the 16s rRNA target:

P2 (SEQ ID No. 129):
5′-AAAACGAGACATGCCGAGCATCCGCTTTCGATGCAACGCGAAGAA
CCT-3′
P2 (SEQ ID No. 130):
5′-/52-Bio/TACGAGCTGACGACAGCCATG-3′

### Protocol Reaction Conditions

100 genomic copies of each organism were spiked into “PCR1 master mix”. PCR1 master mix was prepared in a 0.2 mL tube according to the formulation shown in Table 5.

TABLE 5

| | Supplier | Stock | | Final | |
|---|---|---|---|---|---|
| Reagent | (catalogue no.) | Conc. | | Conc. | |
| Water | Sigma | — | | — | |
| Platinum SuperFi | Thermo Fisher | 2 | units/μL | 0.02 | units/μL |
| DNA polymerase | Scientific | | | | |
| PCR1 primer mix¹ | IDT | 100 | μM² | 0.4 | μM² |
| SuperFi ™ buffer | Thermo Fisher | 5X | | 1X | |
| | Scientific | | | | |
| Deoxynucleotide | New England | 10 | mM⁴ | 0.20 | mM⁴ |
| (dNTP) solution mix³ | Biolabs | | | | |

| | | | | |
|---|---|---|---|---|
| Magnesium Chloride | Thermo Fisher | 1000 | mM | 1.75M |
| (MgCl₂) | Scientific | | | |

¹An equimolar mixture of the primers listed in [02] above;
²Concentration of each primer in the mix
³An equimolar mixture of dATP, dCTP, dGTP and dTTP;
⁴Concentration of each nucleotide in the mix

PCR1-PCR amplification was performed using the following three-step thermal protocol, with steps b and c performed repeatedly in order for 25 cycles:
a. 98° C. for 30 sec (initial denature step), b. 98° C. for 5 sec (denature step), c. 65° C. for 25 sec (anneal/extend step).

Sample dilution—The sample was then diluted 40-fold using molecular grade water (ThermoFisher Scientific) to reduce off-target PCR amplicon levels.

Addition of PCR2 reagents—10 μL of diluted PCR1 material was added to 40 μL of “PCR2 master mix” to create a 1:200 final dilution. PCR2 master mix was prepared according to the formulation shown in Table 6. In this example, PCR2 master mix comprises 1.50 μM primer mix (P2 as shown in [0487]).

TABLE 6

| | | Stock | | Final | |
|---|---|---|---|---|---|
| Reagent | Manufacturer | Conc. | | Conc. | |
| Water | Sigma | — | | — | |
| Platinum SuperFi | Thermo Fisher | 2 | units/μL | 0.02 | units/μL |
| DNA polymerase | Scientific | | | | |
| PCR2 dual | NEB | 100 | μM | 1.50 | μM |
| biotinylated primer | | | | | |
| mix | | | | | |
| SuperFi ™ buffer | Thermo Fisher | 5X | | 1X | |
| | Scientific | | | | |
| Deoxynucleotide | New England | 10 | mM | 0.4 | mM |
| (dNTP) solution mix | Biolabs | | | | |
| Magnesium Chloride | Thermo Fisher | 1000 | mM | 1.75M | |
| (MgCl₂) | Scientific | | | | |

PCR2-The PCR2 amplification was performed using the following three-step thermal protocol, with step b and c performed repeatedly in order for 45 cycles:
a. 98° C. for 30 sec (initial denature step), b. 98° C. for 5 sec (denature step), c. 65° C. for 25 sec (anneal!extend step).

The PCR2 products were confirmed using Bioanalyzer sizing and quantification.

Full capture of PCR product—Streptavidin beads (MyOne Cl, ThermoFisher Scientific) were resuspended at 8.33 mg/mL in bead resuspension buffer [1.50 M NaCl (Invitrogen), 10 mM Tris-HCl (pH 7.5) (Invitrogen), 0.10% o Tween 20 (ThermoFisher Scientific)].

Binding of biotinylated target DNA to streptavidin beads—Equal volumes (120 μL each) of the resuspended beads and the PCR2 product were combined in a 0.2 mL tube. The tube was incubated at room temperature (about 20-26° C.) for 10 minutes with gentle mixing by vortexing, followed by bead binding with a magnetic rack.

Washing of Streptavidin beads with bound template—The beads were then washed three times with 200 μL wash buffer [1 M NaCl, 5 mM Tris-HCl (pH 7.5), 0.050% Tween 20, 0.5 mg/mL BSA], discarding the supermatant between each wash.

Elution with NaOH—50 μL of 40 mM NaOH was added to the 0.2 mL tube, vortexed for 10 seconds, and allowed to stand for 30 seconds. The beads were immobilized on a magnetic rack for two minutes, then the eluate was transferred to a new 0.2 mL tube.

### Results and Conclusion

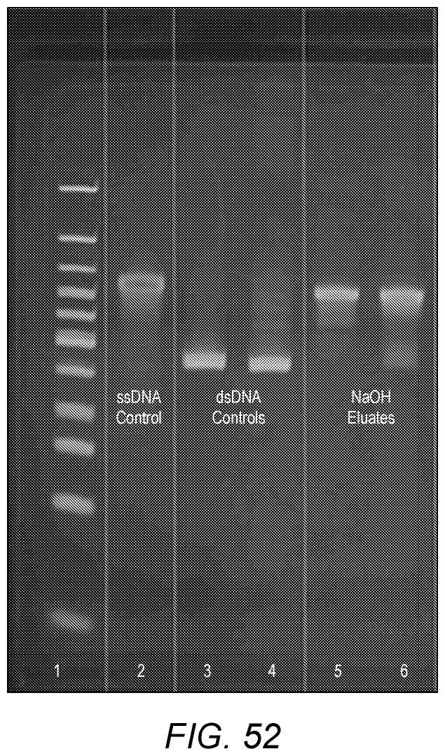

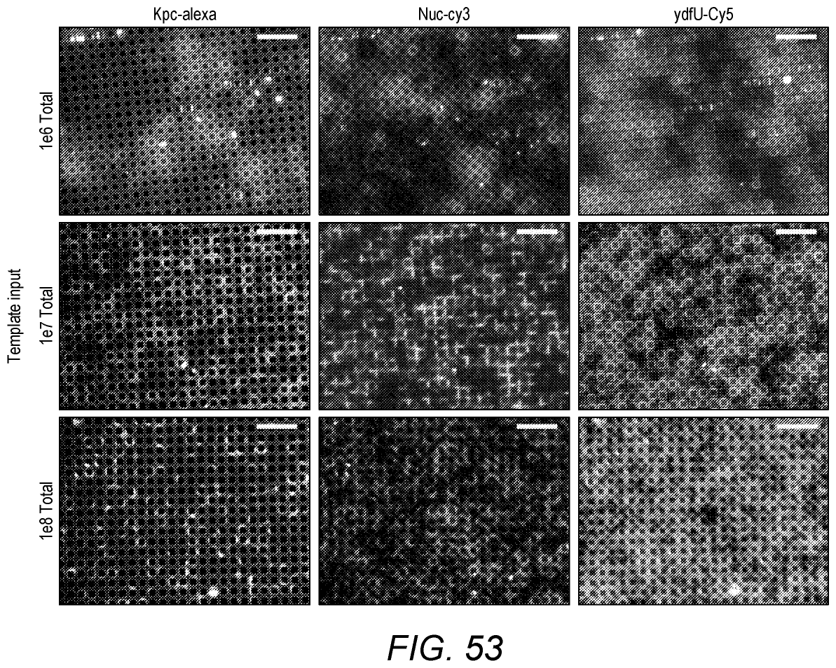

ssDNA elution from the NaOH eluates was confirmed on a TBE gel at 200 V until reference dye reached the bottom of the gel. The gel was then stained in 1× SYBR gold for at least 20 minutes and visualized on a UV station as shown in FIG. 52.

In conclusion, DNA from three target organisms was amplified in PCR 1, then one target amplified in PCR2. Full capture of the PCR2 product was then performed to produce the desired ssDNA for hybridization to primers bound to the surface of the semiconductor chip.

E. Targeted Enrichment (PCR1 and nested PCR2) followed by Copy Control

Oligomers (all supplied by IDT; “iSP18” is Hexaethylene Glycol (HEG) internal spacer; 3BiodT is a 3′ biotin molecule linked to the terminal dT nucleotide; 56-FAM/is a 5′ attachment of 6-FAM (Fluorescein);/ZEN/is a proprietary IDT ZEN quencher molecule;/3IABkFQ/is a 3′ Iowa Black FQ quencher)

The following polymerase chain reaction (PCR) 1 primers were used for the E. Faecium (EFM) target:

P41F (SEQ ID No. 131):
5′-GGCTGCGATATTCAAAGCTCAG-3′
P41R (SEQ ID No. 132):
5′-CTGAACGCGCCGGCTTAAC-3′

The following nested PCR2 primers were used for the EFM target amplicon from PCR1:

P41F (SEQ ID No. 133):
5′-AAAACGAGACATGCCGAGCATCCGCTTGTATGGACAAATCGTTGA
CATACA-3′
P41R (SEQ ID No. 134):
5′-ACCGTGCTGCCTTGGCTTCATTGTGGTCGTAGCTGCCACCGGCCT
AT-3′

The following hairpin oligo sequence (SEQ ID No. 135) was used:

5′-CGCGCGAAAAAAAAAAAAAAAAAAAA/iSp18/TTTTTTTTTTTT
TTTCGCGCGAAAAACTCCTCTGGCACCGTGCTGCCTTGGCTTCATTGT
GGTC-3′

An intervening oligo between the hairpin oligo and bead was used:

polydT oligo sequence (SEQ ID No. 136):
TTTTTTTTTTTTTTTTTTTT/3BiodT/

The following primers were used for quantitative PCR (qPCR):

RPAIF (SEQ ID No. 137):
5′-AAAACGAGACATGCCGAGCATC-3′
RPAlouterR (SEQ ID No. 138):
5′-TCGCGCGAAAAACTCCTCTGG-3′
FAM probe (SEQ ID No. 139):
5′-/56-FAM/TGCTGGGAT/ZEN/AGCTACTCCCGCCTTTTGG/
3IABKFQ/-3′

Control sequence for standard curve (SEQ TD No. 140):

5′-AAAACGAGACATGCCGAGCATCCGCTTGTATGGACAAATCGTTGAC
ATACATCGTTGCGAAAAATGCTGGGATAGCTACTCCCGCCTTTTGGGTT
ATTAATAAAGATGATAGGCCGGTGGCAGCTACGACCACAATGAAGCCAA
GGCAGCACGGTGCCAGAGGAGTTTTTCGCGCGA-3′

### Protocol Reaction Conditions

Sample dilution—EFM genomic DNA (gDNA) was diluted to 1000 copies/L

Addition of PCR1 reagents—10 μL of diluted EFM gDNA was added to 40 μL PCR1 master mix. PCR 1 master mix was prepared according to the formulation shown in Table 7.

TABLE 7

| | | Stock | Final | | Per 40.0 μL | |
|---|---|---|---|---|---|---|
| Component | Manufacturer | Conc. | Conc. | Units | reaction | |
| 5X SuperFi Buffer | ThermoFisher | 5 | 1 | X | 10.0 | μL |
| P41F PCR1 primer | IDT | 100 | 0.4 | μM | 0.20 | μL |
| P41R PCR1 primer | IDT | 100 | 0.4 | μM | 0.20 | μL |
| dNTP Mix | NEB | 10 | 0.4 | mM | 2.00 | μL |
| SuperFi | ThermoFisher | 2 | 0.05 | U/uL | 1.25 | μL |
| Polymerase | | | | | | |
| MgCl₂ | ThermoFisher | 25 | 1.75 | mM | 3.50 | μL |
| UltraPure H₂O | ThermoFisher | — | — | — | 22.9 | μL |

PCR1-PCR amplification was performed using the following three-step thermal protocol, with steps b and c performed repeatedly in order for 30 cycles:
a. 98° C. for 30 sec (initial denature step), b. 98° C. for 5 sec (denature step), c. 65° C. for 25 sec (anneal!extend step).

Sample dilution—2.5 μL of PCR1 reaction was diluted in 97.5 μL water for a 1:40 dilution.

Addition of PCR2 reagents—5 μL of diluted PCR1 material was added to 20 μL PCR2 master mix to create a 1:200 final dilution. PCR2 master mix was prepared according to the formulation shown in Table 8.

TABLE 8

| | | | | | | Per 20.0 μL | |
|---|---|---|---|---|---|---|---|
| Component | Manufacturer | Stock Conc. | | Final Conc. | | reaction | |
| 5X SuperFi Buffer | ThermoFisher | 5X | | 1X | | 5 | μL |
| P41F PCR2 Primer | IDT | 100 | μM | 1.5 | μM | 0.375 | μL |
| P41R PCR2 Primer | IDT | 100 | μM | 1.5 | μM | 0.375 | μL |
| dNTP Mix | NEB | 10 | mM | 0.4 | mM | 1 | μL |
| MgCl₂ | ThermoFisher | 1000 | mM | 1.75 | mM | 0.04375 | μL |
| SuperFi | ThermoFisher | 2 | U/μL | 0.02 | U/μL | 0.25 | μL |
| Polymerase | | | | | | | |

| | | | | | |
|---|---|---|---|---|---|
| EVA Green (X) | VWR | 100 | 1 | 0.25 | μL |
| H₂O | ThermoFisher | — | — | 12.71 | μL |

PCR2-The PCR2 amplification was performed using the following thermal protocol, with steps b, c and d performed repeatedly in order for 45 cycles.
a. 98° C. for 30 sec (initial denature step), b. 98° C. for 5 sec (denature step), c. 55° C. for 10 sec (anneal step), d. 72° C. for 30 sec (extend step), e. Ramp up from 65° C. to 95° C. (inclusive) at a rate of 0.5° C. every cycle (melt step).

The PCR2 products were confirmed using Bioanalyzer sizing and quantification. Stock concentration was found to be 800 ng/μL, which correlates to 1.88×10¹A13 copies per 40 μL. This .L sample was used as the “neat” condition.

PCR2 sample dilution—The sample was also diluted 1:10 for a separate “1:10” input, which would be 1.88×10¹A12 copies per 40 μL.

Addition of extension master mix—60 μL of extension master mix was added to 40 L of either “neat” or “1:10” PCR2 output material and pipette mixed, followed by a quick vortex and quick spin. Extension master mix was prepared according to the formulation shown in Table 9.

TABLE 9

| | | | | | | Per 60.0 μL |
|---|---|---|---|---|---|---|
| Reagent | Manufacturer | Stock Conc. | | Final Conc. | | reaction |
| DeepVent (−) Rxn | NEB | 10X | | 0.4 | | 4.00 μL |
| Buffer | | | | | | |
| DeepVent (−) Enzyme | NEB | 2 | U/μL | 0.02 | | 1.00 μL |
| dNTP mixed | NEB | 10 | mM | 0.012 | | 0.12 μL |
| Hairpin oligo | IDT | 4.64E+11 | | 5.E+09 | copies/μL | 1.08 μL |
| BSA | NEB | 100 | mg/mL | 0.60 | mg/mL | 0.60 μL |
| MgCl₂ | ThermoFisher | 1000 | mM | 1.8 | mM | 0.18 μL |

| | | | | |
|---|---|---|---|---|
| Water | ThermoFisher | — | — | 53.0 μL |

Extension—The extension was performed using the thermal protocol:
a. 92° C. for 2 min b. 64° C. for 2 min c. 68° C. for 10 min.

Addition of hybridization mix—50 μL of hybridization mix was added to the completed extension reaction and pipette mixed. Hybridization mix was prepared according to the formulation shown in Table 10.

TABLE 10

| | | | | Per 50.0 μL |
|---|---|---|---|---|
| Reagent | Manufacturer | Stock Conc. | Final Conc. | reaction |
| Water | ThermoFisher | — | — | 47.1 μL |
| BSA | NEB | 100 mg/mL | 0.33 mg/mL | 0.50 μL |
| NaCl | ThermoFisher | 5M | 0.042M | 1.25 μL |
| polydT oligo | IDT | 4.43E+09 | 3.35E+07 | 1.13 μL |

Preparation and addition of streptavidin-coupled magnetic beads—0.2 mg of streptavidin beads (prepared internally) were washed 3× in 1× wash buffer (Table 11) and resuspended in 50 μL 1× wash buffer in a 0.2 mL tube. Fifty μL of conjugation beads was then added to the extension/hybridization mixture and pipette mixed.

TABLE 11

| Reagent | Manufacturer | Stock Conc. | | Final Conc. | |
|---|---|---|---|---|---|
| NaCl | ThermoFisher | 5M | | 1M | |
| Tris-HCl (pH 7.5) | ThermoFisher | 1000 | mM | 5 | mM |
| EDTA | ThermoFisher | 500 | mM | 0.5 | mM |
| Tween20 | ThermoFisher | 100% | | 0.05% | |
| BSA | NEB | 100 | mg/mL | 0.5 | mg/ml |

| | | | |
|---|---|---|---|
| Water | ThermoFisher | — | — |

The reaction was incubated for 2 minutes at 25° C. and the tubes were then placed on a magnetic stand for 30 seconds.

The supernatant was aspirated, the beads were pipette mixed with 1× wash buffer (Table 5) and placed back on the magnetic stand until clear.

The wash was repeated as above for a total of 3 washes.

After the third wash the supernatant was removed and beads were resuspended in 30 μL of UltraPure water.

Tubes were incubated for 1 minute at 70° C., flicked to mix, spun and returned to 70° C. for 1 minute.

Eluates were removed and quantified using qPCR, 2 μL of input was added to 13 μL of PCR quantification master mix which was prepared according to the formulation shown in Table 12. All reactions were run in triplicate. The control sequence for standard curve was run at 2×10\hat{ }7, 2×10\hat{ }6, 2×10\hat{ }5 and 2×10\hat{ }4 copies.

TABLE 12

| | | Stock | Final | Per 13 μL |
|---|---|---|---|---|
| Reagent | Manufacturer | Conc. | Conc. | reaction |
| PerfeCTa MM | Quantabio | 5 x | 1x | 3.00 μL |
| RPA1F | IDT | 100 μM | 1 μM | 0.15 μL |
| RPA1outerR | IDT | 100 μM | 1 μM | 0.15 μL |
| FAM probe | IDT | 100 μM | 0.3 μM | 0.045 μL |
| Water | ThermoFisher | NA | NA | 9.66 μL |

The qPCR reaction was performed using the following thermal protocol, with steps b, c and d performed repeatedly in order for 40 cycles:
a. 95° C. for 60 sec, b. 95° C. for 10 sec, c. 64° C. for 25 sec, d. 72° C. for 10 sec.

### Results and Conclusion

The target DNA was enriched using PCR reactions. PCR2 output was quantified using the bioanalyzer and was found to be 80.0 ng/μL. This corresponds to 1.88×10—copies per 40 μL (used as the “neat” condition). The sample was also diluted 1:10 for a separate “1:10” input, which was 1.88×10 copies per 40 μL.

The copy control process normalized the outputs of two samples with 10-fold different input concentrations to nearly equal values (only a 6% difference), demonstrating the ability of this process to normalize a wide range of input concentrations to a desired value as shown in Table 13.

TABLE 13

| CC Results | Copies |
|---|---|
| Neat | 3.22E+09 |
| 1:10 | 3.42E+09 |
| Difference | 6% |

F. Capture of Predetermined Amount of Amplicon with a Capture Oligomer Comprising a Capture Sequence and a Complement Thereof

### Oligomers.

PCR to amplify a segment from the E. coli uidA gene was carried out using the primers:

Ec_uidA_F (SEQ ID No. 141):
GTATCAGCGCGAAGTCTTTATACC
Ec_uidA_R (SEQ ID No. 142):
GGCAATAACATACGGAGTGACATC

The primers were designed to generate an amplicon with the following sequence (SEQ ID No. 143):

GTATCAGCGCGAAGTCTTTATACCGAAAGGTTGGGCGGGCCAGCGTATT
GTACTGCGTTTCGATGCGGTCACTCATTACGGCAAAGTGTGGGTAAATA
ATCAGGAAGTGATGGAGCATCAGGGCGGCTATACGCCATTTGAAGCCGA
TGTCACTCCGTATGTTATTGCC

A capture oligomer designated uidA PA_1.2 was provided having the following sequence (SEQ ID No. 144):

AAAAAAAAAAAAAAAAAAAAAAAAAAAAAAAAAAACCTCTA/iSp18/
TTTTTTTTTTTTTTTTTTTTTTTTTTTTTTTTTTTAGACGCAAGCTAC
TGGTGATTTGGCAATAACATACGGAGTGACATCGGCTTC
(iSp18 = Hexaethylene Glycol (HEG) internal
spacer (IDT))

In this oligomer, the poly-sequence is the capture sequence. CC1CIA is a linker sequence. iSpl8 is the internal extension blocker. The poly-T sequence following iSpl8 is the complement of the capture sequence. AGACGCAAGCTACTGGTGATTT is a fourth additional sequence. The target-hybridizing sequence (THS) is GGCAATAACATACGGAGTGACATCGGCTTC, which hybridizes specifically to a segment of the uidA gene sequence in the target amplicon. In this example the THS is longer than the reverse PCR primer (see sequence above), with which it overlaps, to raise the Tm of the THS and give it a competitive advantage over the reverse primer in hybridizing to the target.

A secondary capture reagent with the following sequence was used:

dT20-biotin (SEQ ID No. 145):
TTTTTTTTTTTTTTTTTTTT/3′Biotin

The following primers and probe were used for quantitative PCR (qPCR) analysis of copy control products:

Ec_uidA_F (SEQ ID No. 141):
GTATCAGCGCGAAGTCTTTATACC
uidA Probe (SEQ ID No. 146):
56-FAM/TAGCCGCCCTGATGCTCCATCACTTCCTG/3′IowaBlack
TQ_R (SEQ ID No. 147):
AGACGCAAGCTACTGGTGAT

Protocol/reaction conditions.
(1) PCR amplicon was generated for the target uidA utilising the primers shown above; amplicon was purified using AMPure XP (Beckman Coulter) using the manufacturer's recommended protocols and quantitated via qPCR using the uidA forward and reverse primers along with the uidA_Probe.
(2) Capture oligomer annealing and extension of the amplicon strand—The purified uidA amplicon was diluted 2—, 10- or 100-fold. A 20 μL aliquot of each dilution of the amplicon was added to capture oligomer annealing/extension reactions consisting of 0.07 U/μl SDPol (Bioron), 1× SDPol reaction buffer, 0.17 mM dNTP's, 3 mM MgCl₂, 1 mg/ml BSA and 5×10¹⁰ copies of the capture oligomer for a final volume of 30 μL. The capture oligomer was annealed to the 3′-end of the complementary strand of the uidA amplicon and the amplicon strand was extended using a thermal cycler according to the following thermal profile: 92° C. for 2 minutes, 54° C. for 2 minutes, 68° C. for 10 minutes, 54° C. for 2 minutes followed by a controlled ramp down (0.3° C./second) to 20° C. In this example the 3′-end of the capture oligomer was also extended.
(3) Hybridization of the complement of the capture sequence of the capture oligomer—The entire capture oligomer/amplicon extension reaction mixture was added to 10 μL of a secondary capture reagent at 4× concentration, resulting in a final concentration of 125 mM NaCl, 0.25 mg/ml BSA and 10⁷, 10⁸ or 10⁹ copies of the complement of the capture sequence (i.e., 3 different amounts were tested). Hybridization was carried out by incubating the reaction mix at room temperature (20-24° C.) for 15 minutes.
(4) Capture of the amplicon and capture oligomer extension product/capture oligomer complex—A 5 μL aliquot (50 μg) of MyOneC1 streptavidin beads (ThermoFisher Scientific) in 250 mM NaCl and 1 mg/ml BSA was added to the hybridisation mixture (step 3 above), the capture oligomer/amplicon extension product/complement of the capture sequence complex was captured onto the beads and the beads were washed as per manufacturer's recommendations.
(5) Elution—After the final wash was completed and the wash buffer removed, 10 μL of water was added to the bead pellet, the beads were resuspended and incubated at 70° C. for 2 minutes. The beads were pelleted with a magnet and the eluate was removed.
(6) Quantitation—The amount of eluted product as well as capture oligomer extension products (see step 2 above) were quantified by qPCR using primers targeting the uidA F primer site and the TQ primer-adaptor site along with the uidA_Probe.

### Results and Conclusions:

A 2-fold, 10-fold and 100-fold dilution of the amplicon produced in the targeted enrichment step (see step 1 above) were subjected to the copy control process (see steps 2-5 above) using 10⁷, 10⁸ or 10⁹ copies of capture oligomer. As shown in Table 14, the amount of recovered amplicon was proportional (in approximately the expected ratios; see “Ratio” column in the table) to the amount of capture oligomer added for each PCR dilution. Variation between replicates at each data point was low (see “Std Dev” column).

TABLE 14

| PCR | 2nd CO (# | Output (# | | |
|---|---|---|---|---|
| dilution | copies) | copies*) | Std Dev | Ratio** |
| 2-fold | 1 × 10⁷ | 9.8 × 10⁸ | 6.1 × 10⁷ | 1 |
| | 1 × 10⁸ | 4.4 × 10⁹ | 2.5 × 10⁹ | 4.5 |
| | 1 × 10⁹ | 8.0 × 10¹⁰ | 4.5 × 10⁹ | 82 |
| 10-fold | 1 × 10⁷ | 7.7 × 10⁸ | 8.7 × 10⁷ | 1 |
| | 1 × 10⁸ | 5.7 × 10⁹ | 1.2 × 10⁸ | 7.4 |
| | 1 × 10⁹ | 6.5 × 10¹⁰ | 2.0 × 10⁹ | 84 |
| 100-fold | 1 × 10⁷ | 4.4 × 10⁸ | 8.3 × 10⁷ | 1 |
| | 1 × 10⁸ | 2.6 × 10⁹ | 2.0 × 10⁸ | 5.9 |
| | 1 × 10⁹ | 2.8 × 10¹⁰ | 2.7 × 10⁹ | 64 |

*Average of 3 replicates
**Set the value at 2-fold dilution equal to 1

Furthermore, despite a 50-fold difference in the amplicon input amount, variation in output was 2.36-fold, 3.11-fold and 3.44-fold for 107, 108 and 109 copies of capture oligomer, respectively (Table 1). As above, variations between replicates were low.

These data demonstrate that a capture oligomer described herein can be used to yield a pre-determined amount of target output from a range of different input target amounts.

An additional experiment was performed essentially as described above but in a multiplex format using capture oligomers in which the THS regions were designed to target the uidA gene of E. coli, the nuc gene of Staphylococcus aureus, the vanA gene of Enterococcus faecallis or the rpb7 gene of Candida albicans. (i.e., 4 capture oligomers were used per reaction). The uidA, nuc and vanA genes were each amplified separately using PCR as described above using and the primers shown above for uidA and additional primers designed for the nuc and vanA genes. Resulting amplicons were diluted 10- or 100-fold and a 20 μL aliquot of each dilution of each individual target was added to a separate capture oligomer annealing and extension of amplicon strand reaction containing 5×10¹⁰ copies each of the 4 capture oligomers described above (i.e., 4-plex capture oligos but only 1 target present). The reaction conditions were the same as those described about except with the following thermal profile: 92° C. for 2 minutes, 64° C. for 2 minutes, 68° C. for 10 minutes, 98° C. for 2 minutes, 57° C. for 2 minutes followed by a controlled ramp down (0.3° C./second) to 20° C. After this reaction was completed, 5×10⁸ copies of the complement of the capture sequence of the capture oligomers was added to each reaction. This was followed by capture, wash, elution and quantitation steps as described above.

Output amounts following capture of amplicon with input amounts differing by a factor of 10 are shown in Table 15. The results show that a 10-fold difference in input amplicon level was reduced to no more than a 1.4-fold difference in average output level after copy control for each of the 3 individual target amplicons tested in a multiplex format. Furthermore, the range of average output level after copy control spanned approximately 1.6-fold across all 3 target amplicons whereas their input level spanned more than 270-fold.

TABLE 15

| Target | Input | Output* | Std Dev |
|---|---|---|---|
| nuc | 1.6 × 10⁸ | 1.5 × 10⁷ | 9.0 × 10⁵ |
| | 1.6 × 10⁹ | 1.7 × 10⁷ | 2.0 × 10⁶ |
| uidA | 3.3 × 10⁹ | 1.9 × 10⁷ | 2.3 × 10⁵ |
| | 3.3 × 10¹⁰ | 1.9 × 10⁷ | 3.4 × 10⁶ |
| vanA | 1.2 × 10⁸ | 1.2 × 10⁷ | 1.3 × 10⁶ |
| | 1.2 × 10⁹ | 1.6 × 10⁷ | 1.3 × 10⁶ |

*Average of 3 replicates

An additional experiment was performed essentially as described above in a singleplex format but using a capture oligomer in which the THS anneals to a universal binding site in a tag sequence incorporated into the target of interest during the PCR amplification step. Primers were designed to target the bacterial 23S rRNA gene and PCR amplicon was generated from bacterial genomic DNA. The reverse primer comprised a universal sequence tag that was incorporated into the amplicon during PCR. Capture oligomer annealing and extension of an amplicon strand was performed as described above except that a 40 μL aliquot of neat (i.e., no dilution; approximately 9×10¹² copies) or a 50-fold dilution (approximately 2×10¹¹ copies) were added to the reaction mixture comprised 0.02 U/μL SD Polymerase (Bioron), 0.4× SD Polymerase reaction buffer (Bioron), 0.012 mM of the 4 dNTP's, 1.8 mM MgCl₂, 0.6 mg/mL BSA and 5×10¹⁰ copies of capture oligomer in a final reaction volume of 100 μL. The thermal profile used was 92° C. for 2 minutes, 54° C. for 2 minutes, 68° C. for 10 minutes, 54° C. for 2 minutes followed by a controlled ramp down (0.3° C./second) to 20° C. The entire volume of the above reaction was added to 50 μL of a 3× annealing mix containing 1 mg/ml BSA, 125 mM NaCl, and 5×10⁸ copies of the secondary capture reagent. Annealing is carried out by incubating the reaction mix on a thermal block at 25° C. for 10 minutes. A 50 μL volume (200 μg in this experiment) of MyOneC1 streptavidin beads (ThermoFisher Scientific) in a 4× Wash Buffer consisting of 4M NaCl, 20 mM Tris-HCl pH 7.5, 2 mM EDTA, 0.20% Tween20, 2 mg/mL BSA was added to the 150 μL reaction from above. The resulting complex including the capture oligomer, amplicon extension product, and complement of the capture sequence was captured onto the beads and the beads were washed as per manufacturer's recommendations (using in this case a 1× Wash Buffer; see 4× formulation immediately above). A volume of 20 μL of water was used for elution (protocol otherwise the same as above). qPCR was performed using a specific forward primer and a reverse primer targeted to the universal tag.

Despite a 50-fold difference in amplicon input level, variation in output was only 2.5-fold after the capture oligomer annealing and extension of the amplicon strand step and 1 (i.e., completely normalized) after contacting with the secondary capture reagent and isolation of the resulting complex (see Table 16). Further, these results demonstrate an embodiment of the disclosure in which the THS binds to a universal tag sequence.

TABLE 16

| | PCR | Output (# | # of | | |
|---|---|---|---|---|---|
| Step in process | dilution | copies*) | reps | Std Dev | Ratio** |
| After first | 0-fold | 2.1 × 10¹⁰ | 4 | 4.5 × 10⁸ | 2.5 |
| capture | (neat) | | | | |
| oligomer | 50-fold | 8.3 × 10⁹ | 4 | 1.8 × 10⁸ | |
| After second | 0-fold | 1.7 × 10⁷ | 6 | 3.6 × 10⁵ | 1 |
| capture | (neat) | | | | |
| oligomer | 50-fold | 1.6 × 10⁷ | 9 | 3.5 × 10⁵ | |

*Average of replicates (reps)
**Neat/50-fold dilution output

An additional experiment was performed essentially as described immediately above (singleplex, universal THS) but in a multiplex format using a capture oligomer in which the THS anneals to a universal tag sequence incorporated into each of the targets of interest during the PCR amplification step. Eight different amplicons targeting regions in the bacterial 16S rRNA gene, 23S rRNA gene and the antibiotic resistance marker KPC were generated from K. pneumonia genomic DNA in eight separate singleplex reactions. One amplicon targeting a synthetic Internal Control (IC) DNA was also generated for a total of nine individual amplicons. Equal amounts of all 9 amplicons were pooled and a 54 μL aliquot of neat (i.e., no dilution; approximately 1×10¹³ copies) or a 10-fold dilution (approximately 1×10¹² copies) were added to separate capture oligomer annealing and extension of an amplicon strand reaction mixtures (100 μL final volume for each). The remainder of the workflow was performed essentially the same as described above, except that 5×10¹¹ copies of capture oligomer and 5×10⁹ of the secondary capture reagent were used. qPCR was performed to quantitate each target using a specific forward primer and a reverse primer targeted to the universal tag (9 separate PCRs). The quantities recovered for each individual target were summed to determine the overall recovery of the capture process.

Despite a 10-fold difference in amplicon input level, variation in overall output was only 2.5 after the capture oligomer annealing and extension of the amplicon strand step and 1.6 after contacting with the secondary capture reagent and isolation of the resulting complex (see Table 17). Further, these results demonstrate the embodiment of the invention in which the THS binds to a universal tag sequence in a multiplex format, thus capturing all target amplicons present in the mixture.

TABLE 17

| | PCR | Output (# | # of | | |
|---|---|---|---|---|---|
| Step in process | dilution | copies*) | reps | Std Dev | Ratio** |
| After first | 0-fold | 2.6 × 10¹¹ | 32 | 1.9 × 10¹⁰ | 2.5 |
| capture | (neat) | | | | |
| oligomer | 10-fold | 1.1 × 10¹¹ | 31 | 7.7 × 10⁹ | |
| After second | 0-fold | 1.5 × 10⁹ | 89 | 1.1 × 10⁸ | 1.6 |
| capture | (neat) | | | | |
| oligomer | 10-fold | 9.5 × 10⁸ | 88 | 6.9 × 10⁷ | |

*Average of replicates (reps)
**Neat/50-fold dilution output

G. Capture of Predetermined Amount of Amplicon with a Capture Oligomer Comprising a Capture Sequence, a Complement Thereof, and Clamp Sequences

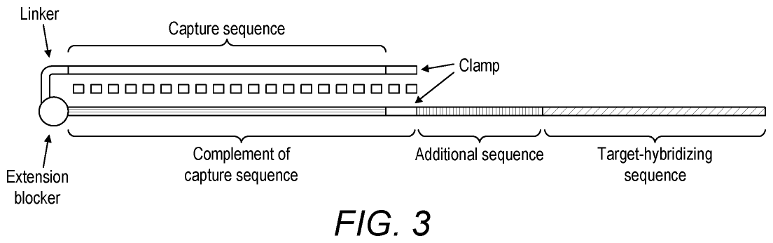

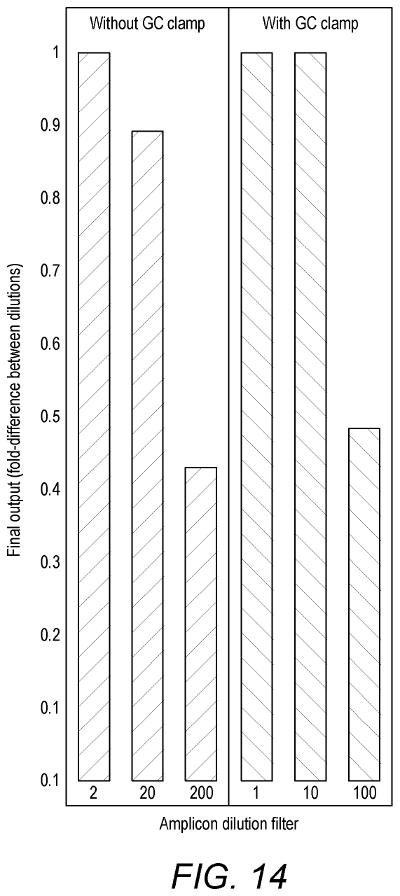

A target amplicon was prepared essentially as described above with respect to uidA and used in experiments with a capture oligomer with or without clamp sequences (GCGCGC) inserted as the first and third additional sequences (see FIG. 3). Capture was performed essentially as described above using undiluted, 10× diluted, and 100× diluted amplicon. The amount of product captured was quantified essentially as described above. Results are shown in FIG. 14. Using a capture oligomer containing clamp sequences improved its ability to normalize output amounts across the different dilutions of amplicon.

H. Capture of Predetermined Amount of Amplicon with a Capture Oligomer and a Complementary Oligomer

### Oligomers.

PCR to amplify a segment from the E. faecium vanA gene was carried out using the primers:

Efm_vanA_F (SEQ ID No. 148):
GGCTGCGATATTCAAAGCTCAG
Efm_vanA_R (SEQ ID No. 149):
CTGAACGCGCCGGCTTAAC

The prmers were designed to generate amplicon with the following sequence (SEQ ID No. 150):

GGCTGCGATATTCAAAGCTCAGCAATTTGTATGGACAAATCGTTGACATA
CATCGTTGCGAAAAATGCTGGGATAGCTACTCCCGCCTTTTGGGTTATTA
ATAAAGATGATAGGCCGGTGGCAGCTACGTTTACCTATCCTGTTTTTGTT
AAGCCGGCGCGTTCAG

A capture oligomer designated CC Blo vanA 001 was provided having the following sequence (SEQ ID No. 151):

AAAAAAAAAAAAAAAAAAAA/iSp18/CTCCTCTGGCACCGTGCTGCCT
TGGCTTCATTGTGGTCCTGAACGCGCCGGCTTAAC
(iSp18 = Hexaethylene Glycol (HEG) internal
spacer (IDT))

This oligomer comprises the elements shown for the exemplary capture oligomer of FIG. 10A. In this oligomer, the 5′ poly-A sequence is the capture sequence having first and second portions. iSp18 is the internal extension blocker.

CTCCTCTGGCACCGTGCTGCCTTGGCTTCATTGTGGTC is a spacer sequence having first and second portions. The target-hybridizing sequence (THS) is CTGAACGCGCCGGCTTAAC, which hybridizes specifically to a segment of the vanA gene sequence in the target amplicon.

A complementary oligo designated Blocker_vanA 001, comprising the elements shown for the exemplary complementary oligomer of FIG. 10A, was provided having the following sequence (SEQ ID No. 152): CGGTGCCAGAGGAGTTTTTTTTTT/invdt/wherein invdt is an inverted T nucleotide which serves as a blocking moiety. In this oligomer, CGGTGCCAGAGGAG is the complement of the first portion of the spacer sequence of the capture oligomer, and TTTTTTTTTT is the complement of the second portion of the capture sequence.

A secondary capture reagent with the following sequence was used:

dT₂₀-biotin (SEQ ID No. 145):
TTTTTTTTTTTTTTTTTTTT/3′Biotin

The following primers and probe were used for quantitative PCR (qPCR) analysis of copy control products:

vanA_PCR2_Fwd (SEQ ID No. 153):
TTGTATGGACAAATCGTTGACATACA
Efm_Probe_FAM (SEQ ID No. 154):
56-FAM/TGCTGGGATAGCTACTCCCGCCTTTTGG/3′IowaBlack
CC_Univ_Inner_Rev (SEQ ID No. 155):
ACCGTGCTGCCTTGGCTTC

### Protocol Reaction Conditions.

(1) PCR amplicon was generated for the target vanA utilising the Efm_vanA_F and Efm_vanA_R primers shown above; amplicon was quantitated via chip-based capillary electrophoresis using the Agilent BioAnalyzer.
(2) Capture oligomer annealing and extension of the amplicon strand—The vanA amplicon was used undiluted (neat) or 10-fold diluted (approximately 2×10¹³ and 2×10¹² copies, respectively). An 80 μL aliquot of each amplicon amount (neat and 10-fold diluted) was combined with 20 μL of capture oligomer annealing/extension reaction mixture yielding a final mixture consisting of 0.02 U/μl Deep Vent (exo-) Pol (NEB), 0.4× Deep Vent Pol reaction buffer, 0.012 mM dNTP's, 1.8 mM MgCl₂, 0.6 mg/ml BSA, 1×10¹¹ copies of the capture oligomer (CC Blo vanA 001) and 1×10¹² copies of the complementary oligomer (Blocker_vanA 001) for a final volume of 100 μL. The capture oligomer was annealed to the 3′-end of the complementary strand of the vanA amplicon and the amplicon strand was extended using a thermal cycler according to the following thermal profile: 92° C. for 2 minutes, 64° C. for 2 minutes and 68° C. for 10 minutes. In this example the 3′-end of the capture oligomer was also extended.
(3) Hybridization of the complement of the capture sequence of the capture oligomer—To the entire capture oligomer/amplicon extension reaction mixture was added 50 μL of a secondary capture reagent at 3× concentration, resulting in a final concentration of 42 mM NaCl, 0.33 mg/ml BSA and 10⁹ copies of the complement of the capture sequence (dT2o-biotin). Hybridization was carried out by incubating the reaction mix at 30° C. for 10 minutes.
(4) Capture of the amplicon and capture oligomer extension product/capture oligomer complex—A 50 μL aliquot (200 μg) of streptavidin-coated magnetic beads was added to the entire hybridization mixture (150 μL), resulting in a final concentration of 1 M NaCl, 5 mM TrisHCl (pH 7.5), 0.5 mM EDTA, 0.05% Tween 20 and 0.5 mg/ml BSA. The complex was captured onto the beads at 25° C. and the beads were washed using a wash reagent with the same composition as detailed immediately above.
(5) Elution—After the final wash was completed and the wash buffer removed, 30 μL of water was added to the bead pellet, the beads were resuspended and incubated at 70° C. for 2 minutes. The beads were pelleted with a magnet and the eluate was removed.
(6) Quantitation—The amount of eluted product as well as capture oligomer extension products (see step 2 above) were quantified by qPCR using primers targeting the vanA PCR2 Fwd primer site and the universal primer-adaptor site (CC Univ Inner Rev) along with the Efn Probe FAM.

### Results and Conclusions:

A 0-fold (neat) and 10-fold dilution of the amplicon produced in step 1 above (PCR) were subjected to the copy control process (see steps 2-5 above) using 10⁹ copies of the secondary capture oligomer. As shown in Table 18, despite a 10-fold difference in the input target amount the output levels were essentially identical.

TABLE 18

| PCR | Output (# | Fold |
|---|---|---|
| Dilution | copies) | Difference |
| Neat | 3.24E+08 | — |
| 10-fold | 3.14E+08 | 1.03 |

These data demonstrate that a capture oligomer and a complementary oligomer as described herein can be used to yield a pre-determined, normalized amount of target output across a 10-fold difference in input target amounts.

An additional experiment was performed essentially as described above with the following differences:
(1) PCR amplicon was purified using the QIAGEN QIAquick PCR Purification kit according to the manufacturer's instructions prior to quantitation via chip-based capillary electrophoresis using the Agilent BioAnalyzer.
(2) Capture oligomer annealing and extension of the amplicon strand—The vanA amplicon was used undiluted (neat), 10-fold diluted and 100-fold diluted (approximately 8×10¹¹, 8×10¹⁰ and 6×10⁹ copies, respectively). Capture oligomer (CC Blo vanA 001) was used at 1×10¹² copies/reaction of the capture oligomer and the complementary oligo (Blocker_vanA 001) was used at zero or 1×10¹³ copies/reaction. The capture oligomer was annealed to the 3′-end of the complementary strand of the vanA amplicon and the amplicon strand was extended using a thermal cycler according to the following thermal profile: 95° C. for 2 minutes and 64° C. for 15 minutes. All other conditions in this step were the same as step 2 above.
(3-6) Steps 3-6 were conducted as described in steps 3-6 above except that in step 4 the complex was captured onto the beads at 30° C. instead of 25° C.

### Results and Conclusions:

A 0-fold (neat), 10-fold and 100-fold dilution of the amplicon produced in step 1 above (PCR) were subjected to the copy control process (see steps 2-5 above). In this experiment, the amounts of various nucleic acid components used were approximately 8×10¹¹, 8×10¹⁰ and 6×10⁹ copies of target, 1×10¹² copies of capture oligomer, zero or 1×10¹³ copies of complementary oligomer and 1×10⁹ copies of the secondary capture oligomer. The results with or without complementary oligomer are shown in Table 19.

TABLE 19

| | Without complementary oligo | | With complementary oligo | |
|---|---|---|---|---|
| Input* (# | Output (# | Fold | Output (# | Fold |
| copies) | copies) | difference | copies) | difference |
| Neat | 9.3 × 10⁷ | — | 3.6 × 10⁸ | — |
| 10-fold | 2.6 × 10⁷ | 3.6 | 1.6 × 10⁸ | 2.3 |
| 100-fold | 5.0 × 10⁵ | 184 | 2.5 × 10⁷ | 14.4 |

*Target amplicon produced in Step 1

In the absence of complementary oligomer, the secondary capture oligomer (dT2o-biotin) can bind to any of the capture oligomer molecules whether or not they are bound to target. In this experiment, 1×10¹² copies of capture oligomer and 1×10⁹ copies of the secondary capture oligomer were used, i.e., there is a 1000-fold difference in these amounts. At the highest target level (neat), the majority of the capture oligomer will be bound to target and therefore the majority of secondary capture oligomer will bind to capture oligomer associated with target and the output copies after capture and elution will be relatively high. This is borne out in the data for the neat target input (without complementary oligo) where a relatively high output (9.3×10⁷ copies) is observed. However, at the 10-fold dilution of target there will be excess capture oligomer and therefore not all of it will bind to target. Some of the secondary capture oligomer will bind to capture oligomer associated with target but some will be to capture oligomer which is not bound to target. Therefore the output will go down, as is indeed observed (output=2.6×10⁷ copies). At the 100-fold dilution of target the majority of the capture oligomer will not be bound to target and likewise the majority of the secondary oligomer will bind to capture oligomer with is not associated with target. Therefore, the expectation under these conditions is a significantly decreased output, which is in fact observed (output=5.0×10⁵ copies).

In the presence of complementary oligomer, capture oligomer that has not bound to target will have the complementary oligomer bound which in turn will block the secondary capture reagent from binding. Conversely, capture oligomer that has bound to target will not have complementary oligomer bound (which has been displaced) which in turn will allow the secondary capture reagent to bind. Therefore, at all levels of target input tested in this experiment the output is expected to be higher in the presence of complementary oligomer than in the absence of complementary oligomer (results of which are discussed above). This is exactly what is observed (see Table 6). Furthermore, the data demonstrate that normalization is occuring, with only about a 2-fold difference between the output of the neat and 10-fold dilution target levels and only a little over a 14-fold difference between the output of neat and 100-fold target levels. The output at 100-fold target dilution is slightly lower than theoretically because the binding kinetics are slower due to this low level of target. Given longer incubation times the output would increase and the normalization factor would improve.

These data demonstrate that a capture oligomer and a complementary oligomer as described herein can be used to yield a pre-determined, normalized amount of target output across a 100-fold difference in input target amounts.

I. Generation of a Capturable Product that Contains Additional Sequence (e.g., Adaptors) at Both Ends of the Target Sequence Using a Capture Oligomer, a Complementary Oligomer, a Displacer Oligomer, and a Forward Primer

### Oligomers.

PCR to amplify a segment from the E. faecium vanA gene was carried out using the primers:

Efm_vanA_F (SEQ ID No. 148):
GGCTGCGATATTCAAAGCTCAG
Efm_vanA_R (SEQ ID No. 149):
CTGAACGCGCCGGCTTAAC

The prmers were designed to generate amplicon with the following sequence (SEQ ID No. 150):

GGCTGCGATATTCAAAGCTCAGCAATTTGTATGGACAAATCGTTGACAT
ACATCGTTGCGAAAAATGCTGGGATAGCTACTCCCGCCTTTTGGGTTAT
TAATAAAGATGATAGGCCGGTGGCAGCTACGTTTACCTATCCTGTTTTT
GTTAAGCCGGCGCGTTCAG A

capture oligomer designated PCR2R_adapter_CC was provided having the following sequence (SEQ ID No. 156):

AAAAAAAAAAAAAAAAAAAA/iSp18/CTCCTCTGGCACCGTGCTGCCT
TGGCTTCATTGTGGTCGTAGCTGCCACCGGCCTAT
(iSp18 = Hexaethylene Glycol (HEG) internal
spacer (IDT))

This oligomer comprises the elements shown for the exemplary capture oligomer of FIG. 10A. In this oligomer, the 5′ poly-A sequence is the capture sequence having first and second portions. iSpl8 is the internal extension blocker.

CTCCTCTGGCACCGTGCTGCCTTGGCTTCATTGTGGTC is a spacer sequence having first and second portions. The target-hybridizing sequence (THS) is GTAGCTGCCACCGGCCTAT, which hybridizes specifically to a segment of the vanA gene sequence in the target amplicon.

A complementary oligo designated Blocker_vanA 001, comprising the elements shown for the exemplary complementary oligomer of FIG. 10A, was provided having the following sequence (SEQ ID No. 152): CGGTGCCAGAGGAGTTTTTTTTTT/invdt/wherein invdt is an inverted T nucleotide which serves as a blocking moiety. In this oligomer, CGGTGCCAGAGGAG is the complement of the first portion of the spacer sequence of the capture oligomer, and TTTTTTTTTT is the complement of the second portion of the capture sequence.

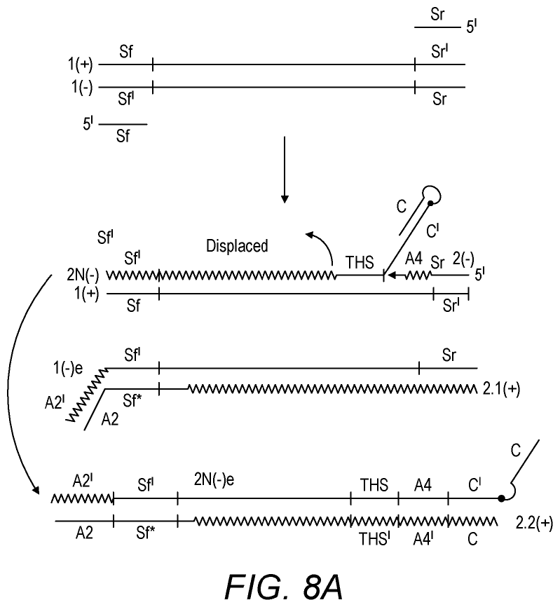

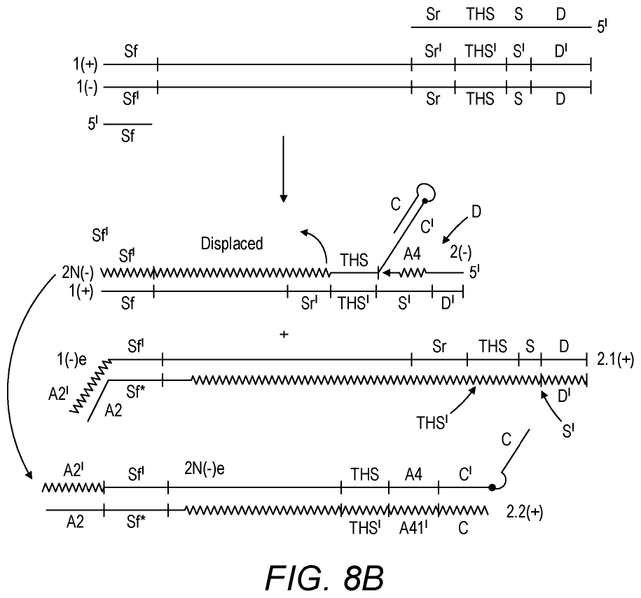

Efm_vanA_R (sequence above), comprising the elements shown for the exemplary displacer oligomer of FIG. 8A, was provided. An oligomer designated PCRIF adapter, comprising the elements shown for the exemplary forward primer with adapter of FIG. 8A, was also provided having the following sequence (SEQ ID No. 157):

AAAACGAGACATGCCGAGCATCCGCGGCTGCGATATTCAAAGCTCAG.

A secondary capture reagent with the following sequence was used:

dT₂₀-biotin (SEQ ID No. 145):
TTTTTTTTTTTTTTTTTTTT/3′Biotin

The following primers and probe were used for quantitative PCR (qPCR) analysis of copy control products:

CCRPA_uni_F (SEQ ID No. 158):
AAAACGAGACATGCCGAGCATC
Efm_Probe_FAM (SEQ ID No. 154):
56-FAM/TGCTGGGATAGCTACTCCCGCCTTTTGG/3′IowaBlack
CC_Univ_Inner_Rev (SEQ ID No. 155):
ACCGTGCTGCCTTGGCTTC

### Protocol Reaction Conditions

PCR amplicon was generated for the target vanA utilising the Efm_vanA_F and Efm_vanA_R primers shown above; amplicon was purified using the QIAGEN QIAquick PCR Purification kit according to the manufacturer's instructions prior to quantitation via chip-based capillary electrophoresis using the Agilent BioAnalyzer.

Capture oligomer, displacer oligomer and forward primer with adapter annealing and extension—An aliquot of the vanA amplicon containing approximately 1×10¹² copies was combined with annealing/extension reaction mixture yielding a final mixture consisting of 0.02 U/μl Deep Vent (exo-) Pol (NEB), 0.4× Deep Vent Pol reaction buffer, 0.012 mM dNTP's, 1.8 mM MgCl₂, 0.6 mg/ml BSA, 5×10¹³ copies of the capture oligomer (PCR2R_adapter_CC), ±1×10¹³ copies of the displacer oligomer (Efm_vanA_R), and 5×10¹³ copies of the forward primer with adapter (PCRIF adapter) for a final volume of 100 μL. Annealing and extension of the capture and displacer oligomers with the input amplicon and annealing and extension of the forward primer with adapter with the extension product of the capture oligomer all occurred in the same annealing/extension reaction using a thermal cycler according to the following thermal profile: 95° C. for 5 minutes then 64° C. for 20 minutes.

Quantitation—Aliquots of each of the annealing extension reactions were diluted 100-fold and the amount of product contained in each was quantified by qPCR using primers CCRPA_uni_F and CC Univ Inner Rev (targeting the universal adapter regions in the forward and reverse directions, respectively) along with the Efn_Probe_FAM.

### Results and Conclusions:

A single-cycle annealing and extension reaction (single-cycle is defined as only 1 denaturation step, e.g., incubation at 95° C.; another cycle would begin with another heat denaturation step) was conducted using the input target and oligomers described above. As shown in Table 20, product was formed that contained universal adapters at both ends of the molecule, as evidenced by amplification using universal primers.

TABLE 20

| Displacer | Output (# |
|---|---|
| Oligo | copies) |
| + | 1.3E+11 |
| − | 2.8E+11 |

These data demonstrate that the embodiment of the present invention depicted in FIG. 8A can be used to generate product with an adapter (or other desired sequences) at both ends of the molecule using a single annealing/extension cycle. Further, these data demonstrate that at least one of the primer-adapter oligomers (PCR2R_adapter_CC in this case) can bind to an internal site in the target and not only the terminus. These data also demonstrate that the desired product can be generated in the absence of the displacer oligomer. Without wishing to be bound by any particular theory, it is possible that different mechanisms can function within the disclosed embodiment to yield the desired product. It is possible that multiple mechanisms are functioning in the presence of displacer oligomer to generate the observed results.

An additional experiment was performed essentially as described above with the following differences:
(2) Capture oligomer, displacer oligomer and forward primer with adapter annealing and extension—An aliquot of the vanA amplicon containing approximately 1×10¹³ copies was combined with annealing/extension reaction mixture yielding a final mixture consisting of 0.02 U/μl Deep Vent (exo-) Pol (NEB), 0.4× Deep Vent Pol reaction buffer, 0.012 mM dNTP's, 1.8 mM MgCl₂, 0.6 mg/ml BSA, 5×10¹⁴ copies of the capture oligomer (PCR2R_adapter_CC) and 5×10¹⁴ copies of the forward primer with adapter (PCRIF adapter) for a final volume of 100 μL. Annealing and extension of this mixture was conducted using a thermal cycler according to the following thermal profile: 95° C. for 5 minutes then 64° C. for 15 minutes. At this point 5×10¹⁴ copies of the displacer oligomer (Efm_vanA_R) was added to some replicates of the reaction mixture and to some only buffer was added, and annealing and extension were continued using the thermal profile 64° C. for 5 minutes, 75° C. for 5 minutes and 72° C. for 15 minutes.

### Results and Conclusions:

A single-cycle annealing and extension reaction (single-cycle is defined as only 1 denaturation step, e.g., incubation at 95° C.; another cycle would begin with another heat denaturation step) was conducted using the input target and oligomers described above. The displacer oligomer was added to the annealing and extension reaction partway through the process to further optimize performance. As shown in Table 21, product was formed that contained universal adapters at both ends of the molecule, as evidenced by amplification using universal primers.

TABLE 21

| Displacer | Output (# |
|---|---|
| Oligo | copies) |
| + | 3.2E+12 |
| − | 1.9E+12 |

As above, these data demonstrate that the embodiment of the present invention depicted in FIG. 8A can be used to generate product with an adapter (or other desired sequences) at both ends of the molecule using a single annealing/extension cycle. Also as above, these data demonstrate that at least one of the primer-adapter oligomers (PCR2R_adapter_CC in this case) can bind to an internal site in the target and not only the terminus. Further, these data demonstrate that by adjusting the annealing and extension temperature profile—and in this case by adding the displacer oligomer partway through the process—that the overall performance can be improved. Of particular note is that under these conditions the amount of the desired product generated was greater when displacer oligomer was present than when it was absent, demonstrating that the displacement scheme is operating as shown if FIG. 8A. Again without wishing to be bound by any particular theory, it is possible that different mechanisms are also functioning within the disclosed embodiment to yield the desired product.

### Protocol Reaction Conditions (2).

PCR amplicon was generated for the target vanA utilizing the Efm_vanA_F and Efm_vanA_R primers shown above; amplicon was purified using the QIAGEN QIAquick PCR Purification kit according to the manufacturer's instructions prior to quantitation via chip-based capillary electrophoresis using the Agilent BioAnalyzer.

Capture oligomer, displacer oligomer and forward primer with adapter annealing and extension—An aliquot of the vanA amplicon containing approximately 1×10¹² copies was combined with annealing/extension reaction mixture yielding a final mixture consisting of 0.02 U/μl Deep Vent (exo-) Pol (NEB), 0.4× Deep Vent Pol reaction buffer, 0.012 mM dNTP's, 1.8 mM MgCl₂, 0.6 mg/ml BSA, 5×10¹³ copies of the capture oligomer (PCR2R_adapter_CC), ±5×10¹² copies of the displacer oligomer (Efm_vanA_R), and 5×10¹³ copies of the forward primer with adapter (PCRIF adapter) for a final volume of 100 μL. Annealing and extension of the capture and displacer oligomers with the input amplicon and annealing and extension of the forward primer with adapter with the extension product of the capture oligomer all occurred in the same annealing/extension reaction using a thermal cycler according to the following thermal profile: 95° C. for 5 minutes then 64° C. for 20 minutes.

Hybridization of the complement of the capture sequence of the capture oligomer—To the entire extension reaction mixture was added 50 μL of a secondary capture reagent at 3× concentration, resulting in a final concentration of 42 mM NaCl, 0.33 mg/ml BSA and 5×10¹⁴ copies of the complement of the capture sequence (dT2o-biotin). Hybridization was carried out by incubating the reaction mix at 30° C. for 10 minutes.

Capture of the amplicon and capture oligomer extension product/capture oligomer complex—A 50 μL aliquot (200 μg) of streptavidin-coated magnetic beads was added to the entire hybridization mixture (150 μL), resulting in a final concentration of 1 M NaCl, 5 mM TrisHCl (pH 7.5), 0.5 mM EDTA, 0.05% Tween 20 and 0.5 mg/ml BSA. The complex was captured onto the beads at 30° C. and the beads were washed using a wash reagent with the same composition as detailed immediately above.

Elution—After the final wash was completed and the wash buffer removed, 30 μL of water was added to the bead pellet, the beads were resuspended and incubated at 70° C. for 2 minutes. The beads were pelleted with a magnet and the eluate was removed.

Quantitation—Eluted product was quantified by qPCR using primers CCRPA_uni_F and CC Univ Inner Rev (targeting the universal adapter regions in the forward and reverse directions, respectively) along with the Efn Probe FAM.

### Results and Conclusions:

A single-cycle annealing and extension reaction was conducted using the input target and oligomers described above, the product was captured, washed, eluted and then quantitated using qPCR. As shown in Table 22, product was formed that contained universal adapters at both ends of the molecule which was captured and eluted, as evidenced by amplification using universal primers.

TABLE 22

| Displacer | Output (# |
|---|---|
| Oligo | copies) |
| + | 1.4E+09 |
| − | 1.5E+10 |

These data demonstrate that the embodiment of the present invention depicted in FIG. 8A can be used to generate product with an adapter (or other desired sequences) at both ends of the molecule using a single annealing/extension cycle and that this product can be isolated via capture onto beads, washing and elution. Further, these data demonstrate that at least one of the primer-adapter oligomers (PCR2R_adapter_CC in this case) can bind to an internal site in the target and not only the terminus. These data also demonstrate that the desired product can be generated in the absence of the displacer oligomer. Without wishing to be bound by any particular theory, it is possible that different mechanisms can function within the disclosed embodiment to yield the desired product. It is possible that multiple mechanisms are functioning in the presence of displacer oligomer to generate the observed results.

An additional experiment was performed essentially as described above with the following differences:

Capture oligomer, displacer oligomer and forward primer with adapter annealing and extension—The annealing/extension reaction mixture was the same as that above except new samples were added that also contained 5×10¹⁴ copies of the complementary oligomer (Blocker_vanA_001). Annealing and extension of the capture and displacer oligomers with the input amplicon, annealing of the complementary oligomer with the capture oligomer and annealing and extension of the forward primer with adapter with the extension product of the capture oligomer all occurred in the same annealing/extension reaction using a thermal cycler according to the following thermal profile:
a. 95° C. for 5 min, b. 75° C. for 30 sec, c. 74° C. for 30 sec, d. 73° C. for 30 sec, e. 72° C. for 30 sec, f. 71° C. for 30 sec, g. 70° C. for 30 sec, h. 69° C. for 2 min, i. 68° C. for 2 min, j. 67° C. for 2 min, k. 66° C. for 2 min, 1. 65° C. for 12 min.

Hybridization of the complement of the capture sequence of the capture oligomer—To conditions were the same as above except 1×10⁹ copies of the complement of the capture sequence (dT2o-biotin) were used.

### Results and Conclusions:

A single-cycle annealing and extension reaction was conducted using the input target and oligomers described above, the product was captured using a pre-defined amount of the complement of the capture sequence (dT2o-biotin), washed, eluted and then quantitated using qPCR. The results are shown in Table 23.

TABLE 23

| PCR | Output (# | Fold |
|---|---|---|
| Dilution | copies) | Difference |
| Neat | 2.0E+06 | — |
| 10-fold | 4.3E+05 | 4.7 |

These data demonstrate that the embodiment of the present invention depicted in FIG. 8A can be used to generate product with an adapter (or other desired sequences) at both ends of the molecule using a single annealing/extension cycle and that this product can be isolated via capture onto beads, washing and elution. Further, the 10-fold difference in input target level was normalized to a 4.7-fold difference, which is over a 2-fold normalization factor. Further, these data demonstrate that at least one of the primer-adapter oligomers (PCR2R_adapter_CC in this case) can bind to an internal site in the target and not only the terminus.

An additional experiment was performed essentially as described above with the following differences:
(2) Capture oligomer, displacer oligomer and forward primer with adapter annealing and extension—The annealing/extension reaction mixture was the same as that above except 1E+13 and 1E+12 (10-fold dilution) copies/reaction of input target was used; new samples were added that also contained 5×10″ copies of the complementary oligomer (Blocker_vanA 001). Annealing and extension of the capture oligomer with the input amplicon, annealing of the complementary oligomer with the capture oligomer and annealing and extension of the forward primer with adapter with the extension product of the capture oligomer all occurred in the same annealing/extension reaction using a thermal cycler according to the following thermal profile: 95° C. for 5 minutes, then 64° C. for 15 minutes.
(3) Hybridization of the complement of the capture sequence of the capture oligomer—To conditions were the same as above except 1×10⁹ copies of the complement of the capture sequence (dT2o-biotin) were used. Hybridization was conducted at 30° C. for 30 minutes.

### Results and Conclusions:

A single-cycle annealing and extension reaction was conducted using the input target and oligomers described above, the product was captured using a pre-defined amount of the complement of the capture sequence (dT2o-biotin), washed, eluted and then quantitated using qPCR. The results are shown in Table 24

TABLE 24

| | With Complementary Oligo | | Without Complementary Oligo | |
|---|---|---|---|---|
| PCR | Output (# | Fold | Output (# | Fold |
| Dilution | copies) | Difference | copies) | Difference |
| Neat | 1.8E+07 | — | 1.3E+06 | — |
| 10-fold | 2.7E+07 | 0.67 | 2.0E+06 | 0.65 |

These data demonstrate that the embodiment of the present invention depicted in FIG. 8A can be used to generate product with an adapter (or other desired sequences) at both ends of the molecule using a single annealing/extension cycle and that this product can be isolated via capture onto beads, washing and elution. Further, the 10-fold difference in input target level was normalized to a 0.67-fold difference (-1) when complementary oligo was present. Without complementary oligo the recovery of product was reduced over 10-fold, as would be expected while normalization was similar. Further, these data demonstrate that at least one of the primer-adapter oligomers (PCR2R_adapter_CC in this case) can bind to an internal site in the target and not only the terminus.

J. In-well amplification of target nucleic acids using Solution-Mediated Recombinase Polymerase Amplification (SM-RPA)

### Oligomers

In-well amplification was performed using a solution primer (HDA72F) and a primer immobilized on the surface of the wells (extHDA72R) with the following sequences:

HDA72F (SEQ ID No. 159):
5′-AAAACGAGACATGCCGAGCATCCGC-3′
extHDA72R (SEQ ID No. 160):
5′-AmmC12-AAAAAAAAAAAAAAAAAAAACCCCCCCCCCCCCCCCCCCC
iSp18-iSp18-iSp18-iSp18-iSp18/-AAAAAAACTCCTCTGGCAC
CGTGCTGCCTTGGCTTCATTGTGGTC-3′
(5′-AmmC12 = 5′-terminal C-12 amine and iSp18 =
Hexaethylene Glycol (HEG) internal spacer
(both from IDT)) Synthetic DNA templates
representing the micrococcal nuclease gene from
Staphylococcus aureus (nuc), the carbapenemase
gene from Klebsiella pneumoniae (Kpc) and Qin
prophage protein YdfU gene from Escherichia coli
(ydfU) were used, each with the following sequence
(includes adapter sequences, which are underlined):
nuc template (SEQ ID No. 161):
5′-AAAACGAGACATGCCGAGCATCCGCTCAGTAATGTTTCGAAAGGGCAATACGCA
AAGAGGTTTTTCTATTTCGCTACTAGTTGCTTAGTGTTAACTTTAGTTGTAGTTTCAA
GTCTAAGTAGCTCAGCAGACCACAATGAAGCCAAGGCAGCACGGTGCCAGAGGAGT
TTTTTT-3′
Kpc template (SEQ ID No. 162):
5′-AAAACGAGACATGCCGAGCATCCGCTAAACTCGAACAGGACTTTGGCGGCTCCA
TCGGTGTGTACGCGATGGATACCGGCTCAGGCGCAACTGTAAGTTACCGCGCTGAG
GAGCGCTTCCCACTGTGCAGCTCATTCGACCACAATGAAGCCAAGGCAGCACGGTG
CCAGAGGAGTTTTTTT-3′
ydfU template (SEQ ID No. 163):
5′-AAAACGAGACATGCCGAGCATCCGCTGCGGGTATTACTTAGACCTGTTCTGGTGC
CTGAGCTTGGGCTGGTGGTCCTTAAGCCGGGCCGTGAATCCATACAGATAGACCACA
ATGAAGCCAAGGCAGCACGGTGCCAGAGGAGTTTTTTT-3′

Synthetic DNA probes were used to detect the amplification products from the templates listed above, each with the following sequence:

nuc probe (SEQ ID No. 164):
5′-Cy3-TCGAAAGGGCAATACGCAAAGAG-3′
(5′-Cy3 = 5′-terminal Cy3 fluorophore)
Kpc probe (SEQ ID No. 165):
5′-Alex488N-TAAACTCGAACAGGACTTTGGCG-3′
(5′-Alex488N = 5′-terminal Alexa488 fluorophore)
ydfU probe (SEQ ID No. 166):
5′-Cy5-TGCGGGTATTACTTAGACCTGTTC-3′
(5′-Cy5 = 5′-terminal Cy5 fluorophore)

### Semiconductor Chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Buffer Reagent Preparation

“Buffer Mix” was prepared in an Eppendorf microfuge tube according to the formulation shown in Table 25. The 20% Carbowax was heated at 65° C. for 5 minutes. The water was added, and the mixture was vortexed to homogeneity and then placed in a cool block for 5 minutes. The DTT and UvsX Reaction Buffer were added and the mixture was vortexed to homogeneity.

TABLE 25

| | | Stock | Final | Per 25 μL |
|---|---|---|---|---|
| Reagent | Supplier | Conc. | Conc. | reaction |
| 20% Carbowax | Sigma Aldrich | 20% | 6% | 7.50 μL |
| Dithiothreitol | Merck Life Science | 1M | 4 mM | 0.10 μL |
| (DTT) | UK Limited | | | |
| PCR-grade water | Merck Life Science | N/A | N/A | 3.62 μL |
| UvsX Reaction | Intact Genomics | 10x | 1x | 2.50 μL |
| buffer | | | | |

“Energy Mix” was prepared in a separate Eppendorf microfuge tube by sequentially adding each of the components recorded in Table 26 (in the order shown) followed by thorough mixing (without vortexing).

TABLE 26

| | | Stock | | Final | | Per 25 μL |
|---|---|---|---|---|---|---|
| Reagent | Supplier (Catalogue no.) | Conc. | | Conc. | | reaction |
| Solution primer | IDT | 100 | μM | 0.6 | μM | 0.15 μL |
| HDA72F | | | | | | |
| dNTPs | Life Technologies/Invitrogen/ | 25 | mM each | 0.46 | mM each | 0.46 μL |
| | Applied Biosystem (R0186) | | | | | |
| ATP | Merck Life Science UK Limited | 100 | mM | 4 | mM | 1.00 μL |
| | (GE27-2056-01) | | | | | |
| Phosphocreatine | Merck Life Science UK Limited | 1000 | mM | 12 | mM | 0.30 μL |
| | (P7936-1G) | | | | | |

“Core Mix” was prepared in a separate Eppendorf microfuge tube by sequentially adding each of the components recorded in Table 27 (in the order shown) followed by thorough mixing (without vortexing).

TABLE 27

| | | Stock | | Final | | Per 25 μL | |
|---|---|---|---|---|---|---|---|
| Reagent | Supplier (Catalogue no.) | Conc. | | Conc. | | reaction | |
| PCR-grade water | Merck Life Science UK | NA | | NA | | 1.00 | μL |
| | Limited (3315932001) | | | | | | |
| gp32 | NEB (M0330L) | 10000 | ng/μL | 250 | ng/μL | 0.63 | μL |
| UvsX DNA | Intact Genomics (3565) | 5000 | ng/μL | 250 | ng/μL | 1.25 | μL |
| Recombinase | | | | | | | |
| T4 UvsY Protein | Intact Genomics (3575) | 2000 | ng/μL | 100 | ng/μL | 1.25 | μL |
| Creatinine Kinase | Merck Life Science UK | 1000 | ng/μL | 60 | ng/μL | 1.50 | μL |
| | Limited (10127566001) | | | | | | |
| Bsu DNA | NEB (M0330L) | 5 | U/μL | 0.75 | U/μL | 3.75 | μL |
| Polymerase | | | | | | | |

Buffer Mix, Energy Mix and Core Mix were all stored at 4° C. until use.

### Protocol/Reaction Conditions

The flow cell was flushed twice with 1× RPA Annealing Buffer (20 mM Tris, pH 7.5, 5 mM Mg(OAc)₂, 150 mM NaCl, 0.01% Tween).

Synthetic DNA template in 1× RPA Annealing Buffer was loaded into the flow cell in a volume of 25 μL. In this example, equimolar mixtures of nuc, Kpc and YdfU were tested at 10⁶, 10⁷ or 10⁸ copies of each target per reaction.

DNA template was annealed to the primer immobilized on the surface of the wells (extHDA72R) using the following thermal protocol:
a. 95° C. for 2 min, b. 60° C. for 5 min, c. 25° C. for 15 min.

The solution was removed and the chip was washed twice with 50 μL of RPA Wash Buffer (0.06×SSC+0.06 Tween20).

The Buffer Mix, Enzyme Mix, Core Mix were combined in the ratio indicated in Tables 1-3 (total amount of each per 25 μL reaction) on a cool block and 25 μL was loaded onto the chip.

Amplification was then performed using the following thermal protocol:
a. 43° C. for 25 min, b. 95° C. for 3 min.

The chip was then incubated on a cool block for 5-10 minutes, the reaction mixture was removed and the chips washed twice with 50 μL RPA Wash Buffer.

The non-immobilized strand of the resulting amplification products was removed by loading 25 μL of 20 mM NaOH into the chip and incubating for 10 min at ambient temperature (about 20-26° C.). The NaOH solution was removed and the chip was washed twice with 50 μL RPA Wash Buffer.

A probe solution was then prepared containing IX RPA Annealing buffer and 2 μM each of the nuc, Kpc and ydfU fluorescently labelled probes described above. Twenty-five microliters of this solution was then loaded onto the chip and the probes were annealed to their respective targets, if present, using the following thermal protocol:
a.95° C. for 2 min, b. 58° C. for 5 min, c. Passive cool-down to ambient temperature (about 20-26° C.) for 15 min.

The reaction mixture was removed and the chip was washed twice with 50 μL RPA Wash Buffer. The chip was dried using a nitrogen gun and then imaged using a fluorescent microscope equipped with filters enabling specific detection of Alexa488, Cy3 and Cy5 fluorophores.

### Results and Conclusions

The fluorescent microscopy images of the 3 individual chips with 10⁶, 10⁷ or 10⁸ starting copies of each synthetic DNA template showed patches of amplified target DNA, distinguishable by the three fluorophore colors (FIG. 53). In conclusion, SM-RPA produced clonal, “sequencing-ready” amplification products on the surface of the semiconductor chip in just 25 minutes of amplification time.

K In-well amplification of target nucleic acids using Rolling Circle Amplification (RCA)

### Oligomers

In-well amplification was performed using two immobilized primers with the following sequences (both purchased from ATDBio):

DBCO_A10H1_Li-dev[1]_R/SplintXL (SEQ ID No. 167):
5′-DBCO-AAAAAAAAAA-isp18-GCTCGAATCAGTCCTGTCAGTC
T*T*T*T-3′
DBCO_A10H1_dev[1]-F (SEQ ID No. 168):
5′-DBCO-AAAAAAAAAA-isp18-TCCGCGGGAGCTTCAACAT*C*G*
C*G-3′
(for both oligos, 5′-DBCO = 5′-terminal
dibenzocyclooctyl, iSp18 = Hexaethylene Glycol
(HEG) internal spacer and * = Thiophosphate
protection)

Synthetic DNA templates (purchased from IDT Technologies) representing the D-alanine--D-alanine ligase gene from Enterococcusfaecalis (ddl) and the Qin prophage protein YdfU gene from Escherichia coli (ydfU) were used, each with the following sequence (includes adapter sequences, which are underlined):

ddl template (SEQ ID No. 169):
5′-5Phos-GACTGATTCGAGCCGCGCTTCAATTCCTTGTTCAACGATTG
CTCGAGAATCATACTGATAGGCTGTTGCTAAAGCATTTTGCAGCTCTTCT
CGGTTTTCCGCGGGAGCTTCAACATCGCGCGAAAAAAAAAAAAAAAGACT
GACAG-3′
ydfU template (SEQ ID No. 170):
5′-5Phos-GACTGATTCGAGCTGCGGGTATTACTTAGACCTGTTCTGGT
GCCTGAGCTTGGGCTGGTGGTCCTTAAGCCGGGCCGTGAATCCATACAGA
TATCCGCGGGAGCTTCAACATCGCGCGAAAAAAAAAAAAAAAGACTGACA
G-3′

Synthetic DNA probes (purchased from IDT Technologies) were used to detect the amplification products from the templates listed above, each with the following sequence:

ddl probe (SEQ ID No. 171):
5′-Cy5-CAACGATTGCTCGAGAATCAT-3′
(5′-Cy5 = 5′-terminal Cy5 fluorophore)
ydfU probe (SEQ ID No. 172):
5′-Cy3-TGCGGGTATTACTTAGACCTGTTC-3′
(5′-Cy3 = 5′-terminal Cy3 fluorophore)

### Semiconductor Chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Buffer Reagent Preparation

“10× RCA Annealing Buffer” was prepared according to the formulation shown in Table 28.

TABLE 28

| | Supplier | | | | |
|---|---|---|---|---|---|
| | (catalogue | Stock | Final | Per 10 | |
| Reagent | no.) | Conc. | Conc. | mL | |
| Tris-HCL pH 7.5 | ThermoFisher | 1000 mM | 500 mM | 5 | mL |
| MgCl₂ | Sigma | 1000 mM | 150 mM | 1.5 | mL |
| NaCl | Sigma | 5000 mM | 1200 mM | 2.4 | mL |
| Tween20 | Sigma | 100% | 0.1% | 0.01 | mL |
| Water | Sigma | — | — | 1.09 | mL |

### Protocol/Reaction Conditions

The flow cell was flushed twice with 1× RCA Annealing Buffer.

Synthetic DNA template in 1× RCA Annealing Buffer was loaded into the flow cell in a volume of 25 μL. In this example, equimolar mixtures of ddl and YdfU were tested at 5×10⁷ and 5×10⁸ copies of each target per reaction.

DNA template was annealed to the reverse primer immobilized on the surface of the wells (DBCO-A10H1-Li-dev[l]_R/SplintXL) using the following thermal protocol shown in Table 29.

TABLE 29

| Step | Temperature (° C.) | Time | Ramp rate |
|---|---|---|---|
| 1 | Heat to 80 from ambient | [See ramp rate] | 1.6° C./s |
| | (about 20-26° C.) | | |
| 2 | 80 | 3 min | Hold |
| 3 | Cool from 80 to 60 | [See ramp rate] | Passive cooling |
| 4 | 60, 58, 56, 54, 52, 50, | Hold 30 sec at each | 0.2° C./s |
| | 48, 46, 44, 42 | temp | |
| 5 | Cool from 42 to 37 | [See ramp rate] | |
| 6 | 37 | 10 min | Hold |
| 7 | Cool from 37 to 20 | [See ramp rate] | 0.2° C./s |
| 8 | 20 | 15 min | Hold |

The solution was removed and the chip was washed twice with 50 μL of RCA Wash Buffer (37.5 mM Tris-HCl pH 7.5, 10 mM MgCl₂, 50 mM KCl, 2 mM (NH4)₂SO₄).

“Ligation Mix” was prepared in an Eppendorf microfuge tube according to the formulation shown in Table 30.

TABLE 30

| | Supplier | | | Final | | Per 25 μL |
|---|---|---|---|---|---|---|
| Reagent | (catalogue no.) | Stock | | Concentration | | reaction |
| Water | Sigma | — | | — | | 21.25 μL |
| T4 ligase | NEB | 10 | X | 1 | X | 2.50 μL |
| buffer | | | | | | |
| T4 Ligase | NEB | 2000 | U/μL | 100 | U/μL | 1.25 μL |

Twenty-five L of the Ligation Mix was loaded onto the chip and incubated at 22° C. for 30 minutes. The solution was removed and the chip was washed twice with 50 μL of RCA Wash Buffer.

“Amplification Mix” was prepared in an Eppendorf microfuge tube according to the formulation shown in Table 31.

TABLE 31

| | Supplier | | | | | |
|---|---|---|---|---|---|---|
| | (catalogue | Stock | | Final | | Per 25 μL |
| Reagent | no.) | Conc. | | Conc. | | reaction |
| PCR-grade water | Sigma | — | | — | | 16.00 μL |
| RCA Wash | — | 10 | X | 1 | X | 2.50 μL |
| Buffer | | | | | | |
| Dithiothreitol | Sigma | 100 | mM | 1 | mM | 0.25 μL |
| (DTT) | | | | | | |
| dNTP | ThermoFisher | 10 | mM | 1 | mM | 2.50 μL |
| EquiPhi29 | Invitrogen | 10 | U/μL | 1.5 | U/μL | 3.75 μL |

| | |
|---|---|
| Total: | 25.00 μL |

Twenty-five μL of the Amplification Mix was loaded onto the chip and incubated at 45° C. for 2 hours. The solution was removed from the chip and the reaction was inactivated by adding 25 μL “Inactivation Mix” (50 mM Tris-HCl pH 7.5, 50 mM EDTA) and pipette up and down twice. The solution was removed and the chip was washed twice with 50 μL of RCA Wash Buffer.

The strands of the amplified material were denatured by loading 25 μL of 20 mM NaOH into the chip and incubating for 5 min at ambient temperature (about 20° C.-26° C.). The NaOH solution was removed and the chip was washed twice with 50 μL RCA Wash Buffer.

A probe solution was then prepared containing 1× RCA Annealing buffer and 1 μM each of the ddl and ydfU fluorescently labelled probes described 004 above. Twenty-five microliters of this solution was then loaded onto the chip and the probes were annealed to their respective targets, if present, using the following thermal protocol:
a. 95° C. for 2 min, b. 54° C. for 15 min, c. Passive cool-down to ambient temperature (about 20-26° C.) for 15 min.

The reaction mixture was removed and the chip was washed twice with 50 μL RCA Wash Buffer. The chip was dried using a nitrogen gun and then imaged using a fluorescent microscope equipped with filters enabling specific detection of Cy3 and Cy5 fluorophores.

### Results and Conclusions

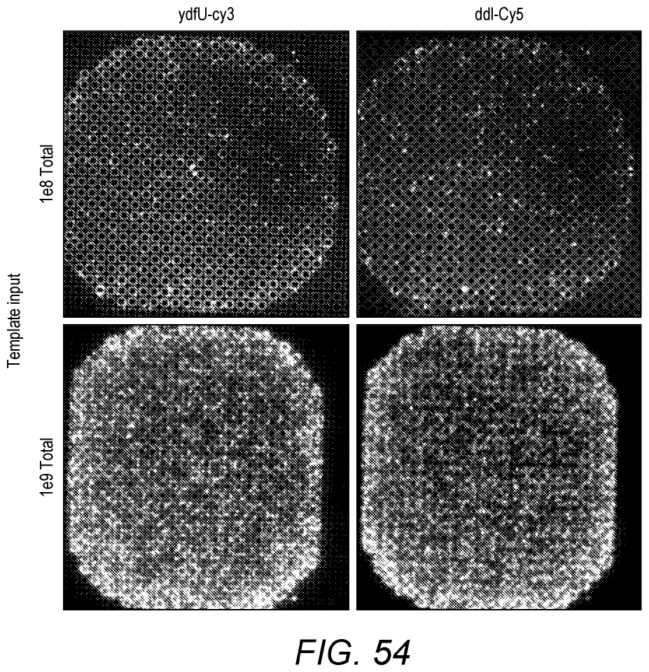

Fluorescent microscopy images of the equimolar duplex branched surface-phase RCA assay showed discrete clusters of amplified target DNA for each of the two targets, distinguishable by two different fluorophores (FIG. 54). The density of the clusters is dependent on the target copy number input. In conclusion, branched surface-phase RCA produced clonal, “sequencing-ready” amplification products on the surface of the semiconductor chip in 2 hours amplification time.

### L. Sequencing of a Synthetic Template Directly Immobilized on the Chip Surface

### Oligomers

Sequencing was performed using a synthetic DNA template (Oligo 1; ATDBio) immobilized on the surface of the wells and a sequencing primer (Oligo 2; IDT) complementary to the 3′-region of the template (nucleotides underlined in Oligo 1) with the following sequences:

Oligo 1 (SEQ ID No. 173):
5′-DBCO-TGTAGCACGATTGCAGCATTGTTAGCAGGATTGCGGGTGCC
AATGTGATCAACGTACAGAGGATCTAGTCGGC-3′
(5′-DBCO = 5′-terminal dibenzocyclooctyl; the
underlined region represents the sequencing primer
binding site; the non-underlined region is a
portion of the N-gene of SARS-COV-2)
Oligo 2 (SEQ ID No. 174):
5′-GCCGACTAGATCCTCTG-3′
(sequencing primer)

### Semiconductor Chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Buffer Reagent Preparation

“Sequencing Solution” (7.5 mM MgCl₂, 200 mM NaCl, 0.02% TERGITOL NP-9) was prepared in a glass bottle using deionized water (18MΩ; Merck Millipore), 1M magnesium chloride solution, 5M sodium chloride solution and TERGITOL NP-9 (neat; Merck). Briefly, the MgCl₂ and NaCl stock solutions were added to the deionized water to yield final concentrations of 7.5 and 200 mM, respectively. A magnetic stirring bar was added and the solution was mixed to homogeneity using a magnetic stirring plate. To ensure dissolved carbon dioxide was removed, the solution was then sparged with nitrogen gas (99.998% minimum nitrogen; BOC part #44-W) by placing a nitrogen supply tube in the bottle, ensuring the tube reached the bottom, and nitrogen was bubbled through the solution for 10 minutes. After this a supply of nitrogen was maintained above the solution to ensure it remained C02-free. A positive displacement pipette was used to add 250 μl of TERGITOL NP-9 to yield a final concentration of 0.02%. The mixture was stirred (under nitrogen) for an additional 10 minutes to fully dissolve the TERGITOL.

A 10 μM solution of each the individual natural nucleotides (dGTP, dCTP, dATP, dTTP) was prepared by adding 12.5 μL of 100 mM stock of the selected nucleotide (Fisher Scientific, 11843933) to 125 mL of Sequencing Solution. Using a pH probe (Sentron SI MicroFET (92270-010)) the pH of each dNTP solution was titrated to pH 8.05+0.01 using 10 mM NaOH (prepared from a 10 M NaOH stock; Merck, 72068). All dNTP solutions were prepared in a CO₂ free, nitrogen-controlled environment with mixing achieved by use of a magnetic stir bar.

Using a pH probe (Sentron SI MicroFET (92270-010)) “Wash Solution” was prepared by titrating the pH of Sequencing Solution to 8.05+0.01 using 10 mM NaOH (prepared from a 10 M NaOH stock; Merck, 72068) in a CO₂ free, nitrogen-controlled environment with mixing achieved by use of a magnetic stir bar.

1× Annealing Buffer was prepared by diluting a 20× stock of saline sodium citrate buffer (Life Technologies) to a final 1× concentration of 150 mM NaCl and 15 mM sodium citrate using Molecular Grade Water (Sigma).

A 5 μM working solution of Oligo 2 (sequencing primer) was prepared by adding 1.25 μL Oligo 2 (100 μM stock) to 5 μL of 1× Annealing Buffer to give a final composition of 5 μM sequencing primer in 0.8× Annealing Buffer.

A 25.4 U/μL working stock of “Sequencing Enzyme” was prepared by diluting 1 μL of IsoPol BST+DNA Polymerase at 2 kU/μL (ArcticZymes, custom preparation) in 79 μL of Sequencing Solution (see preparation details above). The solution was thoroughly mixed by pipetting 20 μL volumes up and down 10 times.

### Protocol/Reaction Conditions

The flow cell was flushed twice with 1× ThermoPol Buffer [4.5 mL 10× ThermoPol buffer (New England Biolabs), 27 μL Tween 20 (100% stock, Merck Life Science, P9416), 40.5 mL Molecular Grade Water (Sigma)].

Sequencing primer (5 μM Oligo 2 in Annealing Buffer; see preparation details above) was loaded into the flow cell. The inlet and outlet of the flow cell was sealed with custom plugs and the primer was annealed to the immobilized template (Oligo 1) using the following thermal protocol:
a. 95° C. for 120 sec, b. 90° C. for 30 sec, c. 85° C. for 30 sec, d. 80° C. for 30 sec, e. 78° C. for 120 sec,
f. Ramp down from 77° C. to 64° C. (inclusive) at a rate of 1° C. every 30 sec, g. 63° C. for 120 sec,
h. Ramp down from 62° C. to 59° C. (inclusive) at a rate of 1° C. every 30 sec, i. 58° C. 15 mins, j.
Passive cool-down to ambient temperature (about 20-26° C.) for 2 min.

The flow cell was then washed with 200 μL of IX ThermoPol buffer to remove any excess primer.

Sequencing Enzyme (25 U/μL IsoPol BST+; see preparation details above) was loaded into the flow cell and incubated for 10 minutes at ambient temperature (about 20-26° C.). The flow cell was flushed with 200 μL 1× ThermoPol Buffer.

A priming step was performed wherein Wash Solution was flowed across the chip at 5 mL/min. An electrical response test was performed by biasing the reference electrode with increasing voltage steps. The resulting change in mV output measured by the ISFETs on the IC was used to determine the relationship between reference electrode potential and the corresponding potential seen on the ISFETs, which then was used to determine the optimal reference electrode potential for the experiment.

Sequencing was then performed cycle by cycle. During each sequencing cycle, each of the 4 individual dNTP solutions (10 μM each; see preparation details above) were flushed sequentially across the chip (15 sec @5 ml/min for each nucleotide), with a wash step separating each nucleotide flow. The wash step was performed in two phases: 1) A “through-wash” to flush the nucleotide solution from the fluidic channels and flow cell, and 2) A purge wash, whereby a wash channel by-passes the nucleotide valving apparatus to wash the chip, whilst the next nucleotide solution is simultaneously directed via a purge channel to the waste receptacle. When a nucleotide is incorporated, the subsequent proton release is detected as a change in voltage by the integrated circuit.

Once the sequencing was finished, the data was analyzed using in-house proprietary algorithms for base-calling and bioinformatics analysis.

### Results and Conclusions

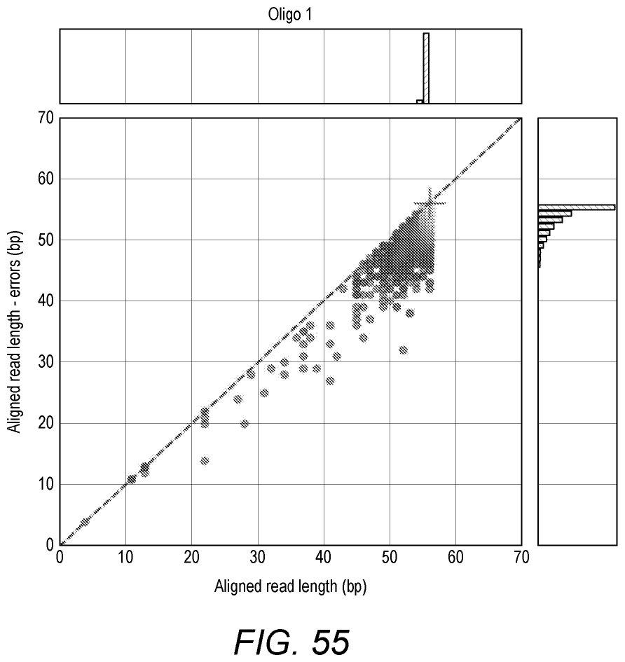

The sequencing results obtained from the immobilized synthetic template are shown in FIG. 55. Individual reads are plotted as Aligned Read Length (ARL; total alignment length from start to end positions) on the x-axis versus Aligned Read Length—Errors (ARL-e; total alignment length from start to end positions minus the number of errors on the y axis). A read with no errors would lie on the diagonal dashed line, where ARL-e=ARL. The location on the plot of a perfect, full-length (56 bp) read is indicated by the crosshairs symbol. The histogram sections shown in FIG. 55 indicate the distribution of reads on the different axes. The histogram across the top of the plot indicate the distribution of reads along the x-axis, i.e., ARL (bp); the histogram on the right-hand side of the plot indicate the distribution of reads along the y-axis, i.e., ARL-e (bp). The total number of reads in this example was 5899. The median ARL-e achieved was 54 bp. The consensus read has an alignment length of 56 bases with an error rate of 0% (Table 32). These data demonstrate that the system as described is capable of sequencing an immobilized template.

TABLE 32
REFR: TACGTTGATCACATTGGCACCCGCAATCCTGCTAACAATGCTGCAATCGTGCTACA
||||||||||||||||||||||||||||||||||||||||||||||||||||||||
READ: TACGTTGATCACATTGGCACCCGCAATCCTGCTAACAATGCTGCAATCGTGCTACA

### M. Sequencing of a Synthetic Template Using the Direct Hybridization Method

### Oligomers

The direct hybridization followed by sequencing method was performed using a solution-phase template (Oligo 3; IDT) and a primer immobilized on the surface of the wells (Oligo 4; ATDBio) complementary to the 3′-region of the template (nucleotides underlined in Oligo 3) with the following sequences:

Oligo 3 (SEQ ID No. 175):
5′-TTTAAGTCCCGCAACGAGCGCAACCCTTATCCTTTGTT
GCCAGCGGTCCGGCCGGGAACTCAAAGGAGACTGCCAGTGA
TAAACTGGAGGAAGGTGGGGATGACGTCAACGG-3′
Oligo 4 (SEQ ID No. 176):
5′-DBCO-CCGTTGACGTCATCCCCACCT-3′
(5′-DBCO = 5′-terminal dibenzocyclooctyl)

### Semiconductor Chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Buffer Reagent Preparation

All buffers/reagents were prepared as described in the example above (except Oligo 2 working solution, which was not used in this experiment).

A 0.5 μM working solution of synthetic DNA template (Oligo 3) was prepared by adding 0.5 μL Oligo 3 (100 μM stock) to 99.5 μL of 1× Annealing Buffer.

### Protocol/Reaction Conditions

Synthetic DNA template (0.5 μM Oligo 3 in Annealing Buffer; see preparation details above) was loaded into the flow cell. The inlet and outlet of the flow cell was sealed with custom plugs and the template was annealed to the immobilized primer (Oligo 4) using the following thermal protocol:
a. 95° C. for 120 sec, b. 90° C. for 30 sec, c. 85° C. for 30 sec, d. 80° C. for 30 sec, e. 78° C. for 120 sec, f. Ramp down from 77° C. to 64° C. (inclusive) at a rate of 1° C. every 30 sec, g. 63° C. for 120 sec, h. Ramp down from 62° C. to 59° C. (inclusive) at a rate of 1° C. every 30 sec, i. 58° C. 15 mins, j. Passive cool-down to ambient temperature (20-26° C.) for 2 min.

The subsequent steps (addition of Sequencing Enzyme, priming step, electrical response test, sequencing and analysis) were performed as described in the preceding working example.

### Results and Conclusions

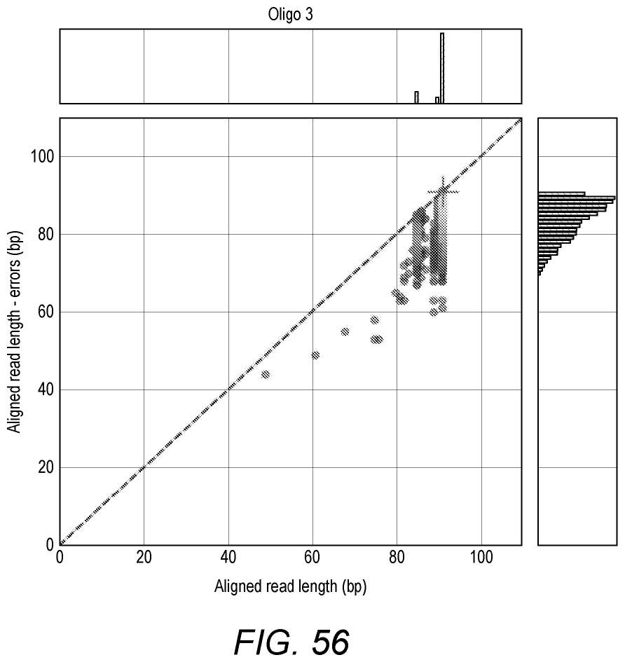

The sequencing results obtained from the in-solution synthetic template hybridized to the immobilized primer (i.e., Direct Hybridization method) are shown in FIG. 56. Individual reads are plotted as ARL on the x-axis versus ARL-e on the y axis (see example above for definition of ARL and ARL-x). A read with no errors would lie on the diagonal dashed line, where ALR-e=ARL. The location on the plot of a perfect, full-length (91 bp) read is indicated by the crosshairs symbol. The histogram sections shown in FIG. 56 indicate the distribution of reads on the different axes. The histogram across the top of the plot indicate the distribution of reads along the x-axis, i.e., ARL (bp); the histogram on the right-hand side of the plot indicate the distribution of reads along the y-axis, i.e., ARL-e (bp). The total number of reads in this example was 3829. The median ARL-e achieved was 85 bp. The consensus read has an alignment length of 97 bases with an error rate of 8.25% (Table 33). These data demonstrate that the system as described is capable of sequencing a template which is hybridized to an immobilized primer.

TABLE 3
REFR: TCCTCCAGTTTATCACTGGCAGTCT-CCTTTGAGTTCCCGGCCGGACCGCTGGCAACAAAGGATA-AGGG-TTGCGC-T-CGTTGCGGGA--CTTAA
|||||||||||||||||||||||||<|||||||||||||||||||||||||||||||||||||||<||||<||||||<|<||||||||||<<||*||
READ: TCCTCCAGTTTATCACTGGCAGTCTCCCTTTGAGTTCCCGGCCGGACCGCTGGCAACAAAGGATANAGGGNTTGCGCNTCCGTTGCGGGAACCTWAA
The symbols between the 2 sequences represent the following: |= perfect match, >= deletion, <= insertions, *= mismatch.

### N. Sequencing of a Template Generated Using In-Well Clonal Amplification

### Oligomers

In-well clonal amplification was performed using the following oligomers:

Oligo W (Solution-phase template to be
amplified) (SEQ ID No. 177):
5′-AAAACGAGACATGCCGAGCATCCGCTGCGGGTATTAC
TTAGACCTGTTCTGGTGCCTGAGCTTGGGCTGGTGGTCCT
TAAGCCGGGCCGTGAATCCATACAGATAGACCACAATGAA
GCCAAGGCAGCACGGTGCCAGAGGAGTTTTTTT-3′
Oligo X (Surface-phase amplification primer)
(SEQ ID No. 178):
5′-DBCO-AAAAACTCCTCTGGCACCGTGCTGCCTTGGC
TTCATTGTGGTC-3′
(5′-DBCO = 5′-terminal dibenzocyclooctyl)
Oligo Y (Solution-phase amplification primer)
(SEQ ID No. 179):
5′-AAAACGAGACATGCCGAGCATCCGC -3′
Oligo Z (Solution-phase sequencing primer)
(SEQ ID No. 180):
5′-GGAAGGCACGCTCTACTATTCAA-3′
Oligo Y = Solution-phase amplification primer;
Oligo Z = Solution-phase sequencing primer

Semiconductor chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Buffer Reagent Preparation

All buffers/reagents were prepared as described below.

“Sequencing Solution” (5 mM MgCl₂, 20 mM NaCl, 0.025% TERGITOL NP-9) was prepared in a glass bottle using deionized water (18MΩ; Merck Millipore), 1 M magnesium chloride solution, 5 M sodium chloride solution and TERGITOL NP-9 (neat; Merck). Briefly, the MgCl₂ and NaCl stock solutions were added to the deionized water to yield final concentrations of 5 and 20 mM, respectively. A magnetic stirring bar was added and the solution was mixed to homogeneity using a magnetic stirring plate. To ensure dissolved carbon dioxide was removed, the solution was then sparged with nitrogen gas (99.998% minimum nitrogen; BOC part #44-W) by placing a nitrogen supply tube in the bottle, ensuring the tube reached the bottom, and nitrogen was bubbled through the solution for 10 minutes. After this a supply of nitrogen was maintained above the solution to ensure it remained C02-free. A positive displacement pipette was used to add 250 μl of TERGITOL NP-9 to yield a final concentration of 0.025%. The mixture was stirred (under nitrogen) for an additional 10 minutes to fully dissolve the TERGITOL.

A 10 μM solution of each the individual natural nucleotides (dGTP, dCTP, DATP, dTTP) was prepared by adding 12.5 μL of 100 mM stock of the selected nucleotide (Fisher Scientific, 11843933) to 125 mL of Sequencing Solution. Using a pH probe (Sentron SI MicroFET (92270-010)) the pH of each dNTP solution was titrated to pH 8.00+0.01 using 10 mM NaOH (prepared from a 10 M NaOH stock; Merck, 72068). All dNTP solutions were prepared in a C02 free, nitrogen-controlled environment with mixing achieved by use of a magnetic stir bar.

Using a pH probe (Sentron SI MicroFET (92270-010)) the “Wash Solution” was prepared by titrating the pH of Sequencing Solution to 8.00+0.01 using 10 mM NaOH (prepared from a 10 M NaOH stock; Merck, 72068) in a CO₂ free, nitrogen-controlled environment with mixing achieved by use of a magnetic stir bar.

“1× ThermoPol Buffer” was prepared according to the following formulation: 4.5 mL 10× ThermoPol buffer (New England Biolabs), 27 μL Tween 20 (neat, Merck Life Science, P9416), 40.5 mL molecular grade water (Sigma).

5× Annealing Buffer” was prepared according to the following formulation: 10 mM Tris pH 7.5 (Life Technologies, 15567), 25 mM Magnesium Acetate (Merck Life Sciences, 63052), 750 mM NaCl, 0.05% Tween 20, 25% DMSO (Merck Life Science, D8418), in molecular grade water.

A 5 μM working solution of Oligo Z (sequencing primer) was prepared by adding 1.25 μL Oligo 2 (100 μM stock) to 5 μL of 5× Annealing Buffer diluted with 18.75 μL molecular grade water to give a final composition of 5 μM sequencing primer in 1× Annealing Buffer.

A 25 U/μL working stock of “Sequencing Enzyme” was prepared by diluting 1 μL of Bst Large Fragment DNA Polymerase at 2,000 U/μL (New England Biolabs, custom preparation) in 79 μL of 1× ThermoPol Buffer. The solution was thoroughly mixed by pipetting 20 μL volumes up and down 10 times.

### Protocol/Reaction Conditions

Clonal amplification and de-hybridization of the opposing strand were performed using the SM-RPA method as described in Example F.

The flow cell was flushed twice with 1× ThermoPol Buffer.

Sequencing primer (5 μM Oligo Z in Annealing Buffer; see preparation details above) was loaded into the flow cell. The inlet and outlet of the flow cell was sealed with custom plugs and the primer was annealed to the amplified template using the following thermal protocol: a. 95° C. for 2 min, b. 58° C. for 5 min, c. Passive cool-down to ambient temperature (about 20-26° C.) for 15 min.

The flow cell was then washed with 200 μL of IX ThermoPol buffer to remove any excess primer.

Sequencing Enzyme (25 U/μL Bst Large Fragment; see preparation details above) was loaded into the flow cell and incubated for 10 minutes at ambient temperature (about 20-26° C.). The flow cell was flushed with 200 μL 1× ThermoPol Buffer and sequencing was conducted essentially as described in Example H.

### Results and Conclusions

The sequencing results derived from a single ISFET on the chip are shown in Table 34. The read is aligned against the reference sequence (REF), with the symbols between the 2 sequences representing the following: |=perfect match, >=deletion, <=insertions,*=mismatch. These data demonstrate that the system as described is capable of sequencing a template generated by the disclosed in-well clonal amplification method.

TABLE 34
REF: TGGTGCCTGAGCTTGGGCTGGTGGTCCTTAAGCCGGGCC-GT-GAATCCATACA-G--ATA-GA-CC-ACAA--TGAAGCCAA
|||||||||||||||||||||*|||||>|||||||||||<*|<|||||||||||<|<<|||<||<||>||||<<||>>|||||
READ: TGGTGCCTGAGCTTGGGCTGGNGGTCC-TAAGCCGGGCCANTGGAATCCATNCATGGAATNGGATCCTA-AAAGTG--GCCAA

Q. Automated Sample-to-Answer Sequencing of Pathogen Spiked into Whole Blood

Bacterial Target Organism

Enterococcus faecium, ATCC strain ATCC® BAA2318™; contains antimicrobial resistance (AMR) gene vancomycin A+(vanA+)

### Oligomers

Specific Target Capture (STC) oligomers (IDT Technologies) (see Table 35 below)

TABLE 35

| Target | Target DNA | | Whole sequence | [oligo]pool |
|---|---|---|---|---|
| Type | Region | Oligo name | (5′-3′) | (pmol/10μL) |
| Bacterial | P2 | P2 BCT348 FOR | /5Biosg/AAAAA TTT | 1.667 |
| 16S | | (SEQ ID No. 181) | CGA TGC AAC GCG | |
| (7- | | | AAG AAC CT/3InvdT/ | |
| plex) | | P2 BCT348 REV | /5Biosg/AAAAA TAC | 1.667 |
| | | (SEQ ID No. 182) | GAG CTG ACG ACA | |
| | | | GCC ATG/3InvdT/ | |
| | P3 | P3 BCT361 FOR | /5Biosg/AAAAA TTT | 1.667 |
| | | (SEQ ID No. 183) | AAG TCC CGC AAC | |
| | | | GAG CGC AA/3InvdT/ | |
| | | P3_BCT361 REV | /5Biosg/AAAAA TTG | 1.667 |
| | | (SEQ ID No. 184) | ACG TCA TCC CCA | |
| | | | CCT TCC TC/3InvdT/ | |
| | P29 | P29_16S-CM03- | /5Biosg/AAAAA TTA | 6.668 |
| | | 04_STC_R-INT | CTC ACC CGT RCG | |
| | | (SEQ ID No. 185) | CCR CT/3InvdT/ | |
| | P30 | P30 16S-CM05- | /5Biosg/AAAAA TGA | 6.668 |
| | | 06_STC F | GAY ACG GGC CAR | |
| | | (SEQ ID No. 186) | ACT CCT ACG | |
| | | | GG/3InvdT/ | |
| Bacterial | P4 | P4 23S-CM15- | /5Biosg/AAAAA TCR | 1.667 |
| 23S | | 16 STC R | TTA CRC CWT TCG | |
| (7- | | (SEQ ID No. 187) | TGC AGG TC/3InvdT/ | |
| plex) | | P4_23S-CM15- | /5Biosg/AAAAA CGC | 1.667 |
| | | 16 STC R-INT | TAC CTT AGG AYS | |
| | | (SEQ ID No. 188) | GTT ATA GTT | |
| | | | AC/3InvdT/ | |
| | P31 | P31 23S- | /5Biosg/AAAAA TGT | 1.667 |
| | | CM17+18 STC F IN | GTA GGA TAG GTG | |
| | | T | GGA GGC/3InvdT/ | |
| | | (SEQ ID No. 189) | | |
| | | P31 23S- | /5Biosg/AAAAA CGG | 0.556 |
| | | CM17+18_STC_F2 S1 | AAA GAC CCC ATG | |
| | | (SEQ ID No. 190) | GAG CTT TAC | |
| | | | T/3InvdT/ | |
| | | P31 23S- | /5Biosg/AAAAA CGG | 0.556 |
| | | CM17+18 STC F2 S2 | AAA GAC CCC GTG | |
| | | (SEQ ID No. 191) | AAC CTT TAC | |
| | | | T/3InvdT/ | |
| | | P31 23S- | /5Biosg/AAAAA CGG | 0.556 |
| | | CM17+18 STC F2 S3 | AAA GAC CCC GTG | |
| | | (SEQ ID No. 192) | GAG CTT TAC | |
| | | | T/3InvdT/ | |
| AMR | KPC | KPC STC F | /5Biosg/AAAAA CAC | 1.667 |
| (4- | | (SEQ ID No. 193) | CGC GCT GAC CAA | |
| plex) | | | CCT C/3InvdT/ | |
| | | KPC STC R | /5Biosg/AAAAA CAC | 1.667 |
| | | (SEQ ID No. 194) | AGC GGC AGC AAG | |
| | | | AAA GC/3InvdT/ | |
| | mecA | mecA STC F-INT | /5Biosg/AAAAA AGG | 1.667 |
| | | (SEQ ID No. 195) | TAC TGC TAT CCA | |
| | | | CCC TCA AAC AGG | |
| | | | T/3InvdT/ | |
| | | mecA STC R2 | /5Biosg/AAAAA TTG | 1.667 |
| | | (SEQ ID No. 196) | AGT TGA ACC TGG | |
| | | | TGA AGT TGT AAT | |
| | | | CTG G/3InvdT/ | |
| | vanA | vanA STC F | /5Biosg/AAAAA ACA | 1.667 |
| | | (SEQ ID No. 197) | AGG TCT GTT TGA | |
| | | | ATT GTC CGG /3InvdT/ | |
| | | vanA STC R | /5Biosg/AAAAA TTG | 1.667 |
| | | (SEQ ID No. 198) | TCT TGC CGA TTC | |
| | | | AAT TGC G/3 InvdT/ | |
| | CTX-M | CTX-M-Gp1_STC_F- | /5Biosg/AAAAA CCG | 1.667 |
| | | INT | TCA CGC TGT TGT | |
| | | (SEQ ID No. 199) | TAG GAA GTG | |
| | | | TGC/3InvdT/ | |
| | Group 1 | CTX-M- | /5Biosg/AAAAA GCC | 1.667 |
| | | Gp1_STC_R SO | ATC ACT TTA CTG | |
| | | (SEQ ID No. 200) | GTG CTG CAC | |
| | | | AT/3InvdT/ | |
| | | CTX-M- | /5Biosg/AAAAA GCC | 1.667 |
| | | Gp1_STC_R S3 | ATA ACT TTA CTG | |
| | | (SEQ ID No. 201) | GTA CTG CAC | |
| | | | AT/3InvdT/ | |

In a first polymerase chain reaction (PCR1), the following primers were used to amplify each of the targets specified:

P1
(SEQ ID No. 25)
TGTAGCGGTGAAATGCGYAGA
P1
(SEQ ID No. 26)
CGGTCGACTTAACGCGTTAGCT
P1
(SEQ ID No. 27)
CGGAGTGCTTAATGCGTTWGCT
P2
(SEQ ID No. 28)
CGCAAGGTTGAAACTCAAAGGAATTG
P2
(SEQ ID No. 29)
CCGCAAGGTTAAAACTCAAATGAATTG
P2
(SEQ ID No. 30)
GGGACTTAACCCAACATYTCAC
P3
(SEQ ID No. 35)
CGTGTGTAGCCCAGGTCATAAGG
P3
(SEQ ID No. 36)
CACGTGTGTAGCCCAAATCATAAGG
P3
(SEQ ID No. 37)
TGTGTAGCCCTGGTCGTAAGG
P3
(SEQ ID No. 38)
TCAGCTCGTGTCGTGAGATGTT
P3
(SEQ ID No. 39)
CGTCAGCTCGTGTTGTGAAATGTT
P4
(SEQ ID No. 46)
ACACAGGTCTCTGCTAAACCGTAAG
P4
(SEQ ID No. 47)
ACACAGGTCTCTGCAAAATCGTAAG
P4
(SEQ ID No. 48)
ACACAGCACTGTGCAAACACGAAAG
P4
(SEQ ID No. 49)
TACCCGACAAGGAATTTCGCTACC
P25
(SEQ ID No. 50)
TGGCAGCTTCACTTTCTCTTGC
P25
(SEQ ID No. 51)
CCAGCTCCAATCACACCAACA
P29
(SEQ ID No. 31)
CCTGGCTCAGAATGAACGCT
P29
(SEQ ID No. 32)
CCTGGCTCAGGACGAACGCT
P29
(SEQ ID No. 33)
GAGTCTGGACCGTGTCTCAGT
P29
(SEQ ID No. 34)
GAGTCTGGGCCGTGTCTCAGT
P30
(SEQ ID No. 40)
CTCCTACGGGAGGCAGCAGT
P30
(SEQ ID No. 41)
CCTCCGTATTACCGCGGCTG
P31
(SEQ ID No. 42)
GAAAGACCCCGTGAACCTTTACT
P31
(SEQ ID No. 43)
GAAAGACCCCGTGGAGCTTTACT
P31
(SEQ ID No. 44)
CCTTCGTGCTCCTCCGTTAC
P31
(SEQ ID No. 45)
CCTTTGAGCGCCTCCGTTAC
P28
(SEQ ID No. 54)
AACCATTCGCTAAACTCGAACAGG
P28
(SEQ ID No. 55)
CCTTGAATGAGCTGCACAGTGG
P40
(SEQ ID No. 70)
CATGAAAAATGATTATGGCTCAGGTAC
P40
(SEQ ID No. 71)
TGGAACTTGTTGAGCAGAGGTTC
P41
(SEQ ID No. 72)
GGCTGCGATATTCAAAGCTCAG
P41
(SEQ ID No. 73)
CTGAACGCGCCGGCTTAAC
P48
(SEQ ID No. 80)
CGGCARCCGTCACGCTGT
P48
(SEQ ID No. 81)
CATCAGCACGATAAAGTATTTGCGA

In a second polymerase chain reaction (PCR2), the following nested primers were used to amplify each of the targets specified:

| Primer name | Sequence |
|---|---|
| P41_RPAv1_F | AAAACGAGACATGCCGAGCAT |
| (SEQ ID No. 202) | CCGCTTGTATGGACAAATCGT |
| | TGACATACA |
| 2Bio_P41_RPA_R | /52-Bio/ACC GTG CTG CCT TGG |
| (SEQ ID No. 203) | CTT CAT TGT GGT CGT AGC |
| | TGC CAC CGG CCT AT |

The chip surface bound primers for these targets were as follows:

P41 (vanA) (SEQ ID No. 126):
5′-GTAGCTGCCACCGGCCTAT-3′

Buffer/Reagent preparation and filling of the sample preparation, library preparation and sequencing cartridges

The reagents shown in Tables 36-38 were prepared and then loaded onto the sample preparation, library preparation and sequencing cartridges, respectively, in the indicated chambers (see FIGS. 22, 25, 27C and 30).

TABLE 36

| | | | | | | Volume | |
|---|---|---|---|---|---|---|---|
| | | | | | | Loaded | |
| | | Stock | | | Location in | into | |
| Reagent | Supplier | Conc. | Final Conc. | | Cartridge | Cartridge | |
| NS4X | DNAe | N/A | 100 mM Tris | | Lysis Buffer | 1.667 | mL |
| | formulation | | pH 8.0, 12% | | chamber | | |
| | | | SDS | | | | |
| Antifoam | Y-30 | N/A | | | Lysis Buffer | 100 | μL |
| | emulsion | | | | chamber | | |
| | Sigma- | | | | | | |
| | Aldrich | | | | | | |
| | A6457- | | | | | | |
| | 100 ML | | | | | | |
| Biotinylated | 11-Plex | N/A | 41.7 | pmol | Lysis Buffer | 10 | μL |
| STC oligos | mixed in | | | | chamber | | |
| | house | | | | | | |
| Proteinase K | Promega | 20 mg/mL | 1 | mg | Lysis Buffer | 50 | μL |
| (ProK) | MC5008 | | | | chamber | | |
| Yttria | Glen Mills | N/A | 4 | g | Mechanical | N/A | |
| stabilized | | | | | Lysis (ML) | | |
| zirconia | | | | | chamber in | | |
| beads (0.1 | | | | | ML fin | | |
| mm) | | | | | | | |
| Dynabeads ™ | ThermoFisher | 10 mg/mL | 1200 | μg | STC Capture | 120 μL | |
| MyOne ™ | | | | | Beads | (unwashed), | |
| Streptavidin | | | | | | then | |
| C1 | | | | | | washed and | |
| | | | | | | resuspended | |
| | | | | | | in 100 μL | |
| | | | | | | wash T | |
| Wash S | DNAe | N/A | 50 mM Tris | | Wash S | 4 | mL |
| | formulation | | pH 8.0, 0.1% | | | | |
| | | | SDS, 150 mM | | | | |
| | | | NaCl | | | | |
| Wash T | DNAe | N/A | 10 mM Tris | | Wash T | 14 | mL |
| | formulation | | pH 8.0, 0.01% | | | | |
| | | | Tween-20 | | | | |
| Elution | Integrated | 10 mM | | | STC Elution | 500 | μL |
| buffer | DNA | Tris pH | | | | | |
| | Technologies | 7.5, 0.1 | | | | | |
| | | mM | | | | | |
| | | EDTA | | | | | |

TABLE 37

| | | | | | Location | Volume |
|---|---|---|---|---|---|---|
| | | Stock | | | in | Loaded into |
| Reagent | Supplier | Conc. | Final Conc. | | Cartridge | Cartridge |
| UltraPure ™ | Invitrogen | N/A | N/A | | PCR1 | 3900 μL |
| Distilled Water | | | | | Dilution | |
| Albumin, | Calbiochem | 100 mg/mL | 2 | mg/mL | PCR1 | 80 |
| Bovine Serum | | | | | Dilution | |
| (BSA) | | | | | | |
| Dynabeads ™ | Thermo | 10 mg/mL | 2000 | μg | Hyb | 200 μL |
| MyOne ™ | Fisher | | | | | (unwashed) |
| Streptavidin C1 | | | | | | |
| Wash Buffer | | | 1M NaCl, 5 mM | | Wash | |
| | | | Tris HCL (pH | | Buffer | |
| | | | 7.5), 0.5 mM | | | |
| | | | EDTA, 0.05% | | | |
| | | | Tween20, | | | |
| | | | 2 mg/mL BSA | | | |
| PCR1 lyo | Argonaut | | | | PCR1 | |
| | | | | | Reagent | |
| PCR2 lyos (no | Argonaut | | | | PCR2 | |
| primer) | | | | | Reagent | |
| Wet primer P41 | IDT | | | | PCR2 | |
| | | | | | Reagent | |
| Elution Buffer | NaOH | 1M | 40 | mM | Elution | 120 μL |
| | | | | | Buffer | |

TABLE 38

| | | | | | Volume | |
|---|---|---|---|---|---|---|
| | | Stock | | Location in | Loaded into | |
| Reagent | Supplier | Conc. | Final Conc. | Cartridge | Cartridge | |
| dNTP | Thermo | 100 mM | 50 μM | Individual | 50 | μL |
| | Scientific | | | dNTP bottles | | |
| | | | | on BB3b | | |
| Antifoam (Y- | Sigma- | Neat | 0.0375% | Wash Bottle | 37.5 | μL |
| 30 emulsion) | Aldrich | | | on BB3b | | |
| | A6457 | | | | | |
| Tergitol | Sigma- | Neat | See S2 Buffer | | | |
| | Aldrich | | | | | |
| S2 Buffer | | | 5 mM MgCl₂, 20 | Wash Bottle | 600 | mL |
| | | | mM NaCl, 0.025% | on BB3b | | |
| | | | Tergitol | | | |
| 10X | NEB | | 1X ThermoPol | Seq Enzyme | 50 | μL |
| ThermoPol | | | Buffer in | chamber | | |
| Buffer | | | Molecular Grade | | | |
| | | | Water, 0.06% | | | |
| | | | Tween 20 | | | |
| BST large | NEB | | | Seq Enzyme | 2 | μL |
| fragment | | | | chamber | | |
| polymerase | | | | | | |
| Annealing | | | 0.02M Tris pH | BB2 Output | 25 | μL |
| Buffer | | | 7.5, 0.005M | chamber | | |
| | | | Magnesium | | | |
| | | | Acetate, 0.15M | | | |
| | | | NaCl, 0.01% | | | |
| | | | Tween 20, 5% | | | |
| | | | DMSO | | | |

### Protocol Reaction Conditions

Five hundred colony-forming units (CFU) of Enterococcus faecium containing the vanA+ gene were spiked into 5 mL of whole blood in a 10 mL vacutainer tube. After sealing the contrived spiked blood sample vacutainer, 1 mL of air was pulled to induce negative pressure. An 18G needle (Becton Dickinson) was utilized in the cartridge to puncture the vacutainer's rubber stopper.

The spiked whole blood sample was transferred from the vacutainer tube (VT) to the Lysis Buffer chamber by aspirating 5 mL of air from the atmosphere and dispensing into the VT, then repeated once, for a total pressurization volume of 10 mL air. The pressure reached 7 psi. The cartridge was vented to atmosphere for 30 sec. to allow the blood in the VT to naturally drain from the local high pressure that was created inside the VT to the atmospheric pressure in the Sample Prep Rotary Valve 1 (SP-RV1). One milliliter was drawn from the VT to aspirate as much volume as possible, then dispensed all the blood volume in the SP-RV into the Lysis Buffer chamber. With 5 mL of blood in the VT, approximately 3-4 mL was drained from this first pressurization. The VT transfer step was run twice, so the remaining blood volume was collected following the same procedure; pressurized with 10 mL of air, passively drained into the SP-RV1, and dispensed that volume into the Lysis Buffer chamber.

The sample was homogenized using turbulent mixing to move the blood back and forth between the Lysis Buffer and Lysis Overflow chambers. The entire sample (buffer and blood) was aspirated and dispensed at 50 mL/minute, allowed to equilibrate for 10 seconds then the process repeated four more times, with the sample ending in the Lysis Buffer chamber.

In the Lysis Buffer chamber, a cylindrical shaped resistive heater was used to heat the sample and activate Proteinase K (Pro K). The heater was set at 110° C. for 10 minutes (the sample temperature did not go above 60° C. for the duration of the step).

The sample was moved by aspirating it into the SP-RV1 from the Lysis Buffer chamber and dispensing it to the ML chamber in the ML fin (in which the zirconium beads were contained). The sample and the beads were mixed for 2.5 minutes using an impeller rotating at 8000 RPM, resulting in pathogen cell lysis.

Using the SP-RV1, the sample was moved to the Lysis Buffer chamber and then into the Sample Prep Mag Sep fin (a serpentine line within the cartridge that sits on top of a rectangular resistive heater). The sample was passed at a flow rate of 0.45 mL/min through the serpentine line and into the STC Hyb 1 Buffer chamber. The sample was incubated at 95° C. (heater set at 110° C. to account for heat loss) as it passed through the line, resulting in denaturation of the dsDNA released from the cells in the lysis step above.

STC oligo annealing—The sample was then transferred to the STC Hyb 1 Buffer chamber where it was cooled rapidly to 25-30° C. It was then transferred to the Lysis Buffer chamber (contained the STC oligos; pre-heated to 60° C.) and incubated at 60° C. for 30 minutes, resulting in annealing of the STC oligos to their target DNA sites.

One milliliter of the sample was aspirated from the Lysis Buffer chamber and dispensed into the STC Capture Beads chamber (contained the paramagnetic streptavidin beads). The beads were resuspended and mixed by aspirating/dispensing the 1 mL sample portion from/to the SP-RV1 to the STC Capture Beads chamber at a rate of 10 mL/minute. This was repeated once more.

The homogenized sample in the STC Capture Beads chamber was transferred to the lysis buffer chamber where it was re-incorporated with the full sample volume by using the SP-RV1 to aspirate and dispense from one chamber to another.

The full sample volume was mixed by aspirating and dispensing (using the SP-RV1) 4 mL of sample at a rate of 30 mL/minute out of and back into the Lysis Buffer chamber to create turbulent mixing (conducted at a sample temperature of 45° C.). This was repeated 14 times to ensure that the beads were fully dispersed throughout the sample, resulting in binding of the capture oligomer/target complexes to the streptavidin paramagnetic particles.

After incubation, the sample and magnetic beads were aspirated into the SP-RV1 and dispensed into the Sample Prep Mag Sep fin. An array of four N52 grade Neodymium magnets (K&J Magnets, Inc.; orientated with alternating polarity, perpendicular to the serpentine channels) were pressed to the thin film on the top surface of the cartridge with a spring-loaded end effector, thus collecting the paramagnetic beads from the suspension.

The beads were washed by aspirating wash-S buffer into the SP-RV1 and then dispensing it through the fin at a rate of 2 mL/minute with the four magnets still engaged (thus removing sample waste from the fin channels). The magnet were then disengaged and the beads were resuspended in 200 μL of wash-S buffer. One hundred microliters of air was aspirated and dispensed 4 times to move the sample back and forth across the bead capture area to clean beads of any remaining sample waste still adhering to the magnetic beads. The magnets were re-engaged to re-capture the beads, then another 200 μL portion of wash buffer-S buffer was pushed through the fin at a rate of 2 mL/minute.

The above step was repeated essentially as described except that wash-S buffer was replaced with wash-T buffer.

The magnets were retracted and 100 μL of elution buffer was aspirated from the STC Elution chamber and dispensed into the Sample Prep Mag Sep fin. Small air volumes (150 μL) were aspirated and dispensed into the Sample Prep Mag Sep fin. This was repeated over the area where the beads were captured. Once resuspended, the fin heaters were turned on and set to a temperature of 110° C. for 3 minutes to elute the target DNA from the beads (internal sample temperature reached 75° C.).

The magnets were re-engaged to re-capture the beads, leaving the eluted DNA (eluate) in solution.

Approximately 85 μL of the eluate was used to resuspend the lyophilized PCR1 reagent inside the PCR1 Reagent chamber. The resulting sample was aspirated from said chamber and dispensed into the PCR1 lyo chamber. This chamber was then pressurized to 27 psi to push sample into the thermal region (PCR 1 in FIG. 25) and PCR amplification (PCR1) was performed using the following four-step thermal protocol, where steps b, c and d were performed repeatedly in order for 25 cycles:
a. 98° C. for 30 sec (initial denature step), b. 98° C. for 5 sec (denature step), c. 58° C. for 25 sec (anneal step), d. 72° C. for 45 sec (extend step).

After PCR1 was completed, the sample was aspirated and dispensed into PCR1 Product chamber. Twenty microliters was then retrieved from this chamber and dispended into the PCR1 Dilution chamber which was pre-filled with 3.98 mL of water (Invitrogen; Ultra-Pure Distilled Water, DNase and RNase-free) (200-fold dilution) and BSA (2 mg/mL; Calbiochem; Albumin, Bovine Serum, Fraction V, Fatty Acid free, Nuclease and Protease free). The sample was then mixed by aspirating and dispensing 2 mL of air back and forth from the PCR1 Dilution chamber.

After mixing, a total of 250 μL of diluted material was transferred to the PCR2 Reagent chamber.

The sample was mixed by aspirating and dispensing 200 μL from the PCR1/CC RV to the PCR2 Reagent chamber, then 125 μL was aspirated and dispensed into the PCR1 lyo chamber and pressurized to 27 psi, then 165 μL was aspirated and dispensed into the Adapter Addition chamber and pressurized to 27 psi and PCR amplification (PCR2) was performed using the following four-step thermal, where steps f, g and h were performed repeatedly in order for 40 cycles:
a. 98° C. for 30 sec (initial denature step), b. 98° C. for 5 sec (denature step), c. 58° C. for 25 sec (anneal step), d.72° C. for 45 sec (extend step).

The sample was aspirated from the PCR 1 and Adapter Addition chambers and dispensed to the Hyb chamber where it was combined with pre-loaded Sav MyOne C1 beads. The sample was incubated at ambient temperature (about 20-26° C.) for 10 minutes with constant mixing (aspirating and dispensing 250 μL back and forth from the PCR1/CC RV to the Hyb chamber).

An array of four N52 grade Neodymium magnets were pressed to the top surface of the cartridge with a spring-loaded end effector. The beads were flowed into the Library Prep Mag Sep fin and the magnet was then engaged to immobilize the streptavidin beads.

The beads were washed by disengaging the magnet, flowing 150 μL of Wash Buffer over the beads and resuspending the beads by moving them back and forth in the channels inside the fin via aspirating/dispensing air. The magnet was then re-engaged, the beads were immobilized and Wash Buffer was pushed to the waste chamber. This process was repeated three times.

After the wash process, the beads were resuspended and the bound DNA eluted by disengaging the magnet, pushing 60 μL of 40 mM NaOH elution buffer into the fin and waiting 60 seconds. The magnet was re-engaged and the beads were immobilized, leaving the eluted DNA in the supernatant.

Antifoam Y-30 was added to 1 μL of S2 Wash Buffer, yielding a final concentration of 0.0375%.

Four Costar® 150 mL Traditional Style Polystyrene Storage Bottles with 45 mm Caps (Corning) were each filled with 100 mL of S2 Wash Buffer and the remaining 600 mL of S2 Wash Buffer was poured into a 1 μL bottle for wash.

Fifty microliters of each dNTP (Thermo Scientific) were added to the 100 mL of S2 Wash Buffer in each of the four 150 mL bottles for a final concentration of 50 μM for each nucleotide.

Two hundred and fifty milliliters soda lime were added to the Soda Lime bottle (500 mL) that had a metal inlet tube pushed to the bottom of the bottle to ensure that air was circulating through the soda lime.

All the bottles and the conical tube were connected to the BB3b cartridge and this was loaded into the BB3 sequencing sub-system (FIG. 30).

Two microliters of BST large fragment polymerase (New England BioLabs) was pre-mixed with 128 μL of 1× ThermoPol Buffer and this was added to the Seq Enzyme chamber of the BB3a cartridge.

Twenty-five microliters of Annealing Buffer was pre-loaded into the BB2 Output chamber.

Twenty-five microliters of the eluted DNA (see above) was pipetted into the BB2 Output chamber and mixed with the pre-loaded annealing buffer. It was then moved to the chip by aspirating and dispensing using Sequencing Rotary Valve 1 and Selector Valve. The chip was then pressurized to 15 psi.

Template hybridization to the immobilized primer was performed using the following thermal protocol shown:
a.95° C. for 2 min, b.Chip depressurized, c. 68° C. for 5 min, d. 63° C. for 5 min, e. 58° C. for 5 min, f.53° C. for 5 min, g. 48° C. for 5 min, h. 43° C. for 5 min.

In parallel with the hybridization step, 10 mM NaOH was used to titrate the 150 mL bottles containing dNTP's and the 1 μL wash bottle to pH 8.00+0.01.

Once the hybridization was complete, the chip was set to 25° C. and 1× ThermoPol Buffer was washed over the chip surface for 10 seconds.

One hundred and thirty microliters of the reagents in the Seq Enzyme chamber were pushed to the chip, which was then pressurized to 12 psi to prevent bubble formation. The enzyme was incubated for 2 minutes.

The chip recording was turned on and the sequencing system was primed.

An electrical response test was performed by biasing the reference electrode with increasing voltage steps. The resulting change in mV output measured by the ISFETs on the IC was used to determine the relationship between reference electrode potential and the corresponding potential seen on the ISFETs, which then was used to determine the optimal reference electrode potential for the experiment. The dNTP lines were primed with wash buffer and the chip washed for 20 seconds to remove any air in the lines.

Solutions were sequentially flowed over the chip surface for 25 cycles in the following order:
a. Wash solution for 25 sec, b. dTTP solution for 10 sec, c. Wash solution for 25 sec, d. dGTP solution for 10 sec, e. Wash solution for 25 sec, f. dCTP solution for 10 sec, g. Wash solution for 25 sec, h. dATP solution for 10 sec.

The chip was washed again, and the chip recording was turned off.

Analysis was performed using in-house proprietary algorithms for base-calling and bioinformatics analysis.

### Results and Conclusions

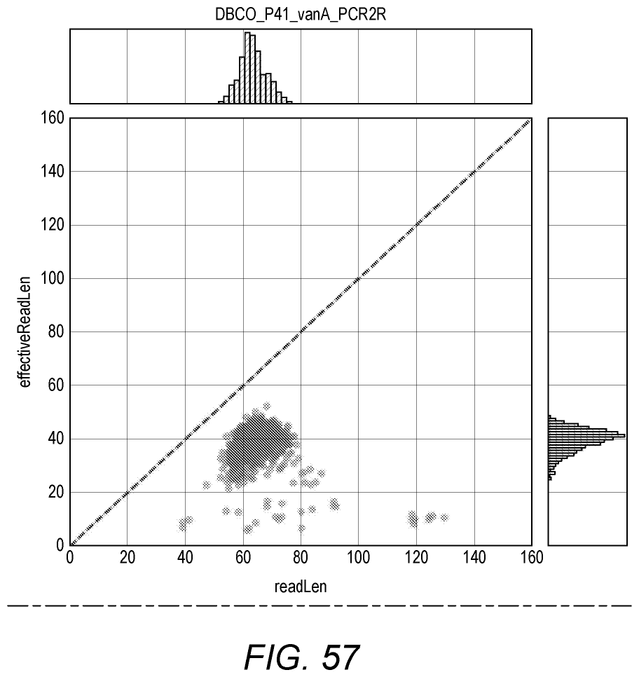

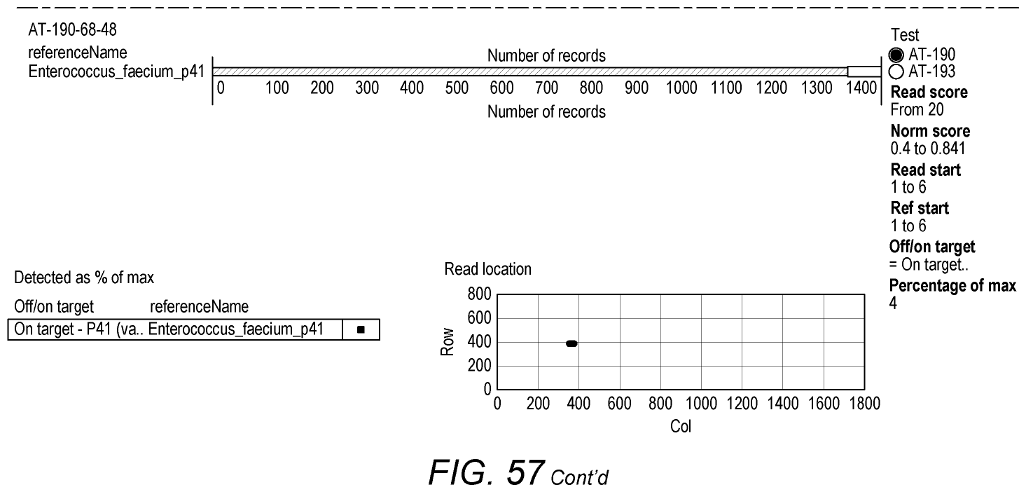

The sequencing results are shown in FIG. 57. Individual reads are plotted as Read Length (the total length of the output read) on the x-axis (labeled “readLen”) versus Effective Read Length (ERL; number of matches to the expected sequence minus the number of errors) on the y axis (labeled “effectiveReadLen”). A read with no errors would lie on the diagonal dashed line, where readLen=ERL. The histogram sections shown in FIG. 57 indicate the distribution of reads on the different axes. The histogram across the top of the plot indicate the distribution of reads along the x-axis, i.e., read length (bp); the histogram on the right-hand side of the plot indicate the distribution of reads along the y-axis, i.e., ERL (bp). The total number of reads in this example was 1442. Of these reads, 94.3% had an ERL>30 bp. The graphics below the plot in FIG. 57 show that over 1300 or the 1442 reads aligned to the expected target, so the final assay call was vanA. Therefore, these data demonstrate the capability of the disclosed system to process a bacterial sample spiked into blood through sample prep, library prep, sequencing and to make a correct call of the spiked target in a short period of time in a cartridge-based, automated system.

### P. Mechanical Lysis

To test mechanical lysis systems compatible with cartridges as described above, lysis of target organisms using a mechanical lysis sub-system was conducted. Multiple replicates of 3 mL blood aliquots in 15 mL conical tubes were prepared. To some of the tubes (sample positive) was added 15 μL of Enterococcus faecium (ATCC BAA-2318) at 200 CFU/μL. To some of the tubes (negative controls), no sample was added. To each tube then added 1 mL Lysis Buffer (100 mM Tris-HCl, pH 8.0, 16.7% (w/v) lithium dodecyl sulfate) followed by 60 μL 100% Antifoam Y30, 30 μL Proteinase K (20 mg/mL) and 10 μL of a pool of specific target capture oligonucleotides (Oligo Pool 38-Plex V2.0, DNAe). All tubes were vigorously vortexed for 30 seconds, inverted multiple times, vigorously vortexed for 30 more seconds and again inverted multiple times. The tubes were centrifuged at 700 rpm for 15 seconds and then incubated at 75° C. for 15 minutes (Proteinase K digestion step).

Mechanical lysis was then performed either using a control method on the bench or in the ML coupon (a cartridge compatible mechanical lysis subsystem as described above) briefly as follows: 1) Control Method on Bench. The contents of multiple sample positive and negative controls tubes from above were transferred to 8 mL mechanical lysis tubes containing 4 g of 0.1 mm Yttria Stabilized Zirconium Oxide bashing beads. The tubes were then placed in the OmniRuptor Elite (SKU 19-042E) and subjected to 3 cycles of 90 seconds on+20 seconds off at 6.6 m/s speed). The contents of the lysis tubes were then transferred back to their corresponding 15 mL conical tube. 2) ML Coupon Method. The contents of multiple sample positive and negative controls tubes from above were transferred to the ML coupon containing 4 g of 0.1 mm Yttria Stabilized Zirconium Oxide bashing beads using a 10 mL syringe and subjected to mechanical lysis using an in-house script. The contents of the ML coupon were then transferred back to their corresponding 15 mL conical tube.

All samples (Control Method on Bench and ML Coupon Method) were processed identically. Briefly, all tubes were incubated at 95° C. for 30 min to denature target nucleic acid and then at 60° C. for 40 min to effect annealing of the specific target capture oligomers to the target. Twelve hundred micrograms of Streptavidin coated magnetic beads (DNAe) were added to each sample, the tubes were vortexed and then incubates at 45° C. with a constant, gentle vortex (1500 rpm) for 10 minutes. The beads were collected with the use of a magnet and the supernatant discarded. The beads were resuspended in 1 mL Wash Buffer (10 mM Tris-HCl, pH 8.0, 0.01% Tween-20) via vortexing. The mixture was transferred to 1.5 mL tube and the beads were collected with the use of a magnet and the supernatant discarded. This wash procedure was repeated 3 more times. After the supernatant was discarded after the final wash, 40 μL of Elution Buffer (same formulation as the) was added top each tube, the tubes were vortexed and then briefly centrifuged to bring the contents to the bottom of the tube. All tubes were incubated at 75° C. for 3 min, vortexed and briefly centrifuged. The beads were collected with the use of a magnetic and the supernatant (i.e., the eluent) was transferred to a 0.5 mL tube.

The samples were all then analyzed using quantitative PCR generally as follows: A 10 μL aliquot of each eluent was added to 15 μL of a Master Mix (0.6 μL SuperFi polymerase, 0.4 μL dNTP Mix, 5 μL SuperFi buffer, 8.2 μL water, 1 μM primer mix (vanA_PCR2_Fwd: (SEQ ID No. 153): TTGTATGGACAAATCGTTGACATACA and vanA_PCR2_Rev (SEQ ID No. 204): GTAGCTGCCACCGGCCTAT) and SYBR Green) in a PCR microtiter plate. PCR was then performed in a thermocycler (98° C. for 30 seconds and then 40 cycles of 98° C. for 5 seconds and 65° C. for 25 seconds). Fluorescence was measure during the run and emergence times were determined for each sample.

The results are shown in FIG. 71. The data demonstrate that lysis of Enterococcus faecium in blood using the ML coupon is equivalent to that achieved with the bench gold standard method. Similar results were also achieved using the above detailed protocol with gram-negative Klebsiellapneumoniae (ATCC BAA-1898) and gram-positive Staphylococcus aureus (ATCC BAA-2094)(data not shown).

Q. Automated Specific Target Capture Direct from Blood

A cartridge-compatible specific target capture (STC) subsystem or fin, as described above, was tested to evaluate the efficacy of extracting and isolating a specific target DNA from whole blood with results compared to a bench positive control direct from blood specific target capture process.

An STC subunit such as the one depicted in FIG. 69 was loaded into a fixture, then using commands that are part of an in-house script, an automatic pipettor aspirated 4 mL of blood spiked with 500 copies of purified gDNA Klebsiellapneumoniae (CDC AR0068) into a pipette tip which was then docked in the sealing pipette interface (SPI) port and the blood dispensed into the fin. All pipetting steps are capable of being performed automatically using, for example, a pipetting gantry within an instrument of the invention as described above. Next, 1 mL Lysis Buffer (100 mM Tris-HCl, pH 8.0, 16.7% (w/v) lithium dodecyl sulfate), 80 μL 100% Antifoam B (JT Baker), 50 μL Proteinase K (20 mg/mL) and 10 μL of a pool of specific target capture oligonucleotides were each added to a 1.5 mL tube using the same pipette tip, then the mixture of all 4 reagents was aspirated into the pipette tip and docked in the SPI port to be dispensed into the fin. The mixture of blood and reagents was placed in the mixing chamber and combined to homogeneity by shuttling the mixture between two chambers. The thermoelectric coolers (TECs) were then turned on and the sample was incubated at 75° C. for 15 minutes (Proteinase K digestion step), during which period shuttle mixing was continued in order to distribute the heat evenly. Utilizing the SPI and pipette tip, the sample was aspirated/drained from the fin and dispensed into an 8 mL mechanical lysis tube containing 4 g of 0.1 mm Yttria Stabilized Zirconium Oxide bashing beads. The lysis tube was then placed in the OmniRuptor Elite (SKU 19-042E) and subjected to 3 cycles of 90 seconds on+20 seconds off at 6.6 m/s speed. The sample was then centrifuged to bring the lysis beads to the bottom of the tube and the sample was transferred to a 5 mL tube through a mesh filter. The sample was aspirated using the pipettor on the fixture and docked back to the SPI port to transfer it back to the mixing chambers. Once in the chamber, the TECs were then turned on and the sample was incubated at 95° C. for 25 minutes (denaturation step), during which period shuttle mixing was continued in order to distribute the heat evenly. The sample was then incubated at 60° C. for 32 minutes (annealing step), again with continuous mixing.

Twelve hundred micrograms of Streptavidin coated magnetic beads (DNAe) were added to the sample using the fixture pipettor and SPI port and the sample was incubated at 45° C. for 15 minutes (binding to beads), again with continuous mixing. The sample was drained by inserting the pipette tip into the SPI port and aspirating. The magnet was engaged in the serpentine area and the sample passed by at a flowrate of 0.5 ml/min, resulting in collection of the beads and discarding of the supernatant. The beads were resuspended in 0.5 mL Wash Buffer (10 mM Tris-HCl, pH 8.0, 0.01% Tween-20) via shuttle mixing in the serpentine using the pipette tip and SPI port. The beads in the mixture were re-collected with the use of a magnet and the supernatant discarded. This wash procedure was repeated 1 more time. On the third and final wash, the resuspended beads were transferred from the Mixing Chamber Serpentine SPI to the Elution SPI, the beads were re-captured by the magnet and the supernatant was discarded. The beads resuspended with 115 μL of Elution Buffer (same formulation as the Wash Buffer) was added to the fin via the Elution SPI port and the sample was then pushed into the elution chamber and incubated at 75° C. for 5 minutes. The TECs were set to 25° C. for 3 minutes to allow liquid to cool down before final magnetic capture of the streptavidin beads. The magnet was engaged on the serpentine and the beads were collected and the supernatant (i.e., the eluent) was aspirated using a pipette tip and then dispensed into a 0.5 mL tube.

Sixty microliters of the eluate (total ˜100 μL) was added to a master mix (10 μL of SuperFi Buffer 1OX, 20 μL of a 39-plex primer pool, 1.6 μL dNTP mix (0.4 mM for each nucleotide), 2.5 μL of SuperFi Enzyme @0.05 U/μL, 1.8 μL of MgSO4 @1.75 mM). The mixture was added to a 0.2 mL PCR tube and cycled on the Applied Biosystems™ Veriti™ 96Well Thermal Cycler using the following conditions: 95° C. for 30 second, followed by 25 cycles of 5 seconds at 95° C. (denature), 10 seconds at 55° C. (annealing) and 30 seconds at 72° C. (extension).

After PCR, the samples were removed from the thermocycler and a 1:80 dilution was prepared by aliquoting 6.25 μL of the sample and diluting it by adding 93.75 μL of water. Ten microliters of the PCR1 diluted product were added to a master mix (10 μL of SuperFi Buffer 5×, 0.8 μL of the forward primer @1.5 nM and 0.8 μL of the reverse primer @1.5 nM, 0.8 μL dNTP mix (0.4 mM for each nucleotide), 1.3 μL of SuperFi Enzyme @0.05 U/μL, 0.9 μL of MgSO4 @1.75 mM and 0.5 μL SYBR Green @1× and 25.1 μL of water). The mixture was added to a 0.2 mL PCR tube and cycled on the CFX96 Touch Real-Time PCR Detection System using the following conditions: 95° C. for 30 second, followed by 40 cycles of 5 seconds at 95° C. (denature), 10 seconds at 55° C. (annealing) and 30 seconds at 72° C. (extension).

Results between the cartridge-based STC and the bench standard control were comparable as shown in FIG. 72

R. Automated Nested PCR with Cartridge PCR System

Nested PCR was tested using a cartridge-compatible PCR subsystem as described above and illustrated in FIGS. 63 and 70 with 1000 copies of S. aureus gDNA using the PCR system (A3.0 PCR fixture and PCR fin), the run consisted of one PCR1 reaction (39-plex), a dilution step (1:80) and 10 PCR2 reactions (4 plex each).

The S. aureus gDNA stock (127,325 copies/μl) was diluted with water to a final concentration of 10 copies/μl. A total of 100 μl was used to rehydrate the lyophilized master mix that was inserted into the “lyo pocket” in the PCR fin, this fin was placed on the A3.0 PCR fixture. The sample (gDNA and MM) was pushed to the thermal zone, using a combination of commands in KeySharp to meter, pressurize and place the sample in the correct area for thermocycling. All such steps can be carried out by an instrument of the invention through pneumatic pressures via an interface with a cartridge as described above. The cycle used was as follows, hot start: 95° C. for 60 second, followed by 25 cycles of 5 seconds at 95° C. (denature), 10 seconds at 55° C. (annealing) and 30 seconds at 72° C. (extension).

After the cycles ended, the sample was retrieved by depressurizing the chamber and aspirating the sample. A 1:80 dilution was performed by manually aliquoting 15.6 μl of the sample and diluting it by adding 1209.4 μl of water and 25 μl of BSA.

650 μl of the diluted PCR1 sample was aspirated by the instrument and metered in each of PCR2 channels (n=10) for 50 μl. The overage sample was removed from cartridge and the metered sample was moved through the lyo pockets containing the Master Mix lyophilized reagents using a combination of commands in KeySharp to pressurize and place the sample in the thermal zone to start thermocycling, the cycle used was a hot start at 95° C. for 60 second, followed by 40 cycles of 5 seconds at 95° C. (denature), 10 seconds at 55C (annealing) and 30 seconds at 72° C. (extension).

At the end of the thermocycle, the chambers were depressurized, and the samples were retrieved utilizing a common channel. All 10 reactions were retrieved and pooled together for a final PCR2 sample, that was analyzed on the bioanalyzer and compared with a bench negative sample and a bench positive sample.

The bench positive sample utilized the same gDNA concentration and master mix reagents (in lyophilized form) as used with the PCR fin (described above). For the negative controls, water was cycled with the master mix reagents (in lyophilized form). Both PCR1 and PCR2 reactions were performed on a CFX Bio-Rad thermocycler that mimicked the same cycle used on the A3 PCR fixture.

Target amplification was observed for all targets (10/10) in the panel for S.aureus (BAAC 2094) as shown in the electropherogram overlays of the Positive Bench Control, the Negative Bench Control (NTC) and the A3.0 PCR system (AT-401-04) provided in FIG. 73 with the observed peaks matching the expected target peaks in Table 39 below:

TABLE 39

| | | Conc. [ng/μl] | | |
|---|---|---|---|---|
| | Size | Positive Bench | Negative Bench | A3.0 PCR |
| | [bp] | Control | Control | system |
| Lower Marker | 15 | 4.2 | 4.2 | 4.2 |
| P1 | 102 | 4.65 | — | 2.7 |
| P29/mecA | 110 | 27.51 | 2.85 | 4.73 |
| gyrA | 119 | 7.23 | — | 3.5 |
| P3 | 124 | 3.56 | — | 3.5 |
| P4 | 128 | 17.98 | 2.26 | 7.36 |
| P30/P2 | 152 | 22.43 | 1.63 | 4.9 |
| gyrB | 159 | 1.3 | 2.68 | 2.97 |
| P31 | 168 | 12.49 | 0.77 | 3.99 |
| Upper Marker | 1,500 | 2.1 | 2.1 | 2.1 |

### S. Sequencing of an Amplified Template Using the Direct Hybridization Method With Automated pH Adjustment and CO₂ Removal System

### Oligomers

The direct hybridization followed by sequencing method was performed using single stranded DNA templates prepared using PCR (Sequencing Template 1 and Sequencing Template 2; in-house preparations) hybridized to primers immobilized on the surface of the wells (Oligo 18 and 20; ATDBio) The immobilized primers are complementary to the 3′-regions of Sequencing Templates 1 and 2 respectively (see underlined sections).

Sequencing Template 1 (SEQ ID No. 205):
5′-CTACGCCTCCGCTTTCCTCTCTATGGGCAGTCGGTGATGAAT
CTCCATTTTAGCACTTACCTGTGACTCCATAGAAAATCTTTCTCC
TGCTCAGTGATTTCACAGAGAGGATCTCGTGTACTGCGATTAGAA
CGTACCTGTCGTCAGCTCGTACGGC-3′
Sequencing Template 2 (SEQ ID No. 206):
5′-CTACGCCTCCGCTTTCCTCTCTATGGGCAGTCGGTGATGAAT
CTCCATTTTAGCACTTACCTGTGACTCCATAGAAAATCTTTCTCC
TGCTCAGTGATTTCAGAGAGAGGATCTCGTGTAGCAACTAGCGAA
CGTACTGTGTTAGGCATGCCCGG -3′
Oligo 18 (SEQ ID No. 207):
5′-DBCO-GCCGTACGAGCTGACGACAG-3′
(5′-DBCO = 5′-terminal dibenzocyclooctyl)
Oligo 20 (SEQ ID No. 208):
5′-DBCO-CCGGGCATGCCTAACACA-3′
(5′-DBCO = 5′-terminal dibenzocyclooctyl)

### Semiconductor Chip

The DNAe semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus was mounted on top of the IC and well assembly to facilitate the delivery of fluids over the surface of the chip. Oligonucleotides were coupled to the surface of the wells by first activating the well surface with an acrylamide-based polymer coating followed by the covalent attachment of 5′ modified oligonucleotides using standard Click chemistry.

### Protocol/Reaction Conditions

Briefly, preparation of Amplified Sequencing Templates comprised the following steps: PCR1; purification of product from PCR1; PCR2; capture of PCR2 product and elution of single-stranded DNA product.

### PCR1 Protocol

Oligonucleotide details for are shown in Table 40

TABLE 40

| Oligo Name | Sequence (5′ > 3′) |
|---|---|
| Oligo 1 | CACAGTACGTTCGCTAGTTGCTACAC |
| (Template 1 | GAGATCCTCTCTCTGAAATCACTGAG |
| PCR1 F Primer) | CAGGAG |
| (SEQ ID No. 209) | |
| Oligo 2 | GACAGGTACGTTCTAATCGCAGTACA |
| (Template 2 | CGAGATCCTCTCTGTGAAATCACTGA |
| PCR1 F Primer) | GCAGGAG |
| (SEQ ID No. 210) | |
| Oligo 3 | TCTATGGGCAGTCGGTGATGAATCTC |
| (Universal PCR1 | CATTTTAGCACTTACCTGTGACTCCA |
| R Primer) | TAG |
| (SEQ ID No. 211) | |

100 μL of PCR1 Master Mix (1× Platinum SuperFi PCR Master Mix (Thermo Fisher); 1 μM Oligo 1 (Integrated DNA Technologies (IDT)); 1 μM Oligo 2 (IDT); 1 μM Oligo 3 (IDT); 3000 copies DNA template (IDT); PCR Grade Water) was thermocycled (Mastercycler 505, Eppendorf) according to the following conditions: 1 cycle at 98° C. for 5 minutes; 2 cycles at 98° C. for 10 seconds; 68° C. for 2 minutes and 72° C. for 30 seconds; followed by 2 minutes at 72° C.

### PCR1 Purification Protocol

PCR1 product was purified with AMPure XP Beads (Beckman Coulter) as follows. 100 μL of PCR1 product was added to 150 μL of AMPure beads. The mixture was vortexed and incubated at room temperature for 5 minutes and repeated. The tubes were then placed on a DynaMag magnet for 5 minutes and the supernatant was discarded. The tube with the pelleted beads remained on the DynaMag magnet and was washed with 150 μL of 80% ethanol. The washed beads were incubated at room temperature (20-22° C.) for 30 seconds. The supernatant was again discarded and ethanol washing repeated as described above. The ethanol supernatant was removed and the tubes were then left on the DynaMag magnet to air-dry the beads for 5 minutes. The tubes were removed from the magnet and 54 μL of TE pH 8.0 low EDTA (AppliChem GmbH) was added directly to the pellet. The mixture was then vortexed thoroughly to disperse the beads, before incubating for 5 minutes and briefly centrifuged to collect the contents. The tubes were placed onto the magnetic rack and incubated for a further 2 minutes. 50 μL of the purified PCR product was then transferred to the PCR2 reaction tubes as described below.

### PCR2 Protocol

Oligonucleotide details for are shown in Table 41.

TABLE 41

| Oligo Name | Sequence (5′ > 3′) |
|---|---|
| Oligo 4 | /52- |
| (Template 1 PCR2 F Primer) | Bio/CCGGGCATGCCTAACAC |
| (SEQ ID No. 212) | AGTACGTTCGCTAGTTGC |
| Oligo 5 | /52- |
| (Template 2 PCR2 F Primer) | Bio/GCCGTACGAGCTGACGA |
| (SEQ ID No. 213) | CAGGTACGTTCTAATCGCAG |
| Oligo 6 | CTACGCCTCCGCTTTCCTCTC |
| (Universal PCR2 R Primer) | TATGGGCAGTCGGTGAT |
| (SEQ ID No. 214) | |

PCR product from PCR1, prepared as previously described, was used as input template material for PCR2. 112 μL of PCR2 Master Mix (1× Platinum SuperFi PCR Master Mix (ThermoFisher); 1.5 μM Oligo 4 (IDT); 1.5 μM Oligo 5 (IDT); 1.5 μM Oligo 6 (IDT); 50 μL PCR1 product) was thermocycled (Mastercycler 505, Eppendorf) according to the following conditions: 1 cycle at 98° C. for 5 minutes; 30 cycles of the following: 98° C. for 15 seconds, 68° C. for 15 seconds and 72° C. for 30 seconds; followed by 5 minutes at 72° C.

### Capture and Elution Protocol

Preparation of capture beads, subsequent capture of PCR2 product onto beads and elution of single-stranded DNA product was performed as follows. Capture beads were prepared by adding 110 μL of Bead Washing Buffer (1 M NaCl (Sigma Aldrich); 5 mM Tris-HCl (pH 7.5, PanReac); 0.5 mM EDTA (Sigma Aldrich); 0.05% Tween20 (Sigma Aldrich); 1 mg/mL BSA (Sigma Aldrich); Molecular Grade Water (Sigma Aldrich)) to 110 μL of MyOne C1 beads (Thermo Fisher) and vortexing for 5 seconds. The tube was capped and placed on a magnetic rack. The Bead Washing Buffer supernatant was removed, discarded, and removed from the magnetic rack. This was performed a total 3 times. Finally, 132 μL of Resuspension Buffer (1.5 mM NaCl; 10 mM Tris-HCl (pH 7.5); 1 mM EDTA; 0.1% Tween20; 1 mg/mL BSA; Molecular Grade Water (suppliers as above) was added to the washed beads. This mixture is referred to as

### Capture Beads.

110 μL of Capture Beads was combined with 110 μL PCR2 product in a 1.5 mL tube (Eppendorf). The tube was incubated for 10 minutes at room temperature with continuous mixing on a rotator (SB3, Stuart). The PCR product from PCR2 consists of double-stranded DNA, of which a single strand of the duplex is biotinylated at the 5′ end due to the use of biotinylated forward primers in the PCR2 reaction mix (described previously). During the incubation of the PCR2 product with the Capture Beads, the biotinylated strand of the DNA duplex binds to the streptavidin coated MyOne C1 beads. Following incubation, the reaction tube was placed on a magnetic rack for 2 minutes. The supernatant was removed and discarded, and the beads were washed by adding 200 μL of 1× wash buffer and vortexed for 5 seconds. The washing process was repeated 3 times. The tube was removed from the magnetic rack and 50 μL of 40 mM NaOH was pipetted into the tube and vortexed for 5 seconds to resuspend the beads. The resuspended beads were incubated at room temperature for 1 minute. The presence of 40 mM NaOH denatures the DNA duplex, such that the biotinylated strand remains bound to the streptavidin coated MyOne C1 beads, whilst the un-biotinylated strand of the duplex dissociates to become free in solution. The tube was then placed onto the magnetic rack for 2 minutes. The supernatant, containing the dissociated ssDNA, was transferred to a collection tube and prepared for use as input template into the sequencing reaction. Single stranded DNA prepared from PCR2 for use in sequencing is hereafter referred to as Amplified Sequencing Template. The sequencing templates used in this example are Sequencing Template 1 and Sequencing Template 2.

### Sequencing Protocol

The concentration of Amplified Sequencing Template was quantified using a Nanodrop 2000 (ThermoFisher) and the total concentration of DNA per sample adjusted to 1.1 μM by the addition of DNA-free water.

A 0.4 μM final concentration of each DNA template was prepared by adding 17.3 μL of Amplified Sequencing Template (1.1 μM) to 6.7 μL of Neutralizing Buffer (0.4 M NaCl; 75 mM Tris pH 7; Molecular Grade Water).

The template-containing Neutralizing Buffer was loaded into the flow cell assembled over the IC as previously described. The inlet and outlet of the flow cell was sealed with custom plugs and the templates were annealed to the surface-immobilized primers, complementary to the Amplified Sequencing Material (Oligo 18 and 20), using the following thermal protocol: a) 95° C. for 120 sec, b) 90° C. for 30 sec, c) 85° C. for 30 sec, d) 80° C. for 30 sec, e) 78° C. for 120 sec, f) Ramp down from 77° C. to 64° C. (inclusive) at a rate of 1° C. every 30 sec, g) 63° C. for 120 sec, h) Ramp down from 62° C. to 59° C. (inclusive) at a rate of 1° C. every 30 sec, i) 58° C. 15 mins, j) Passive cool-down to ambient temperature (20-26° C.) for 2 min. Sequencing enzyme (IsoPol BST+, ArcticZymes) and sequencing solutions were formulated as described in Example L.

Chip-Based pH Adjustment Protocol pH titration of sequencing solutions, including nucleotide-containing solutions, was performed using an automated process using the proprietary Chip-Based pH Adjuster (CBA). The automated CBA module used the mV output of the IC as a relative measure of pH, employing a feedback loop to dose appropriate volumes of 0.04 mM NaOH to the sequencing solutions in order to achieve a target pH (pH 8 0.1). Briefly, a reference pH solution (Tris, pH 8.0) was pumped across the surface of the IC and the mV output was recorded. Sequencing wash solution was then pumped across the chip, and the mV output recorded. The difference in mV output between the reference solution and the sequencing wash solution informed the appropriate NaOH dose to be added to the bulk volume of sequencing wash solution. The NaOH dose was added to the bulk volume of sequencing wash solution using an automated syringe pump. The process was repeated until the mV output of the sequencing wash solution matched the mV output previously recorded for the reference pH solution. The entire process was then repeated for each of the individual nucleotide solutions.

The subsequent steps (addition of Sequencing Enzyme, priming step, electrical response test, sequencing and analysis) were performed as described in Example L, with the following exception that the atmosphere in the sequencing solutions was maintained as C02-free using a proprietary CO₂ removal unit referred to as a ‘CO₂ Scrubber’. Briefly, the CO₂ scrubber eliminated the requirement to have a source of compressed nitrogen to maintain an inert atmosphere over the sequencing solutions, which is required in order to prevent undesirable acidification of the sequencing solutions The CO₂ Scrubber employed a column of soda-lime particles over which air was pumped using standard equipment. The soda-lime particles reacted with atmospheric CO₂, removing it from the air.

### Results and Conclusions

The sequencing results obtained from the amplified template using the Direct Hybridization method are shown in FIG. 74. Individual reads are plotted as ARL on the x-axis versus ARL-e on the y axis (unit of measure is base pairs; see Example L above for definition of ARL and ARL-x). A read with no errors would lie on the diagonal dashed line, where ALR-e=ARL. The histogram across the top of the plot indicate the distribution of reads along the x-axis, i.e., ARL (bp); the histogram on the right-hand side of the plot indicate the distribution of reads along the y-axis, i.e., ARL-e (bp). The total number of reads in this example was 6772. The median ARL-e achieved was 97 bp. The consensus read shown has an alignment length of 97 bases with an error rate of 3.1% (Table 42). These data demonstrate that the system as described is capable of sequencing a template which is hybridized to an immobilized primer.

TABLE 42
REFR: GTACGTTCGCTAGTTGCTACACGAGATCCTCTCTCTGAAATCACTGAGCAGGAGAAAGATTTTCTATGGAGTCACAGGTAAGTGCTAAAAT-GGAGA
|||||||||||||||||||||||||||||||||||||||||||||||||||||||||||>|||||||||||||||||||||||||||||||<|||||
CONS: GTACGTTCGCTAGTTGCTACACGAGATCCTCTCTCTGAAATCACTGAGCAGGAGAAAGA-TTTCTATGGAGTCACAGGTAAGTGCT-AAATGGGAGA
The symbols between the 2 sequences represent the following: | = perfect match, >= deletion, <= insertions, * = mismatch; CONS = consensus.

T. Sample Preparation Target Capture of Low Copy Number of Bacterial Genomes From 3 mL of Whole Blood with Shortened Incubation Protocols

### Specific Target Capture (STC) Oligomers

Specific Target Capture from whole blood was performed with modified oligomers complementary to target sequence containing a biotin linked to the 5′ end via a 5 adenosine (AAAAA) base nucleotide linker and a 3′ modification with an inverted-dT. The STC oligomers were ordered from IDT (Integrated DNA Technologies) with standard desalt purification and reconstituted in IDTE buffer (IDT) to 100 micromolar concentration:

KPC target STC oligomer for both strands of the kpc gene target region in Klebsiella pneumoniae bacterial strain:

KPC_STC_F2 (SEQ ID No. 215):
/5BiosG/AAAAAACCTCGTCGCGGAACC
ATTCGCTAAACTCGAACAGG/3InvdT/
KPC_STC_R2 (SEQ ID No. 216):
/5BiosG/AAAAACAGCACAGCGGCAGCA
AGAAAGCCCTTGAATGAGCT/3InvdT/

mecA target STC oligomers for both strands of the mecA gene target region in Staphylococcus aureus bacterial strain:

mecA STC_F-INT (SEQ ID No. 217):
/5BiosG/AAAAAAGGTACTGCTATC
CACCCTCAAACAGGT/3InvdT/
mecA STC R2 (SEQ ID No. 218):
/5BiosG/AAAAATTGAGTTGAACCT
GGTGAAGTTGTAATCTGG/3InvdT/

### Bacterial Target Genomic DNA Preparation

Target genomic DNA for capture in the Sample Preparation was isolated from bacterial organism strains obtained from CDC (Klebsiellapneumoniae ATCC BAA-1898, KPC+strain) and ATCC (Staphylococcus aureus BAA-2094, mecA+strain). The strain stocks were grown up and genomic DNA was isolated using a commercially available bacterial genomic DNA isolation kit and the DNA was quantified by digital PCR and further diluted to genomic equivalent (GE) copies.

### Streptavidin Beads

Capture of target DNA via the biotinylated STC oligomers from whole blood was performed with DNAe manufactured Streptavidin conjugated paramagnetic beads.

### Proteinase K

Cell lysis and protein digestion was performed using a commercially available Proteinase K enzyme (Roche/Sigma or Thermo Fisher Scientific).

### Lysis Buffer Preparation

Cell lysis and protein denaturation was performed using a Lysis Buffer with an ionic detergent.

### Wash and Elution Buffer

Streptavidin Beads were washed, and target DNA was eluted using a Wash and Elution Buffer containing a mild and low concentration surfactant in buffer.

### Whole Blood

Whole blood was obtained from BioIVT vendor.

### Antifoam

To reduce foaming during the Sample Preparation process either on benchtop or on the instrument cartridge, Antifoam B Silicone Emulsion (Sigma or J. T. Baker) was used.

### Mechanical Lysis Beads

For cell lysis of hard-to-lyse organisms such as fungal cells or Gram-positive cells, very hard density (VHD) zirconium oxide beads are used to shear open the cells on the OMNI International Bead Ruptor Elite instrument.

### Sample Preparation Reaction Setup

The Sample Preparation reaction was prepared by combining the following reagents as shown in the table below (Table 43) at room temperature. The reaction mixture was mixed thoroughly. For both benchtop and instrument cartridge process, the reaction setup was prepared in a 5 mL screw cap conical tube (Eppendorf, 30122348).

TABLE 43

| Reagent | Amount | |
|---|---|---|
| Whole Blood | 3 | mL |
| Target | 9 | GE per organism |
| Proteinase K | 1 | mg |
| STC oligo pool | 5 | pmoles per oligo |
| Antifoam B | 60 | μL |
| Lysis Buffer | 1 | mL |

### Sample Preparation Protocol: Lysis Through Target/Bead Capture

The Sample Preparation procedure requires several heating/cooling and incubation steps as outline in the table below (Table 44). The 3 mL of blood sample was mixed with the components from Table 1 which includes a lysis buffer with an ionic detergent and Proteinase K.

The mixture was incubated at 75° C. for 5 minutes for proteinase K enzyme digestion and then lysed mechanically for hard-to-lyse cells using the zirconium oxide beads.

After completion of lysis and protein digestion, the sample was heated to 95-100° C. for 10 minutes to denature the proteinase K enzyme and to convert double stranded DNA (dsDNA) to single stranded DNA (ssDNA) form.

The sample was then gradually cooled from 95-100° C. to 60° C. over a course of 5 minutes to hybridize biotinylated-STC-oligos to their respective ssDNA targets.

After the hybridization step, Streptavidin paramagnetic beads were added to the sample and the sample was mixed for 10 minutes at 1500 RPM at 45° C. to capture the STC oligo/target hybrids as well as free STC oligos via the strong biotin-Streptavidin interaction between the biotinylated STC oligos and the Streptavidin conjugated paramagnetic beads.

For the benchtop process, heating/cooling and mixing was performed in 5 mL heating blocks (Benchmark Scientific, H5000-5MT) on the thermal shaker instrument (Benchmark Scientific, H5000-HC).

For the instrument process, the heating and cooling was performed in a cartridge.

TABLE 44

| Process Step | Description | Temperature | Time | Mixing |
|---|---|---|---|---|
| Lysis/Proteinase K | Cell lysis, protein | 75° C. | 5 minutes | Benchtop: Not |
| Digestion | solubilization/digestion | | | applicable |
| Mechanical Lysis | Hard-to-lyse cells lysis | n/a | 5 minutes | OMNI Ruptor: 3 |
| | (only with target cells, | | | cycles of 6.6 m/s |
| | not with target gDNA) | | | for 90 sec. |
| | | | | mixing, 30 sec. |
| | | | | rest in between |
| | | | | cycles |
| Denaturation | DNA denaturation to | 95-100° | 10 minutes | Benchtop: Not |
| | ssDNA and Proteinase | C. | | applicable |
| | K deactivation | | | |
| Target Capture | Target DNA | Ramp down | 5 minutes | Benchtop: Not |
| | hybridization to STC | from 95-100° | | applicable |
| | oligos | C. to 60° C. | | |
| Streptavidin Bead | Add 1.2 mg of DNAe | Benchtop: Off | n/a | n/a |
| addition | Streptavidin beads to | instrument | | |
| | reaction | | | |
| Bead Capture | Capture Target/STC | 45° C. | 10 minutes | Benchtop: 1500 |
| | oligos to Streptavidin | | | RPM |
| | beads | | | |

Sample Preparation Protocol: Reaction Clean up through Elution

### Bead Washes

After the Bead Capture step, the clean-up of the beads was performed. The reaction tube was removed from the heating incubation instrument (Benchmark Scientific H5000-HC) and the tubes were placed on a 5-mL tube magnetic rack (Thermo Fisher, Dynamag-5) for 5 minutes to magnetize the Streptavidin Bead bound Target DNA/STC oligos complex to the side of the tube. The reaction supernatant was carefully removed and discarded without disturbing the beads. The beads were subsequently washed by resuspension of the beads with 1 mL of Wash and Elution Buffer. Resuspension of the beads in the reaction tube was performed off the magnetic rack. The resuspended beads were transferred to a 1.5 mL reaction tube (Eppendorf, 22431021). After resuspension, the reaction tube was placed back on a magnetic rack (Thermo Fisher, Dynamag-2) for 1 minute. The wash solution supernatant was removed and discarded. Three more washes were performed in the same manner as the first wash in the same 1.5 mL tube, for a total of 4×1 mL washes.

### Target Elution

After the final wash was removed, the beads were resuspended in 100 μL of Wash and Elution Buffer. The resuspended beads in the 1.5 mL tube were then placed on a thermal shaker (Benchmark Scientific, H5000-HC) containing a 1.5-mL tube heating block (Benchmark Scientific, H5000-CMB) pre-heated to 75° C. The target DNA was eluted by incubating the tube for 3 minutes at 75° C. Upon completion of the elution, the 1.5 mL reaction tube was transferred back to the 1.5 mL magnetic rack for 1 minute. The target DNA eluate was then transferred to a new 1.5 mL tube.

### Target DNA Detection in the Sample Preparation Eluate

Target DNA detection and quantitation from the Sample Preparation eluate was performed by nested PCR with final PCR being a quantitative PCR (qPCR) using a SuperFi qPCR mastermix, SYBR Green dye, and PCR primers specific to target regions of interest. First PCR amplification (PCR1) amplified target for 25 cycles using outer PCR primers. A 1:80 dilution of the PCR1 product was added to the second quantitative PCR (PCR2) using inner “nested” PCR primers with 40 cycles of amplification. Target detection is reported as Cq values from PCR2 which are compared against a no-target negative control Sample Prep reaction.

Additionally, a melt curve analysis was performed to confirm identity of the qPCR amplicon and rule out primer-dimer products.

TABLE 44
PCR1 and PCR2 primer sequences:

| | | SEQ | |
|---|---|---|---|
| Tar- | | ID | |
| get | Primer | No. | Sequence (5′ to 3′) |
| mecA | PCR1 Fwd | 13 | CATGAAAAATGATTATGGCTCAGGTAC |
| | PCR1 Rev | 14 | TGGAACTTGTTGAGCAGAGGTTC |
| KPC | PCR1 Fwd | 11 | AACCATTCGCTAAACTCGAACAGG |
| | PCR1 Rev | 12 | CCTTGAATGAGCTGCACAGTGG |
| mecA | PCR2 Fwd | 17 | GCTATCCACCCTCAAACAGGTGAAT |
| | PCR2 Rev | 18 | ATTCTTCGTTACTCATGCCATACATA |
| KPC | PCR2 Fwd | 15 | CTTTGGCGGCTCCATCGG |
| | PCR2 Rev | 16 | CTCCTCAGCGCGGTAACTTAC |

### Results and Conclusions

Quantitative PCR amplification data shows detection of the antimicrobial resistance gene targets (mecA and KPC) in Table 45, with bolded values in the table indicating at least 6 Cts above background signal and italicized values with 2 to 3 Cts above background signal. Background signal (underlined values) is denotated in PCR1+2 and PCR2 cells on the right for each of the two experiments performed.

TABLE 45
Sample Preparation Detection (qPCR Data) of Targets from 9 Genomic Copies per 3 mL of Blood

| | Sample Preparation Workflow | | | | | |
|---|---|---|---|---|---|---|
| | Sample Prep | | Sample Prep Cartridge | | | |
| | Benchtop | | Portion | | | |
| Workflow Incubation Duration Condition | Long | Short | Long | Short | | |
| Proteinase K Digestion Incubation Duration | 15 | 5 | 15 | 5 | PCR1 + 2 | PCR2 |
| | min. | min. | min. | min. | Negative | only |
| Denaturation Incubation Duration | 30 | 10 | 20 | 10 | Template | Negative |
| | min. | min. | min. | min. | Control | Template |
| SC Oligo Hybridization Duration | 40 | 5 | 30 | 5 | | Control |
| | min. | min. | min. | min. | | |

| | | Organism | | qPCR | Sample Preparation Replicates (PCR2 Ct Values) | | | | | | | | | |
|---|---|---|---|---|---|---|---|---|---|---|---|---|---|---|
| Target | Organism | Strain | Experiment | Replicate | Rep 1 | Rep 1 | Rep 2 | Rep 1 | Rep1 | Rep2 | Rep 3 | Rep 4 | NTC | NTC |
| mecA | S. | ATCC | 1 | Rep 1 | 21.6 | 34.3 | 24.4 | 25.1 | 26.4 | 24.4 | 23.7 | 24.7 | 34.1 | 34.2 |
| | aureus | BAA- | | Rep 2 | 21.6 | 34.8 | 24.3 | 25.2 | 26.3 | 24.5 | 23.7 | 24.6 | 34.7 | 34.4 |
| | | 2094 | 2 | Rep 1 | 26.8 | 31.0 | 33.3 | 33.8 | 36.8 | 28.8 | 28.9 | | 38.4 | ND |
| | | | | Rep 2 | 26.8 | 30.8 | 32.5 | 33.3 | 36.9 | 29.0 | 28.8 | | 38.9 | 39.3 |
| KPC | K. | ATCC | 1 | Rep 1 | 19.7 | 18.3 | 18.9 | 20.4 | 21.9 | 22.5 | 20.8 | 20.8 | ND | ND |
| | pneumoniae | BAA- | | Rep 2 | 19.6 | 18.4 | 18.9 | 20.3 | 22.1 | 22.6 | 20.7 | 20.6 | ND | 39.6 |
| | | 1898 | 2 | Rep 1 | 19.4 | 20.2 | 20.1 | 24.9 | 23.0 | 21.2 | 21.6 | | ND | ND |
| | | | | Rep 2 | 19.4 | 20.3 | 20.0 | 24.7 | 23.0 | 21.3 | 21.7 | | ND | ND |

The data also confirms that not only can the Sample Preparation cartridge portion perform equivalently to the benchtop process, but the Sample Preparation is robust enough with shortened incubation process steps (digestion, denaturation and hybridization) and still able to recover and detect targets at a very low (9 genomic equivalent) copies of target organism in 3 mL whole blood input volume.

### U. Cartridge Assay/Instrument Run

An exemplary assay performed using a cartridge and instrument exemplified in FIGS. 58A-61, 63-66, 69 and 70 is described herein with pipette tips stored onboard the cartridge and actuated via a gantry as described above.

Summary of Assay Steps for Each Pipette Tip
Tip 1, 5 mL: Sample Input through Denature and hybridization.
Tip 2, 5 mL: STC Bead Capture through Magnetic Bead Wash.
Tip 3, 1 mL: STC Elution through Copy Control Extension.
Tip 4, 1 mL: Preparing additional Copy Control reagents through loading Copy Control eluent.
Tip 5, 1 mL: Preparing Clonal Amp reagents through RCA/RPA Wash II.
Tip 6, 1 mL: Preparing amplification pellet through Sequencing Dehybridization Wash.
Tip 7, 1mLSequencing Hybridization through Sequencing Enzyme Binding.

Summary of SPI Interfaces:
SPI-V: Connection to vacutainer chamber.
SPI-ML: Connection to Mechanical Lysis chamber.
SPI-STC: Connection to STC chambers and bead capture serpentine.
SPI-PCR1: Connection to PCR1 chamber.
SPI-AUX: Auxiliary chamber will be used for several steps (PCR1 and CC dilution, PCR2 pooling).
SPI-PCR2: Connection to PCR2 network of channels and chambers.
SPI-CC: Connection to Copy Control chambers.
SPI-FC: Connection to Flow Cell.

Step #1: Enzymatic Lysis and Sample Retrieval
Pipettor picks up Tip 1 (volume 1-5 mL).
Tip 1 moved to Reagent Cartridge and pierces foil over Lysis Buffer.
Lysis Buffer is aspirated.
Tip 1 is docked to SPI-STC (STC chambers).
Dispense buffer into thermal mixing chambers, plus additional air to clear inlet channel, rehydrating lyos located in path or in chamber.
Tip 1 docks with SPI-V (vacutainer).
Vacutainer is pressurized via the pipettor and confirm adequate sample is present.
Sample is aspirated into pipette tip.
Tip 1 docked to SPI-STC.
Sample is dispensed into chambers, plus additional air to clear inlet channel.
Shuttle mix between thermal mixing chambers or between chambers and pipette tip to ensure lyos are fully rehydrated and reagents mixed.
Push all fluids into thermal mixing chambers.
Shuttle mix between thermal mixing chambers via pneumatic manifold ports while heating for Pro-K incubation. Periodic pushes from SPI to keep fluid in STC chambers.

Step #2: Mechanical Lysis
Reaction fluid is aspirated into Tip 1.
Tip 1 is docked with SPI-ML (Mechanical Lysis Chamber).
Fluid is pushed into the ML chamber, plus additional air to clear inlet channel.
Tip 1 is docked to SPI-STC.
Remaining fluid is aspirated into Tip 1.
Tip 1 is docked with SPI-ML (Mechanical Lysis Chamber).
Fluid is pushed into the ML chamber, plus additional air to clear inlet channel.
Mechanical lysis motor actuated.

Step #3: Denature/Hybridization
Reaction fluid is aspirated into Tip 1.
Tip 1 is docked with SPI-STC.
Dispense fluid into thermal mixing chambers, plus additional air to clear inlet channel.
Shuttle mix between thermal mixing chambers via pneumatic manifold ports while incubating at denature temperature.
Continue shutting mixing while incubating at hybridization temperature. Periodic pushes from SPI to keep fluid in STC chambers.
Tip 1 is dropped off in holding location.

Step #4: Bead Target Capture
Tip 2 is loaded by pipettor.
Tip 2 moves to Reagent Cartridge over Wash Buffer T and pierces foil.
Tip 2 aspirates volume of buffer required for rehydrating Magnetic Target Capture lyo.
Tip 2 moves to Magnetic Target Capture lyo and pierces foil.
Fluid is dispensed into lyo pocket to rehydrate bead.
Aspirate/dispense cycle occurs as necessary to ensure reagent is fully resuspended.
Magnetic Target Capture is aspirated into Tip 2.
Tip 2 docked to SPI-STC.
Magnetic Target Capture dispensed into thermal mixing chambers.
Blood solution is shuttle mixed between chambers and tip to suspend any beads that may have been lost in the channel. Solution is eventually pushed back into thermal mixing chambers.
Shuttle mix between thermal mixing chambers via pneumatic manifold ports while incubating at binding temperature. Periodic pushes from SPI to keep fluid in STC chambers.
STC magnet actuated against cartridge serpentine.
Fluid is aspirated into Tip 2, beads are pelleted against STC magnet.
Waste fluid is dispensed into Mechanical Lysis chamber.

Steps #5-6: Magnetic Bead Wash
Repeat 4×:
o Tip 2 moved to Wash Buffer T.
   Wash Buffer T is aspirated by Tip 2.
   Tip 2 docks to SPI-STC.
   STC magnet disengaged from cartridge.
   Wash Buffer T is shuttle mixed between pipette tip and thermal mixing chambers to resuspend beads.
   STC magnet is actuated against cartridge.
   Waste fluid is aspirated into Tip 2, beads are pelleted against STC magnet as they pass through the serpentine channel.
   Tip 2 is docked with SPI-ML.
   Waste fluid dispensed into ML chamber.

Loading PCR1 Dilution Chambers
Move Tip 2 to Reagent Cartridge and pierce foil over Water.
PCR1 Diluent is aspirated into Tip 2.
Tip 2 is docked to SPI-AUX (Auxiliary Chamber).
Diluent is dispensed into chamber.

Step #7: Elution
Tip 2 is dropped off in holding location.
Tip 3 is loaded by pipettor.
Tip 3 is moved to Reagent Cartridge over Wash Buffer T.
Wash Buffer T is aspirated.
Tip 3 is docked to SPI-STC.
STC magnet is disengaged from cartridge.
Eluent is shuttle mixed between pipette tips and thermal mixing chambers to resuspend beads.
Eluent and beads are pushed into thermal mixing chambers.
Eluent is shuttle mixed between thermal mixing chambers via pneumatic manifold ports while incubating at elution temperature. Periodic pushes from SPI may be necessary to keep fluid in chambers.
Eluent is shuttle mixed between thermal mixing chambers via pneumatic manifold ports while cooling.
STC magnet is engaged to cartridge.
STC eluate is aspirated into Tip 3, beads are pelleted against STC magnet as they pass through the serpentine channel.

Step #8: PCR 1 Inclusivity
Tip 3 is docked to SPI-PCR1.
Eluate is dispensed through channel, rehydrating PCR1 lyo, while PCR1 vent is open.
Once fluid is in reaction chamber, PCR1 vent is closed.
Chamber is pressurized via pipette tip.
TEMs are thermal cycled to perform PCR.

Step #9: PCR 1 Dilution
PCR1 chamber is depressurized via the pipettor.
A portion of PCR1 product is aspirated by Tip 3.
Tip 3 is docked to SPI-AUX.
PCR1 product is dispensed into dilution chamber.
PCR1 product and diluent are shuttle mixed between chamber and pipette tip to evenly dilute.

Step #10: PCR 2 Exclusivity
Tip 3 aspirates diluted PCR1 product.
Tip 3 docks with SPI-PCR2.
Diluted PCR1 product is dispensed into common line channel.
Each metering channel is filled, aided by optical sensors and control of valves on pneumatic manifold connected to vent lines.
Remaining fluid is aspirated to clear common line (bypass channel is vented, and all reaction chambers are vents are closed).
Reaction chambers are loaded by opening vent valves and pulling fluid from metering chambers to reaction chambers, rehydrating lyos.
Tip 3 is docked to SPI-ML.
Waste fluid is dispensed into ML chamber.
All reaction chamber vents are closed.
Reaction chambers are pressurized via the bypass channel.
TEMs are thermal cycled to perform PCR.
While PCR2 thermal cycling occurs:
   Tip 3 docks to SPI-AUX.
   Unused diluted PCR1 product is aspirated.
   Tip 3 docks to SPI-ML.
   Waste fluid dispensed into ML chamber.
   pH Auto-titration could occur (likely requires switching back to Tip 1 or using a fresh tip).
PCR2 reaction chambers are depressurized via the bypass channel.
Tip 3 is docked to SPI-PCR2.
PCR2 product is pooled into tip.
Tip 3 is docked to SPI-AUX.

“PCR2 product is dispensed into chamber.
Shuttle mixing between chamber and tip to fully mix PCR2 products and combine back into a single fluid slug.
Aliquot of pooled PCR2 product is aspirated into Tip 3.

Step #11: Copy Control Extension
Tip 3 is docked to SPI-CC.
Pooled PCR2 product is dispensed into channel, plus additional air to push fluid into thermal mixing chambers and rehydrating Hairpin lyo as fluid passes.
Fluid is shuttle mixed between thermal mixing chambers via pneumatic manifold ports while Copy Control TEMs are thermal cycled to perform extension.
While thermal cycle occurs, pipettor prepares additional reagents needed for Copy Control.
Tip 3 is docked to SPI-AUX.
Tip 3 aspirates the left over PCR2 product.
Tip 3 is docked to SPI-ML.
Remaining PCR2 product is dispensed into the ML chamber.
Tip 3 is dropped off in holding spot.
Tip 4 is loaded by pipettor.
Tip 4 is moved to Reagent Cartridge and pierces foil over Magnetic Bead Wash.
Tip 4 is moved to Water.
Volume of fluid required to rehydrate Hybridization lyo and Magnetic Target Capture lyo is aspirated.
Tip 4 is moved to Reagent Cartridge and pierces foil over Hybridization lyo and Magnetic Target Capture lyo.
Rehydrating fluid is dispensed into lyo wells to rehydrate reagents.
Aspirate/dispense cycle occurs as necessary to ensure reagent is fully resuspended.
Rehydrated Hybridization lyo is aspirated into Tip 4.

Step #12: Copy Control Hybridization
Tip 4 is docked with SPI-CC.
Hybridization reagent is dispensed into chamber, plus additional air required to clear inlet channel.
Fluid is shuttle mixed between thermal mixing chambers via pneumatic manifold ports.

Step #13: Copy Control Bead Target Capture
Tip 4 is moved to Reagent Cartridge over previously rehydrated Magnetic Target Capture.
Magnetic Target Capture reagent is aspirated into Tip 4.
Tip 4 is docked with SPI-CC.
Reagent is dispensed into chamber, plus additional air required to clear inlet channel.
Fluid is shuttle mixed between thermal mixing chambers via pneumatic manifold ports while TEMs are set to incubation temperature.
CC magnet is engaged against cartridge.
Tip 4 aspirates fluid into tip, beads are pelleted against CC magnet as they pass through the serpentine channel.
Tip 4 is docked to SPI-ML.
Waste fluid is dispensed into Mechanical Lysis chamber.

Step #14: Copy Control Magnetic Bead Wash
Repeat 3×:
   Tip 4 is moved to Reagent Cartridge and pre-pierced Magnetic Bead Wash.
   Wash buffer is aspirated into Tip 4.
   Tip 4 docks to SPI-CC.
   CC magnet is disengaged from cartridge.
   Fluid is shuttle mixed between pipette tip and thermal mixing chambers to resuspend beads.
   CC magnet is actuated against cartridge.
   Fluid is aspirated into Tip 4, beads are pelleted against CC magnet as they pass through the serpentine channel.
   Tip 4 is docked with SPI-ML.
   Waste fluid dispensed into ML chamber

Step #15: Copy Control Elution
Tip 4 moved to Reagent Cartridge over pre-pierced Water.
Eluent volume is aspirated.
Tip 4 is moved to Assay Cartridge and docked with SPI-CC.
CC magnet is disengaged from cartridge.
Eluent is dispensed into chamber and shuttle mixed between pipette tip and CC chambers to resuspend the beads.
Bead solution is then pushed into CC chambers and shuttle mixed via pneumatic manifold ports while TEMs are held at elution temperature.
While thermal mixing occurs, pipettor prepares Clonal Amp reagents.
Tip 4 is dropped off at its holding location on the Assay Cartridge.
Tip 5 is loaded by pipettor.
Tip 5 is moved to Reagent Cartridge and pierces several reagent foils.
Tip 5 is moved to Water.
Fluid to rehydrate Hybridization Buffer lyos is aspirated by Tip 5.
Fluid is dispensed into wells containing RCA Hyb Buffer lyo.
Aspirate/dispense cycle occurs as necessary to ensure reagent is fully resuspended.
Pre-pierced RCA Ligation Buffer is aspirated into Tip 5.
Fluid is dispensed into RCA Ligation lyo.
Aspirate/dispense cycle occurs as necessary to ensure reagent is fully resuspended.
Hybridization Buffer is aspirated into Tip 5.

Step #16: RCA/RPA Priming
Tip 5 is moved to the Assay Cartridge and is docked with SPI-FC (Flow Cell Cartridge).
Buffer is dispensed across the Flow Cell.

Step #17: RCA/RPA Hybridization (including Copy Control dilution)
Auxiliary Chamber washing process may need multiple repeats.
Tip 5 is moved to the Reagent Cartridge over pre-pierced Water.
Water is aspirated by Tip 5.
Tip 5 is docked with SPI-AUX.
Water is dispensed into the Auxiliary Chamber and shuttle mixed between tip and chamber to dilute and rinse any remaining PCR2 product.
Water is aspirated into Tip 5.
Tip 5 is docked with SPI-ML.
Waste is dispensed into the Mechanical Lysis chamber.
Tip 5 is moved to Reagent Cartridge over Hybridization Buffer.
Hybridization Buffer is aspirated into Tip 5.
Buffer is dispensed into Auxiliary Chamber.
Tip 5 is docked to SPI-CC.
All eluate is aspirated into Tip 5.
Tip 5 is docked to SPI-AUX.
Eluate is dispensed into Auxiliary Chamber and shuttle mixed between chamber and pipette tip to ensure thorough mixing with diluent.
Diluted CC eluate is aspirated into Tip 5.
Tip 5 is docked to SPI-FC.
Diluted CC eluate is dispensed across the Flow Cell.
Flow Cell TEM performs one thermal cycle.

Step #17: RCA I Wash (this step is eliminated for RPA)
Tip 5 is moved to the Reagent Cartridge over pre-pierced RCA Wash Buffer.
Wash Buffer is aspirated by Tip 5.
Tip 5 is moved back to the Assay Cartridge and is docked with SPI-FC.
Wash buffer is dispensed across flow cell chip
Waste is pulled through sequencing manifold towards Reagent Cartridge waste chamber.

Step #17: RCA II Circularization (this step is eliminated for RPA)
Tip 5 is moved to the Reagent Cartridge over previously rehydrated RCA Ligation.
RCA Ligation reagent is aspirated by Tip 5.
Tip 5 is moved to the Assay Cartridge and docks to SPI-FC.
RCA Ligation reagent is dispensed across the Flow Cell.
Fluid is incubated in Flow Cell.

Step #18: RCA/RPA Wash II
Tip 5 is moved back to the Reagent Cartridge over pre-pierced RCA Wash Buffer.
Wash buffer is aspirated by Tip 5.
Tip 5 is moved back to the Assay Cartridge and docks with SPI-FC.
Wash buffer is dispensed across flow cell chip.
Waste is pulled through sequencing manifold towards Reagent Cartridge waste chamber.

Step #19: RCA/RPA Reaction (this step has multiple lyos for RPA)
Tip 5 is dropped off at holding location.
Tip 6 is loaded by pipettor.
Tip 6 is moved to the Reagent Cartridge and pierces foil over RCA Amplification lyo.
RCA Wash Buffer is aspired into Tip 6.
Fluid is dispensed into RCA Amplification lyo.
Aspirate/dispense cycle occurs as necessary to ensure reagent is fully resuspended.
Tip 6 aspirates rehydrated RCA Amplification.
Reagent is aspirated by Tip 6.
Tip 6 is moved back to Assay Cartridge and docked with SPI-FC.
Reagent is dispensed across Flow Cell.
Chip TEM is set to isothermal amplification temperature.

Step #20A: RCA/RPA Wash
TEM is set to cooling temperature
While TEM and Flow Cell are cooling:
   Tip 6 is moved to the Reagent Cartridge over pre-pierced RCA Wash Buffer.
   Wash buffer is aspirated by Tip 6.
Once cooling has finished, Tip 6 is moved back to the Assay Cartridge and docks to SPI-FC.
Buffer is dispensed across Flow Cell.
Waste is pulled through sequencing manifold towards Reagent Cartridge waste chamber.

Step #20B: RCA Inactivation (this step is removed for RPA)
Tip 6 is moved to Reagent Cartridge over pre-pierced RCA Inactivation Buffer.
Inactivation Buffer is aspirated by Tip 6.
Tip 6 is moved to the Assay Cartridge and docks with SPI-FC.
Inactivation Buffer is dispensed across the Flow Cell.
TEM temperature is controlled.

Step #20C: RCA Wash II (this step is removed for RPA)
Tip 6 is moved to the Reagent Cartridge over pre-pierced RCA Wash Buffer.
Wash buffer is aspirated by Tip 6.
Tip 6 is moved back to the Assay Cartridge and docks with SPI-FC.
Wash buffer is dispensed across flow cell chip.
Waste is pulled through sequencing manifold towards Reagent Cartridge waste chamber.

Step #21: Dehybridization
Tip 6 is moved to the Reagent Cartridge over pre-pierced NaOH.
NaOH is aspirated by Tip 6.
Tip 6 is moved to Assay Cartridge and docks with SPI-FC.
NaOH is dispensed across the Flow Cell.
Waste is pulled through sequencing manifold towards Reagent Cartridge waste chamber.

Step #22: Wash
Tip 6 is moved to the Reagent Cartridge over pre-pierced Pre-Wash Buffer.
Wash buffer is aspirated by Tip 6.
Tip 6 is moved to the Assay Cartridge and docks with SPI-FC.
Wash buffer is pushed across the Flow Cell.
Waste is pulled through sequencing manifold towards Reagent Cartridge waste chamber.

Step #24: Sequencing Hybridization
Tip 6 is ejected into its holding location.
Pipettor picks up Tip 7.
Tip 7 is moved to the Reagent Cartridge and pierces foil over Sequencing Primer lyo, and Sequencing Hybridization Buffer.
Sequencing Hybridization Buffer is aspirated into Tip 7.
Buffer is dispensed into Sequencing Primer lyo.
Aspirate/dispense cycle occurs as necessary to ensure reagent is fully resuspended.
Tip 7 pierces foil over Sequencing Enzyme.
Tip 7 moves to Reagent Cartridge over pre-pierced Pre-Wash Buffer.
Wash Buffer is aspirated by Tip 7.
Fluid is dispensed into Sequencing Enzyme.
Aspirate/dispense cycle occurs as necessary to ensure reagent is fully resuspended.
Tip 7 is moved to previously rehydrated Sequencing Primer lyo.
Sequencing Primer is aspirated into Tip 7.
Tip 7 moves to Assay Cartridge and docks with SPI-FC.
Primer solution is dispensed across Flow Cell.
Additional air is dispensed to pressurize Flow Cell.
Chip TEMS perform hybridization thermal cycle.
Pipettor is depressurized.
Waste is pulled through sequencing manifold towards Reagent Cartridge waste chamber.

Step #25: Sequencing Wash
Tip 7 is moved to the Reagent Cartridge over pre-pierced Pre-Wash Buffer.
Wash buffer is aspirated by Tip 7.
Tip 7 is moved to Assay Cartridge and docks with SPI-FC.
Wash buffer is pushed across the Flow Cell.
Waste is pulled through sequencing manifold towards Reagent Cartridge waste chamber.

Step #26: Enzyme Binding
Tip 7 is moved to the Reagent Cartridge and over previously rehydrated Sequencing Enzyme.
Sequencing Enzyme is aspirated into Tip 7.
Tip 7 is moved to the Assay Cartridge and docks to SPI-FC.
Tip 7 dispenses reagent across Flow Cell.
Waste is pulled through sequencing manifold towards Reagent Cartridge waste chamber.
Chip TEM set to enzyme incubation temperature.

Sequencing is then controlled via the instrument/cartridge sequencing manifold and associated pumps and valves.

### Additional Embodiments

Various further embodiments of instruments, cartridges, systems, and/or methods are contemplated according to certain aspects of the invention.
Embodiment 1 is a method of preparing a target polynucleotide in a sample for further downstream processing or analysis, the method comprising:
   providing a sample comprising target polynucleotide; and;
   treating the sample or the target polynucleotide to obtain target nucleotide ready for further downstream processing or analysis.
Embodiment 2 is the method of claim 1, wherein the target polynucleotide comprises at least one of DNA (e.g., genomic DNA, cfDNA, ctDNA) and RNA (e.g., mRNA, rRNA, tRNA).
Embodiment 3 is the method of any one of the prior embodiments, wherein the method comprises filtering, concentrating, solubilizing, dissolving, homogenizing, or digesting the sample or otherwise altering the physical or chemical properties of the sample to aid in the preparation of target polynucleotide.
Embodiment 4 is the method of any one of the prior claims, wherein the sample comprises a biological or clinical sample.
Embodiment 5 is the method of embodiment 4, wherein the sample comprises whole blood, plasma, serum, buffy coat, white cells, red cells or platelets.
Embodiment 6 is the method of any one of the prior embodiments, wherein the sample comprises a cell that comprises the target polynucleotide.
Embodiment 7 is the method of embodiment 6, wherein the method further comprises tagging, labeling, capturing, concentrating, isolating or otherwise processing one or more cells, cell types or cell populations within a sample.
Embodiment 8 is the method of embodiment 7, wherein the method further comprises tagging, labeling, capturing, concentrating, isolating or otherwise processing cells suspected of containing target polynucleotide.
Embodiment 9 is the method of embodiments 6-8, wherein the method comprises lysing, digesting, rupturing, partially dissolving, shearing or otherwise manipulating cells containing or otherwise associated with target polynucleotide to render the target polynucleotide accessible or otherwise more available for further processing or analysis.
Embodiment 10 is the method of any one of the prior embodiments, wherein the method comprises lysing, digesting, rupturing, partially dissolving, shearing or otherwise manipulating structures containing or otherwise associated with target polynucleotide to render the target polynucleotide accessible or otherwise more available for further processing or analysis.
Embodiment 11 is the method of any one of the prior embodiments, wherein the method comprises tagging, labeling, annealing an oligonucleotide thereto, concentrating, enriching, capturing, separating, and/or isolating target polynucleotide.
Embodiment 12 is the method of embodiment 11, wherein the method further comprises tagging or labeling individual target polynucleotide molecules or subsets of target polynucleotide molecules with unique molecular identifiers (UMIs).
Embodiment 13 is the method of embodiment 12, wherein the UMIs are incorporated via ligation.
Embodiment 14 is the method of embodiment 12, wherein the UMIs are incorporated via extension of an UMI-tagged oligomer annealed to the target polynucleotide.
Embodiment 15 is the method of any one of embodiments 11-14, wherein the method further comprises annealing at least one capture oligomer to at least one target polynucleotide to generate at least one capture oligomer/target complex.
Embodiment 16 is the method of embodiment 15, wherein the at least one capture oligomer anneals to the at least one target polynucleotide at a target locus conserved across multiple different species.
Embodiment 17 is the method of embodiment 16, wherein the target locus comprises at least one of 16S, 23S, or 28S genomic DNA or a 5-untranslated region.
Embodiment 18 is the method of any one of embodiments 11-17, wherein the method further comprises extending with a polymerase the 3′-end of at least one capture oligomer in at least one capture oligomer/target complex.
Embodiment 19 is the method of any one of embodiments 11-18, wherein the at least one or more capture oligomers comprise any one or more capture oligomers described herein.
Embodiment 20 is the method of any one of embodiments 11-19, wherein the method further comprises isolating the at least one capture oligomer/target complex, thus isolating the target polynucleotide.
Embodiment 21 is the method of embodiment 20, wherein the method further comprises immobilizing the at least one capture oligomer/target complex onto a solid substrate.
Embodiment 22 is the method of any one of embodiments 11-21, wherein the at least one capture oligomer comprises a first ligand of a ligand pair conjugated thereto, the ligand pair comprises a second ligand bound to a solid substrate, and the isolating the at least one capture oligomer-target complex comprises binding the first ligand to the second ligand to generate an immobilized ligand complex.
Embodiment 23 is the method of embodiment 22, wherein the ligand pair comprises biotin/avidin or biotin/streptavidin.
Embodiment 24 is the method of embodiment 22, wherein the ligand pair comprises of a nucleic acid sequence and its complement.
Embodiment 25 is the method of embodiment 12-24, wherein the at least one capture oligomer comprises a tag sequence comprising a nucleic acid sequence that does not anneal to the target polynucleotide under a defined set of conditions.
Embodiment 26 is the method of embodiment 25, wherein at least one additional oligomer is provided and wherein the at least one additional oligomer comprises a nucleic acid sequence complementary to the tag sequence of the at least one capture oligomer and further comprises a first ligand of a ligand pair, annealing the additional oligomer to the tag of the capture oligomer of the capture oligomer/target complex, the ligand pair comprises a second ligand bound to a solid substrate, and the isolating the at least one capture oligomer-target complex comprises binding the first ligand to the second ligand to generate an immobilized ligand complex.
Embodiment 27 is the method of embodiment 26, wherein the capture oligomer is bound to the target polynucleotide and the additional oligomer is bound to the tag of the capture oligomer simultaneously.
Embodiment 28 is the methods of embodiments 26-27, wherein the additional oligomer is provided in a defined and limited amount.
Embodiment 29 is the method of embodiment 26-28, wherein the ligand pair comprises biotin/avidin or biotin/streptavidin.
Embodiment 30 is the method of embodiments 26-28, wherein the ligand pair is comprised of a nucleic acid sequence and its complement.
Embodiment 31 is the method of any of the embodiments 21-30, wherein the method further comprises washing of the solid substrate after immobilization of the complex comprising the target polynucleotide.
Embodiment 32 is the method of any of the embodiments 21-31, wherein the method further comprises eluting the target polynucleotide from the solid substrate.
Embodiment 31 is the method of any one of embodiments 11-32, wherein the method further comprises annealing one or more primers, blocker oligomers, displacer oligomers or cleavage site specific oligomers.
Embodiment 34 is the method of any of embodiments 1-11, wherein the target polynucleotide is used for downstream processing of analysis directly from sample without prior capturing, separating and/or isolating.
Embodiment 35 is a method of preparing a nucleic acid library, the method comprising amplifying a portion of a target polynucleotide to obtain a first amplicon.
Embodiment 36 is the method of embodiment 35, wherein the target polynucleotide is the product of the method of any one of embodiments 1-33.
Embodiment 37 is the method of embodiment 35, wherein the target polynucleotide is used directly from sample.
Embodiment 38 is the method of any of embodiments 35-37, wherein the amplifying comprises amplifying at least one portion of the target polynucleotide with at least one first primer to generate at least one first amplicon.
Embodiment 39 is the method of embodiment 38, wherein the said at least one first primer comprises at least one capture oligomer or other oligomer type of any one of embodiments 14-33.
Embodiment 40 is the method of embodiment 38, wherein the at least one first primer hybridizes to at least one tag sequence incorporated into the target polynucleotide or a fragment or fragments thereof.
Embodiment 41 is the method of any of embodiments 38-40, wherein the said at least one first primer comprises at least one tag.
Embodiment 42 is the method of any of embodiments 38-41, wherein the said at least one first primer comprises at least one primer that is positive (+) sense and at least one primer that is minus (−) sense.
Embodiment 43 is the method of embodiment 42, wherein the at least one plus sense primer hybridizes to at least one tag sequence and the at least one minus sense primer hybridizes to the target polynucleotide or a fragment or fragments thereof, the at least one minus sense primer hybridizes to at least one tag sequence and the at least one plus sense primer hybridizes to the target polynucleotide or a fragment or fragments thereof, or the at least one plus sense primer hybridizes to at least one tag sequence and the at least one minus sense primer hybridizes to at least one different tag sequence.
Embodiment 44 is the methods of any of the embodiments 35-43, wherein more than one first amplicon is produced.
Embodiment 45 is the method of any of the embodiments 38-44, wherein the said at least one first primer comprises any primer or one or more groups of opposed primers described herein.
Embodiment 46 is the method of any of the embodiments 35-45, wherein the amplification method is PCR.
Embodiment 47 is the method of any of the embodiments 35-46, wherein the at least one first amplicon is purified.
Embodiment 48 is the method of any of the embodiments 35-47, further comprising amplifying at least a portion of the at least one first amplicon with at least one second primer to generate at least one second amplicon.
Embodiment 49 is the method of embodiment 48, wherein the said at least one second primer hybridizes to at least one tag sequence incorporated into the at least one first amplicon or a fragment or fragments thereof.
Embodiment 50 is the method of any of embodiments 48-49, wherein the said at least one second primer comprises a at least one tag.
Embodiment 51 is the method of any of the embodiments 48-50, wherein the at least one second primer is nested by at least one nucleotide within the at least one first primer.
Embodiment 52 is the method of any of embodiments 48-51, wherein the said at least one second primer comprises at least one primer that is positive (+) sense and at least one primer that is minus (−) sense.
Embodiment 53 is the method of embodiment 52, wherein the at least one plus sense primer hybridizes to at least one tag sequence and the at least one minus sense primer hybridizes to the said at least one first amplicon or a fragment or fragments thereof, the at least one minus sense primer hybridizes to at least one tag sequence and the at least one plus sense primer hybridizes to the said at least one first amplicon or a fragment or fragments thereof, or the at least one plus sense primer hybridizes to at least one tag sequence and the at least one minus sense primer hybridizes to at least one different tag sequence.
Embodiment 54 is the methods of any of the embodiments 48-53, wherein more than one second amplicon is produced.
Embodiment 55 is the method of embodiment 54, wherein at least one second primer is nested within the at least one first primer and at least one other second primer is not nested within the at least one said first primer.
Embodiment 56 is the method of any of the embodiments 48-55, wherein the said at least one second primer comprises any primer or one or more groups of opposed primers described herein.
Embodiment 57 is the method of any of the embodiments 48-56, wherein the amplification method is PCR.
Embodiment 58 is the method of any of the embodiments 48-57, wherein the at least one first amplicon is purified.
Embodiment 59 is the method of any of the embodiments 48-57, wherein the at least one second amplicon is purified.
Embodiment 60 is the method of embodiment 58, wherein the at least one second amplicon is also purified.
Embodiment 61 is a method of preparing a nucleic acid library, the method comprising ligating a sequence tag to the target polynucleotide.
Embodiment 62 is the method of embodiment 61, wherein the tag comprises an adaptor molecule.
Embodiment 63 is the method of embodiment 62, wherein the tag further comprises additional sequence.
Embodiment 64 is the method of any one of embodiment 61-63, wherein the target polynucleotide is used directly from sample.
Embodiment 65 is the method of any one of embodiments 61-63, wherein the target polynucleotide is the product of the method of any one of embodiments 1-33.
Embodiment 66 is a capture oligomer comprising, in the 5′ to 3′ direction: a capture sequence, an internal extension blocker, a complement of the capture sequence, and a target-hybridizing sequence, wherein the complement of the capture sequence is configured to anneal to the capture sequence in the absence of an extended target sequence annealed to the target-hybridizing sequence and the complement of the capture sequence.
Embodiment 67 is the capture oligomer of embodiment 66, wherein the capture oligomer has the formula 5′-A1-C-L-B-A2-C′-A3-RB-A4-THS-X-3′, wherein A1 is an optionally present first additional sequence; C is the capture sequence, L is an optionally present linker, B is the internal extension blocker, A2 is an optionally present second additional sequence, C′ is the complement of the capture sequence, A3 is an optionally present third additional sequence, RB is an optionally present reversible extension blocker, A4 is an optionally present fourth additional sequence, THS is the target-hybridizing sequence; and X is an optionally present blocking moiety.
Embodiment 68 is a combination comprising a capture oligomer and a complementary oligomer, wherein: (a) the capture oligomer comprises, in the 5′ to 3′ direction: a capture sequence comprising first and second portions, an internal extension blocker, a spacer sequence comprising first and second portions, and a target-hybridizing sequence; and (b) the complementary oligomer comprises, in the 3′ to 5′ direction: a complement of the second portion of the capture sequence, and a complement of at least the first portion of the spacer sequence, wherein the complement of the second portion of the capture sequence and the complement of the at least first portion of the spacer sequence are configured to anneal simultaneously to the capture oligomer in the absence of a complement of the spacer sequence.
Embodiment 69 is the combination of embodiment 68, wherein the capture oligomer has the formula: 5′-A1-C1-C2-B-A2-S1-S2-A3-RB-A4-THS-X-3′, wherein A1 is an optionally present first additional sequence, C1 is the first portion of the capture sequence, C2 is the second portion of the capture sequence, B is the internal extension blocker, A2 is an optionally present second additional sequence, S1 is the first portion of the spacer sequence, S2 is the second portion of the spacer sequence, A3 is an optionally present third additional sequence, RB is an optionally present reversible extension blocker, A4 is an optionally present fourth additional sequence, THS is the target-hybridizing sequence, and X is an optionally present blocking moiety.
Embodiment 70 is the combination of any one of embodiments 68 or 69, wherein the complementary oligomer has the formula: 5′-S1′-A2′-L-C2′-X-3′, wherein S1′ is the complement of at least the first portion of the spacer sequence, A2′ is an optionally present complement of a second additional sequence which is optionally present in the capture oligomer; L is an optionally present linker, C2′ is the complement of the second portion of the capture sequence, and X is an optionally present blocking moiety.
Embodiment 71 is a method of capturing a target polynucleotide from a composition, the method comprising: contacting the target polynucleotide with the capture oligomer of any one of embodiments 66 or 67, wherein the target-hybridizing sequence of the capture oligomer anneals to the target polynucleotide at a site comprising the 3′ end of the target polynucleotide; extending the 3′ end of the target polynucleotide with a DNA polymerase with strand-displacement activity, thereby forming a complement of the complement of the capture sequence, which is annealed to the capture oligomer, such that the capture sequence of the capture oligomer is available for binding; contacting the capture sequence of the capture oligomer with a secondary capture reagent comprising a complement of the capture sequence and (i) a binding partner or (ii) a solid support, thereby forming a complex comprising the target polynucleotide, the capture oligomer, and the secondary capture reagent; and isolating the complex from the composition, thereby capturing the target polynucleotide.
Embodiment 72 is a method of capturing a target polynucleotide from a composition, the method comprising: contacting the composition with the combination of any one of embodiments68-70, wherein the target-hybridizing sequence of the capture oligomer anneals to the target polynucleotide at a site comprising the 3′ end of the target polynucleotide; extending the 3′ end of the target polynucleotide with a DNA polymerase with strand-displacement activity, thereby forming a complement of the spacer sequence, which is annealed to the capture oligomer, such that the complementary oligomer is displaced to an extent sufficient for the capture sequence of the capture oligomer to be available for binding; contacting the capture sequence of the capture oligomer with a secondary capture reagent comprising a complement of the capture sequence and (i) a binding partner or (ii) a solid support, thereby forming a complex comprising the target polynucleotide, the capture oligomer, and the secondary capture reagent; and isolating the complex from the composition, thereby capturing the target polynucleotide.
Embodiment 73 is a method of capturing a target polynucleotide from a composition, the method comprising: contacting the target polynucleotide with a capture oligomer comprising, in the 5′ to 3′ direction: a capture sequence, an optional internal extension blocker, an optional spacer sequence, and a target hybridizing sequence that is configured to anneal to the target polynucleotide; contacting the capture oligomer with a first capture reagent comprising a complement of the capture sequence (before, while, or after contacting the target polynucleotide with the capture oligomer); providing a second capture reagent comprising a complement of a sequence in the capture oligomer other than the capture sequence, wherein if some or all of the capture oligomer is not annealed to the target polynucleotide, the second capture reagent contacts the capture oligomer that is not annealed to the target polynucleotide; isolating first and second complexes from the composition, wherein the first complex comprises the target polynucleotide and the second complex comprises capture oligomer not annealed to the target polynucleotide; and selectively eluting the target polynucleotide or a subcomplex comprising the target polynucleotide from the first complex; optionally wherein (a) the first capture reagent comprises (i) a binding partner and (e.g., biotin) or (ii) a solid support (e.g., bead or surface) and/or (b) the second capture reagent comprises (i) a binding partner and (e.g., biotin) or (ii) a solid support (e.g., bead or surface).
Embodiment 74 is the method of any one of embodiments 71-73, wherein the input material is the output material of any one of claims 1-34.
Embodiment 75 is the method of any one of embodiments 71-73, wherein the input material is the output material of any one of claims 35-65.
Embodiment 76 is a method of generating a nucleic acid cluster immobilized on a solid support, the method comprising amplifying a target polynucleotide in a reaction mixture comprising solid support-immobilized first primer and non-immobilized second primer to generate solid support-immobilized target nucleic acid amplicon and free target nucleic acid amplicon.
Embodiment 77 is the method of embodiment 76, wherein the reaction mixture is devoid of non-immobilized first primer.
Embodiment 78 is the method of embodiment 76, wherein the reaction mixture further comprises non-immobilized first primer.
Embodiment 79 is the method of embodiment 78, wherein the relative ratio of the concentrations of the said non-immobilized second and first primers is about one-to-one.
Embodiment 80 is the method of embodiment 78, wherein the relative ratio of the concentrations of the said non-immobilized second and first primers is between about two-to-one and one thousand-to-one or more.
Embodiment 81 is the method of embodiment 78, wherein the relative ratio of the concentrations of the said non-immobilized first and second primers is between about two-to-one and one thousand-to-one or more.
Embodiment 82 is the method of any one of embodiments 76-81, wherein said target polynucleotide is added to the reaction mixture at a concentration that approximates a Poisson distribution level on the solid support.
Embodiment 83 is the method of any one of embodiments 76-82, wherein the method comprises hybridizing the target polynucleotide to the said solid support-immobilized first primer before amplification is initiated.
Embodiment 84 is the method of embodiment 83, wherein the solid support is washed after hybridization of the target polynucleotide to the said solid support-immobilized first primer and before amplification is initiated.
Embodiment 85 is the method of any one of embodiments 76-84, wherein target nucleic acid amplicon that is not immobilized is generated in addition to the said surface-immobilized target nucleic acid amplicon.
Embodiment 86 is the method of embodiment 85, wherein one or more strands of the said target nucleic acid amplicon that is not immobilized hybridizes to solid support-immobilized first primer and is amplified to generate additional solid support-immobilized target nucleic acid.
Embodiment 87 is the method of embodiment 86, wherein the said hybridization of the said target nucleic acid amplicon that is not immobilized to the said solid support-immobilized first primer occurs in close proximity to the site of generation of solid-support-immobilized target nucleic acid amplicon in preceding stages of amplification.
Embodiment 88 is the method of any one of embodiments 76-87, wherein the cluster is monoclonal.
Embodiment 89 is the method of any one of embodiments 76-88, wherein the target polynucleotide comprises at least 2 different target polynucleotides and the amplifying comprises:
   hybridizing the different target polynucleotides to the solid support-immobilized first primers at spatially distanced locations on the solid support;
   generating a solid support-immobilized target nucleic acid amplicon cluster of each hybridized target polynucleotides at and around each of the spatially distanced locations, wherein at least a portion of each cluster does not overlap with at least a portion of a neighboring cluster.
Embodiment 90 is the method of any one of embodiments 76-89, wherein the said solid support-immobilized first primer and/or the said non-immobilized second or first primers comprise any primer or one or more groups of opposed primers described herein.
Embodiment 91 is the method of any one of embodiments 76-90, wherein the amplification method is Recombinase Polymerase Amplification.
Embodiment 92 is the method of any one of embodiments 76-91, wherein the target polynucleotide comprises the target nucleic acid output of any one of the claims in embodiments 1-75.
Embodiment 93 is the method of any one of embodiments 76-91, wherein the target polynucleotide is used directly from the sample.
Embodiment 94 is the method of any one of embodiments 76-91, wherein the target polynucleotide comprises a first-primer binding sequence and/or a second-primer binding sequence added as the tag sequence of embodiment 22, or the tag sequence recited in embodiments 41 or 50.
Embodiment 95 is the method of any one of embodiments 76-93, wherein the solid support comprises the surface of a semi-conductor chip.
Embodiment 96 is the method of embodiment 94, wherein the surface of a semi-conductor chip further comprises an array of 3-dimensional features.
Embodiment 97 is the method of embodiment 95, wherein the 3-dimensional features comprise wells.
Embodiment 98 is the method of any one of embodiment 94-96, wherein the semiconductor chip further comprises an array of ISFET sensors.
Embodiment 99 is a method of generating a nucleic acid cluster immobilized on a solid support, the method comprising amplifying a target polynucleotide, the method further comprising:
   providing linear target nucleic acid comprising a first partial portion of a first-primer binding sequence on a first portion of the linear target nucleic acid, a second partial portion of the first-primer binding sequence on a second portion of the linear target nucleic acid, and a second primer sequence between the first portion and the second portion of the linear target nucleic acid;
   providing a solid support comprising first primers and second primers immobilized on the surface of the solid support, wherein the first primers comprise the first-primer sequence and the second primers comprise the second-primer sequence;
   hybridizing the first partial portion and the second partial portion of the first-primer binding sequence of the target nucleic acid to one of the immobilized first primers;
   ligating the first partial portion and the second partial portion of the first-primer binding sequence of the target nucleic acid to generate a circularized target nucleic acid comprising the first-primer binding sequence and the second primer sequence;
   extending the one of the immobilized first primers along the circularized target nucleic acid as a template to generate a first extended target nucleic acid strand comprising at least one copy of the first primer sequence and at least one copy of the second-primer binding sequence;
   hybridizing the at least one copy of the second-primer binding sequence on the first extended target nucleic acid strand to at least one of the immobilized second primers;
   extending the at least one copy of the one of the immobilized second primers along the first extended target nucleic acid strand to generate a second extended target nucleic acid strand comprising at least one copy of the second primer sequence and at least one copy of the first-primer binding sequence; and
   hybridizing the at least one of the first-primer binding sequence on the second extended target nucleic acid strand to another of the immobilized first primers; and
   extending the other of the immobilized first primers along the second extended target nucleic acid strand as a template to generate a third extended target nucleic acid strand comprising at least one copy of the first primer sequence and at least one copy of the second-primer binding sequence.
Embodiment 100 is the method of embodiment 99, wherein the amplification method is Rolling Circle Amplification.
Embodiment 101 is the method of any one of the embodiments 99-100, wherein the target polynucleotide comprises the target nucleic acid output of any one of the claims in sections 1-75.
Embodiment 102 is the method of any one of the embodiments 99-100, wherein the target polynucleotide is used directly from the sample.
Embodiment 103 is the method of any one of embodiments 99-102, wherein the target polynucleotide comprises a first-primer binding sequence and/or a second-primer sequence added as the tag sequence of embodiment 22, or the tag sequence recited in embodiments 41 or 50.
Embodiment 104 is the method of embodiment 103, wherein the one or more additional sequences added to the target polynucleotide include the first partial portion of the first-primer binding sequence on a first end of the target polynucleotide, a partial or whole second primer sequence in the internal region of the target polynucleotide and a second partial portion of the first-primer binding sequence on a second end of the target polynucleotide.
Embodiment 105 is the method of any one of embodiments 99-104, wherein the solid support comprises the surface of a semi-conductor chip.
Embodiment 106 is the method of embodiment 105, wherein the surface of a semi-conductor chip further comprises an array of 3-dimensional features.
Embodiment 107 is the method of embodiment 106, wherein the 3-dimensional features comprise wells.
Embodiment 108 is the method of any one of embodiments 105-107, wherein the semiconductor chip further comprises an array of ISFET sensors.
Embodiment 109 is a method of generating a nucleic acid cluster immobilized on a solid support, the method comprising hybridizing a target polynucleotide in a reaction mixture comprising solid support-immobilized first oligonucleotide to generate solid support-immobilized target polynucleotide.
Embodiment 110 is the method of embodiment 109, wherein the said solid support-immobilized first oligonucleotide comprises a capture oligonucleotide, a primer or a tethering oligonucleotide.
Embodiment 111 is the method of any one of the embodiments 109-110, wherein the said solid support-immobilized first oligonucleotide is affixed in a discrete area of the surface of the said solid support.
Embodiment 112 is the method of embodiment 111, wherein the affixing comprises spotting or direct synthesis of the oligonucleotide on the surface of the solid support.
Embodiment 113 is the method of any one of the embodiments 109-112, wherein the immobilized nucleic acid cluster is monoclonal.
Embodiment 114 is the method of any one of the embodiments 109-112, wherein the method further comprises at least a first and a second solid support-immobilized oligonucleotide, and further wherein at least a first and second nucleic acid cluster is generated.
Embodiment 115 is the method of embodiment 114, wherein the at least first and second nucleic acid clusters are monoclonal.
Embodiment 116 is the method of any one of embodiments 109-115, wherein the solid support comprises the surface of a semi-conductor chip.
Embodiment 117 is the method of embodiment 116, wherein the surface of a semi-conductor chip further comprises an array of 3-dimensional features.
Embodiment 118 is the method of embodiment 117, wherein the 3-dimensional features comprise wells.
Embodiment 119 is the method of any one of embodiments 116-118, wherein the semiconductor chip further comprises an array of ISFET sensors.
Embodiment 120 is a method of generating a population of nucleic acid clusters immobilized on a solid support, the method comprising amplifying a population of target polynucleotides by Recombinase Polymerase Amplification in a reaction mixture comprising solid support-immobilized first primer and non-immobilized second primer to generate solid support-immobilized target nucleic acid amplicon and free target nucleic acid amplicon, wherein at least a portion of each cluster does not overlap with at least a portion of a neighboring cluster.
Embodiment 121 is the method of embodiment 120, wherein one or more strands of the said free target nucleic acid amplicon that is not immobilized hybridizes to solid support-immobilized first primer and is amplified to generate additional solid support-immobilized target nucleic acid.
Embodiment 122 is the method of embodiments 120 or 121, wherein the target polynucleotides have universal regions at the 3′ and/or 5′ ends.
Embodiment 123 is the method according to any one of embodiments 120-122, wherein the solid support comprises wells and the majority of the wells contain clonal amplicons.
Embodiment 124 is the method according to embodiment 123, wherein the amplicons in the wells are sequenced.
Embodiment 125 is the method according to embodiment 124 wherein the sequencing is performed using a semiconductor chip comprising an array of ISFET sensors.
Embodiment 126 is a method of generating a population of nucleic acid clusters immobilized on a solid support, the method comprising amplifying a population of target polynucleotides by Recombinase Polymerase Amplification in a reaction mixture comprising solid support-immobilized first primer and non-immobilized second primer to generate solid support-immobilized target nucleic acid amplicon and free target nucleic acid amplicon, wherein the clusters are randomly distributed on the solid support and at least a portion of each cluster does not overlap with at least a portion of a neighboring cluster.
Embodiment 127 is the method of embodiment 126, wherein the solid support comprises 3-dimensional features.
Embodiment 128 is the method of embodiment 127, wherein the 3-dimensional features are wells.
Embodiment 129 is the method of any one of embodiments 126-128, wherein the solid support comprises a semi-conductor chip.
Embodiment 130 is the method of embodiment 129, wherein the chip comprises an array of ISFET sensors.
131 Embodiment 131 is a method of determining the nucleotide sequence of a target polynucleotide, the method comprising:
   Immobilizing the target polynucleotide or a derivative thereof to a solid support;
   optionally amplifying the immobilized target polynucleotide or derivative thereof to produce a cluster;
   annealing a sequencing primer to the immobilized polynucleotide, derivative thereof or amplicon product thereof to create a target nucleic acid/sequencing primer complex;
   binding a sequencing enzyme to the target nucleic acid/sequencing primer complex;
   sequentially adding nucleotides and measuring a signal after each addition to determine the nucleotide sequence of a target polynucleotide.
Embodiment 132 is the method of any one of embodiments 131, wherein the target polynucleotide is used directly from sample.
Embodiment 133 is the method of any one of embodiments 131 or 132, wherein the target polynucleotide is prepared from a sample.
Embodiment 134 is the method of embodiment 133, wherein preparing the sample comprises the method of any one of embodiments 1-33, embodiments 35-65, embodiments 66-75, embodiments 76-130.
Embodiment 135 is the method of any one of embodiments 131-134, wherein the immobilized oligonucleotide is a sequencing primer.
Embodiment 136 is the method of embodiment 135, wherein the immobilization step and hybridization of a sequencing primer step are one and the same.
Embodiment 137 is the method of any one of the embodiments 131-134, wherein the immobilizing and optionally amplifying the target polynucleotide or derivative thereof comprises the method of any one of embodiments 76-108.
Embodiment 138 is the method of any one of the embodiments 131-134 or 137, wherein the annealing a sequencing primer to the immobilized polynucleotide, derivative thereof or amplicon product thereof to create a target nucleic acid/sequencing primer complex and binding a sequencing enzyme to the target nucleic acid/sequencing primer complex occur in the same step.
Embodiment 139 is the method of embodiments 131-138, wherein the sequencing enzyme is heat stable.
Embodiment 140 is the method of any one of the embodiments 131-139, wherein the method further comprises a flow cell that facilitates delivery and removal of fluids to and from the solid support.
Embodiment 141 is the method of any one of the embodiments 131-140, wherein the solid support comprises the surface of a semi-conductor chip.
Embodiment 142 is the method of embodiment 141, wherein the surface of a semi-conductor chip further comprises an array of 3-dimensional features.
Embodiment 143 is the method of embodiment 142, wherein the 3-dimensional features comprise wells.
Embodiment 144 is the method of any one of embodiments 141-143, wherein the semiconductor chip further comprises an array of ISFET sensors.
Embodiment 145 is the method of embodiment 144, wherein measuring a signal after each addition to determine the nucleotide sequence of a target polynucleotide comprises detection by the ISFET sensors.
Embodiment 146 is the method of any one of embodiments 131-145, wherein the determining the sequence comprises use of computerized analysis algorithms.
Embodiment 147 is a system comprising an instrument and an assay cartridge removably insertable within the instrument.
Embodiment 148 is the system of embodiment 147, wherein the assay cartridge comprises any one or more, in any combination, of:
   a sample input unit;
   an incubation unit
   a lysis unit;
   a magnetic separation]unit (MSU),
   a library preparation unit;
   a copy control (CC) unit; and
   a cluster generation/sequencing unit.
Embodiment 149 is the system of embodiment 148, wherein the sample input unit comprises a sample intake, an input/output valve, and a fluid channel between and in fluid connection with the sample intake and the input/output valve.
Embodiment 150 is the system of any one of embodiments 148-149, further comprising a fluid channel and in fluid connection between the sample intake and at least one chamber in the cartridge.
Embodiment 151 is the system of embodiment 150, wherein the at least one chamber in the cartridge comprises at least one lysis chamber.
Embodiment 152 is the system of any one of embodiments 148-151, wherein the sample input is a blood-collection tube (e.g., VACUTAINER (Becton Dickinson)) input.
Embodiment 153 is the system of any one of embodiments 148-152, wherein the incubation unit comprises at least one incubation chamber, at least one input/output valve, a fluid channel between and in fluid connection with at least one incubation chamber and at least one input/output valve, and a fluid channel between and in fluid connection with the least one incubation chamber and at least one additional incubation chamber when 2 of more incubation chambers exist.
Embodiment 154 is the system of embodiment 153, wherein the at least one incubation chamber interfaces with a heating element when the assay cartridge is inserted within the instrument.
Embodiment 155 is the system of any one of embodiments 153-154, wherein the at least one incubation chamber comprises at least one lysis chamber.
Embodiment 156 is the system of any one of embodiments 153-155, wherein the system comprises more than one incubation units.
Embodiment 157 is the system of any one of embodiments 147-156, wherein the lysis unit comprises at least one lysis chamber, an input/output valve, a fluid channel between and in fluid connection with the at least one lysis chamber and the input/output valve, and a fluid channel between and in fluid connection with the at least one lysis chamber and at least one additional lysis chamber when 2 of more lysis chambers exist.
Embodiment 158 is the system of embodiment 157, wherein the at least one lysis chamber comprises a spinning paddle or impeller.
Embodiment 159 is the system of embodiment 158, wherein the spinning paddle or impeller operationally interfaces with an actuator on the instrument when the assay cartridge is inserted within instrument.
Embodiment 160 is the system of any one of embodiment 147-159, wherein the MS unit comprises one or more MS chambers.
Embodiment 161 is the system of embodiment 160, wherein the one or more MS chambers interface with a heating element on the instrument when the assay cartridge is inserted within the instrument.
Embodiment 162 is the system of any one of embodiment 160-161, wherein the one or more MS chambers interface with a magnetic element on the instrument when the assay cartridge is inserted within the instrument.
Embodiment 163 is the system of any one of embodiments 160-162, wherein the MS unit further comprises an input/output valve and a fluid channel between and in fluid connection with the one or more MS chambers and the input/output valve.
Embodiment 164 is the system of any one of embodiments 160-163, wherein the MS unit further comprises one or more pneumatic ports in fluid connection with the one or more MS chambers, wherein the one or more pneumatic ports operationally interface with pneumatic manifold on the instrument when the assay cartridge is inserted within the instrument.
Embodiment 165 is the system of embodiment 164, wherein the MS unit further comprises one or more condensation trap chambers between and in fluid connection with the one or more MS chambers and the one or more pneumatic ports.
Embodiment 166 is the system of any one of embodiments 164-165, wherein:
the one or more MS chambers comprise two or more MS chambers in fluid connection with a one or more input/output valves via one or more fluid channels between the two or more MS chambers and the one or more input/output valves;
each MS chamber of the two or more MS chambers is in fluid connection with a separate pneumatic port; and optionally, each MS chamber of the two or more MS chambers is in fluid connection with a separate condensation trap chamber disposed between the MS chamber and the pneumatic port.
Embodiment 167 is the system of any one of embodiments 147-166, wherein the library preparation unit comprises one or more amplification reaction chambers.
Embodiment 168 is the system of embodiment 167, wherein the one or more amplification reaction chambers interface with a heating element on the instrument when the assay cartridge is inserted within the instrument.
Embodiment 169 is the system of any one of embodiments 167-168, wherein the library preparation unit further comprises one or more input/output valves and one or more fluid channels between and in fluid connection with the one or more amplification reaction chambers and the one or more input/output valves.
Embodiment 170 is the system of any one of embodiments 167-169, wherein the library preparation unit further comprises lyophilized amplification reagents in the one or more amplification reaction chambers or in the one or more fluid channels.
Embodiment 171 is the system of any of embodiments 167-170, wherein the library preparation unit further comprises one or more pneumatic ports in fluid connection with the one or more amplification reaction chambers, wherein the one or more pneumatic ports operationally interface with a pneumatic manifold on the instrument when the assay cartridge is inserted within the instrument.
Embodiment 172 is the system of embodiment 171, wherein the library preparation unit further comprises one or more amplification aliquoting chambers between and in fluid connection with the one or more amplification reaction chambers and the one or more input/output valves.
Embodiment 173 is the system of any one of embodiments 171-172, wherein:
   the one or more amplification reaction chambers comprise two or more amplification reaction chambers in fluid connection with a single input/output valve via a single fluid channel between the two or more amplification reaction chambers and the single input/output valve;
   each amplification reaction chamber of the two or more amplification reaction chambers is in fluid connection with a separate pneumatic port; and
   optionally, each amplification reaction chamber of the two or more amplification reaction chambers is in fluid connection with a separate amplification aliquoting chamber disposed between the amplification reaction chamber and the single input/output valve.
174 Embodiment 174 is the system of embodiment 173, wherein:
   the one or more amplification reaction chambers comprise an additional amplification reaction chamber separate from the two or more amplification reaction chambers;
   the additional amplification reaction chamber is in fluid connection with an additional input/output valve via an additional fluid channel between the additional amplification reaction chamber and the additional input/output valve, wherein the additional input/output value and the additional fluid channel is separate from the input/output valve and the fluid channel in fluid connection with the two or more chambers; and
   optionally, is in fluid connection with an additional amplification aliquoting chamber disposed between the additional amplification reaction chamber and the additional input/output valve, wherein the additional amplification aliquoting chamber is separate from the amplification aliquoting chambers in fluid connection with the two or more amplification reaction chambers.
Embodiment 175 is the system of any one of embodiments 147-174, wherein the CC unit comprises one or more CC chambers.
Embodiment 176 is the system of embodiment 175, wherein the one or more CC chambers interface with a heating element on the instrument when the assay cartridge is inserted within the instrument.
Embodiment 177 is the system of any one of embodiments 175-176, wherein the one or more CC chambers interface with a magnetic element on the instrument when the assay cartridge is inserted within the instrument.
Embodiment 178 is the system of any one of embodiments 175-177, wherein the CC unit further comprises an input/output valve and a fluid channel between and in fluid connection with the one or more CC chambers and the input/output valve.
Embodiment 179 is the system of any one of embodiments 175-178, wherein the CC unit further comprises one or more pneumatic ports in fluid connection with the one or more CC chambers, wherein the one or more pneumatic ports operationally interface with a pneumatic manifold on the instrument when the assay cartridge is inserted within the instrument.
Embodiment 180 is the system of embodiment 179, wherein the CC unit further comprises one or more condensation trap chambers between and in fluid connection with the one or more CC chambers and the one or more pneumatic ports.
Embodiment 181 is the system of any one of embodiments 179-180, wherein:
   the one or more CC chambers comprise two or more CC chambers in fluid connection with a single input/output valve via a single fluid channel between the two or more CC chambers and the input/output valve;
   each CC chamber of the two or more CC chambers is in fluid connection with a separate pneumatic port; and
   optionally, each CC chamber of the two or more CC chambers is in fluid connection with a separate condensation trap chamber disposed between the CC chamber and the pneumatic port.
Embodiment 182 is the system of any one of embodiments 147-181, wherein the cluster generation/sequencing unit comprises a flow cell capable of delivering fluids to and removing fluids from a solid support, further wherein at the surface of the solid support is within the boundaries of the flow cell and in contact with the fluids within the flow cell.
Embodiment 183 is the system of embodiment 182, wherein the flow cell is in fluid connection with one or more input/output valves.
Embodiment 184 is the system of any one of embodiments 182-183, wherein the flow cell is in fluid connection with one or more sequencing-manifold ports, wherein the one or more sequencing-manifold ports interface with a sequencing manifold on the instrument when the assay cartridge is inserted within the instrument.
Embodiment 185 is the system of any one of embodiments 182-184, wherein the flow cell or solid support interface with a heating element on the instrument when the assay cartridge is inserted within the instrument.
Embodiment 186 is the system of any one of embodiments 185, wherein the solid support further comprises an integrated heating element.
Embodiment 187 is the system of any one of embodiments 182-184, wherein the solid support further comprises an integrated heating element.
Embodiment 188 is the system of any one of embodiments 182-187, wherein the flow cell is operationally connected to an semiconductor chip and further wherein at least a portion of the surface of the chip is in contact with at least a portion of the liquid within the flow cell.
Embodiment 189 is the method of embodiment 188, wherein the surface of the semiconductor chip further comprise an array of 3-dimensional features.
Embodiment 190 is the method of embodiment 189, wherein the 3-dimensional features comprise wells.
Embodiment 191 is the method of any one of embodiments 188-190, wherein the semiconductor chip further comprises an array of ISFET sensors.
Embodiment 192 is the system of any one of embodiments 147-191, further comprising a fluid channel and in fluid connection between at least one chamber in the cartridge and at least one other chamber in the cartridge.
Embodiment 193 is the system of any one of embodiments 147-192, further comprising a fluid channel and in fluid connection between at least one chamber other than a waste chamber in the cartridge and at least one waste chamber in the cartridge.
Embodiment 194 is the system of embodiment 193, further comprising an input/output valve, a fluid channel between and in fluid connection with the at least one chamber other than a waste chamber and the input/output valve and a fluid channel between and in fluid connection with the at least one waste chamber and the input/output valve.
Embodiment 195 is the system of any one of embodiments 147-191, further comprising a reagent cartridge removably insertable within the instrument.
Embodiment 196 is the system of embodiment 192, wherein the reagent cartridge comprises one or more reagent chambers in fluid connection with one or more input/output valves.
Embodiment 197 is the system of any one of embodiments 147-193, wherein one, some, or each of the input/output valves are a sealing pneumatic/pipette interface (SPI) valve.
Embodiment 198 is the system of any one of embodiments 147-194, wherein one, some, or each of the chambers are in fluid connection with an SPI valve.
Embodiment 199 is the system of any one of embodiments 147-195, wherein the sample input is in fluid connection with an SPI valve.
Embodiment 200 is the system of any one of claims embodiments 194-196, wherein the SPI valve comprises a flexible, solid valve body comprising a separable portion that separates and seals around a pipette tip to maintain a pressure differential on opposite sides of the valve when the pipette tip is inserted therethrough and seals upon itself to maintain a pressure differential on opposite sides of the valve when the pipette tip is removed therefrom.
Embodiment 201 is the system of any one of embodiments 147-200, wherein the instrument comprises one or more of the following in any combination:
   an assay cartridge interface;
   a reagent cartridge interface;
   a thermal device and interface
   a magnet device and interface
   a mechanical lysis device and interface
   a sonication device and interface
   an assay cartridge fluidic manifold;
   a reagent cartridge fluidic manifold;
   a pneumatic device and interface
   a solid support interface;
   one or more sensors;
   one or more CPU's and associated devices and electronics; and
   a user interface
Embodiment 202 is the system of any one of embodiments 147-201, wherein the instrument comprises a liquid handler comprising a pipette configured to transfer material from each input/output valve.
Embodiment 203 is the system of embodiment 202, wherein the instrument comprises at least one robotic gantry arm capable of motion in the x, y and z dimensions.
Embodiment 204 is a method of analyzing a target polynucleotide in a sample as claimed in any one of embodiments 131-146 in the system of any one of embodiments 147-203 claims, the method comprising any one or more, in any combination, of:
   a. processing a sample in any one of or any combination thereof (including all) of the sample input, incubation, lysis, copy control and/or MS units to obtain a prepared target polynucleotide;
   b. amplifying in the library preparation unit a portion of the prepared target polynucleotide to obtain amplified target nucleic acid;
   c. controlling the number of output copies in the copy control unit;
   d. generating one or more clusters in the cluster generation/sequencing unit;
   e. sequencing in the cluster generation/sequencing unit the target nucleic acid prepared in any one or any combination of steps a) through d).
Embodiment 204 is a method of analyzing a target polynucleotide in a sample as claimed in any one of embodiments 131-146 in the system of any one of embodiments 147-203 claims, the method comprising any one or more, in any combination, of:
   a) processing a sample in any one of or any combination thereof (including all) of the sample input, incubation, lysis, copy control and/or MS units to obtain a prepared target polynucleotide;
   b) amplifying in the library preparation unit a portion of the prepared target polynucleotide to obtain amplified target nucleic acid;
   c) controlling the number of output copies in the copy control unit;
   d) generating one or more clusters in the cluster generation/sequencing unit;
   e) sequencing in the cluster generation/sequencing unit the target nucleic acid prepared in any one or any combination of steps a) through d).
Embodiment 205 is the method of embodiment 204, wherein the preparing the target polynucleotide in a sample comprises the method of any one of embodiments 1-34.
Embodiment 206 is the method of any one of embodiments 204-205, wherein the preparing the target polynucleotide in a sample comprises:
   inputting a sample in the sample input unit;
   transferring the sample to at least one incubation chamber in the incubation unit, wherein at least one incubation chamber is pre-loaded with lysis reagents;
   mixing the sample with the lysis reagents to generate a sample-lysis reagent mixture; heating the sample-lysis reagent mixture in at least one incubation chamber;
   transferring the sample-lysis reagent mixture from the at least one incubation chamber to at least one lysis chamber in the lysis unit, wherein at least one lysis chamber is pre-loaded with additional lysis reagents;
   lysing cells present in the sample-lysis reagent mixture to generate a lysate; passing the lysate from the at least one lysis chamber through one or more first MS chambers with at least one heating element engaged and into one or more second MS chambers, wherein one or more second MS chambers is pre-loaded with target capture reagents comprising at least one capture oligonucleotide;
   mixing the lysate with the target capture reagents to create a lysate-target capture reagent mixture;
   transferring the lysate-target capture reagent mixture to at least one incubation chamber;
   heating the lysate-target capture reagent mixture in at least one incubation chamber to permit annealing of the capture oligonucleotides to target polynucleotide to generate a capture oligomer-target complex;
   transferring the capture oligomer-target complex into one or more third MS chambers, wherein one or more third MS chambers is pre-loaded with capture beads (e.g., magnetic capture beads)
   mixing the capture oligomer-target complex and capture beads to generate a capture oligomer-target-capture bead complex;
   immobilizing the capture beads by passing the capture oligomer-target-capture bead complex through one or more first MS chambers with at least one magnetic element engaged, removing the lysate from the immobilized capture beads, adding wash buffer to and then removing same from the immobilized capture beads to wash the immobilized capture beads, and adding elution buffer to the immobilized capture beads to elute target polynucleotide from the immobilized capture beads to generate the prepared target polynucleotide.
Embodiment 207 is the method of embodiment 206, wherein transferring comprises passage through an input/output valve.
Embodiment 208 is the method of any one of embodiments 206-207, wherein mixing comprises selectively pressurizing and depressurizing two or more chambers via two or more pneumatic ports in separate fluid connections thereto.
Embodiment 209 is the method of any one of the embodiments 206-208, wherein pre-loaded reagents comprise lyophilized reagents.
Embodiment 210 is the method of any one of embodiments 206-209, wherein the lysis reagent comprises Proteinase K.
Embodiment 211 is the method of any one of embodiments 206-210 wherein heating the sample-lysis reagent mixture in at least one incubation chamber comprises heating at about 60° C.
Embodiment 212 is the method of any one of embodiments 206-211, wherein the additional lysis reagents comprise zirconium beads.
Embodiment 213 is the method of any one of embodiments 206-212, wherein lysing cells present in the sample-lysis reagent mixture comprises mixing using a spinning paddle or impeller.
Embodiment 214 is the method of any one of embodiments 206-213, wherein the one or more first MS chambers comprises a serpentine channel.
Embodiment 215 is the method of any one of embodiments 206-214, wherein passing the lysate from one or more lysis chambers through one or more MS chambers comprises heating at about 95° C.
Embodiment 216 is the method of embodiment 215, wherein heating comprises denaturing double stranded nucleic acid if present.
Embodiment 217 is the method of any one of embodiments 206-216, wherein heating the lysate-target capture reagent mixture in at least one incubation chamber comprises heating at about 60° C.
Embodiment 218 is the method of any one of embodiments 204-217, wherein the amplifying comprises the method of any one of claims 2.1-2.26.
Embodiment 219 is the method of any one of embodiments 204-218, wherein the amplifying comprises:
   transferring the isolated target nucleic acid from one or more STC chambers to a first of the one or more PCR chambers;
   generating a first target nucleic acid amplicon in the first of the one or more PCR chambers;
   transferring the first target nucleic acid amplicon from the first of the one or more PCR chambers and to a second of the one or more PCR chambers;
   optionally diluting the first target nucleic acid amplicon; and
   generating a second target nucleic acid amplicon in the second of the one or more PCR chambers.
Embodiment 220 is the method of any one of embodiments 204-219, further comprising:
   transferring the amplified target nucleic acid from the PCR unit to the CC unit; and
   controlling, in the CC unit, the copy number of the amplified target nucleic acid to obtain about a pre-determined copy level of the target nucleic acid therein.
Embodiment 221 is the method of embodiment 220, wherein the controlling the copy number comprises the method of any one of embodiments 66-75.
Embodiment 222 is the method of any one of embodiments 204-221, further comprising transferring the target nucleic acid from any of the other units in the assay cartridge to the cluster generation/sequencing unit and generating clusters of target nucleic acid immobilized on a surface within the boundaries of the flow cell in the cluster generation/sequencing unit.
Embodiment 223 is the method of embodiment 222, wherein the generating the clusters comprises the method of any one of embodiments 76-130.
Embodiment 224 is the method of any one of embodiments 204-223, wherein all processes are conducted within the system in an automated fashion in a continuous workflow with no human intervention after initial introduction of the sample and the run is started.
Embodiment 225 is a method of analyzing a target polynucleotide in a sample as claimed in any one of embodiments 131-146 in the system of any one of embodiments 147-203, wherein the target polynucleotide is analyzed directly from sample.
Embodiment 226 is an instrument comprising any one or more, in any combination, of:
   a cartridge interface;
   a thermal device and interface;
   a magnet device and interface;
   a mechanical lysis device and interface;
   a sonication device and interface;
   a cartridge fluidic manifold;
   a pneumatic device and interface;
   a solid support interface;
   one or more sensors;
   one or more CPU's and associated devices and electronics; and
   a user interface
Embodiment 227 is one or more cartridges comprising any one or more, in any combination, of:
   a sample input unit;
   an incubation unit;
   a lysis unit;
   a magnetic separation]unit (MSU);
   a library preparation unit;
   a copy control (CC) unit;
   a cluster generation/sequencing unit;
   a liquid waste unit;
   a dry reagent storage unit;
   a liquid reagent storage unit for assay-specific reagents;
   a liquid reagent storage unit for bulk and sequencing-specific reagents; and
   a liquid waste unit

What is claimed is:

1. A method for analyzing a target in a sample, the method comprising:
introducing a sample to a cartridge;
introducing the cartridge to an instrument operable to manipulate the sample within the cartridge to automatically:
isolate a target nucleic acid from the sample;
amplify the isolated target nucleic acid; and
sequence the amplified target nucleic acid using next generation sequencing,
wherein the sample remains within the cartridge throughout the isolating, amplifying, and sequencing steps.

2. The method of claim 1 wherein the isolating, amplifying, and sequencing steps are performed within 8 or less hours after introducing the sample to the cartridge.

3. The method of claim 1 wherein the target nucleic acid comprises fungal nucleic acid present in the sample at levels as low as 3 copies.

4. The method of claim 1 wherein the target nucleic acid comprises bacterial nucleic acid present in the sample at levels as low as 3 copies.

5. The method of claim 1 wherein the target nucleic acid comprises viral nucleic acid present in the sample at levels as low as a single copy.

6. The method of claim 1 wherein the cartridge has an exterior volume of about 3 liters or less.

7. The method of claim 6 wherein the cartridge has an exterior volume of about 2.5 liters or less.

8. The method of claim 7 wherein the cartridge has an exterior volume of about 2.1 liters or less.

9. The method of claim 6 wherein the cartridge has a longest linear dimension of about 200 mm or less.

10. The method of claim 9 wherein the cartridge has a longest linear dimension of about 160 mm or less.

11. The method of claim 1 wherein the sample is selected from the group consisting of a biological sample, a clinical sample, an environmental sample, and a food sample.

12. The method of claim 11 wherein the sample is a biological sample obtained from a subject and untreated prior to introduction to the cartridge.

13. The method of claim 1 wherein the isolating step comprises digesting proteins in the sample.

14. The method of claim 13 comprising digesting the proteins using proteinase K.

15. The method of claim 1 wherein the isolating step comprises lysing an organism to release the target nucleic acid.

16. The method of claim 15 wherein lysing comprises mechanical lysis.

17. The method of claim 16 wherein the mechanical lysis comprises flowing the sample into a lysis chamber within the cartridge and rotating a paddle within the lysis chamber.

18. The method of claim 17 wherein the mechanical lysis further comprises adding zirconium beads to the lysis chamber before rotating the paddle within the lysis chamber.

19. The method of claim 1 wherein the isolating step comprises denaturing the target nucleic acid.

20. The method of claim 19 wherein denaturing comprises thermal denaturing.

21. The method of claim 1 wherein the isolating step comprises capturing the target nucleic acid by:
annealing target capture oligonucleotides to the target nucleic acid to form a complex;
binding the complex on a solid support; and
removing unbound material from the solid support.

22. The method of claim 21 wherein removing unbound material comprises washing the solid support bound complex with a wash reagent.

23. The method of claim 21 wherein the amplifying step is performed on the solid support-bound target nucleic acid.

24. The method of claim 21 further comprising eluting the target nucleic acid from the washed solid support to prepare the isolated target nucleic acid.

25. The method of claim 23 wherein the amplifying step is performed directly on the eluted target nucleic acid without intervening steps.

26. The method of claim 20 wherein the isolating step automatically isolates the target nucleic acid from a sample having a volume between about 1 mL and about 25 mL.

27. The method of claim 1 wherein the isolating step comprises only one purification step.

28. The method of claim 1 wherein the isolated nucleic acid is amplified without quantification.

29. The method of claim 1 wherein the amplifying step comprises:
performing a first amplification of the isolated target nucleic acid using a first primer set to produce a first amplification product;
diluting the first amplification product and aliquoting into a plurality of aliquots;
performing a second amplification of the target nucleic acid in the plurality of aliquots using a plurality of second primer sets to produce a plurality of second amplification products; and
pooling the second amplification products.

30. The method of claim 29 wherein one or more primers in the first primer set are identical to one or more primers in the plurality of second primer sets.

31. The method of claim 30 wherein the amplifying step further comprises purifying the pooled second amplification products to produce the amplified target nucleic acid.

32. The method of claim 30 wherein one or more of the first and second amplifications comprise PCR amplification.

33. The method of claim 30 wherein the plurality of aliquots comprises at least 10 separate aliquots.

34. The method of claim 30 wherein the first PCR amplification and second PCR amplification are performed without quantification.

35. The method of claim 30 wherein one or more of the plurality of second primer sets are nested relative to the first primer set.

36. The method of claim 1 wherein the amplifying step comprises performing copy control on the amplified target nucleic acid before the sequencing step.

37. The method of claim 1 wherein the amplifying step comprises only one purification step.

38. The method of claim 1 wherein the amplified target nucleic acid is sequenced without quantification.

39. The method of claim 1 wherein the sequencing step comprises:
immobilizing the amplified target nucleic acid above a semiconductor surface within the cartridge comprising an ion-sensitive field-effector transistor (ISFET) sensor.

40. The method of claim 39 wherein all products of the amplifying step are flowed over the semiconductor surface without intervening steps.

41. The method of claim 39 wherein the amplified target nucleic acid is immobilized by a capture oligomer bound above the ISFET sensor wherein said capture oligomer hybridizes to a portion of the target nucleic acid.

42. The method of claim 41 wherein the surface comprises an array of ISFET sensors each with a well positioned above it.

43. The method of claim 41 wherein at least one of the wells is positioned above a plurality of ISFET sensors in the array of ISFET sensors.

44. The method of claim 42 wherein one or more of the wells comprise a surface-bound forward primer that hybridizes to a portion of the target nucleic acid and a surface-bound reverse primer that hybridizes to a portion of the target nucleic acid, and wherein the sequencing step comprises paired-end sequencing.

45. The method of claim 42 wherein one or more of the wells or an interstitial space between one or more of the wells comprises a plurality of bound inert oligomers that do not hybridize to the target nucleic acid.

46. The method of claim 39 wherein the amplified target nucleic acid is immobilized by a universal capture oligomer bound above the ISFET sensor wherein said universal capture oligomer hybridizes to a universal binding site.

47. The method of claim 46 wherein the amplifying step comprises amplifying the isolated target nucleic acid using a primer comprising the universal binding site.

48. The method of claim 46 wherein the amplifying step comprises ligating an adapter onto the isolated target nucleic acid, said adapter comprising the universal binding site.

49. The method of claim 46 wherein the sequencing step comprises clonal amplification of the immobilized target nucleic acid.

50. The method of claim 49 wherein the clonal amplification comprises recombinase polymerase amplification.

51. The method of claim 49 wherein the clonal amplification comprises rolling circle amplification.

52. The method of claim 49 wherein the clonal amplification comprises bridge PCR, strand displacement amplification, or loop-mediated isothermal amplification.

53. A system comprising:
a sample cartridge comprising:
a sample input;
a sample preparation unit operable to receive sample from the sample input and isolate a target nucleic acid from the sample;
a library preparation unit operable to receive the isolated target nucleic acid from the sample preparation unit and amplify the isolated target nucleic acid; and
a sequencing unit operable to receive the amplified target nucleic acid from the library preparation unit and sequence the amplified target nucleic acid; and
an instrument comprising:
a cartridge interface comprising physical and electronic connections through which the instrument is operable to drive movement of the sample and reagents within the cartridge and to communicate with the sequencing units.

54. The system claim 53 wherein one or more reagents required for isolating the target nucleic acid, amplifying the isolated target nucleic acid, and sequencing the amplified nucleic acid are dried reagents, the instrument operable to reconstitute the one or more reagents.

55. The system of claim 53 further comprising one or more reagent cartridges containing one or more reagents required for isolating the target nucleic acid, amplifying the isolated target nucleic acid, and sequencing the amplified nucleic acid;
the instrument operable to transfer reagents from the one or more reagent cartridges to the sample cartridge.

56. The system of claim 55 wherein the sample cartridge and the one or more reagent cartridges comprise sealing pneumatic interface (SPI) ports, and
wherein the instrument is operable to transfer the one or more reagents via the SPI ports from the one or more reagent cartridges to the sample cartridge using one or more pipettes.

57. The system of claim 56 wherein the instrument comprises a 3-degree-of-freedom pipette gantry operable to transfer the one or more reagents.

58. The system of claim 53 operable to isolate, amplify, and sequence a target fungal nucleic acid in the sample at levels as low as 3 copies.

59. The system of claim 53 operable to isolate, amplify, and sequence a target bacterial nucleic acid present in the sample at levels as low as 3 copies.

60. The system of claim 53 operable to isolate, amplify, and sequence a target viral nucleic acid present in the sample at levels as low as a single copy.

61. The system of claim 53 wherein the cartridge has an exterior volume of about 3 liters or less.

62. The system of claim 61 wherein the cartridge has an exterior volume of about 2.5 liters or less.

63. The system of claim 62 wherein the cartridge has an exterior volume of about 2.1 liters or less.

64. The system of claim 61 wherein the cartridge has a longest linear dimension of about 200 mm or less.

65. The system of claim 64 wherein the cartridge has a longest linear dimension of about 160 mm or less.

66. The system of claim 53 wherein the instrument has a volume of about 150 liters or less.

67. The system of claim 66 wherein the instrument has a volume of about 135 liters or less.

68. The system of claim 53 wherein the instrument has a longest linear dimension of about 700 mm or less.

69. The system of claim 68 wherein the instrument has a longest linear dimension of about 650 mm or less.

70. The system of claim 53 wherein the sample cartridge is operable to receive biological, clinical, environmental, and food samples.

71. The system of claim 53 wherein the sample cartridge is operable to receive untreated biological samples.

72. The system of claim 53 wherein isolating the target nucleic acid comprises digesting proteins in the sample.

73. The system of claim 72 wherein the instrument is operable to expose the sample to proteinase K in the sample preparation unit.

74. The system of claim 53 wherein the sample preparation unit is operable to lyse an organism to release the target nucleic acid.

75. The system of claim 74 wherein the sample preparation unit comprises a lysis chamber comprising a rotating paddle, the instrument operable to flow the sample into the lysis chamber and interface with the sample cartridge to rotate the rotating paddle to mechanically lyse organisms in the sample.

76. The system of claim 75 further comprising zirconium beads in the lysis chamber.

77. The system of claim 53 wherein isolating the target nucleic acid comprises denaturing the target nucleic acid.

78. The system of claim 77 wherein the instrument is operable to provide thermal energy to the sample preparation unit to denature nucleic acid therein.

79. The system of claim 53 wherein the instrument is operable to:
expose the sample to target capture oligonucleotides and a solid support in the sample preparation unit to anneal the target capture oligonucleotides to the target nucleic acid to form a complex and bind the complex to the solid support.

80. The system of claim 79 wherein the instrument is further operable to introduce a wash buffer to the solid support bound complexes and separate the solid support bound complexes from unbound sample.

81. The system of claim 80 wherein the instrument is operable to transfer the separated solid support bound complexes to the library preparation unit and amplify the solid support bound target nucleic acid.

82. The system of claim 79 wherein the instrument is operable to introduce elution buffer to the separated solid support bound complexes to elute the target nucleic acid from the solid support and transfer the eluted target nucleic acids to the library preparation unit for amplification.

83. The system of claim 79 wherein the instrument is operable to introduce amplification reagents to the solid support bound complexes and amplify the target nucleic acid within the sample preparation unit.

84. The system of claim 53 operable to automatically accommodate a sample having a volume between about 1 mL and about 25 mL received through the sample input.

85. The system of claim 53 wherein the instrument is operable to interface with the library preparation unit of the sample cartridge to introduce required reagents and provide thermal energy to:
perform a first amplification of the isolated target nucleic acid using a first primer set to produce a first amplification product;
dilute the first amplification product and aliquot it into a plurality of aliquots;
perform a second amplification of the target nucleic acid in the plurality of aliquots using a plurality of second primer sets to produce a plurality of second amplification products; and
pool the second amplification products.

86. The system of claim 85 wherein one or more primers in the first primer set are identical to one or more primers in the plurality of second primer sets.

87. The system of claim 85 wherein the instrument is further operable to purify the pooled second amplification products to produce the amplified target nucleic acid.

88. The system of claim 85 wherein one or more of the first and second amplifications comprise PCR amplification.

89. The system of claim 85 wherein the plurality of aliquots comprises at least 10 separate aliquots.

90. The system of claim 53 further operable to perform a copy control on one or more of the isolated target nucleic acid and the amplified target nucleic acid and control a number of output copies transferred to the library preparation unit or the sequencing unit respectively.

91. The system of claim 53 wherein the sequencing unit comprises a semiconductor surface comprising an array of ion-sensitive field-effector transistor (ISFET) sensors each with a well positioned above it, the instrument operable to immobilize the amplified target nucleic acid above the array of ISFET sensors, the array of ISFET sensors in electronic communication with the instrument through the electronic connections of the cartridge interface when a sample cartridge is positioned therein.

92. The system of claim 91 wherein the instrument is operable to flow all output from the library preparation unit into the wells over the semiconductor surface.

93. The system of claim 91 comprising a capture oligomer bound above the array of ISFET sensors wherein said capture oligomer is configured to hybridize to a portion of the target nucleic acid.

94. The system of claim 91 wherein at least one of the wells is positioned above a plurality of ISFET sensors in the array of ISFET sensors.

95. The system of claim 91 wherein one or more of the wells comprise a surface bound forward primer that hybridizes to a portion of the target nucleic acid and a surface bound reverse primer that hybridizes to a portion of the target nucleic acid, the instrument operable to perform paired-end sequencing.

96. The system of claim 91 wherein one or more of the wells and interstitial space between the wells comprises a plurality of bound inert oligomers that do not hybridize to the target nucleic acid.

97. The system of claim 91 comprising a universal capture oligomer bound above the array of ISFET sensors wherein said universal capture oligomer is configured to hybridize to a universal binding site.

98. The system of claim 97 wherein the instrument is operable to interface with the library preparation unit to amplify the isolated target nucleic acid using a primer comprising the universal binding site.

99. The system of claim 97 wherein the instrument is operable to ligate an adapter onto the isolated target nucleic acid in the sample preparation unit or the library preparation unit, said adapter comprising the universal binding site.

100. The system of claim 97 wherein the instrument is operable to interface with the sequencing unit to perform clonal amplification of the immobilized target nucleic acid.

101. The system of claim 100 wherein the clonal amplification comprises recombinase polymerase amplification.

102. The system of claim 100 wherein the clonal amplification comprises rolling circle amplification.

103. The system of claim 100 wherein the clonal amplification comprises bridge PCR, strand displacement amplification, or loop-mediated isothermal amplification.

104. The system of claim 53 wherein the physical and electrical connections comprise a pneumatic system for driving fluid movement within the sample cartridge.

105. The system of claim 53 wherein the cartridge interface further comprises physical and electronic connections through which the instrument is operable communicate with one or more of the sample preparation unit and the library preparation unit.


SEQUENCE LISTING (218 sequences)

SEQ ID NO: 1 - DNA, 22 bp, synthetic construct
  source 1..22: mol_type = other DNA
acttgcatgtgttaggcatgcc

SEQ ID NO: 2 - DNA, 15 bp, synthetic construct
  source 1..15: mol_type = other DNA
ggcatgcctaacaca

SEQ ID NO: 3 - DNA, 22 bp, synthetic construct
  source 1..22: mol_type = other DNA
acttgcatgtgttaggcatgcc

SEQ ID NO: 4 - DNA, 18 bp, synthetic construct
  source 1..18: mol_type = other DNA
ccgggcatgcctaacaca

SEQ ID NO: 5 - DNA, 15 bp, synthetic construct
  source 1..15: mol_type = other DNA
ggcatgcctaacaca

SEQ ID NO: 6 - DNA, 25 bp, synthetic construct
  source 1..25: mol_type = other DNA
  modified_base <1: mod_base = OTHER; note = Biotinylated 5' end
  modified_base 25: mod_base = OTHER; note = Inverted dT
aaaaacaccgcgctgaccaacctct

SEQ ID NO: 7 - DNA, 26 bp, synthetic construct
  source 1..26: mol_type = other DNA
  modified_base <1: mod_base = OTHER; note = Biotinylated 5' end
  modified_base 26: mod_base = OTHER; note = Inverted dT
aaaaacacagcggcagcaagaaagct

SEQ ID NO: 8 - DNA, 34 bp, synthetic construct
  source 1..34: mol_type = other DNA
  modified_base <1: mod_base = OTHER; note = Biotinylated 5' end
  modified_base 34: mod_base = OTHER; note = Inverted dT
aaaaaaggtactgctatccaccctcaaacaggtt

SEQ ID NO: 9 - DNA, 37 bp, synthetic construct
  source 1..37: mol_type = other DNA
  modified_base <1: mod_base = OTHER; note = Biotinylated 5' end
  modified_base 37: mod_base = OTHER; note = Inverted dT
aaaaattgagttgaacctggtgaagttgtaatctggt

SEQ ID NO: 10 - DNA, 24 bp, synthetic construct
  source 1..24: mol_type = other DNA
aaccattcgctaaactcgaacagg

SEQ ID NO: 11 - DNA, 22 bp, synthetic construct
  source 1..22: mol_type = other DNA
ccttgaatgagctgcacagtgg

SEQ ID NO: 12 - DNA, 27 bp, synthetic construct
  source 1..27: mol_type = other DNA
catgaaaaatgattatggctcaggtac

SEQ ID NO: 13 - DNA, 23 bp, synthetic construct
  source 1..23: mol_type = other DNA
tggaacttgttgagcagaggttc

SEQ ID NO: 14 - DNA, 18 bp, synthetic construct
  source 1..18: mol_type = other DNA
ctttggcggctccatcgg

SEQ ID NO: 15 - DNA, 21 bp, synthetic construct
  source 1..21: mol_type = other DNA
ctcctcagcgcggtaacttac

SEQ ID NO: 16 - DNA, 25 bp, synthetic construct
  source 1..25: mol_type = other DNA
gctatccaccctcaaacaggtgaat

SEQ ID NO: 17 - DNA, 26 bp, synthetic construct
  source 1..26: mol_type = other DNA
attcttcgttactcatgccatacata

SEQ ID NO: 18 - DNA, 48 bp, synthetic construct
  source 1..48: mol_type = other DNA
aaaacgagacatgccgagcatccgctttaagtcccgcaacgagcgcaa

SEQ ID NO: 19 - DNA, 51 bp, synthetic construct
  source 1..51: mol_type = other DNA
  modified_base <1: mod_base = OTHER; note = Two Biotin groups on 5' end
accgtgctgccttggcttcattgtggtcttgacgtcatccccaccttcctc

SEQ ID NO: 20 - DNA, 48 bp, synthetic construct
  source 1..48: mol_type = other DNA
aaaacgagacatgccgagcatccgccgcatgtgtaggataggtgggag

SEQ ID NO: 21 - DNA, 48 bp, synthetic construct
  source 1..48: mol_type = other DNA
aaaacgagacatgccgagcatccgccgcatgtacaggataggtaggag

SEQ ID NO: 22 - DNA, 20 bp, synthetic construct
  source 1..20: mol_type = other DNA
  modified_base <1: mod_base = OTHER; note = Two Biotin groups on 5' end
gagaccgccccagtcaaact

SEQ ID NO: 23 - DNA, 45 bp, synthetic construct
  source 1..45: mol_type = other DNA
  modified_base <1: mod_base = OTHER; note = Two Biotin groups on 5' end
aaaacgagacatgccgagcatccgctgttaggaagtgtgccgctg

SEQ ID NO: 24 - DNA, 47 bp, synthetic construct
  source 1..47: mol_type = other DNA
accgtgctgccttggcttcattgtggtctcccgactgcygctctaat

SEQ ID NO: 25 - DNA, 21 bp, synthetic construct
  source 1..21: mol_type = other DNA
tgtagcggtgaaatgcgyaga

SEQ ID NO: 26 - DNA, 22 bp, synthetic construct
  source 1..22: mol_type = other DNA
cggtcgacttaacgcgttagct

SEQ ID NO: 27 - DNA, 22 bp, synthetic construct
  source 1..22: mol_type = other DNA
cggagtgcttaatgcgttwgct

SEQ ID NO: 28 - DNA, 26 bp, synthetic construct
  source 1..26: mol_type = other DNA
cgcaaggttgaaactcaaaggaattg

SEQ ID NO: 29 - DNA, 27 bp, synthetic construct
  source 1..27: mol_type = other DNA
ccgcaaggttaaaactcaaatgaattg

SEQ ID NO: 30 - DNA, 22 bp, synthetic construct
  source 1..22: mol_type = other DNA
gggacttaacccaacatytcac

SEQ ID NO: 31 - DNA, 20 bp, synthetic construct
  source 1..20: mol_type = other DNA
cctggctcagaatgaacgct

SEQ ID NO: 32 - DNA, 20 bp, synthetic construct
  source 1..20: mol_type = other DNA
cctggctcaggacgaacgct

SEQ ID NO: 33 - DNA, 21 bp, synthetic construct
  source 1..21: mol_type = other DNA
gagtctggaccgtgtctcagt

SEQ ID NO: 34 - DNA, 21 bp, synthetic construct
  source 1..21: mol_type = other DNA
gagtctgggccgtgtctcagt

SEQ ID NO: 35 - DNA, 23 bp, synthetic construct
  source 1..23: mol_type = other DNA
cgtgtgtagcccaggtcataagg

SEQ ID NO: 36 - DNA, 25 bp, synthetic construct
  source 1..25: mol_type = other DNA
cacgtgtgtagcccaaatcataagg

SEQ ID NO: 37 - DNA, 21 bp, synthetic construct
  source 1..21: mol_type = other DNA
tgtgtagccctggtcgtaagg

SEQ ID NO: 38 - DNA, 22 bp, synthetic construct
  source 1..22: mol_type = other DNA
tcagctcgtgtcgtgagatgtt

SEQ ID NO: 39 - DNA, 24 bp, synthetic construct
  source 1..24: mol_type = other DNA
cgtcagctcgtgttgtgaaatgtt

SEQ ID NO: 40 - DNA, 20 bp, synthetic construct
  source 1..20: mol_type = other DNA
ctcctacgggaggcagcagt

SEQ ID NO: 41 - DNA, 20 bp, synthetic construct
  source 1..20: mol_type = other DNA
cctccgtattaccgcggctg

SEQ ID NO: 42 - DNA, 23 bp, synthetic construct
  source 1..23: mol_type = other DNA
gaaagaccccgtgaacctttact

SEQ ID NO: 43 - DNA, 23 bp, synthetic construct
  source 1..23: mol_type = other DNA
gaaagaccccgtggagctttact

SEQ ID NO: 44 - DNA, 20 bp, synthetic construct
  source 1..20: mol_type = other DNA
ccttcgtgctcctccgttac

SEQ ID NO: 45 - DNA, 20 bp, synthetic construct
  source 1..20: mol_type = other DNA
cctttgagcgcctccgttac

SEQ ID NO: 46 - DNA, 25 bp, synthetic construct
  source 1..25: mol_type = other DNA
acacaggtctctgctaaaccgtaag

SEQ ID NO: 47 - DNA, 25 bp, synthetic construct
  source 1..25: mol_type = other DNA
acacaggtctctgcaaaatcgtaag

SEQ ID NO: 48 - DNA, 25 bp, synthetic construct
  source 1..25: mol_type = other DNA
acacagcactgtgcaaacacgaaag

SEQ ID NO: 49 - DNA, 24 bp, synthetic construct
  source 1..24: mol_type = other DNA
tacccgacaaggaatttcgctacc

SEQ ID NO: 50 - DNA, 22 bp, synthetic construct
  source 1..22: mol_type = other DNA
tggcagcttcactttctcttgc

SEQ ID NO: 51 - DNA, 21 bp, synthetic construct
  source 1..21: mol_type = other DNA
ccagctccaatcacaccaaca

SEQ ID NO: 52 - DNA, 20 bp, synthetic construct
  source 1..20: mol_type = other DNA
cagctgctgcagtggatggt

SEQ ID NO: 53 - DNA, 19 bp, synthetic construct
  source 1..19: mol_type = other DNA
ccggsgtatcccgcagata

SEQ ID NO: 54 - DNA, 24 bp, synthetic construct
  source 1..24: mol_type = other DNA
aaccattcgctaaactcgaacagg

SEQ ID NO: 55 - DNA, 22 bp, synthetic construct
  source 1..22: mol_type = other DNA
ccttgaatgagctgcacagtgg

SEQ ID NO: 56 - DNA, 23 bp, synthetic construct
  source 1..23: mol_type = other DNA
gccgtaatagtacctggtttgaa

SEQ ID NO: 57 - DNA, 22 bp, synthetic construct
  source 1..22: mol_type = other DNA
gccyttygggtgttttgttagg

SEQ ID NO: 58 - DNA, 24 bp, synthetic construct
  source 1..24: mol_type = other DNA
tctgcaacaccaatccttataacg

SEQ ID NO: 59 - DNA, 26 bp, synthetic construct
  source 1..26: mol_type = other DNA
catcatatcgcttaaaatacgcaggc

SEQ ID NO: 60 - DNA, 20 bp, synthetic construct
  source 1..20: mol_type = other DNA
agattgccgagcgacttggc

SEQ ID NO: 61 - DNA, 20 bp, synthetic construct
  source 1..20: mol_type = other DNA
caactttggcccgctcaagg

SEQ ID NO: 62 - DNA, 24 bp, synthetic construct
  source 1..24: mol_type = other DNA
acagaatatgtgccagcctctaca

SEQ ID NO: 63 - DNA, 25 bp, synthetic construct
  source 1..25: mol_type = other DNA
catggcttctcctagtgtcatgtct

SEQ ID NO: 64 - DNA, 20 bp, synthetic construct
  source 1..20: mol_type = other DNA
gcggtagcaaaggaatggca

SEQ ID NO: 65 - DNA, 19 bp, synthetic construct
  source 1..19: mol_type = other DNA
tgcttggttcgcccgttta

SEQ ID NO: 66 - DNA, 23 bp, synthetic construct
  source 1..23: mol_type = other DNA
aacgaagcacacactacgggtgt

SEQ ID NO: 67 - DNA, 23 bp, synthetic construct
  source 1..23: mol_type = other DNA
tgctcaaggccgatcaaagcatt

SEQ ID NO: 68 - DNA, 22 bp, synthetic construct
  source 1..22: mol_type = other DNA
gcaatgactggaacaaagccta

SEQ ID NO: 69 - DNA, 20 bp, synthetic construct
  source 1..20: mol_type = other DNA
accagcatgtaacgcagcga

SEQ ID NO: 70 - DNA, 27 bp, synthetic construct
  source 1..27: mol_type = other DNA
catgaaaaatgattatggctcaggtac

SEQ ID NO: 71 - DNA, 23 bp, synthetic construct
  source 1..23: mol_type = other DNA
tggaacttgttgagcagaggttc

SEQ ID NO: 72 - DNA, 22 bp, synthetic construct
  source 1..22: mol_type = other DNA
ggctgcgatattcaaagctcag

SEQ ID NO: 73 - DNA, 19 bp, synthetic construct
  source 1..19: mol_type = other DNA
ctgaacgcgccggcttaac

SEQ ID NO: 74 - DNA, 23 bp, synthetic construct
  source 1..23: mol_type = other DNA
gtatggaagctatgcaagaagcc

SEQ ID NO: 75 - DNA, 21 bp, synthetic construct
  source 1..21: mol_type = other DNA
catgcaaaaccgggaaagcca

SEQ ID NO: 76 - DNA, 22 bp, synthetic construct
  source 1..22: mol_type = other DNA
gcggtattatcccgtgttgacg

SEQ ID NO: 77 - DNA, 21 bp, synthetic construct
  source 1..21: mol_type = other DNA
tcactcatggttatggcagca

SEQ ID NO: 78 - DNA, 24 bp, synthetic construct
  source 1..24: mol_type = other DNA
gataaagttgcaggaccacttctg

SEQ ID NO: 79 - DNA, 23 bp, synthetic construct
  source 1..23: mol_type = other DNA
ccccgtcrtgtagataactacga

SEQ ID NO: 80 - DNA, 18 bp, synthetic construct
  source 1..18: mol_type = other DNA
cggcarccgtcacgctgt

SEQ ID NO: 81 - DNA, 25 bp, synthetic construct
  source 1..25: mol_type = other DNA
catcagcacgataaagtatttgcga

SEQ ID NO: 82 - DNA, 18 bp, synthetic construct
  source 1..18: mol_type = other DNA
tgcatgcgcagrcgaaca

SEQ ID NO: 83 - DNA, 22 bp, synthetic construct
  source 1..22: mol_type = other DNA
ccttactggtactgcacatcgc

SEQ ID NO: 84 - DNA, 20 bp, synthetic construct
  source 1..20: mol_type = other DNA
ttgctggtgctgcacatcgc

SEQ ID NO: 85 - DNA, 20 bp, synthetic construct
  source 1..20: mol_type = other DNA
taccaccacgccrttagcga

SEQ ID NO: 86 - DNA, 20 bp, synthetic construct
  source 1..20: mol_type = other DNA
acaacccacgatgtgggtag

SEQ ID NO: 87 - DNA, 22 bp, synthetic construct
  source 1..22: mol_type = other DNA
gtgctttatcgcggtgatgaac

SEQ ID NO: 88 - DNA, 20 bp, synthetic construct
  source 1..20: mol_type = other DNA
gttaaccagatcggcaggct

SEQ ID NO: 89 - DNA, 22 bp, synthetic construct
  source 1..22: mol_type = other DNA
actgtacttgtgcgctatcggt

SEQ ID NO: 90 - DNA, 23 bp, synthetic construct
  source 1..23: mol_type = other DNA
tcctcagtaacggcgagtgaagc

SEQ ID NO: 91 - DNA, 20 bp, synthetic construct
  source 1..20: mol_type = other DNA
ccgtcttgaaacacggacca

SEQ ID NO: 92 - DNA, 24 bp, synthetic construct
  source 1..24: mol_type = other DNA
gtttcctctggcttcaccctattc

SEQ ID NO: 93 - DNA, 20 bp, synthetic construct
  source 1..20: mol_type = other DNA
aacaactcaccggccgaatg

SEQ ID NO: 94 - DNA, 23 bp, synthetic construct
  source 1..23: mol_type = other DNA
atggaacctttccccacttcagt

SEQ ID NO: 95 - DNA, 26 bp, synthetic construct
  source 1..26: mol_type = other DNA
ccctgttgagcttgactctagtttga

SEQ ID NO: 96 - DNA, 25 bp, synthetic construct
  source 1..25: mol_type = other DNA
ctgcgttatggtttaacagatgtgc

SEQ ID NO: 97 - DNA, 24 bp, synthetic construct
  source 1..24: mol_type = other DNA
gacgcctatctgattgayactcca

SEQ ID NO: 98 - DNA, 27 bp, synthetic construct
  source 1..27: mol_type = other DNA
catttgttaattcagatgcataygtgg

SEQ ID NO: 99 - DNA, 24 bp, synthetic construct
  source 1..24: mol_type = other DNA
gaggcttacctaattgacactcca

SEQ ID NO: 100 - DNA, 26 bp, synthetic construct
  source 1..26: mol_type = other DNA
ctgaagcttatctaattgacactcca

SEQ ID NO: 101 - DNA, 26 bp, synthetic construct
  source 1..26: mol_type = other DNA
ctgatgcctatataattgacactcca

SEQ ID NO: 102 - DNA, 27 bp, synthetic construct
  source 1..27: mol_type = other DNA
cattagttaattcagacgcatacgtgg

SEQ ID NO: 103 - DNA, 27 bp, synthetic construct
  source 1..27: mol_type = other DNA
gcaaatttagaagcttggccaaagtcy

SEQ ID NO: 104 - DNA, 26 bp, synthetic construct
  source 1..26: mol_type = other DNA
gcctttactttcatttagccctttaa

SEQ ID NO: 105 - DNA, 25 bp, synthetic construct
  source 1..25: mol_type = other DNA
aaatgttgaagcatggccacattcg

SEQ ID NO: 106 - DNA, 27 bp, synthetic construct
  source 1..27: mol_type = other DNA
gccttttgctttcattaagccctttta

SEQ ID NO: 107 - DNA, 23 bp, synthetic construct
  source 1..23: mol_type = other DNA
ggtgtttggtcgcatatcgcaac

SEQ ID NO: 108 - DNA, 21 bp, synthetic construct
  source 1..21: mol_type = other DNA
gcgatcgtcatgaaagtgcgt

SEQ ID NO: 109 - DNA, 22 bp, synthetic construct
  source 1..22: mol_type = other DNA
tcctataaagtgtccggcggtc

SEQ ID NO: 110 - DNA, 18 bp, synthetic construct
  source 1..18: mol_type = other DNA
tctcgccggtaaccgcca

SEQ ID NO: 111 - DNA, 18 bp, synthetic construct
  source 1..18: mol_type = other DNA
aaccaggcgattctgccg

SEQ ID NO: 112 - DNA, 19 bp, synthetic construct
  source 1..19: mol_type = other DNA
gcagcttgtccgggttgta

SEQ ID NO: 113 - DNA, 26 bp, synthetic construct
  source 1..26: mol_type = other DNA
gcaccatttagtgtgggaaattgtcg

SEQ ID NO: 114 - DNA, 20 bp, synthetic construct
  source 1..20: mol_type = other DNA
taacttcgacagctggacgt

SEQ ID NO: 115 - DNA, 22 bp, synthetic construct
  source 1..22: mol_type = other DNA
ggcggtggcggatacaaagtat

SEQ ID NO: 116 - DNA, 24 bp, synthetic construct
  source 1..24: mol_type = other DNA
acctgtcttatcagttgtgccaac

SEQ ID NO: 117 - DNA, 21 bp, synthetic construct
  source 1..21: mol_type = other DNA
tagaacaccgatggcgaaggc

SEQ ID NO: 118 - DNA, 22 bp, synthetic construct
  source 1..22: mol_type = other DNA
tcgtggactaccagggtatcta

SEQ ID NO: 119 - DNA, 23 bp, synthetic construct
  source 1..23: mol_type = other DNA
tttcgatgcaacgcgaagaacct

SEQ ID NO: 120 - DNA, 21 bp, synthetic construct
  source 1..21: mol_type = other DNA
tacgagctgacgacagccatg

SEQ ID NO: 121 - DNA, 18 bp, synthetic construct
  source 1..18: mol_type = other DNA
ctttggcggctccatcgg

SEQ ID NO: 122 - DNA, 21 bp, synthetic construct
  source 1..21: mol_type = other DNA
ctcctcagcgcggtaacttac

SEQ ID NO: 123 - DNA, 23 bp, synthetic construct
  source 1..23: mol_type = other DNA
cggtatgctcgttggcttagatg

SEQ ID NO: 124 - DNA, 21 bp, synthetic construct
  source 1..21: mol_type = other DNA
gtgattgcccatttggtgcag

SEQ ID NO: 125 - DNA, 26 bp, synthetic construct
  source 1..26: mol_type = other DNA
ttgtatggacaaatcgttgacataca

SEQ ID NO: 126 - DNA, 19 bp, synthetic construct
  source 1..19: mol_type = other DNA
gtagctgccaccggcctat

SEQ ID NO: 127 - DNA, 20 bp, synthetic construct
  source 1..20: mol_type = other DNA
aatggatggcgctcaagcgt

SEQ ID NO: 128 - DNA, 23 bp, synthetic construct
  source 1..23: mol_type = other DNA
actgccaccaagatctgcactag

SEQ ID NO: 129 - DNA, 48 bp, synthetic construct
  source 1..48: mol_type = other DNA
aaaacgagacatgccgagcatccgctttcgatgcaacgcgaagaacct

SEQ ID NO: 130 - DNA, 21 bp, synthetic construct
  source 1..21: mol_type = other DNA
  modified_base <1: mod_base = OTHER; note = Two Biotin groups on 5' end
tacgagctgacgacagccatg

SEQ ID NO: 131 - DNA, 22 bp, synthetic construct
  source 1..22: mol_type = other DNA
ggctgcgatattcaaagctcag

SEQ ID NO: 132 - DNA, 19 bp, synthetic construct
  source 1..19: mol_type = other DNA
ctgaacgcgccggcttaac

SEQ ID NO: 133 - DNA, 51 bp, synthetic construct
  source 1..51: mol_type = other DNA
aaaacgagacatgccgagcatccgcttgtatggacaaatcgttgacataca

SEQ ID NO: 134 - DNA, 47 bp, synthetic construct
  source 1..47: mol_type = other DNA
accgtgctgccttggcttcattgtggtcgtagctgccaccggcctat

SEQ ID NO: 135 - DNA, 90 bp, synthetic construct
  source 1..90: mol_type = other DNA
  modified_base 26^27: mod_base = OTHER; note = Hexaethylene Glycol internal spacer
cgcgcgaaaaaaaaaaaaaaaaaaaatttttttttttttttcgcgcgaaaaactcctctggcaccgtgctgccttggcttcattgtggtc

SEQ ID NO: 136 - DNA, 21 bp, synthetic construct
  source 1..21: mol_type = other DNA
  modified_base >21: mod_base = OTHER; note = Biotinylated 3' end
ttttttttttttttttttttt

SEQ ID NO: 137 - DNA, 22 bp, synthetic construct
  source 1..22: mol_type = other DNA
aaaacgagacatgccgagcatc

SEQ ID NO: 138 - DNA, 21 bp, synthetic construct
  source 1..21: mol_type = other DNA
tcgcgcgaaaaactcctctgg

SEQ ID NO: 139 - DNA, 28 bp, synthetic construct
  source 1..28: mol_type = other DNA
  modified_base <1: mod_base = OTHER; note = 6-Carboxyfluorescein on 5' end
  modified_base 9^10: mod_base = OTHER; note = IDT ZEN quencher
  modified_base >28: mod_base = OTHER; note = Iowa Black FQ quencher on 3' end
tgctgggatagctactcccgccttttgg

SEQ ID NO: 140 - DNA, 177 bp, synthetic construct
  source 1..177: mol_type = other DNA
aaaacgagacatgccgagcatccgcttgtatggacaaatcgttgacatacatcgttgcgaaaaatgctgggatagctactcccgccttttgggttattaataaagatgataggccggtggcagctacgaccacaatgaagccaaggcagcacggtgccagaggagtttttcgcgcga

SEQ ID NO: 141 - DNA, 24 bp, synthetic construct
  source 1..24: mol_type = other DNA
gtatcagcgcgaagtctttatacc

SEQ ID NO: 142 - DNA, 24 bp, synthetic construct
  source 1..24: mol_type = other DNA
ggcaataacatacggagtgacatc

SEQ ID NO: 143 - DNA, 169 bp, synthetic construct
  source 1..169: mol_type = other DNA
gtatcagcgcgaagtctttataccgaaaggttgggcgggccagcgtattgtactgcgtttcgatgcggtcactcattacggcaaagtgtgggtaaataatcaggaagtgatggagcatcagggcggctatacgccatttgaagccgatgtcactccgtatgttattgcc

SEQ ID NO: 144 - DNA, 128 bp, synthetic construct
  source 1..128: mol_type = other DNA
  modified_base 41^42: mod_base = OTHER; note = Hexaethylene Glycol internal spacer
aaaaaaaaaaaaaaaaaaaaaaaaaaaaaaaaaaacctctatttttttttttttttttttttttttttttttttttagacgcaagctactggtgatttggcaataacatacggagtgacatcggcttc

SEQ ID NO: 145 - DNA, 20 bp, synthetic construct
  source 1..20: mol_type = other DNA
  modified_base >20: mod_base = OTHER; note = Biotinylated 3' end
tttttttttttttttttttt

SEQ ID NO: 146 - DNA, 29 bp, synthetic construct
  source 1..29: mol_type = other DNA
  modified_base <1: mod_base = OTHER; note = 6-Carboxyfluorescein on 5' end
  modified_base >29: mod_base = OTHER; note = Iowa Black FQ quencher on 3' end
tagccgccctgatgctccatcacttcctg

SEQ ID NO: 147 - DNA, 20 bp, synthetic construct
  source 1..20: mol_type = other DNA
agacgcaagctactggtgat

SEQ ID NO: 148 - DNA, 22 bp, synthetic construct
  source 1..22: mol_type = other DNA
ggctgcgatattcaaagctcag

SEQ ID NO: 149 - DNA, 19 bp, synthetic construct
  source 1..19: mol_type = other DNA
ctgaacgcgccggcttaac

SEQ ID NO: 150 - DNA, 166 bp, synthetic construct
  source 1..166: mol_type = other DNA
ggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcag

SEQ ID NO: 151 - DNA, 77 bp, synthetic construct
  source 1..77: mol_type = other DNA
  modified_base 20^21: mod_base = OTHER; note = Hexaethylene Glycol internal spacer
aaaaaaaaaaaaaaaaaaaactcctctggcaccgtgctgccttggcttcattgtggtcctgaacgcgccggcttaac

SEQ ID NO: 152 - DNA, 25 bp, synthetic construct
  source 1..25: mol_type = other DNA
  modified_base 25: mod_base = OTHER; note = Inverted dT
cggtgccagaggagttttttttttt

SEQ ID NO: 153 - DNA, 26 bp, synthetic construct
  source 1..26: mol_type = other DNA
ttgtatggacaaatcgttgacataca

SEQ ID NO: 154 - DNA, 28 bp, synthetic construct
  source 1..28: mol_type = other DNA
  modified_base <1: mod_base = OTHER; note = 6-Carboxyfluorescein on 5' end
  modified_base >28: mod_base = OTHER; note = Iowa Black FQ quencher on 3' end
tgctgggatagctactcccgccttttgg

SEQ ID NO: 155 - DNA, 19 bp, synthetic construct
  source 1..19: mol_type = other DNA
accgtgctgccttggcttc

SEQ ID NO: 156 - DNA, 77 bp, synthetic construct
  source 1..77: mol_type = other DNA
  modified_base 20^21: mod_base = OTHER; note = Hexaethylene Glycol internal spacer
aaaaaaaaaaaaaaaaaaaactcctctggcaccgtgctgccttggcttcattgtggtcgtagctgccaccggcctat

SEQ ID NO: 157 - DNA, 47 bp, synthetic construct
  source 1..47: mol_type = other DNA
aaaacgagacatgccgagcatccgcggctgcgatattcaaagctcag

SEQ ID NO: 158 - DNA, 22 bp, synthetic construct
  source 1..22: mol_type = other DNA
aaaacgagacatgccgagcatc

SEQ ID NO: 159 - DNA, 25 bp, synthetic construct
  source 1..25: mol_type = other DNA
aaaacgagacatgccgagcatccgc

SEQ ID NO: 160 - DNA, 85 bp, synthetic construct
  source 1..85: mol_type = other DNA
  modified_base <1: mod_base = OTHER; note = C-12 amine on 5' end
  modified_base 40^41: mod_base = OTHER; note = Triacontaethylene glycol internal spacer
aaaaaaaaaaaaaaaaaaaaccccccccccccccccccccaaaaaaactcctctggcaccgtgctgccttggcttcattgtggtc

SEQ ID NO: 161 - DNA, 174 bp, synthetic construct
  source 1..174: mol_type = other DNA
aaaacgagacatgccgagcatccgctcagtaatgtttcgaaagggcaatacgcaaagaggtttttctatttcgctactagttgcttagtgttaactttagttgtagtttcaagtctaagtagctcagcagaccacaatgaagccaaggcagcacggtgccagaggagttttttt

SEQ ID NO: 162 - DNA, 182 bp, synthetic construct
  source 1..182: mol_type = other DNA
aaaacgagacatgccgagcatccgctaaactcgaacaggactttggcggctccatcggtgtgtacgcgatggataccggctcaggcgcaactgtaagttaccgcgctgaggagcgcttcccactgtgcagctcattcgaccacaatgaagccaaggcagcacggtgccagaggagttttttt

SEQ ID NO: 163 - DNA, 150 bp, synthetic construct
  source 1..150: mol_type = other DNA
aaaacgagacatgccgagcatccgctgcgggtattacttagacctgttctggtgcctgagcttgggctggtggtccttaagccgggccgtgaatccatacagatagaccacaatgaagccaaggcagcacggtgccagaggagttttttt

SEQ ID NO: 164 - DNA, 23 bp, synthetic construct
  source 1..23: mol_type = other DNA
  modified_base <1: mod_base = OTHER; note = Cy3 fluorophore on 5' end
tcgaaagggcaatacgcaaagag

SEQ ID NO: 165 - DNA, 23 bp, synthetic construct
  source 1..23: mol_type = other DNA
  modified_base <1: mod_base = OTHER; note = Alexa488 fluorophore on 5' end
taaactcgaacaggactttggcg

SEQ ID NO: 166 - DNA, 24 bp, synthetic construct
  source 1..24: mol_type = other DNA
  modified_base <1: mod_base = OTHER; note = Cy5 fluorophore on 5' end
tgcgggtattacttagacctgttc

SEQ ID NO: 167 - DNA, 36 bp, synthetic construct
  source 1..36: mol_type = other DNA
  modified_base <1: mod_base = OTHER; note = dibenzocyclooctyl group on 5' end
  modified_base 10^11: mod_base = OTHER; note = Hexaethylene Glycol internal spacer
  modified_base 33..36: mod_base = OTHER; note = Phosphorothioate bonds between residues
aaaaaaaaaagctcgaatcagtcctgtcagtctttt

SEQ ID NO: 168 - DNA, 33 bp, synthetic construct
  source 1..33: mol_type = other DNA
  modified_base <1: mod_base = OTHER; note = dibenzocyclooctyl group on 5' end
  modified_base 10^11: mod_base = OTHER; note = Hexaethylene Glycol internal spacer
  modified_base 29..33: mod_base = OTHER; note = Phosphorothioate bonds between residues
aaaaaaaaaatccgcgggagcttcaacatcgcg

SEQ ID NO: 169 - DNA, 146 bp, synthetic construct
  source 1..146: mol_type = other DNA
  modified_base <1: mod_base = OTHER; note = 5' end phosphorylation
gactgattcgagccgcgcttcaattccttgttcaacgattgctcgagaatcatactgataggctgttgctaaagcattttgcagctcttctcggttttccgcgggagcttcaacatcgcgcgaaaaaaaaaaaaaaagactgacag

SEQ ID NO: 170 - DNA, 142 bp, synthetic construct
  source 1..142: mol_type = other DNA
  modified_base <1: mod_base = OTHER; note = 5' end phosphorylation
gactgattcgagctgcgggtattacttagacctgttctggtgcctgagcttgggctggtggtccttaagccgggccgtgaatccatacagatatccgcgggagcttcaacatcgcgcgaaaaaaaaaaaaaaagactgacag

SEQ ID NO: 171 - DNA, 21 bp, synthetic construct
  source 1..21: mol_type = other DNA
  modified_base <1: mod_base = OTHER; note = Cy5 fluorophore on 5' end
caacgattgctcgagaatcat

SEQ ID NO: 172 - DNA, 24 bp, synthetic construct
  source 1..24: mol_type = other DNA
  modified_base <1: mod_base = OTHER; note = Cy3 fluorophore on 5' end
tgcgggtattacttagacctgttc

SEQ ID NO: 173 - DNA, 73 bp, synthetic construct
  source 1..73: mol_type = other DNA
  modified_base <1: mod_base = OTHER; note = dibenzocyclooctyl group on 5' end
tgtagcacgattgcagcattgttagcaggattgcgggtgccaatgtgatcaacgtacagaggatctagtcggc

SEQ ID NO: 174 - DNA, 17 bp, synthetic construct
  source 1..17: mol_type = other DNA
gccgactagatcctctg

SEQ ID NO: 175 - DNA, 112 bp, synthetic construct
  source 1..112: mol_type = other DNA
tttaagtcccgcaacgagcgcaacccttatcctttgttgccagcggtccggccgggaactcaaaggagactgccagtgataaactggaggaaggtggggatgacgtcaacgg

SEQ ID NO: 176 - DNA, 21 bp, synthetic construct
  source 1..21: mol_type = other DNA
  modified_base <1: mod_base = OTHER; note = dibenzocyclooctyl group on 5' end
ccgttgacgtcatccccacct

SEQ ID NO: 177 - DNA, 150 bp, synthetic construct; identical to SEQ ID NO: 163
  source 1..150: mol_type = other DNA

SEQ ID NO: 178 - DNA, 43 bp, synthetic construct
  source 1..43: mol_type = other DNA
  modified_base <1: mod_base = OTHER; note = dibenzocyclooctyl group on 5' end
aaaaactcctctggcaccgtgctgccttggcttcattgtggtc

SEQ ID NO: 179 - DNA, 25 bp, synthetic construct
  source 1..25: mol_type = other DNA
aaaacgagacatgccgagcatccgc

SEQ ID NO: 180 - DNA, 23 bp, synthetic construct
  source 1..23: mol_type = other DNA
ggaaggcacgctctactattcaa

SEQ ID NO: 181 - DNA, 29 bp, synthetic construct
  source 1..29: mol_type = other DNA
  modified_base <1: mod_base = OTHER; note = Biotinylated 5' end
  modified_base 29: mod_base = OTHER; note = Inverted dT
aaaaatttcgatgcaacgcgaagaacctt

SEQ ID NO: 182 - DNA, 27 bp, synthetic construct
  source 1..27: mol_type = other DNA
  modified_base <1: mod_base = OTHER; note = Biotinylated 5' end
  modified_base 27: mod_base = OTHER; note = Inverted dT
aaaaatacgagctgacgacagccatgt

SEQ ID NO: 183 - DNA, 29 bp, synthetic construct
  source 1..29: mol_type = other DNA
  modified_base <1: mod_base = OTHER; note = Biotinylated 5' end
  modified_base 29: mod_base = OTHER; note = Inverted dT
aaaaatttaagtcccgcaacgagcgcaat

SEQ ID NO: 184 - DNA, 29 bp, synthetic construct
  source 1..29: mol_type = other DNA
  modified_base <1: mod_base = OTHER; note = Biotinylated 5' end
  modified_base 29: mod_base = OTHER; note = Inverted dT
aaaaattgacgtcatccccaccttcctct

SEQ ID NO: 185 - DNA, 26 bp, synthetic construct
  source 1..26: mol_type = other DNA
  modified_base <1: mod_base = OTHER; note = Biotinylated 5' end
  modified_base 26: mod_base = OTHER; note = Inverted dT
aaaaattactcacccgtrcgccrctt

SEQ ID NO: 186 - DNA, 32 bp, synthetic construct
  source 1..32: mol_type = other DNA
  modified_base <1: mod_base = OTHER; note = Biotinylated 5' end
  modified_base 32: mod_base = OTHER; note = Inverted dT
aaaaatgagayacgggccaractcctacgggt

SEQ ID NO: 187 - DNA, 29 bp, synthetic construct
  source 1..29: mol_type = other DNA
  modified_base <1: mod_base = OTHER; note = Biotinylated 5' end
  modified_base 29: mod_base = OTHER; note = Inverted dT
aaaaatcrttacrccwttcgtgcaggtct

SEQ ID NO: 188 - DNA, 32 bp, synthetic construct
  source 1..32: mol_type = other DNA
  modified_base <1: mod_base = OTHER; note = Biotinylated 5' end
  modified_base 32: mod_base = OTHER; note = Inverted dT
aaaaacgctaccttaggaysgttatagttact

SEQ ID NO: 189 - DNA, 27 bp, synthetic construct
  source 1..27: mol_type = other DNA
  modified_base <1: mod_base = OTHER; note = Biotinylated 5' end
  modified_base 27: mod_base = OTHER; note = Inverted dT
aaaaatgtgtaggataggtgggaggct

SEQ ID NO: 190 - DNA, 31 bp, synthetic construct
  source 1..31: mol_type = other DNA
  modified_base <1: mod_base = OTHER; note = Biotinylated 5' end
  modified_base 31: mod_base = OTHER; note = Inverted dT
aaaaacggaaagaccccatggagctttactt

SEQ ID NO: 191 - DNA, 31 bp, synthetic construct
  source 1..31: mol_type = other DNA
  modified_base <1: mod_base = OTHER; note = Biotinylated 5' end
  modified_base 31: mod_base = OTHER; note = Inverted dT
aaaaacggaaagaccccgtgaacctttactt

SEQ ID NO: 192 - DNA, 31 bp, synthetic construct
  source 1..31: mol_type = other DNA
  modified_base <1: mod_base = OTHER; note = Biotinylated 5' end
  modified_base 31: mod_base = OTHER; note = Inverted dT
aaaaacggaaagaccccgtggagctttactt

SEQ ID NO: 193 - DNA, 25 bp, synthetic construct
  source 1..25: mol_type = other DNA
  modified_base <1: mod_base = OTHER; note = Biotinylated 5' end
  modified_base 25: mod_base = OTHER; note = Biotinylated 5' end
aaaaacaccgcgctgaccaacctct

SEQ ID NO: 194 - DNA, 26 bp, synthetic construct
  source 1..26: mol_type = other DNA
  modified_base <1: mod_base = OTHER; note = Biotinylated 5' end
  modified_base 26: mod_base = OTHER; note = Inverted dT
aaaaacacagcggcagcaagaaagct

SEQ ID NO: 195 - DNA, 34 bp, synthetic construct; identical to SEQ ID NO: 8
  source 1..34: mol_type = other DNA
  modified_base <1: mod_base = OTHER; note = Biotinylated 5' end
  modified_base 34: mod_base = OTHER; note = Inverted dT

SEQ ID NO: 196 - DNA, 36 bp, synthetic construct
  source 1..36: mol_type = other DNA
  modified_base <1: mod_base = OTHER; note = Biotinylated 5' end
  modified_base 36: mod_base = OTHER; note = Inverted dT
aaaaattgagttgaacctggtgaagttgtaatctgg

SEQ ID NO: 197 - DNA, 30 bp, synthetic construct
  source 1..30: mol_type = other DNA
  modified_base <1: mod_base = OTHER; note = Biotinylated 5' end
  modified_base 30: mod_base = OTHER; note = Inverted dT
aaaaaacaaggtctgtttgaattgtccggt

SEQ ID NO: 198 - DNA, 28 bp, synthetic construct
  source 1..28: mol_type = other DNA
  modified_base <1: mod_base = OTHER; note = Biotinylated 5' end
  modified_base 28: mod_base = OTHER; note = Inverted dT
aaaaattgtcttgccgattcaattgcgt

SEQ ID NO: 199 - DNA, 33 bp, synthetic construct
  source 1..33: mol_type = other DNA
  modified_base <1: mod_base = OTHER; note = Biotinylated 5' end
  modified_base 33: mod_base = OTHER; note = Inverted dT
aaaaaccgtcacgctgttgttaggaagtgtgct

SEQ ID NO: 200 - DNA, 32 bp, synthetic construct
  source 1..32: mol_type = other DNA
  modified_base <1: mod_base = OTHER; note = Biotinylated 5' end
  modified_base 32: mod_base = OTHER; note = Inverted dT
aaaaagccatcactttactggtgctgcacatt

SEQ ID NO: 201 - DNA, 32 bp, synthetic construct
  source 1..32: mol_type = other DNA
  modified_base <1: mod_base = OTHER; note = Biotinylated 5' end
  modified_base 32: mod_base = OTHER; note = Inverted dT
aaaaagccataactttactggtactgcacatt

SEQ ID NO: 202 - DNA, 51 bp, synthetic construct; identical to SEQ ID NO: 133
  source 1..51: mol_type = other DNA

SEQ ID NO: 203 - DNA, 47 bp, synthetic construct; identical to SEQ ID NO: 134
  source 1..47: mol_type = other DNA

SEQ ID NO: 204 - DNA, 19 bp, synthetic construct
  source 1..19: mol_type = other DNA
gtagctgccaccggcctat

SEQ ID NO: 205 - DNA, 157 bp, synthetic construct
  source 1..157: mol_type = other DNA
ctacgcctccgctttcctctctatgggcagtcggtgatgaatctccattttagcacttacctgtgactccatagaaaatctttctcctgctcagtgatttcacagagaggatctcgtgtactgcgattagaacgtacctgtcgtcagctcgtacggc

SEQ ID NO: 206 - DNA, 155 bp, synthetic construct
  source 1..155: mol_type = other DNA
ctacgcctccgctttcctctctatgggcagtcggtgatgaatctccattttagcacttacctgtgactccatagaaaatctttctcctgctcagtgatttcagagagaggatctcgtgtagcaactagcgaacgtactgtgttaggcatgcccgg

SEQ ID NO: 207 - DNA, 20 bp, synthetic construct
  source 1..20: mol_type = other DNA
  modified_base <1: mod_base = OTHER; note = dibenzocyclooctyl group on 5' end
gccgtacgagctgacgacag

SEQ ID NO: 208 - DNA, 18 bp, synthetic construct
  source 1..18: mol_type = other DNA
  modified_base <1: mod_base = OTHER; note = dibenzocyclooctyl group on 5' end
ccgggcatgcctaacaca

SEQ ID NO: 209 - DNA, 58 bp, synthetic construct
  source 1..58: mol_type = other DNA
cacagtacgttcgctagttgctacacgagatcctctctctgaaatcactgagcaggag

SEQ ID NO: 210 - DNA, 59 bp, synthetic construct
  source 1..59: mol_type = other DNA
gacaggtacgttctaatcgcagtacacgagatcctctctgtgaaatcactgagcaggag

SEQ ID NO: 211 - DNA, 55 bp, synthetic construct
  source 1..55: mol_type = other DNA
tctatgggcagtcggtgatgaatctccattttagcacttacctgtgactccatag

SEQ ID NO: 212 - DNA, 35 bp, synthetic construct
  source 1..35: mol_type = other DNA
  modified_base <1: mod_base = OTHER; note = Two Biotin groups on 5' end
ccgggcatgcctaacacagtacgttcgctagttgc

SEQ ID NO: 213 - DNA, 37 bp, synthetic construct
  source 1..37: mol_type = other DNA
  modified_base <1: mod_base = OTHER; note = Two Biotin groups on 5' end
gccgtacgagctgacgacaggtacgttctaatcgcag

SEQ ID NO: 214 - DNA, 38 bp, synthetic construct
  source 1..38: mol_type = other DNA
ctacgcctccgctttcctctctatgggcagtcggtgat

SEQ ID NO: 215 - DNA, 42 bp, synthetic construct
  source 1..42: mol_type = other DNA
  modified_base <1: mod_base = OTHER; note = Biotinylated 5' end
  modified_base 42: mod_base = OTHER; note = Inverted dT
aaaaaacctcgtcgcggaaccattcgctaaactcgaacaggt

SEQ ID NO: 216 - DNA, 42 bp, synthetic construct
  source 1..42: mol_type = other DNA
  modified_base <1: mod_base = OTHER; note = Biotinylated 5' end
  modified_base 42: mod_base = OTHER; note = Inverted dT
aaaaacagcacagcggcagcaagaaagcccttgaatgagctt

SEQ ID NO: 217 - DNA, 34 bp, synthetic construct; identical to SEQ ID NO: 8
  source 1..34: mol_type = other DNA
  modified_base <1: mod_base = OTHER; note = Biotinylated 5' end
  modified_base 34: mod_base = OTHER; note = Inverted dT

SEQ ID NO: 218 - DNA, 37 bp, synthetic construct; identical to SEQ ID NO: 9
  source 1..37: mol_type = other DNA
  modified_base <1: mod_base = OTHER; note = Biotinylated 5' end
  modified_base 37: mod_base = OTHER; note = Inverted dT